(19) United States
(12) Patent Application Publication
(10) Pub. No.: US 2026/0274883 A1
(43) Pub. Date: Sep. 17, 2026

(54) LIPID MONOMERS FOR THERAPEUTIC DELIVERY OF RNA

(71) Applicant: Janssen Biotech, Inc., Horsham, PA (US)

(72) Inventors: Marija PRHAVC, Spring House, PA (US); Yi JIN, Spring House, PA (US); Mahesh RAMASESHAN, Spring House, PA (US)

(21) Appl. No.: 18/847,068

(22) PCT Filed: Mar. 15, 2023
(86) PCT No.: PCT/IB2023/052532

§ 371 Date: Sep. 13, 2024

Related U.S. Application Data
(60) Provisional application No. 63/320,689, filed on Mar. 16, 2022.

(51) Int. Cl. C07H 19/10; C07D 207/46; C07D 409/12; C07D 413/12; C07D 513/08; C07H 15/04; C12N 15/113

(52) U.S. Cl. CPC C07H 19/10; C07D 207/46; C07D 409/12; C07D 413/12; C07D 513/08; C07H 15/04; C12N 15/113; C12N 2310/14; C12N 2310/3515

(57) ABSTRACT

Provided herein are compounds that contain one or more lipophilic moieties and can be used to prepare one or more lipophilic monomers. Also provided herein are lipophilic monomers that can be conjugated to one or more positions on at least one strand of an oligonucleotide, and oligonucleotides comprising at least one said lipophilic monomer.

### BACKGROUND

Efficient delivery of therapeutic RNA beyond the liver is the fundamental obstacle preventing its clinical utility. Conjugate-mediated delivery is emerging as the clinically dominant delivery paradigm for siRNAs. Lipids are a major class of conjugates widely used for improving siRNA delivery as lipid conjugation increases plasma half-life and enhances tissue accumulation and cellular uptake of siRNAs. siRNA is highly hydrophilic that possesses poor pharmacological properties. Lipid conjugation modulates the hydrophobicity of siRNA that governs the pharmacokinetic behavior and tissue biodistribution by driving selective, in-situ incorporation into endogenous lipoprotein pathways. Though siRNA's have been conjugated to fatty acids, cholesterol, tocopherol but still there is an ongoing need for development of efficient and robust lipid delivery systems that can balance the safety and efficacy towards a better therapeutic margin. The present application relates to siRNA conjugated to variety of lipid class for delivering the siRNA to other tissues beyond the liver.

### SUMMARY

In one embodiment, provided herein are compounds that contain one or more lipophilic moieties and can be used to prepare one or more lipophilic monomers. In one embodiment, provided herein are lipophilic monomers that can be conjugated to one or more positions on at least one strand of an oligonucleotide, optionally via a linker or carrier.

In one embodiment, provided herein is a compound of Formula (I):

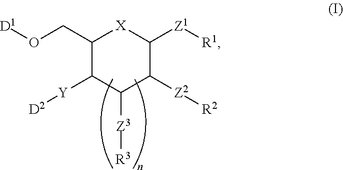

or a stereoisomer thereof, or a pharmaceutically acceptable salt thereof, wherein X, Y, D¹, D², Z¹, Z², Z³, R¹, R², R³, and n are as defined herein or elsewhere.

In one embodiment, provided herein is a compound of Formula (III):

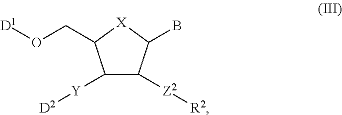

or a stereoisomer thereof, or a pharmaceutically acceptable salt thereof, wherein X, Y, D¹, D², Z², R², and B are as defined herein or elsewhere.

In one embodiment, provided herein is a compound of Formula (IV):

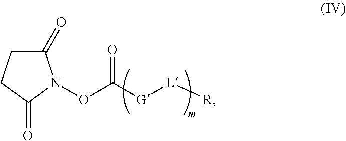

or a stereoisomer thereof, or a pharmaceutically acceptable salt thereof, wherein G′, L′, R, and m are as defined herein or elsewhere.

In one embodiment, provided herein is an oligonucleotide comprising at least one lipophilic monomer of the following formula:

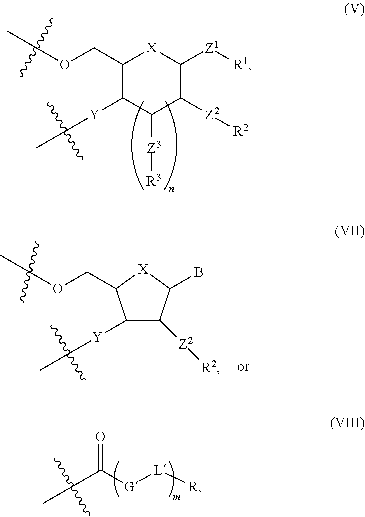

wherein X, Y, Z¹, Z², Z³, R¹, R², R³, B, G′, L′, R, n, and m are as defined herein or elsewhere.

Also provided herein are methods of reducing the expression of a target gene in a cell or a subject, using the oligonucleotides provided herein.

### DETAILED DESCRIPTION

### Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein, and in the specification and the accompanying claims, the indefinite articles “a” and “an” and the definite article “the” include plural as well as single referents, unless the context clearly indicates otherwise.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, the phrase “and/or” as used in a phrase such as “A and/or B” herein is intended to include both A and B; A or B; A (alone); and B (alone). Likewise, the phrase “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, and unless otherwise specified, the term “polynucleotide” or “nucleic acid,” as used interchangeably herein, refers to polymers of nucleotides of any length and includes, e.g., DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. Nucleic acid can be in either single- or double-stranded forms. As used herein and unless otherwise specified, “nucleic acid” also includes nucleic acid mimics such as locked nucleic acids (LNAs), peptide nucleic acids (PNAs), and morpholinos. As used herein, and unless otherwise specified, the term “oligonucleotide” refers to short synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

As used herein, the term “double stranded RNA agent” or “dsRNA agent” means an agent containing an RNA or RNA-like (e.g., chemically modified RNA) oligonucleotide molecule that is capable of inhibiting a gene expression in a sequence specific manner. The sense strand and/or antisense strand of a dsRNA agent may comprise another moiety (e.g., a lipid moiety). For example, a lipid moiety may be incorporated into the sense strand of a dsRNA agent. The sense strand and/or antisense strand of a dsRNA agent may be linked or conjugated, directly or indirectly, to another moiety (e.g., a lipid moiety). For example, the sense strand of the dsRNA agent may be linked or conjugated, directly or indirectly, to another moiety (e.g., a lipid moiety). In a specific embodiment, a dsRNA agent is a double-stranded RNA or RNA-like (e.g., chemically modified RNA) oligonucleotide comprising a sense stand and an antisense strand forming a double-stranded region. The double-stranded region may be the entire length of the sense strand, antisense strand, or both. Alternatively, the double-stranded region may be less than the entire length of the sense strand, antisense strand, or both. The double-stranded region may be the result of the antisense strand being fully complementary, partially complementary, or substantially complementary to the sense strand. In a specific embodiment, the antisense strand of a dsRNA agent is partially complementary to a target RNA transcript. In another specific embodiment, the antisense strand of a dsRNA agent is substantially complementary to a target RNA transcript. In another specific embodiment, the antisense strand of a dsRNA agent is fully complementary to a target RNA transcript.

The two strands forming the double-stranded region or duplex structure may be different portions of one larger RNA molecule, or they may be separate RNA molecules. Where the two strands are part of one larger RNA molecule, and therefore are connected by a contiguous chain of nucleotides between the 3′-end of one strand and the 5′-end of the respective other strand forming the double-stranded region or duplex structure, the connecting RNA chain is referred to as a “hairpin loop.” A hairpin loop can comprise at least one unpaired nucleotide. In some embodiments, the hairpin loop can comprise at least 4, at least 5, at least 6, at least 7, at least 8, at least 9, at least 10, at least 20, at least 23 or more unpaired nucleotides or nucleotides not directed to the target site of the dsRNA agent. In some embodiments, the hairpin loop can be 1 to 10 unpaired nucleotides. In some embodiments, the hairpin loop can be 1 to 8 unpaired nucleotides. In some embodiments, the hairpin loop can be 4 to 10 unpaired nucleotides. In some embodiments, the hairpin loop can be 4 to 8 unpaired nucleotides.

Where the two substantially complementary strands of a dsRNA agent are comprised by separate RNA molecules, those molecules need not, but can be covalently connected. In certain embodiments where the two strands are connected covalently by means other than a contiguous chain of nucleotides between the 3′-end of one strand and the 5′-end of the respective other strand forming the duplex structure, the connecting structure is referred to as a “linker”. The RNA strands of a dsRNA agent may have the same or a different number of nucleotides.

In some embodiments, one or both strands of a dsRNA agent comprise an overhang. In other embodiments, the dsRNA agent is blunt ended.

In a specific embodiment, a dsRNA agent described herein mediates messenger RNA (mRNA) degradation or inhibition of translation of the mRNA in a sequence-specific manner. In specific embodiments, a dsRNA agent described herein inhibits gene expression via an RNA-induced silencing complex (RISC) pathway. In some embodiments, without being bound by theory, a dsRNA agent described herein functions like short interfering RNA (siRNA). In a specific embodiment, a dsRNA agent described herein is a siRNA.

As used herein, term “complementary,” when used to describe a first nucleotide sequence (e.g., a sense strand of a dsRNA agent, or targeted sequence) in relation to a second nucleotide sequence (e.g., antisense strand of a dsRNA agent, or a single-stranded antisense oligonucleotide), means the ability of an oligonucleotide or polynucleotide including the first nucleotide sequence to hybridize (form base pair hydrogen bonds under mammalian physiological conditions (or similar conditions in vitro)) and form a duplex or double helical structure under certain conditions with an oligonucleotide or polynucleotide including the second nucleotide sequence. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, fA and mA are complementary to U (or T) and identical to A for the purposes of determining identity or complementarity.

As used herein, the term “fully complementary” in the context of two nucleotide sequences means that all (100%) of the bases in a contiguous sequence of a first nucleotide sequence will hybridize to the same number of bases in a contiguous sequence of a second nucleotide sequence to form a duplex. Where two nucleotide sequences are designed to form, upon hybridization, one or more single stranded overhangs, such overhangs shall not be regarded as mismatches with regard to the determination of complementarity. For example, a dsRNA agent comprising one oligonucleotide 21 nucleotides in length and another oligonucleotide 23 nucleotides in length, wherein the 23 nucleotides oligonucleotide comprises a sequence of 21 nucleotides that is fully complementary to the 21 nucleotides oligonucleotide, are considered “fully complementary” for the purposes described herein. In a specific embodiment, two nucleotide sequences are “fully complementary” when all (100%) of the bases of a first nucleotide sequence hybridize to all (100%) of the bases in a second nucleotide sequence to form a duplex. In one specific embodiment, the two nucleotide sequences hybridize under stringent conditions. In another specific embodiment, the two nucleotide sequences hybridize under very stringent conditions.

As used herein, the term “partially complementary” in the context of two nucleotide sequences means that at least 65% but less than 80% of the bases in a contiguous sequence of a first nucleotide sequence will hybridize to the same number of bases in a contiguous sequence of a second nucleotide sequence to form a duplex. In one specific embodiment, the two nucleotide sequences hybridize under stringent conditions. In another specific embodiment, the two nucleotide sequences hybridize under very stringent conditions.

As used herein, the term “substantially complementary” in the context of two nucleotide sequences means that at least 80% but less than 100% of the bases in a contiguous sequence of a first nucleotide sequence will hybridize to the same number of bases in a contiguous sequence of a second nucleotide sequence to form a duplex. In some embodiments, two nucleotide sequences are substantially complementary when at least 85%, at least 90%, at least 91%, at least 92%, at least 93%, at least 94%, at least 95%, at least 96%, at least 97%, at least 98%, but less than 100% of the bases in a contiguous sequence of a first oligonucleotide will hybridize to the same number of bases in a contiguous sequence of a second oligonucleotide to form a duplex. In one specific embodiment, the two nucleotide sequences hybridize under stringent conditions. In another specific embodiment, the two nucleotide sequences hybridize under very stringent conditions.

As used herein, the terms “about” and “approximate” when referring to a numerical value encompass the recited numerical value and variations within +/−20%. For example, about 20% would encompass 16% to 24% and values in between, including 20%. In one embodiment, the terms “about” and “approximate” when referring to a numerical value encompass the recited numerical value and variations within +/−15%. In another embodiment, the terms “about” and “approximate” when referring to a numerical value encompass the recited numerical value and variations within +/−10%. In another embodiment, the terms “about” and “approximate” when referring to a numerical value encompass the recited numerical value and variations within +/−5%.

As used herein, the term “stringent” when referring to hybridization means that under “stringent conditions”, or “stringent hybridization conditions”, a first nucleotide sequence will hybridize to a second nucleotide, with minimal hybridization to other sequences. In a specific embodiment, an antisense sequence will hybridize under stringent conditions to its target sequence, with minimal targeting to other sequences. Stringent conditions are sequence dependent (e.g., sequence length, complementarity), and vary under different environmental parameters (e.g., assay conditions, physiological environment). An example of stringent hybridization conditions may include: 400 mM NaCl, 40 mM PIPES pH 6.4, 1 mM EDTA, 50° C. or 70° C. for 12-16 hours followed by washing. A skilled person will understand that variations in the stringency of hybridization are inherently described.

As used herein, the term “very stringent” when referring to hybridization means that under “very stringent conditions” or “very stringent hybridization conditions”, a first nucleotide sequence will only be observed to hybridize to a second nucleotide. In a specific embodiment, an antisense sequence will be observed to only hybridize to its target sequence under very stringent conditions. Also, very stringent conditions may not allow hybridization to occur between partially complementary sequences. Very stringent conditions are sequence dependent (e.g., sequence length, complementarity), and vary under different environmental parameters (e.g., assay conditions, physiological environment). Very stringent conditions may include a higher temperature, lower ionic strength, and/or shorter reaction time compared to stringent conditions under the same circumstance. For example, very stringent conditions may include a hybridization temperature of about 71° C., about 72° C., about 73° C., about 74° C., about 75° C., about 76° C., about 77° C., about 78° C., about 79° C., about 80° C., or higher. A skilled person will understand that variations in the stringency of hybridization are inherently described.

As used herein, “target sequence” refers to a contiguous portion of a nucleotide sequence of a RNA molecule formed during the transcription of a gene, including mRNA that is a product of RNA processing of a primary transcription product (e.g., mRNA resulting from alternate splicing). In one embodiment, the contiguous portion of the nucleotide sequence is at least long enough to serve as a substrate for RNAi-directed cleavage at or near that portion of the nucleotide sequence of an mRNA molecule formed during the transcription of a gene. In specific embodiments, the target sequence is about 15-30 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. In certain embodiments, the target sequence is 19-25 nucleotides in length. In some embodiments, the target sequence is 21-23 nucleotides in length. Ranges and lengths intermediate to the above recited ranges and lengths are also contemplated to be part of the disclosure.

As used herein, the phrase “nucleotide sequence corresponding to any one of the antisense strand nucleotide sequences”, “nucleotide sequence corresponding to the antisense strand nucleotide sequence”, “nucleotide sequence corresponding to any one of the sense strand nucleotide sequences”, “nucleotide sequence corresponding to the sense strand nucleotide sequence”, or “nucleotide sequence corresponding to any one of the nucleotide sequences” refers to an oligonucleotide comprising a chain of nucleotides comprising the recited unmodified nucleotides, or one or more modified nucleotides, or one or more conjugated moieties (e.g., a moiety described herein, such as, e.g., a lipid, or a modified nucleotide conjugated to a moiety described herein). A skilled person is aware that the recited unmodified nucleotide may be replaced by other moieties without substantially altering the base pairing properties of an oligonucleotide comprising a nucleotide bearing such replacement moiety. For example, a nucleotide comprising inosine as its base can base pair with nucleotides containing adenine, cytosine, or uracil. Thus, a nucleotide containing uracil, guanine, or adenine can be replaced by a nucleotide containing, for example, inosine. In another example, adenine and cytosine may be replaced with guanine and uracil, respectively to form G-U Wobble base pairing with the target mRNA.

As used herein, the phrase “non-naturally occurring double stranded ribonucleic acid (dsRNA) agent”, “non-naturally occurring double stranded ribonucleic acid agent”, or “non-naturally occurring dsRNA agent” refers to a dsRNA agent that is not found in nature. The non-naturally occurring dsRNA may contain one or more modified nucleotides.

As used herein, the term “overhang” in the context of a 5′ or 3′ nucleotide overhang refers to at least one unpaired nucleotide that protrudes from the duplex structure of a dsRNA agent. For example, when a 3′-end of one strand of a dsRNA agent extends beyond the 5′-end of the other strand, or vice versa, there is a nucleotide overhang. In some embodiments, the overhang is present at the 3′-end of the sense strand, antisense strand, or both strands. In one embodiment, the 3′-overhang is present in the antisense strand. In another embodiment, the 3′-overhang is present in the sense strand. In some embodiments, the overhang is present at the 5′-end of the sense strand, antisense strand, or both strands. In one embodiment, the 5′-overhang is present in the antisense strand. In another embodiment, the 5′-overhang is present in the sense strand. The overhangs may be due to one strand being longer than the other, or the result of two strands of the same length being staggered. In some embodiments, the overhang forms a mismatch with the target sequence. In other embodiments, the overhang is complementary to the gene sequences being targeted. The nucleotides in the overhang region of a dsRNA agent may each independently be a modified or unmodified nucleotide (e.g., 2′-fluoro-modified nucleotide, 2′-O-methyl modified nucleotide, deoxynucleotide, or a combination thereof).

In some embodiments, the 5′- or 3′-overhangs of the sense strand or antisense strand of a dsRNA agent are phosphorylated. In certain embodiments, the 5′- or 3′-overhangs of the sense strand and the antisense strand of a dsRNA agent are phosphorylated. In some embodiments, the overhang region(s) contains two (or more) nucleotides having a phosphorothioate between the two (or more) nucleotides, and those two (or more) nucleotides can be the same or different.

In some embodiments, a dsRNA agent contains only a single overhang, which can strengthen the interference activity of the dsRNA agent, without affecting its overall stability. For example, the single-stranded overhang may be located at the 3-terminal end of the sense strand of the dsRNA agent, or, alternatively, at the 3-terminal end of the antisense strand of the dsRNA agent. The dsRNA agent may also have a blunt end, located at the 5′-end of the antisense strand (or the 3′-end of the sense strand) or vice versa. In some embodiments, the antisense strand of the dsRNA agent has a nucleotide overhang at the 3′-end, and the 5′-end is blunt ended.

In some embodiments, one strand of a dsRNA agent comprises a 5′-end, a 3′-end, or both a 5′-end and a 3′-end overhang of at least 1 nucleotide, at least 2 nucleotides, or at least 3 nucleotides. In certain embodiments, one strand of a dsRNA agent comprises a 5′-end, a 3′-end, or both a 5′-end and a 3′-end overhang of at least 1 nucleotide, at least 2 nucleotides, or at least 3 nucleotides, but no more than 5 nucleotides. In some embodiments, one strand of a dsRNA agent comprises a 5′-end, a 3′-end, or both a 5′-end and a 3′-end overhang of 1 nucleotide, 2 nucleotides, or 3 nucleotides. In certain embodiments, one strand of a dsRNA agent comprises a 5′-end, a 3′-end, or both a 5′-end and a 3′-end overhang of 1 to 2 nucleotides, 1 to 3 nucleotides, 1 to 4 nucleotides, or 1 to 5 nucleotides. In some embodiments, one strand of a dsRNA agent comprises a 5′-end, a 3′-end, or both a 5′-end and a 3′-end overhang of 2 to 3 nucleotides, 2 to 4 nucleotides, or 2 to 5 nucleotides. In certain embodiments, one strand of a dsRNA agent comprises a 5′-end, a 3′-end, or both a 5′-end and a 3′-end overhang of 3 to 4 nucleotides, or 4 to 5 nucleotides. The strand may be an antisense strand or a sense strand. A nucleotide overhang may comprise or consist of a nucleotide analog or a nucleoside analog.

In some embodiments, each strand of a dsRNA agent comprises a 5′-end, a 3′-end, or both a 5′-end and a 3′-end overhang of at least 1 nucleotide, at least 2 nucleotides, or at least 3 nucleotides. In certain embodiments, each strand of a dsRNA agent comprises a 5′-end, a 3′-end, or both a 5′-end and a 3′-end overhang of at least 1 nucleotide, at least 2 nucleotides, or at least 3 nucleotides, but no more than 5 nucleotides. In some embodiments, each strand of a dsRNA agent comprises a 5′-end, a 3′-end, or both a 5′-end and a 3′-end overhang of 1 nucleotide, 2 nucleotides, or 3 nucleotides. In certain embodiments, each strand of a dsRNA agent comprises a 5′-end, a 3′-end, or both a 5′-end and a 3′-end overhang of 1 to 2 nucleotides, 1 to 3 nucleotides, 1 to 4 nucleotides, or 1 to 5 nucleotides. In some embodiments, each strand of a dsRNA agent comprises a 5′-end, a 3′-end, or both a 5′-end and a 3′-end overhang of 2 to 3 nucleotides, 2 to 4 nucleotides, or 2 to 5 nucleotides. In certain embodiments, each strand of a dsRNA agent comprises a 5′-end, a 3′-end, or both a 5′-end and a 3′-end overhang of 3 to 4 nucleotides, or 4 to 5 nucleotides. A nucleotide overhang may comprise or consist of a nucleotide analog or a nucleoside analog.

As used herein, the term “blunt” or “blunt ended” in the context of a dsRNA agent means that there are no unpaired nucleotides or nucleotide analogs at a given terminal end of a dsRNA, i.e., no nucleotide overhang. In some embodiments, one end of a dsRNA agent is blunt ended. In other words, the 5′-end of one strand and the 3′-end of the other strand do not include an unpaired nucleotide or nucleotide analog. In some embodiments, both ends of a dsRNA agent are blunt ended. In other words, there is no nucleotide overhang at either end of the dsRNA agent.

As used herein, term “antisense strand” or “guide strand” in the context of a dsRNA agent refers to the strand which includes a region that is complementary to a target sequence.

As used herein, the term “sense strand” or “passenger strand” in the context of a dsRNA agent refers to the strand of a dsRNA agent that includes a region that is complementary to a region of the antisense strand.

As used herein, the term “modified” in the context of a nucleobase of a dsRNA agent, refers to a nucleobase not found in nature in a RNA molecule. Naturally occurring RNA sequences include purine bases adenine (A) and guanine (G), and pyrimidine bases cytosine (C) and uracil (U).

As used herein, the term “modified” in the context of a nucleotide of a RNA sequence, such as a dsRNA agent, refers to a nucleotide not found in nature in a RNA molecule.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

In one embodiment, each component is “pharmaceutically acceptable” in the sense of being compatible with the other ingredients of a pharmaceutical composition, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

As used herein, and unless otherwise specified, the term “pharmaceutically acceptable salts” refers to salts prepared from pharmaceutically acceptable, relatively non-toxic acids, including inorganic acids and organic acids. In certain embodiments, suitable acids include, but are not limited to, acetic, benzenesulfonic, benzoic, camphorsulfonic, carbonic, citric, dihydrogenphosphoric, ethenesulfonic, fumaric, galactunoric, gluconic, glucuronic, glutamic, hydrobromic, hydrochloric, hydriodic, isobutyric, isethionic, lactic, maleic, malic, malonic, mandelic, methanesulfonic, monohydrogencarbonic, monohydrogen-phosphoric, monohydrogensulfuric, mucic, nitric, pamoic, pantothenic, phosphoric, phthalic, propionic, suberic, succinic, sulfuric, tartaric, toluenesulfonic acid, and the like (see, e.g., S. M. Berge et al., J. Pharm. Sci., 66:1-19 (1977); and Handbook of Pharmaceutical Salts: Properties, Selection and Use, P. H. Stahl and C. G. Wermuth, Eds., (2002), Wiley, Weinheim). In certain embodiments, suitable acids are strong acids (e.g., with pKa less than about 1), including, but not limited to, hydrochloric, hydrobromic, sulfuric, nitric, methanesulfonic, benzene sulfonic, toluene sulfonic, naphthalene sulfonic, naphthalene disulfonic, pyridine-sulfonic, or other substituted sulfonic acids. Also included are salts of other relatively non-toxic compounds that possess acidic character, including amino acids, such as aspartic acid and the like, and other compounds, such as aspirin, ibuprofen, saccharin, and the like. Acid addition salts can be obtained by contacting the neutral form of a compound with a sufficient amount of the desired acid, either neat or in a suitable solvent. As solids, salts can exist in crystalline or amorphous forms, or mixtures thereof. Salts can also exist in polymorphic forms.

As used herein, and unless otherwise specified, the term “protecting group” refers to a chemical group that blocks a reactive function group or groups (such as, without limitation, carboxy, hydroxy, and amino moieties) in a compound from undesirable reaction. Protecting groups known to those skilled in the art are provided herein, such as those set forth in Protective Groups in Organic Synthesis, Greene, T. W.; Wuts, P. G. M., John Wiley & Sons, New York, N.Y., (5^{th} Edition, 2014), which can be added or removed using the procedures set forth therein. Examples of protected hydroxyl groups include, but are not limited to, silyl ethers such as those obtained by reaction of a hydroxyl group with a reagent such as, but not limited to, t-butyldiphenylchlorosilane, t-butyldimethyl-chlorosilane, trimethylchlorosilane, triisopropylchlorosilane, triethylchlorosilane; substituted methyl and ethyl ethers such as, but not limited to methoxymethyl ether, methythiomethyl ether, benzyloxymethyl ether, t-butoxymethyl ether, 2-methoxyethoxymethyl ether, tetrahydropyranyl ethers, 1-ethoxyethyl ether, allyl ether, benzyl ether, 2-cyanoethyl ether; esters such as, but not limited to, benzoylformate, formate, acetate, trichloroacetate, and trifluoracetate. Examples of protecting groups include, but are not limited to, amidines (e.g., Me₂N—CH═).

As used herein, and unless otherwise specified, the term “reactive phosphorus group” refers to a chemical group or moiety that is useful for forming internucleoside linkages including for example phosphodiester and phosphorothioate internucleoside linkages. Such reactive phosphorus groups are known in the art and contain phosphorus atoms in P III or P V valence state including, but not limited to, phosphoramidite, H-phosphonate, phosphate triesters and phosphorus containing chiral auxiliaries. In one embodiment, solid phase synthesis utilizes phosphoramidites (P III chemistry) as reactive phosphites. The intermediate phosphite compounds are subsequently oxidized to the P V state using known methods to yield, in some embodiments, phosphodiester or phosphorothioate internucleotide linkages.

As used herein, and unless otherwise specified, the term “internucleoside linkage” or “internucleoside linking group” is meant to include all manner of internucleoside linking groups known in the art including but not limited to, phosphorus containing internucleoside linking groups such as phosphodiester and phosphorothioate, non-phosphorus containing internucleoside linking groups such as formacetyl and methyl eneimino, and neutral non-ionic internucleoside linking groups such as 3′-CH₂—C(═O)—N(H)-5′ or 3′-CH₂—N(H)—C(═O)-5′.

As used herein, and unless otherwise specified, the term “alkyl” refers to a straight or branched hydrocarbon chain radical consisting solely of carbon and hydrogen atoms, which is saturated. In one embodiment, the alkyl group has, for example, from one to forty carbon atoms (C₁-C₄₀ alkyl), from one to twenty-four carbon atoms (C₁-C₂₄ alkyl), four to twenty carbon atoms (C₄-C₂₀ alkyl), six to sixteen carbon atoms (C₆-C₁₆ alkyl), six to nine carbon atoms (C₆-C₉ alkyl), one to fifteen carbon atoms (C₁-C₁₅ alkyl), one to twelve carbon atoms (C₁-C₁₂ alkyl), one to eight carbon atoms (C₁-C₈ alkyl) or one to six carbon atoms (C₁-C₆ alkyl) and which is attached to the rest of the molecule by a single bond. Examples of alkyl groups include, but are not limited to, methyl, ethyl, n-propyl, 1-methylethyl (isopropyl), n-butyl, n-pentyl, 1,1-dimethylethyl (t-butyl), 3-methylhexyl, 2-methylhexyl, pentadecyl, hexadecyl, (1s,3r,5R,7S)-1-heptyl-3-octyladamantane, and the like. Unless otherwise specified, an alkyl group is optionally substituted.

As used herein, and unless otherwise specified, the term “alkenyl” refers to a straight or branched hydrocarbon chain radical consisting solely of carbon and hydrogen atoms, which contains one or more carbon-carbon double bonds. The term “alkenyl” also embraces radicals having “cis” and “trans” configurations, or alternatively, “E” and “Z” configurations, as appreciated by those of ordinary skill in the art. In one embodiment, the alkenyl group has, for example, from two to forty carbon atoms (C₂-C₄₀ alkenyl), from two to twenty-four carbon atoms (C₂-C₂₄ alkenyl), four to twenty carbon atoms (C₄-C₂₀ alkenyl), six to sixteen carbon atoms (C₆-C₁₆ alkenyl), six to nine carbon atoms (C₆-C₉ alkenyl), two to fifteen carbon atoms (C₂-C₁₅ alkenyl), two to twelve carbon atoms (C₂-C₁₂ alkenyl), two to eight carbon atoms (C₂-C₈ alkenyl) or two to six carbon atoms (C₂-C₆ alkenyl) and which is attached to the rest of the molecule by a single bond. Examples of alkenyl groups include, but are not limited to, ethenyl, prop-1-enyl, but-1-enyl, pent-1-enyl, penta-1,4-dienyl, (4Z,7Z,10Z,13Z)-nonadeca-4,7,10,13-tetraene, (4Z,7Z,10Z,13Z)-16,16-dimethylicosa-4,7,10,13-tetraene, and the like. Unless otherwise specified, an alkenyl group is optionally substituted.

As used herein, and unless otherwise specified, the term “alkylene” or “alkylene chain” refers to a straight or branched multivalent (e.g., divalent or trivalent) hydrocarbon chain linking the rest of the molecule to a radical group (or groups), consisting solely of carbon and hydrogen, which is saturated. In one embodiment, the alkylene has, for example, from one to twenty-four carbon atoms (C₁-C₂₄ alkylene), one to fifteen carbon atoms (C₁-C₁₅ alkylene), one to twelve carbon atoms (C₁-C₁₂alkylene), one to eight carbon atoms (C₁-C₈ alkylene), one to six carbon atoms (C₁-C₆ alkylene), two to four carbon atoms (C₂-C₄ alkylene), one to two carbon atoms (C₁-C₂ alkylene). Examples of alkylene groups include, but are not limited to, methylene, ethylene, propylene, n-butylene, and the like. The alkylene chain is attached to the rest of the molecule through a single bond and to the radical group through a single bond. The points of attachment of the alkylene chain to the rest of the molecule and to the radical group(s) can be through one carbon or any two (or more) carbons within the chain. Unless otherwise specified, an alkylene chain is optionally substituted.

As used herein, and unless otherwise specified, the term “alkenylene” refers to a straight or branched multivalent (e.g., divalent or trivalent) hydrocarbon chain linking the rest of the molecule to a radical group (or groups), consisting solely of carbon and hydrogen, which contains one or more carbon-carbon double bonds. In one embodiment, the alkenylene has, for example, from two to twenty-four carbon atoms (C₂-C₂₄ alkenylene), two to fifteen carbon atoms (C₂-C₁₅ alkenylene), two to twelve carbon atoms (C₂-C₁₂ alkenylene), two to eight carbon atoms (C₂-C₈ alkenylene), two to six carbon atoms (C₂-C₆ alkenylene) or two to four carbon atoms (C₂-C₄ alkenylene). Examples of alkenylene include, but are not limited to, ethenylene, propenylene, n-butenylene, and the like. The alkenylene is attached to the rest of the molecule through a single or double bond and to the radical group through a single or double bond. The points of attachment of the alkenylene to the rest of the molecule and to the radical group(s) can be through one carbon or any two (or more) carbons within the chain. Unless otherwise specified, an alkenylene is optionally substituted.

As used herein, and unless otherwise specified, the term “cycloalkyl” refers to a non-aromatic monocyclic or polycyclic hydrocarbon radical consisting solely of carbon and hydrogen atoms, and which is saturated. Cycloalkyl group may include fused, bridged, or spiro ring systems. In one embodiment, the cycloalkyl has, for example, from 3 to 15 ring carbon atoms (C₃-C₁₅ cycloalkyl), from 3 to 10 ring carbon atoms (C₃-C₁₀ cycloalkyl), or from 3 to 8 ring carbon atoms (C₃-C₈ cycloalkyl). The cycloalkyl is attached to the rest of the molecule by a single bond. Examples of monocyclic cycloalkyl radicals include, but are not limited to, cyclopropyl, cyclobutyl, cyclopentyl, cyclohexyl, cycloheptyl, and cyclooctyl. Examples of polycyclic cycloalkyl radicals include, but are not limited to, adamantyl, norbornyl, decalinyl, 7,7-dimethyl-bicyclo[2.2.1]heptanyl, and the like. Unless otherwise specified, when a cycloalkyl radical is fused to an aromatic ring, the resulted fused ring is still considered a cycloalkyl. Unless otherwise specified, a cycloalkyl group is optionally substituted.

As used herein, and unless otherwise specified, the term “cycloalkylene” is a multivalent (e.g., divalent or trivalent) cycloalkyl group. Unless otherwise specified, a cycloalkylene group is optionally substituted.

As used herein, and unless otherwise specified, the term “heterocyclyl” refers to a non-aromatic radical monocyclic or polycyclic moiety that contains one or more (e.g., one, one or two, one to three, or one to four) heteroatoms independently selected from nitrogen, oxygen, phosphorous, and sulfur. The heterocyclyl may be attached to the main structure at any heteroatom or carbon atom. A heterocyclyl group can be a monocyclic, bicyclic, tricyclic, tetracyclic, or other polycyclic ring system, wherein the polycyclic ring systems can be a fused, bridged or spiro ring system. Heterocyclyl polycyclic ring systems can include one or more heteroatoms in one or more rings. A heterocyclyl group can be saturated or partially unsaturated. Saturated heterocycloalkyl groups can be termed “heterocycloalkyl”. Partially unsaturated heterocycloalkyl groups can be termed “heterocycloalkenyl” if the heterocyclyl contains at least one double bond, or “heterocycloalkynyl” if the heterocyclyl contains at least one triple bond. In one embodiment, the heterocyclyl has, for example, 3 to 18 ring atoms (3- to 18-membered heterocyclyl), 4 to 18 ring atoms (4- to 18-membered heterocyclyl), 5 to 18 ring atoms (3- to 18-membered heterocyclyl), 4 to 8 ring atoms (4- to 8-membered heterocyclyl), or 5 to 8 ring atoms (5- to 8-membered heterocyclyl). Whenever it appears herein, a numerical range such as “3 to 18” refers to each integer in the given range; e.g., “3 to 18 ring atoms” means that the heterocyclyl group can consist of 3 ring atoms, 4 ring atoms, 5 ring atoms, 6 ring atoms, 7 ring atoms, 8 ring atoms, 9 ring atoms, 10 ring atoms, etc., up to and including 18 ring atoms. Examples of heterocyclyl groups include, but are not limited to, imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl. Unless otherwise specified, a heterocyclyl group is optionally substituted.

When the groups described herein are said to be “substituted,” they may be substituted with any appropriate substituent or substituents. Illustrative examples of substituents include, but are not limited to, those found in the exemplary compounds and embodiments provided herein, as well as: a halogen atom such as F, Cl, Br, or I; cyano; oxo (═O); hydroxyl (—OH); alkyl; alkenyl; alkynyl; cycloalkyl; aryl; —(C═O)OR′; —O(C═O)R′; —C(═O)R′; —OR′; —S(O)ₓR′; —S—SR′; —C(═O)SR′; —SC(═O)R′; —NR′R′; —NR′C(═O)R′; —C(═O)NR′R′; —NR′C(═O)NR′R′; —OC(═O)NR′R′; —NR′C(═O)OR′; —NR'S(O)ₓNR′R′; —NR'S(O)ₓR′; and —S(O)ₓNR′R′, wherein: R′ is, at each occurrence, independently H, C₁-C₁₅ alkyl or cycloalkyl, and x is 0, 1 or 2. In some embodiments the substituent is a C₁-C₁₂ alkyl group. In other embodiments, the substituent is a cycloalkyl group. In other embodiments, the substituent is a halo group, such as fluoro. In other embodiments, the substituent is a hydroxyl group. In other embodiments, the substituent is an alkoxy group (—OR′). In other embodiments, the substituent is a carboxyl group. In other embodiments, the substituent is an amino group (—NR′R′).

As used herein, and unless otherwise specified, the term “optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

A compound provided herein may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. Unless otherwise specified, a compound provided herein is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

As used herein, and unless otherwise specified, the term “isomer” refers to different compounds that have the same molecular formula. “Stereoisomers” are isomers that differ only in the way the atoms are arranged in space. “Atropisomers” are stereoisomers from hindered rotation about single bonds. “Enantiomers” are a pair of stereoisomers that are non-superimposable mirror images of each other. A mixture of a pair of enantiomers in any proportion can be known as a “racemic” mixture. “Diastereoisomers” are stereoisomers that have at least two asymmetric atoms, but which are not mirror-images of each other.

“Stereoisomers” can also include E and Z isomers, or a mixture thereof, and cis and trans isomers or a mixture thereof. In certain embodiments, a compound described herein is isolated as either the E or Z isomer. In other embodiments, a compound described herein is a mixture of the E and Z isomers.

It should be noted that if there is a discrepancy between a depicted structure and a name for that structure, the depicted structure is to be accorded more weight.

### Compounds

Unless otherwise specified, the descriptions provided herein apply to all the formulas provided herein (e.g., Formulas (I) to (VIII), including their sub-formulas), to the extent that they are applicable.

In one embodiment, provided herein is a compound of Formula (I):

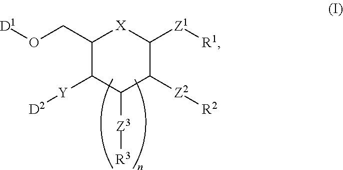

wherein:
X is O or S;
Y is O or NR^{a};
D¹ is H, a hydroxyl protecting group, or a reactive phosphorus group;
D² is H, a hydroxyl protecting group, or a reactive phosphorus group;
or D¹ and D² together form a protecting group for both the oxygen and Y they are attached to;
Z¹, Z², and Z³ are each independently —O—, —NR^{a}—, *—OC(═O)—, *—NR^{a}C(═O)—, —OC(═O)O—, *—NR^{a}C(═O)O—, *—OC(═O)NR^{b}—, *—NR^{a}C(═O)NR^{b}—, *—OC(═S)—, *—NR^{a}C(═S)—, —OC(═S)O—, *—NR^{a}C(═S)O—, *—OC(═S)NR^{b}—, *—NR^{a}C(═S)NR^{b}—, *—OC(═O)S—, *—NR^{a}C(═O)S—, *—OS(O)ₓ—, or *—NR^{a}S(O)ₓ—; wherein * refers to the direction toward the ring containing X;
R¹, R², and R³ are each independently H, C₁-C₆ alkyl optionally substituted with C₁-C₆ alkoxy, or -(G-L)ₘ-R, provided that at least one of R¹, R², and R³ is -(G-L)ₘ-R;
each instance of G is independently C₁-C₈ alkylene;
each instance of L is independently —O—, —NR^{a}—, —C(═O)—, *—OC(═O)—, *—C(═O)O—, *—NR^{a}C(═O)—, *—C(═O)NR^{b}—, —OC(═O)O—, *—NR^{a}C(═O)O—, *—OC(═O)NR^{b}—, *—NR^{a}C(═O)NR^{b}—, *—OC(═S)—, *—C(═S)O—, *—NR^{a}C(═S)—, *—C(═S)NR^{b}—, —OC(═S)O—, *—NR^{a}C(═S)O—, *—OC(═S)NR^{b}—, *—NR^{a}C(═S)NR^{b}—, *—OC(═O)S—, *—SC(═O)O—, *—NR^{a}C(═O)S—, *—SC(═O)NR^{b}—, *—OS(O)ₓ—, *—S(O)ₓO—, *—NR^{a}S(O)ₓ—, or *—S(O)ₓNR^{b}—; wherein * refers to the direction toward the ring containing X;
each instance of R^{a} is independently H or C₁-C₆ alkyl;
each instance of R^{b} is independently H or C₁-C₆ alkyl; and
each instance of R is independently C₁₂-C₃₂ alkyl or C₁₂-C₃₂ alkenyl, or when more than one of R¹, R², and R³ are -(G-L)ₘ-R, then one or more R is independently C₁₂-C₃₂ alkyl, C₁₂-C₃₂ alkenyl, or an optionally protected mannose;
x is 1 or 2;
n is 0 or 1;
m is 0, 1, or 2;
   or a stereoisomer thereof, or a pharmaceutically acceptable salt thereof.

In one embodiment, X is O. In one embodiment, X is S.

In one embodiment, Y is O. In one embodiment, Y is NR^{a}. In one embodiment, Y is NH.

In one embodiment, Z¹, Z², and Z³ (if present) are each independently —O—, —NR^{a}—, *—OC(═O)—, *—NR^{a}C(═O)—, —OC(═O)O—, *—NR^{a}C(═O)O—, *—OC(═O)NR^{b}—, or *—NR^{a}C(═O)NR^{b}—. In one embodiment, Z¹, Z², and Z³ (if present) are each independently —O—, —NH—, *—OC(═O)—, *—NHC(═O)—, —OC(═O)O—, *—NHC(═O)O—, *—OC(═O)NH—, or —NHC(═O)NH—. In one embodiment, Z¹, Z², and Z³ (if present) are all —O—.

In one embodiment, n is 0. In one embodiment, the compound is a compound of Formula (I-A):

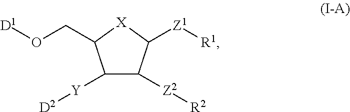

or a stereoisomer thereof, or a pharmaceutically acceptable salt thereof.

In one embodiment, the compound is a compound of Formula (I-A-1):

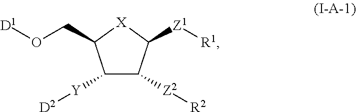

or a pharmaceutically acceptable salt thereof.

In one embodiment, the compound is a compound of Formula (II-A):

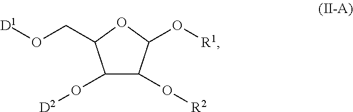

or a stereoisomer thereof, or a pharmaceutically acceptable salt thereof.

In one embodiment, the compound is a compound of Formula (II-A-1):

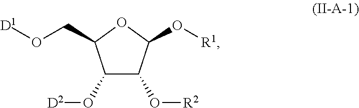

or a pharmaceutically acceptable salt thereof.

In one embodiment, n is 1. In one embodiment, the compound is a compound of Formula (I-B):

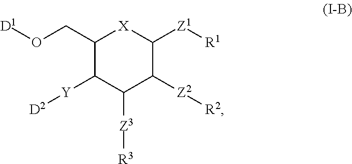

or a stereoisomer thereof, or a pharmaceutically acceptable salt thereof.

In one embodiment, the compound is a compound of Formula (I-B-1):

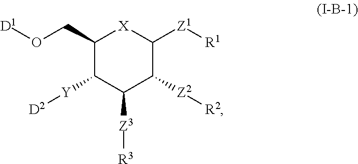

or a pharmaceutically acceptable salt thereof.

In one embodiment, the compound is a compound of Formula (II-B):

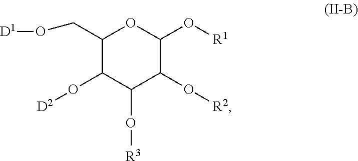

or a stereoisomer thereof, or pharmaceutically acceptable salt thereof.

In one embodiment, the compound is a compound of Formula (II-B-1):

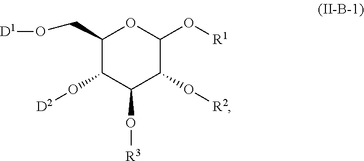

or pharmaceutically acceptable salt thereof.

Unless otherwise specified, when the stereochemistry of a position in a formula provided herein is not specified, a compound of the formula can be a stereoisomer at that position, or a mixture thereof. For example, in one embodiment, a compound of Formula (I-B-1) or Formula (II-B-1) is an alpha-isomer at 1-position; in another embodiment, the compound is a beta-isomer at 1-position; and yet in another embodiment, the compound is a mixture of the alpha- and beta-isomers.

In one embodiment, R¹ is H. In one embodiment, R¹ is C₁-C₆ alkyl. In one embodiment, R¹ is methyl. In one embodiment, R¹ is ethyl. In one embodiment, R¹ is n-propyl. In one embodiment, R¹ is isopropyl. In one embodiment, R¹ is n-butyl. In one embodiment, R¹ is n-pentyl. In one embodiment, R¹ is n-hexyl. In one embodiment, the alkyl is substituted with C₁-C₆ alkoxy. In one embodiment, the alkoxy is methoxy. In one embodiment, the alkoxy is ethoxy. In one embodiment, the alkoxy is n-propoxy. In one embodiment, R¹ is 2-methoxyethyl (MOE).

In one embodiment, R² is H. In one embodiment, R² is C₁-C₆ alkyl. In one embodiment, R² is methyl. In one embodiment, R² is ethyl. In one embodiment, R² is n-propyl. In one embodiment, R² is isopropyl. In one embodiment, R² is n-butyl. In one embodiment, R² is n-pentyl. In one embodiment, R² is n-hexyl. In one embodiment, the alkyl is substituted with C₁-C₆ alkoxy. In one embodiment, the alkoxy is methoxy. In one embodiment, the alkoxy is ethoxy. In one embodiment, the alkoxy is n-propoxy. In one embodiment, R² is 2-methoxyethyl (MOE).

In one embodiment, R³ is H. In one embodiment, R³ is C₁-C₆ alkyl. In one embodiment, R³ is methyl. In one embodiment, R³ is ethyl. In one embodiment, R³ is n-propyl. In one embodiment, R³ is isopropyl. In one embodiment, R³ is n-butyl. In one embodiment, R³ is n-pentyl. In one embodiment, R³ is n-hexyl. In one embodiment, the alkyl is substituted with C₁-C₆ alkoxy. In one embodiment, the alkoxy is methoxy. In one embodiment, the alkoxy is ethoxy. In one embodiment, the alkoxy is n-propoxy. In one embodiment, R³ is 2-methoxyethyl (MOE).

In one embodiment, R¹ is -(G-L)ₘ-R. In one embodiment, R² is -(G-L)ₘ-R. In one embodiment, R³ is -(G-L)ₘ-R. In one embodiment, R¹ and R² are -(G-L)ₘ-R. In one embodiment, R¹, R² and R³ are -(G-L)ₘ-R. In one embodiment, R¹ and R² are -(G-L)ₘ-R, where R in R¹ is C₁₂-C₃₂ alkyl or C₁₂-C₃₂ alkenyl and R in R² is an optionally protected mannose. In one embodiment, R¹ and R³ are -(G-L)ₘ-R, where R in R¹ is C₁₂-C₃₂ alkyl or C₁₂-C₃₂ alkenyl and R in R³ is an optionally protected mannose. In one embodiment, R¹, R² and R³ are -(G-L)ₘ-R, where R in R¹ is C₁₂-C₃₂ alkyl or C₁₂-C₃₂ alkenyl and R in R² and R³ are an optionally protected mannose. In some embodiments, the optionally protected mannose is protected by acetyl moieties.

In one embodiment of where n is 0, R¹ is -(G-L)ₘ-R, and R² is C₁-C₆ alkyl optionally substituted with C₁-C₆ alkoxy. In one embodiment, R² is -(G-L)ₘ-R, and R¹ is C₁-C₆ alkyl optionally substituted with C₁-C₆ alkoxy. In one embodiment, R¹ and R² are each independently -(G-L)ₘ-R.

In one embodiment of where n is 1, R¹ is -(G-L)ₘ-R, and R² and R³ are each independently C₁-C₆ alkyl optionally substituted with C₁-C₆ alkoxy. In one embodiment, R² is -(G-L)ₘ-R, and R¹ and R³ are each independently C₁-C₆ alkyl optionally substituted with C₁-C₆ alkoxy. In one embodiment, R³ is -(G-L)ₘ-R, and R¹ and R² are each independently C₁-C₆ alkyl optionally substituted with C₁-C₆ alkoxy. In one embodiment, R¹ and R² are each independently -(G-L)ₘ-R, and R³ is C₁-C₆ alkyl optionally substituted with C₁-C₆ alkoxy. In one embodiment, R¹ and R³ are each independently -(G-L)ₘ-R, and R² is C₁-C₆ alkyl optionally substituted with C₁-C₆ alkoxy. In one embodiment, R² and R³ are each independently -(G-L)ₘ-R, and R¹ is C₁-C₆ alkyl optionally substituted with C₁-C₆ alkoxy. In one embodiment, R¹, R², and R³ are each independently -(G-L)ₘ-R.

In one embodiment, m is 0. In one embodiment, -(G-L)ₘ-R is —R.

In one embodiment, m is 1. In one embodiment, m is 2.

In one embodiment, each instance of G is independently C₁-C₈ alkylene. In one embodiment, each instance of G is independently C₁-C₄ alkylene. In one embodiment, the alkylene is C₁ alkylene. In one embodiment, the alkylene is C₂ alkylene. In one embodiment, the alkylene is C₃ alkylene. In one embodiment, the alkylene is C₄ alkylene. In one embodiment, the alkylene is C₅ alkylene. In one embodiment, the alkylene is C₆ alkylene. In one embodiment, the alkylene is —CH₂—. In one embodiment, the alkylene is —CH₂CH₂—. In one embodiment, the alkylene is —CH₂CH₂CH₂—.

In one embodiment, each instance of L is independently —O—, —NH—, *—OC(═O)—, *—C(═O)O—, *—NHC(═O)—, *—C(═O)NH—, —OC(═O)O—, *—NHC(═O)O—, *—OC(═O)NH—, or *—NHC(═O)NH—. In one embodiment, each instance of L is independently *—NHC(═O)—.

In one embodiment, -(G-L)ₘ-R is —(C₂-C₆ alkylene)-NH—C(═O)—R. In one embodiment, -(G-L)ₘ-R is —(C₂-C₃ alkylene)-NH—C(═O)—R. In one embodiment, -(G-L)ₘ-R is —CH₂CH₂—NH—C(═O)—R. In one embodiment, -(G-L)ₘ-R is —CH₂CH₂CH₂—NH—C(═O)—R.

In one embodiment, R^{a} is H. In one embodiment, R^{a} is C₁-C₆ alkyl. In one embodiment, R^{a} is methyl.

In one embodiment, R^{b} is H. In one embodiment, R^{b} is C₁-C₆ alkyl. In one embodiment, R^{b} is methyl.

In one embodiment, provided herein is a compound of Formula (III):

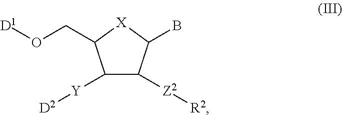

wherein:
B is a modified or unmodified nucleobase;
X is O or S;
Y is O or NR^{a};
D¹ is H, a hydroxyl protecting group, or a reactive phosphorus group;
D² is H, a hydroxyl protecting group, or a reactive phosphorus group;
or D¹ and D² together is a protecting group for both the oxygen and Y they are attached to;
Z² is —O—, —NR^{a}—, *—OC(═O)—, *—NR^{a}C(═O)—, —OC(═O)O—, *—NR^{a}C(═O)O—, *—OC(═O)NR^{b}—, *—NR^{a}C(═O)NR^{b}—, *—OC(═S)—, *—NR^{a}C(═S)—, —OC(═S)O—, *—NR^{a}C(═S)O—, *—OC(═S)NR^{b}—, *—NR^{a}C(═S)NR^{b}—, *—OC(═O)S—, *—NR^{a}C(═O)S—, *—OS(O)ₓ—, or *—NR^{a}S(O)ₓ—; wherein * refers to the direction toward the ring containing X;
R² is -(G-L)ₘ-R;
each instance of G is independently C₁-C₈ alkylene or C₃-C₁₀ cycloalkylene;
each instance of L is independently —O—, —NR^{a}—, —C(═O)—, *—OC(═O)—, *—C(═O)O—, *—NR^{a}C(═O)—, *—C(═O)NR^{b}—, —OC(═O)O—, *—NR^{a}C(═O)O—, *—OC(═O)NR^{b}—, *—NR^{a}C(═O)NR^{b}—, *—OC(═S)—, *—C(═S)O—, *—NR^{a}C(═S)—, *—C(═S)NR^{b}—, —OC(═S)O—, *—NR^{a}C(═S)O—, *—OC(═S)NR^{b}—, *—NR^{a}C(═S)NR^{b}—, *—OC(═O)S—, *—SC(═O)O—, *—NR^{a}C(═O)S—, *—SC(═O)NR^{b}—, *—OS(O)ₓ—, *—S(O)ₓO—, *—NR^{a}S(O)ₓ—, or *—S(O)ₓNR^{b}—; wherein * refers to the direction toward the ring containing X;
each instance of R^{a} is independently H or C₁-C₆ alkyl;
each instance of R^{b} is independently H or C₁-C₆ alkyl; and
R is C₁₂-C₃₂ alkyl or C₁₂-C₃₂ alkenyl;
x is 1 or 2; and
m is 0, 1, or 2;
provided that when X is O, Y is O, and Z² is —O—, then
(i) R is substituted with -L″-(CH₂)₀₋₃—R″, wherein:
   L″ is absent, —O—, —NR^{a}—, —C(═O)—, *—OC(═O)—, *—C(═O)O—, *—NR^{a}C(═O)—, *—C(═O)NR^{b}—, —OC(═O)O—, *—NR^{a}C(═O)O—, *—OC(═O)NR^{b}—, *—NR^{a}C(═O)NR^{b}—, *—OC(═S)—, *—C(═S)O—, *—NR^{a}C(═S)—, *—C(═S)NR^{b}—, —OC(═S)O—, *—NR^{a}C(═S)O—, *—OC(═S)NR^{b}—, *—NR^{a}C(═S)NR^{b}—, *—OC(═O)S—, *—SC(═O)O—, *—NR^{a}C(═O)S—, *—SC(═O)NR^{b}—, *—OS(O)ₓ—, *—S(O)ₓO—, *—NR^{a}S(O)ₓ—, or *—S(O)ₓNR^{b}—; wherein * refers to the direction toward R; and
   R″ is a monocyclic, fused, bridged or spiro ring moiety, wherein the ring moiety is optionally substituted with one or more C₁-C₆ alkyl, oxo, or phenyl, wherein the alkyl or phenyl is optionally substituted with one or more halogen, phenyl, or phenoxy, wherein the phenyl or phenoxy is optionally substituted with one or more halogen or C₁-C₆ alkoxy;
(ii) R is C₁₂-C₃₂ alkenyl;
(iii) m is 1 or 2; and L is —O— or *—C(═O)NR^{b}—; or
(iv) m is 1 or 2, and at least one G is C₃-C₁₀ cycloalkylene;
   or a stereoisomer thereof, or a pharmaceutically acceptable salt thereof.

In one embodiment, X is O. In one embodiment, X is S.

In one embodiment, Y is O. In one embodiment, Y is NR^{a}. In one embodiment, Y is NH.

In one embodiment, the compound is a compound of Formula (III-A):

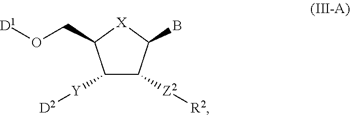

or a pharmaceutically acceptable salt thereof.

In one embodiment, the compound is a compound of Formula (III-A-1):

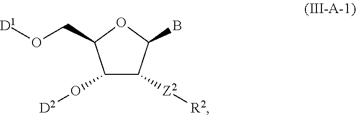

or a pharmaceutically acceptable salt thereof.

In one embodiment, Z² is —O—, *—OC(═O)—, *—NR^{a}C(═O)—, —OC(═O)O—, *—NR^{a}C(═O)O—, *—OC(═O)NR^{b}—, *—NR^{a}C(═O)NR^{b}—, *—OC(═O)S—, or *—NR^{a}C(═O)S—. In one embodiment, Z² is —OC(═O)NH—. In one embodiment, Z² is —NHC(═O)—. In one embodiment, Z² is —NHC(═O)NH—. In one embodiment, Z² is —NHC(═O)S—. In one embodiment, Z² is —O—.

In one embodiment, when X is O, Y is O, and Z² is —O—, then R is C₁₂-C₃₂ alkenyl. In one embodiment, m is 0 and R is C₁₂-C₃₂ alkenyl.

In one embodiment, when X is O, Y is O, and Z² is —O—, then R is substituted with -L″-(CH₂)₀₋₃—R″. In one embodiment, m is 0 and R is substituted with -L″-(CH₂)₀₋₃—R″. In one embodiment, m is 0, and R is substituted with a fused, bridged or spiro cycloalkyl. In one embodiment, R is substituted with adamantyl. In one embodiment, the adamantyl is 1-adamantyl. In one embodiment, the adamantyl is 2-adamantyl. In one embodiment, R is substituted with

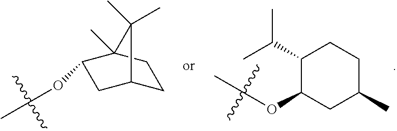 [SMILES: *O[C@H]1CC2CCC1(C)C2(C)C ; *O[C@@H]1C[C@H](C)CC[C@H]1C(C)C]

In one embodiment, when X is O, Y is O, and Z² is —O—, then m is 1 or 2; and L is —O— or *—C(═O)NR^{b}—. In one embodiment, m is 1, and L is *—C(═O)NR^{b}—. In one embodiment, m is 1, and L is —O—. In one embodiment, when X is O, Y is O, and Z² is —O—, then R² is —(C₂-C₆ alkylene)-C(═O)NH—R. In one embodiment, R² is —(C₂-C₃ alkylene)-C(═O)NH—R. In one embodiment, R² is —CH₂CH₂—C(═O)NH—R. In one embodiment, R² is —CH₂CH₂CH₂—C(═O)NH—R. In one embodiment, when X is O, Y is O, and Z² is —O—, then R² is —(C₂-C₆ alkylene)-O—R. In one embodiment, R² is —(C₂-C₃ alkylene)-O—R. In one embodiment, R² is —CH₂CH₂—O—R. In one embodiment, R² is —CH₂CH₂CH₂—O—R.

In one embodiment, when X is O, Y is O, and Z² is —O—, then m is 1 or 2, and at least one G is C₃-C₁₀ cycloalkylene. In one embodiment, m is 1 and G is C₃-C₁₀ cycloalkylene.

In one embodiment, m is 0. In one embodiment, m is 1. In one embodiment, m is 2.

In one embodiment, each instance of G is independently C₁-C₈ alkylene. In one embodiment, each instance of G is independently C₁-C₄ alkylene. In one embodiment, the alkylene is C₁ alkylene. In one embodiment, the alkylene is C₂ alkylene. In one embodiment, the alkylene is C₃ alkylene. In one embodiment, the alkylene is C₄ alkylene. In one embodiment, the alkylene is C₅ alkylene. In one embodiment, the alkylene is C₆ alkylene. In one embodiment, the alkylene is —CH₂—. In one embodiment, the alkylene is —CH₂CH₂—. In one embodiment, the alkylene is —CH₂CH₂CH₂—.

In one embodiment, each instance of G is independently C₃-C₁₀ cycloalkylene. In one embodiment, each instance of G is independently C₃-C₈ cycloalkylene. In one embodiment, the cycloalkylene is cyclopropylene. In one embodiment, the cycloalkylene is cyclobutylene. In one embodiment, the cycloalkylene is cyclopentylene. In one embodiment, the cycloalkylene is cyclohexylene. In one embodiment, the cycloalkylene is cycloheptylene. In one embodiment, the cycloalkylene is cyclooctylene.

In one embodiment, each instance of L is independently —O—, —NH—, *—OC(═O)—, *—C(═O)O—, *—NHC(═O)—, *—C(═O)NH—, —OC(═O)O—, *—NHC(═O)O—, *—OC(═O)NH—, or *—NHC(═O)NH—. In one embodiment, each instance of L is independently *—C(═O)NH—.

In one embodiment, -(G-L)ₘ-R is —(C₂-C₆ alkylene)-C(═O)NH—R. In one embodiment, -(G-L)ₘ-R is —(C₂-C₃ alkylene)-C(═O)NH—R. In one embodiment, -(G-L)ₘ-R is —CH₂CH₂—C(═O)NH—R. In one embodiment, -(G-L)ₘ-R is —CH₂CH₂CH₂—C(═O)NH—R.

In one embodiment, R^{a} is H. In one embodiment, R^{a} is C₁-C₆ alkyl. In one embodiment, R^{a} is methyl.

In one embodiment, R^{b} is H. In one embodiment, R^{b} is C₁-C₆ alkyl. In one embodiment, R^{b} is methyl.

In one embodiment, B is adenine (A), guanine (G), thymine (T), cytosine (C), or uracil (U). In one embodiment, B is an unmodified (natural) nucleobase. In one embodiment, B is an modified (natural) nucleobase, as provided herein or known in the art.

In one embodiment, D¹ is a hydroxyl protecting group. In one embodiment, D¹ is 4,4′-dimethoxytrityl chloride (DMTr). In one embodiment, D¹ is 4-monomethoxytrityl (MMTr).

In one embodiment, D² is a reactive phosphorus group. In one embodiment, D² is

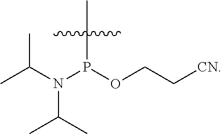 [SMILES: *P(OCCC#N)N(C(C)C)C(C)C]

In one embodiment, D¹ is H and D² is H.

In one embodiment, provided herein is a compound of Formula (IV):

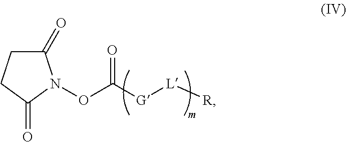

wherein:
each instance of G′ is independently C₁-C₈ alkylene or C₃-C₈ cycloalkylene;
each instance of L′ is independently *—NR^{c}C(═O)—; wherein * refers to the direction toward the pyrrolidine-2,5-dione ring;
each instance of R^{c} is independently H, C₁-C₃₂ alkyl, or C₂-C₃₂ alkenyl; R is C₄-C₃₂ alkyl or C₄-C₃₂ alkenyl; and
m is 0, 1, or 2;
or a stereoisomer thereof, or a pharmaceutically acceptable salt thereof.

In one embodiment, m is 0. In one embodiment, m is 1. In one embodiment, m is 2.

In one embodiment, each instance of G′ is independently C₁-C₈ alkylene. In one embodiment, each instance of G′ is independently C₁-C₄ alkylene. In one embodiment, the alkylene is C₁ alkylene. In one embodiment, the alkylene is C₂ alkylene. In one embodiment, the alkylene is C₃ alkylene. In one embodiment, the alkylene is C₄ alkylene. In one embodiment, the alkylene is C₅ alkylene. In one embodiment, the alkylene is C₆ alkylene. In one embodiment, the alkylene is —CH₂—. In one embodiment, the alkylene is —CH₂CH₂—. In one embodiment, the alkylene is —CH₂CH₂CH₂—.

In one embodiment, each instance of G′ is independently C₃-C₈ cycloalkylene. In one embodiment, each instance of G′ is independently C₄-C₆ cycloalkylene. In one embodiment, the cycloalkylene is cyclopropylene. In one embodiment, the cycloalkylene is cyclobutylene. In one embodiment, the cycloalkylene is cyclopentylene. In one embodiment, the cycloalkylene is cyclohexylene. In one embodiment, the cycloalkylene is cycloheptylene. In one embodiment, the cycloalkylene is cyclooctylene.

In one embodiment, -(G′-L′)ₘ-R is

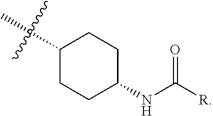

In one embodiment, -(G′-L′)ₘ-R is

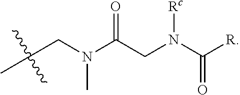

The descriptions for R provided herein apply to all the formulas provided herein to the extent that they are applicable.

In one embodiment, R is C₄-C₃₂ alkyl. In one embodiment, R is C₆-C₃₂ alkyl. In one embodiment, R is C₈-C₃₂ alkyl. In one embodiment, R is C₁₀-C₃₂ alkyl. In one embodiment, R is C₁₂-C₃₂ alkyl. In one embodiment, R is C₁₂-C₂₄ alkyl. In one embodiment, R is C₁₄-C₁₈ alkyl. In one embodiment, R is C₄ alkyl. In one embodiment, R is C₆ alkyl. In one embodiment, R is C₈ alkyl. In one embodiment, R is C₁₀ alkyl. In one embodiment, R is C₁₂ alkyl. In one embodiment, R is C₁₄ alkyl. In one embodiment, R is C₁₅ alkyl. In one embodiment, R is C₁₆ alkyl. In one embodiment, R is C₁₇ alkyl. In one embodiment, R is C₁₈ alkyl. In one embodiment, the alkyl is straight alkyl. In one embodiment, the alkyl is branched alkyl.

In one embodiment, R is C₄-C₃₂ alkenyl. In one embodiment, R is C₆-C₃₂ alkenyl. In one embodiment, R is C₈-C₃₂ alkenyl. In one embodiment, R is C₁₀-C₃₂ alkenyl. In one embodiment, R is C₁₂-C₃₂ alkenyl. In one embodiment, R is C₁₂-C₂₄ alkenyl. In one embodiment, R is C₁₄-C₁₈ alkenyl. In one embodiment, R is C₄ alkenyl. In one embodiment, R is C₆ alkenyl. In one embodiment, R is C₈ alkenyl. In one embodiment, R is C₁₀ alkenyl. In one embodiment, R is C₁₂ alkenyl. In one embodiment, R is C₁₄ alkenyl. In one embodiment, R is C₁₅ alkenyl. In one embodiment, R is C₁₆ alkenyl. In one embodiment, R is C₁₇ alkenyl. In one embodiment, R is Cis alkenyl. In one embodiment, the alkenyl is straight alkenyl. In one embodiment, the alkenyl is branched alkenyl.

In one embodiment, R is unsubstituted. In one embodiment, R is —(CH₂)₁₃₋₁₇CH₃.

In one embodiment, R is substituted with -L″-(CH₂)₀₋₃-R″, wherein: L″ is absent, —O—, —NR^{a}—, —C(═O)—, *—OC(═O)—, *—C(═O)O—, *—NR^{a}C(═O)—, *—C(═O)NR^{b}—, —OC(═O)O—, *—NR^{a}C(═O)O—, *—OC(═O)NR^{b}—, *—NR^{a}C(═O)NR^{b}—, *—OC(═S)—, *—C(═S)O—, *—NR^{a}C(═S)—, *—C(═S)NR^{b}—, —OC(═S)O—, *—NR^{a}C(═S)O—, *—OC(═S)NR^{b}—, *—NR^{a}C(═S)NR^{b}—, *—OC(═O)S—, *—SC(═O)O—, *—NR^{a}C(═O)S—, *—SC(═O)NR^{b}—, *—OS(O)ₓ—, *—S(O)ₓO—, *—NR^{a}S(O)ₓ—, or *—S(O)ₓNR^{b}—; wherein * refers to the direction toward R;
each instance of R^{a} is independently H or C₁-C₆ alkyl;
each instance of R^{b} is independently H or C₁-C₆ alkyl;
x is 1 or 2; and
R″ is a monocyclic, fused, bridged or spiro ring moiety, wherein the ring moiety is optionally substituted with one or more C₁-C₆ alkyl, oxo, or phenyl, wherein the alkyl or phenyl is optionally substituted with one or more halogen, phenyl, or phenoxy, wherein the phenyl or phenoxy is optionally substituted with one or more halogen or C₁-C₆ alkoxy.

In one embodiment, the ring moiety is a C₃-C₁₀ cycloalkyl. In one embodiment, R″ is a monocyclic cycloalkyl. In one embodiment, R″ is a fused cycloalkyl. In one embodiment, R″ is a bridged cycloalkyl. In one embodiment, R″ is a spiro cycloalkyl. In one embodiment, the cycloalkyl is a C₃-C₁₂ cycloalkyl. In one embodiment, the cycloalkyl is cyclohexyl. In one embodiment, the cycloalkyl is bicyclo[2.2.1]heptyl. In one embodiment, the cycloalkyl is adamantyl. In one embodiment, the cycloalkyl is substituted with one or more C₁-C₆ alkyl. In one embodiment, the cycloalkyl is substituted with one or more methyl or isopropyl.

In one embodiment, the ring moiety is a 4- to 10-membered heterocyclyl containing 1 to 4 heteroatoms independently selected from O, N, and S.

In one embodiment, R is substituted with -L″-R″, wherein L″ is absent, —O—, or —C(═O)O—, and R″ is a monocyclic, fused, bridged or spiro cycloalkyl, which is optionally substituted with one or more C₁-C₆ alkyl. In one embodiment, R is substituted with R″. In one embodiment, R is substituted with —O—R″. In one embodiment, R is substituted with —C(═O)O—R″. In one embodiment, the substituent is placed on the terminal position of R. In one embodiment, R is —(CH₂)₁₄₋₁₈-L″-R″.

In one embodiment, R″ is

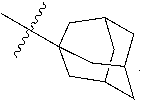 [SMILES: *C12CC3CC(CC(C3)C1)C2]

In one embodiment, R″ is

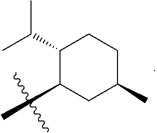

In one embodiment, R″ is

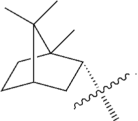

In one embodiment, R is substituted with

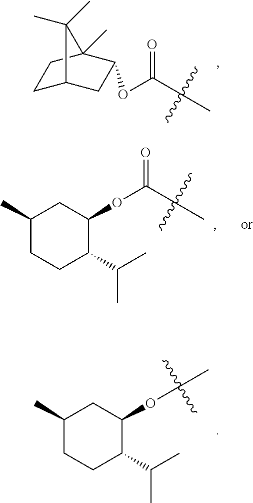 [SMILES: *C(=O)O[C@@H]1CC2CCC1(C)C2(C)C ; *C(=O)O[C@@H]1C[C@H](C)CC[C@H]1C(C)C ; *O[C@@H]1C[C@H](C)CC[C@H]1C(C)C]

In one embodiment, R is substituted at a terminal position.

In one embodiment, the compound is a compound in Table 1, or a stereoisomer thereof, or pharmaceutically acceptable salt thereof (wherein B is a modified or unmodified nucleobase).

TABLE 1

| | |
|---|---|
| 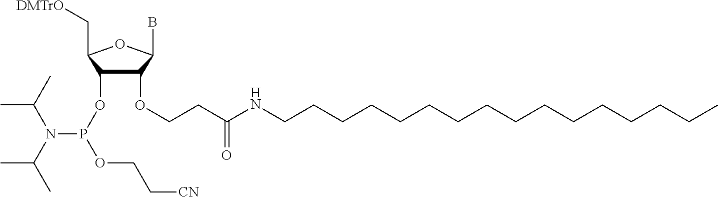 | Compound 1 |
| 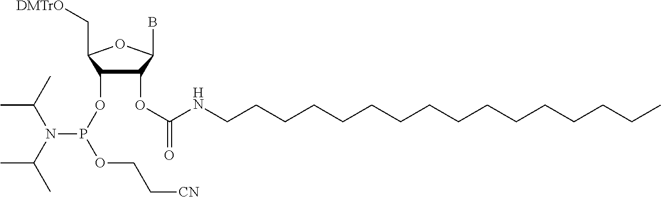 | Compound 2 |
| 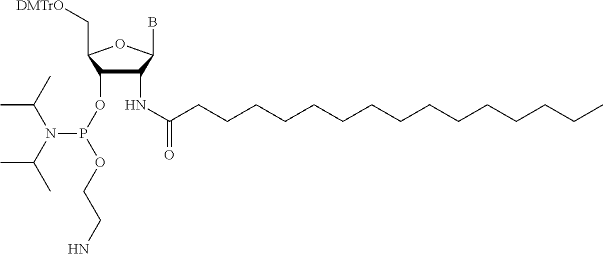 | Compound 3 |
| 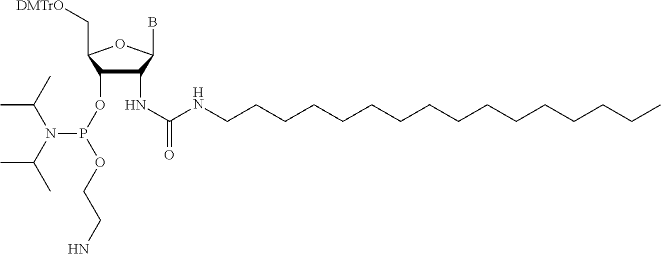 | Compound 4 |
| 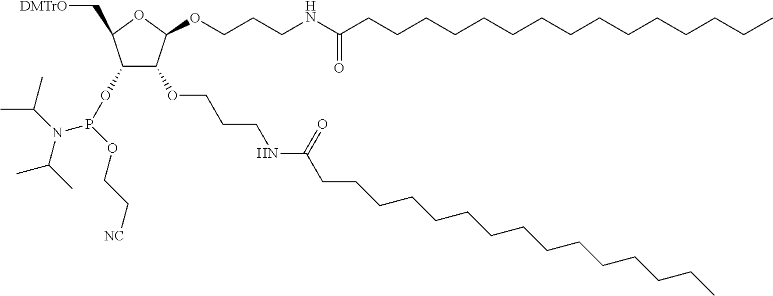 | Compound 5 |
| 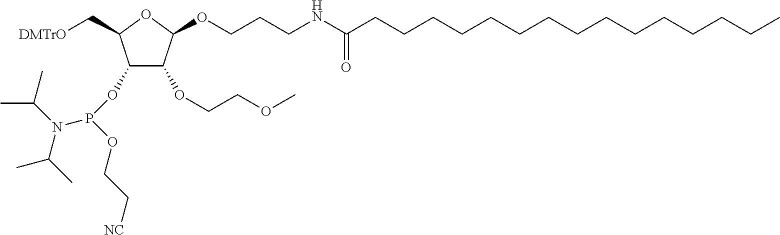 | Compound 6 |
| 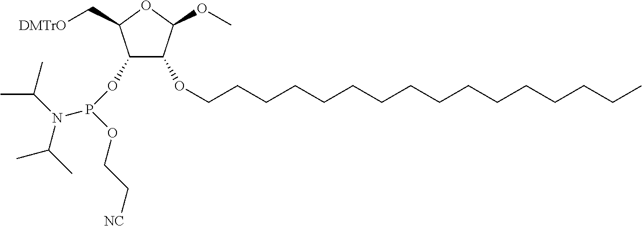 | Compound 7 |
| 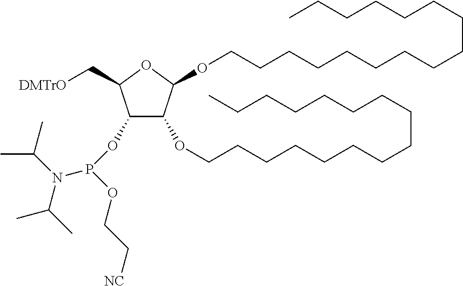 | Compound 8 |
| 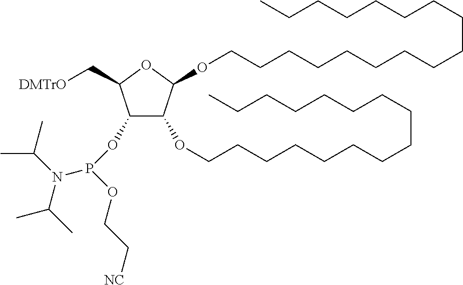 | Compound 9 |
| 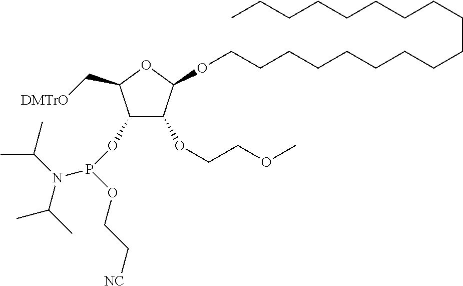 | Compound 10 |
| 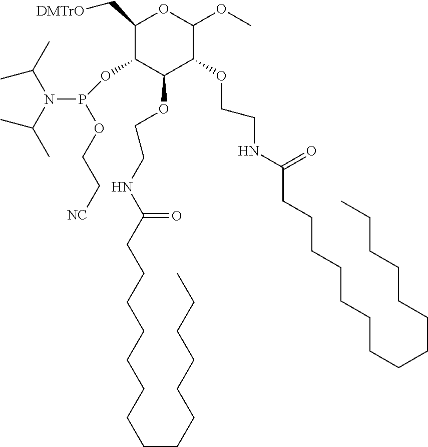 | Compound 11 |
| 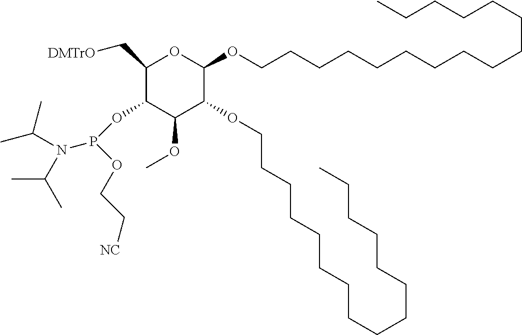 | Compound 12 |
| 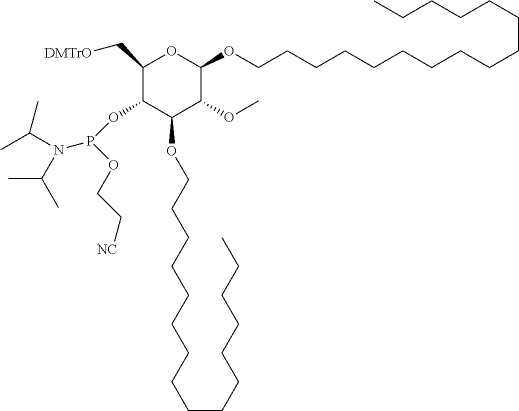 | Compound 13 |
| 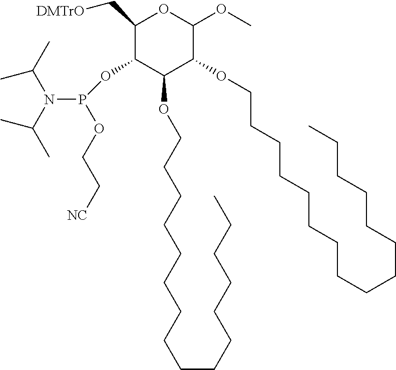 | Compound 14 |
| 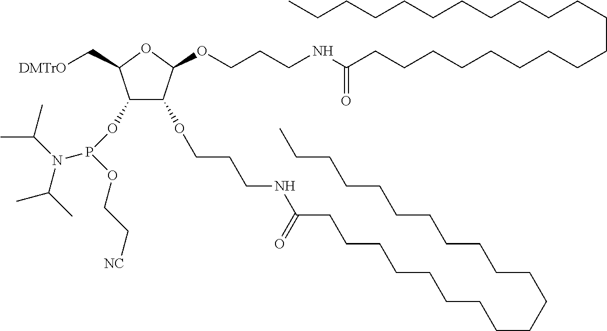 | Compound 15 |
| 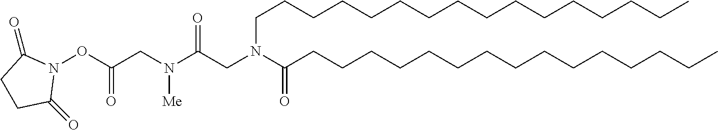 [SMILES: CCCCCCCCCCCCCCCCN(CC(=O)N(C)CC(=O)ON1C(=O)CCC1=O)C(=O)CCCCCCCCCCCCCCC] | Compound 16 |
| 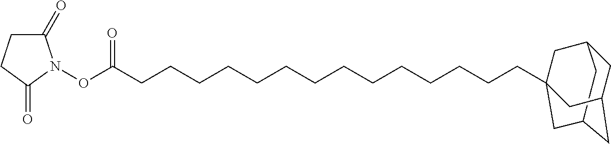 [SMILES: O=C(CCCCCCCCCCCCCCC12CC3CC(CC(C3)C1)C2)ON1C(=O)CCC1=O] | Compound 17 |
| 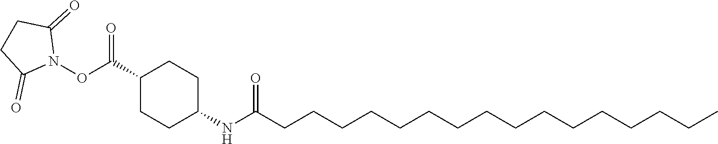 [SMILES: CCCCCCCCCCCCCCCCC(=O)N[C@H]1CC[C@@H](C(=O)ON2C(=O)CCC2=O)CC1] | Compound 18 |
| 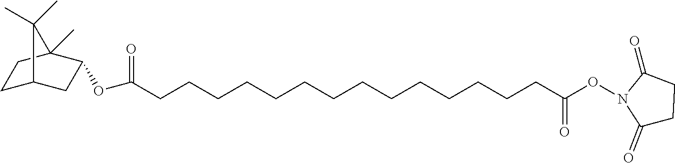 [SMILES: CC1(C)C2CCC1(C)[C@H](OC(=O)CCCCCCCCCCCCCCC(=O)ON1C(=O)CCC1=O)C2] | Compound 19 |
| 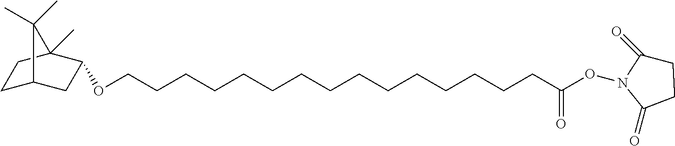 [SMILES: CC1(C)C2CCC1(C)[C@H](OCCCCCCCCCCCCCCCC(=O)ON1C(=O)CCC1=O)C2] | Compound 20 |
| 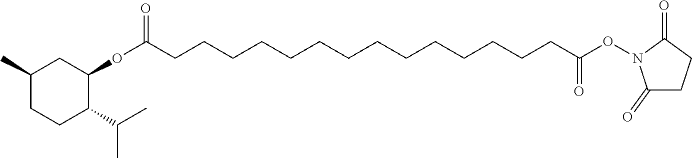 [SMILES: CC(C)[C@@H]1CC[C@@H](C)C[C@H]1OC(=O)CCCCCCCCCCCCCCC(=O)ON1C(=O)CCC1=O] | Compound 22 |
| 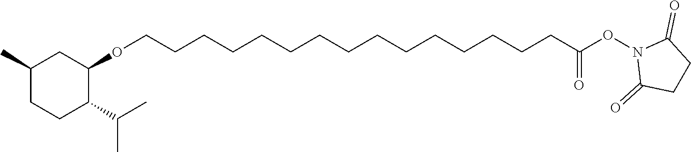 [SMILES: CC(C)[C@@H]1CC[C@@H](C)C[C@H]1OCCCCCCCCCCCCCCCC(=O)ON1C(=O)CCC1=O] | Compound 23 |
| 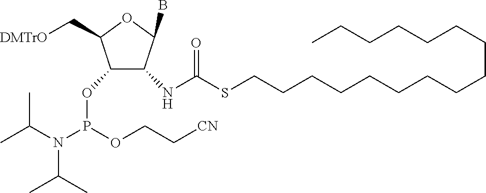 | Compound 24 |
| 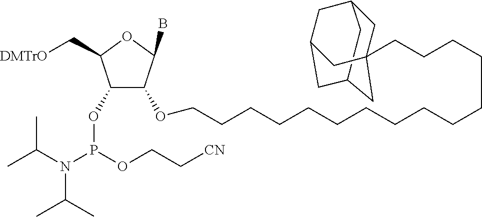 | Compound 25 |
| 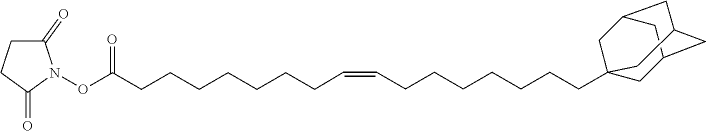 [SMILES: O=C(CCCCCCC/C=C\CCCCCCCC12CC3CC(CC(C3)C1)C2)ON1C(=O)CCC1=O] | Compound 26 |
| 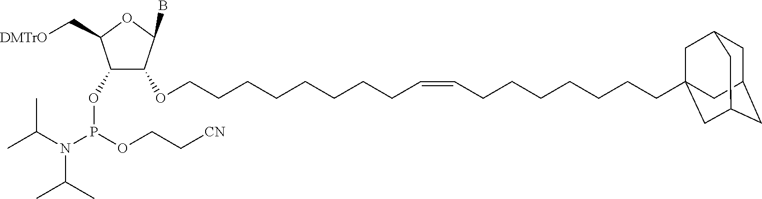 | Compound 27 |
| 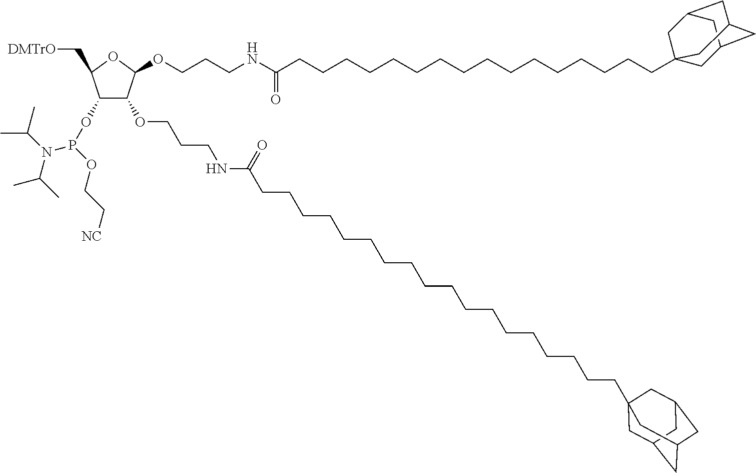 | Compound 28 |
| 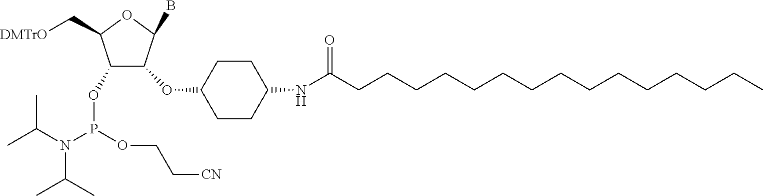 | Compound 29 |
| 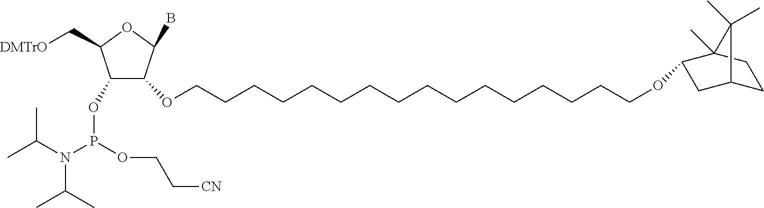 | Compound 30 |
| 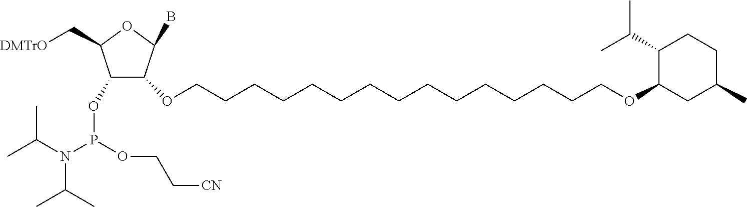 | Compound 31 |
| 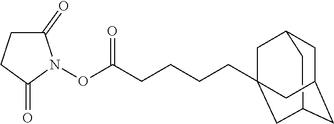 [SMILES: O=C(CCCCC12CC3CC(CC(C3)C1)C2)ON1C(=O)CCC1=O] | Compound 32 |
| 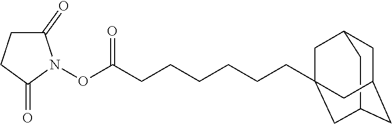 [SMILES: O=C(CCCCCCC12CC3CC(CC(C3)C1)C2)ON1C(=O)CCC1=O] | Compound 33 |
| 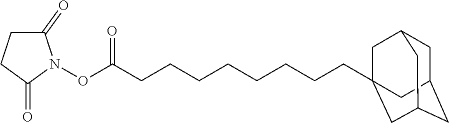 [SMILES: O=C(CCCCCCCCC12CC3CC(CC(C3)C1)C2)ON1C(=O)CCC1=O] | Compound 34 |
| 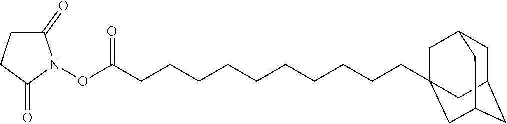 [SMILES: O=C(CCCCCCCCCCC12CC3CC(CC(C3)C1)C2)ON1C(=O)CCC1=O] | Compound 35 |
| 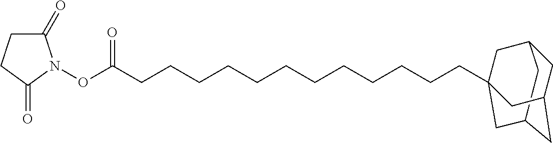 [SMILES: O=C(CCCCCCCCCCCCC12CC3CC(CC(C3)C1)C2)ON1C(=O)CCC1=O] | Compound 36 |
| 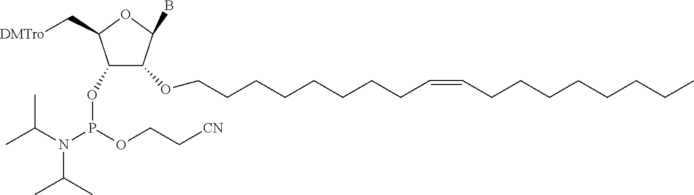 | Compound 37 |
| 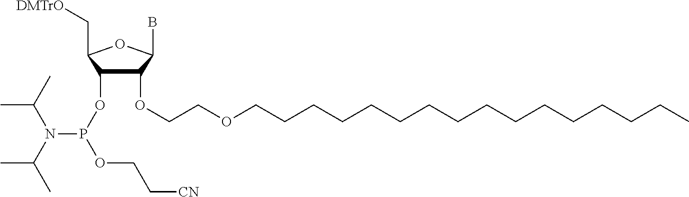 | Compound 38 |
| 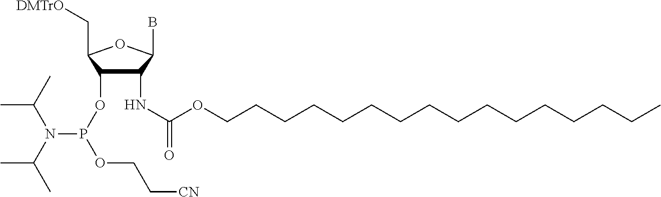 | Compound 39 |
| 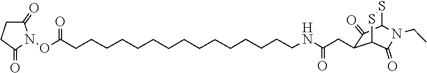 [SMILES: CCN1C(=O)C2SSC1C(=O)C2CC(=O)NCCCCCCCCCCCCCCCC(=O)ON1C(=O)CCC1=O] | Compound 40 |
| 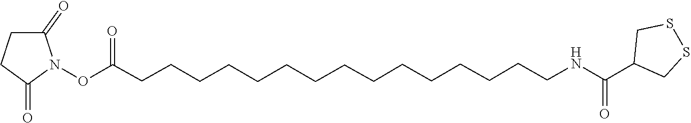 [SMILES: O=C(CCCCCCCCCCCCCCCNC(=O)C1CSSC1)ON1C(=O)CCC1=O] | Compound 41 |
| 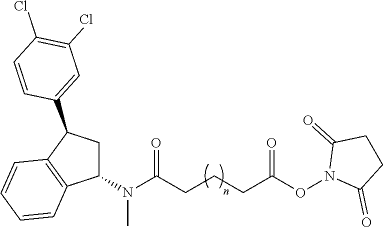 | Compound 42 |
| (n = 1 or n = 12) | |
| 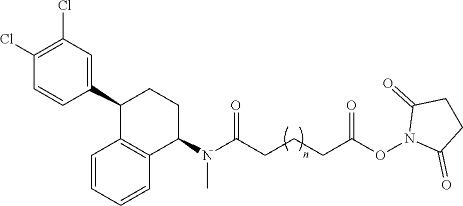 | Compound 43 |
| (n = 1 or n = 12) | |
| 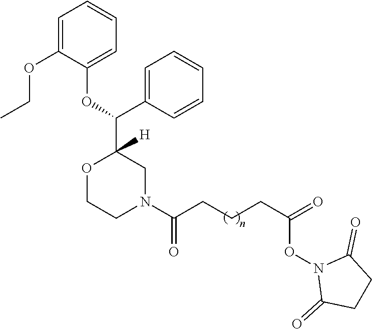 | Compound 44 |
| (n = 1 or n = 12) | |
| 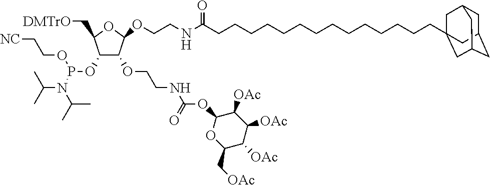 | Compound 45 |
| 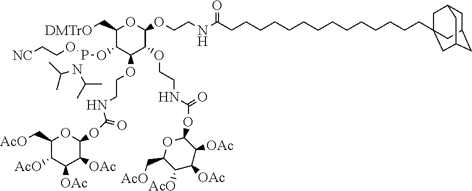 | Compound 46 |
| 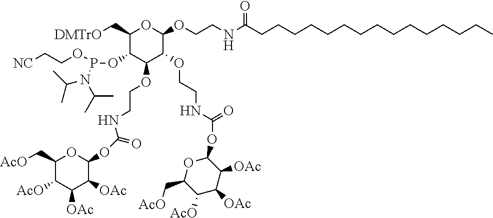 | Compound 47 |
| 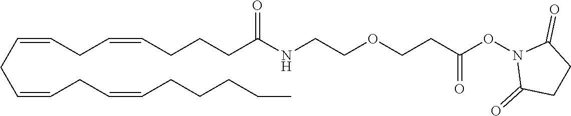 [SMILES: CCCCC/C=C\C/C=C\C/C=C\C/C=C\CCCC(=O)NCCOCCC(=O)ON1C(=O)CCC1=O] | Compound 47 |
| 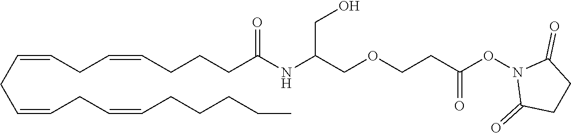 [SMILES: CCCCC/C=C\C/C=C\C/C=C\C/C=C\CCCC(=O)NC(CO)COCCC(=O)ON1C(=O)CCC1=O] | Compound 48 |
| 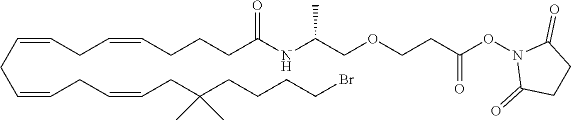 [SMILES: C[C@H](COCCC(=O)ON1C(=O)CCC1=O)NC(=O)CCC/C=C\C/C=C\C/C=C\C/C=C\CC(C)(C)CCCCBr] | Compound 49 |
| 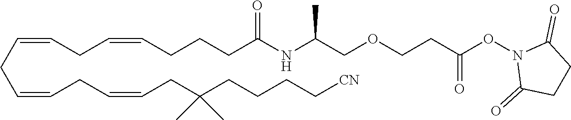 [SMILES: C[C@@H](COCCC(=O)ON1C(=O)CCC1=O)NC(=O)CCC/C=C\C/C=C\C/C=C\C/C=C\CC(C)(C)CCCCC#N] | Compound 50 |
| 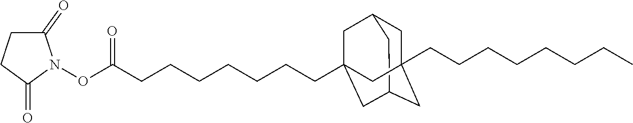 [SMILES: CCCCCCCCC12CC3CC(C1)CC(CCCCCCCC(=O)ON1C(=O)CCC1=O)(C3)C2] | Compound 51 |

For the compounds in Table 1 that have a nucleobase B (e.g., Compound 1), it is to be understood that the corresponding compounds where the “B” is replaced by a specific nucleobase provided herein are also specifically provided herein. In one embodiment, specifically provided herein are the corresponding compounds where the “B” is replaced by a uracil (U). In one embodiment, specifically provided herein are the corresponding compounds where the “B” is replaced by a cytosine (C). In one embodiment, specifically provided herein are the corresponding compounds where the “B” is replaced by an adenine (A). In one embodiment, specifically provided herein are the corresponding compounds where the “B” is replaced by a guanine (G). In some embodiments, reference to “Table 1” in this application includes these nucleobase-modified compounds.

### Oligonucleotides

In one embodiment, provided herein is an oligonucleotide comprising at least one lipophilic monomer of the following formula:

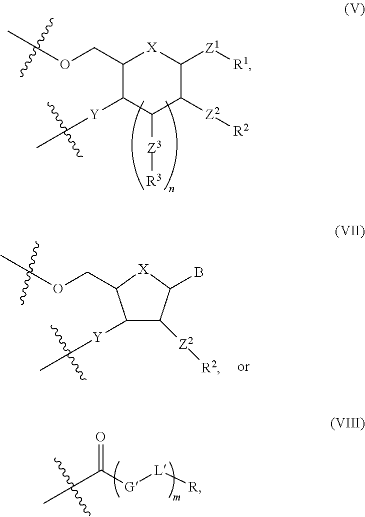

wherein X, Y, Z¹, Z², Z³, R¹, R², R³, B, G′, L′, R, n, and m are as defined herein or elsewhere (e.g., as defined in Formulas (I), (III), (IV), respectively, including subformulas thereof).

In one embodiment, the oligonucleotide comprises at least one lipophilic monomer of Formula (V):

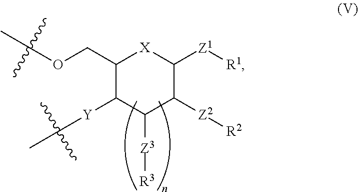

or a stereoisomer thereof, or a pharmaceutically acceptable salt thereof

In one embodiment, the oligonucleotide comprises at least one lipophilic monomer of Formula (V-A):

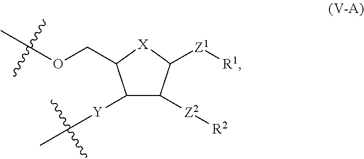

or a stereoisomer thereof, or a pharmaceutically acceptable salt thereof.

In one embodiment, the oligonucleotide comprises at least one lipophilic monomer of Formula (V-A-1):

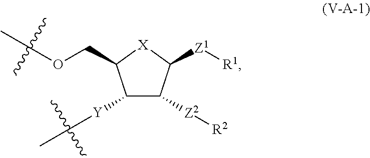

or a pharmaceutically acceptable salt thereof.

In one embodiment, the oligonucleotide comprises at least one lipophilic monomer of Formula (VI-A):

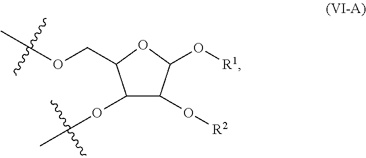

or a stereoisomer thereof, or a pharmaceutically acceptable salt thereof.

In one embodiment, the oligonucleotide comprises at least one lipophilic monomer of Formula (VI-A-1):

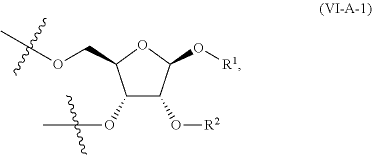

or a pharmaceutically acceptable salt thereof.

In one embodiment, the oligonucleotide comprises at least one lipophilic monomer of Formula (V-B):

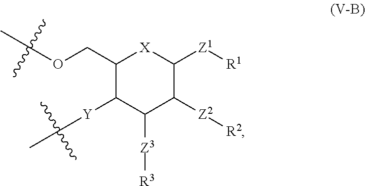

or a stereoisomer thereof, or a pharmaceutically acceptable salt thereof.

In one embodiment, the oligonucleotide comprises at least one lipophilic monomer of Formula (V-B-1):

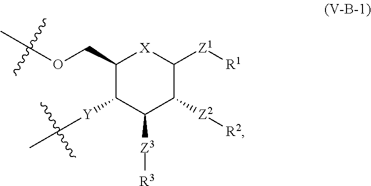

or a pharmaceutically acceptable salt thereof.

In one embodiment, the oligonucleotide comprises at least one lipophilic monomer of Formula (VI-B):

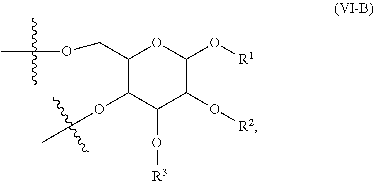

or a stereoisomer thereof, or pharmaceutically acceptable salt thereof.

In one embodiment, the oligonucleotide comprises at least one lipophilic monomer of Formula (VI-B-1):

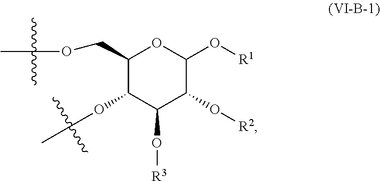

or pharmaceutically acceptable salt thereof

In one embodiment, the oligonucleotide comprises at least one lipophilic monomer of Formula (VII):

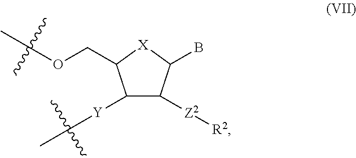

or a stereoisomer thereof, or a pharmaceutically acceptable salt thereof.

In one embodiment, the oligonucleotide comprises at least one lipophilic monomer of (VII-A):

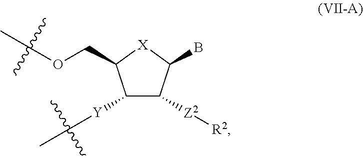

or a pharmaceutically acceptable salt thereof.

In one embodiment, the oligonucleotide comprises at least one lipophilic monomer of (VII-A-1):

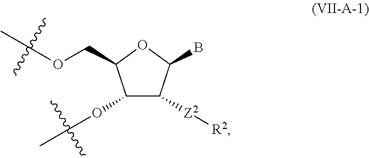

or a pharmaceutically acceptable salt thereof.

In one embodiment, the oligonucleotide comprises at least one lipophilic monomer of Formula (VIII):

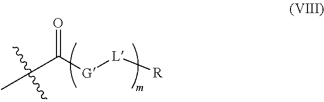

or a stereoisomer thereof, or a pharmaceutically acceptable salt thereof.

In one embodiment, the oligonucleotide comprises at least one lipophilic monomer in Table 2, or a stereoisomer thereof, or pharmaceutically acceptable salt thereof (wherein B is a modified or unmodified nucleobase).

TABLE 2

| | |
|---|---|
| 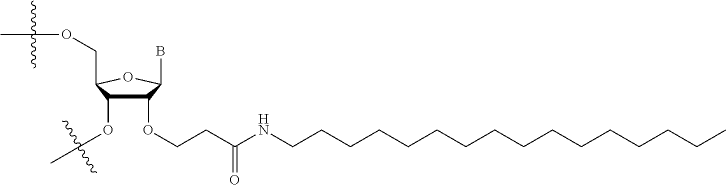 | Compound 1′ |
| (L5 when B is U) | |
| 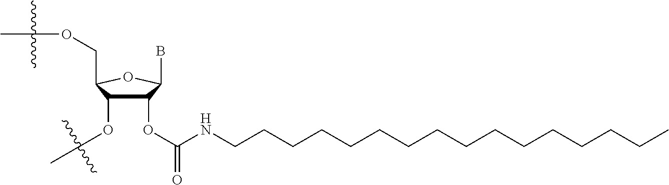 | Compound 2′ |
| (L4 when B is U; L21 when B is A; L22 | |
| when B is C; L23 when B is G) | |
| 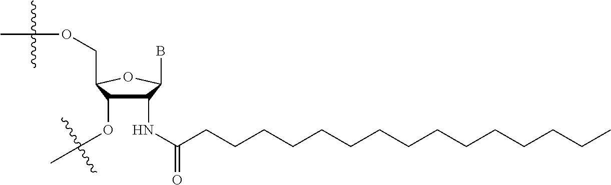 | Compound 3′ |
| (L3 when B is U) | |
| 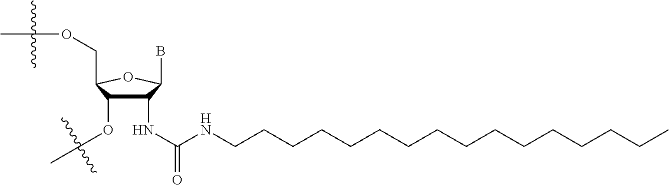 | Compound 4′ |
| (L2) | |
| 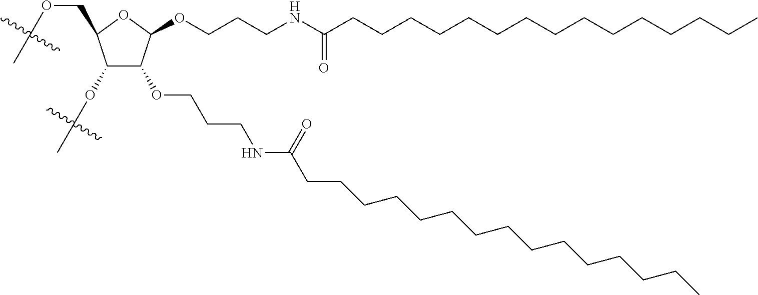 [SMILES: *OC[C@H]1O[C@@H](OCCCNC(=O)CCCCCCCCCCCCCCC)[C@H](OCCCNC(=O)CCCCCCCCCCCCCCC)[C@@H]1O*] | Compound 5′ |
| 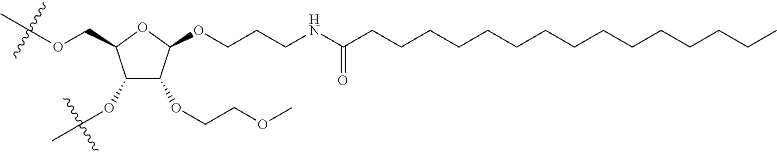 [SMILES: *OC[C@H]1O[C@@H](OCCCNC(=O)CCCCCCCCCCCCCCC)[C@H](OCCOC)[C@@H]1O*] | Compound 6′ |
| 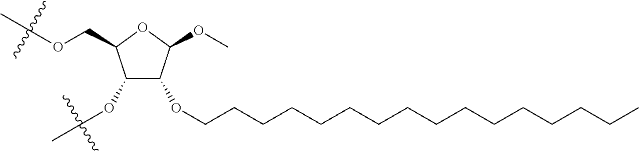 [SMILES: *OC[C@H]1O[C@@H](OC)[C@H](OCCCCCCCCCCCCCCCC)[C@@H]1O*] | Compound 7′ |
| (L9) | |
| 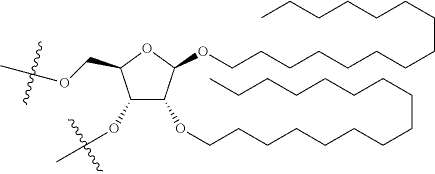 [SMILES: *OC[C@H]1O[C@@H](OCCCCCCCCCCCCCCC)[C@H](OCCCCCCCCCCCCCCCC)[C@@H]1O*] | Compound 8′ |
| (L11) | |
| 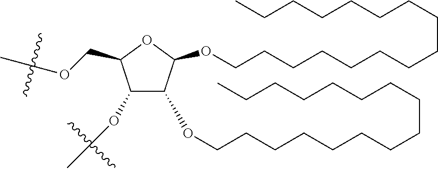 [SMILES: *OC[C@H]1O[C@@H](OCCCCCCCCCCCCCCCC)[C@H](OCCCCCCCCCCCCCCCC)[C@@H]1O*] | Compound 9′ |
| (L10) | |
| 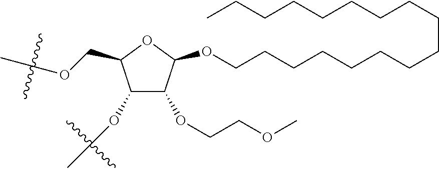 [SMILES: *OC[C@H]1O[C@@H](OCCCCCCCCCCCCCCCCC)[C@H](OCCOC)[C@@H]1O*] | Compound 10′ |
| (L8) | |
| 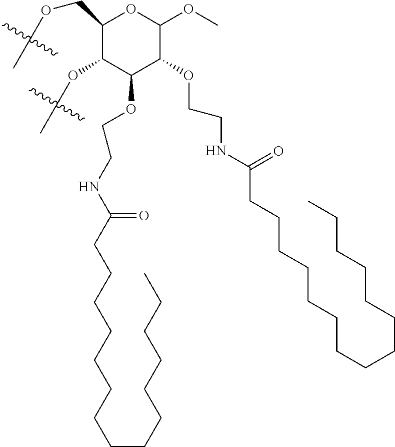 [SMILES: *OC[C@H]1OC(OC)[C@H](OCCNC(=O)CCCCCCCCCCCCCCC)[C@@H](OCCNC(=O)CCCCCCCCCCCCCCC)[C@@H]1O*] | Compound 11′ |
| 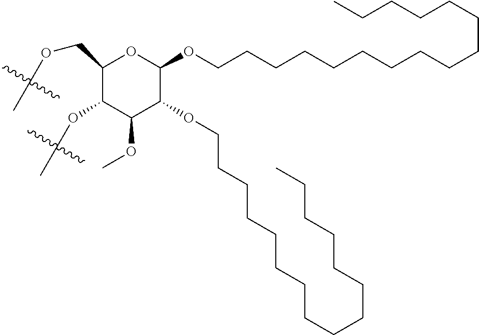 [SMILES: *OC[C@H]1O[C@@H](OCCCCCCCCCCCCCCCC)[C@H](OCCCCCCCCCCCCCCCC)[C@@H](OC)[C@@H]1O*] | Compound 12′ |
| (L12) | |
| 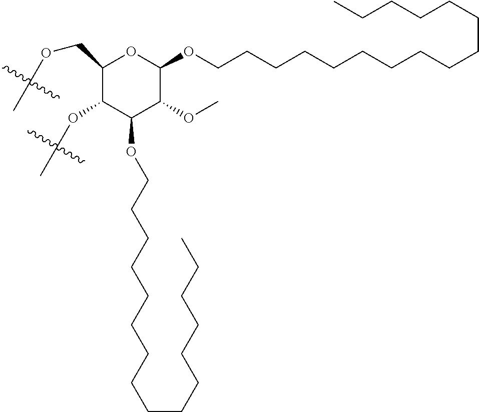 [SMILES: *OC[C@H]1O[C@@H](OCCCCCCCCCCCCCCCC)[C@H](OC)[C@@H](OCCCCCCCCCCCCCCCC)[C@@H]1O*] | Compound 13′ |
| (L24) | |
| 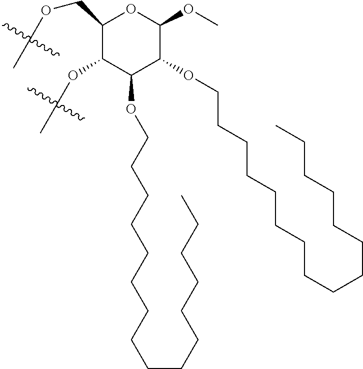 [SMILES: *OC[C@H]1O[C@@H](OC)[C@H](OCCCCCCCCCCCCCCCC)[C@@H](OCCCCCCCCCCCCCCCC)[C@@H]1O*] | Compound 14′ |
| 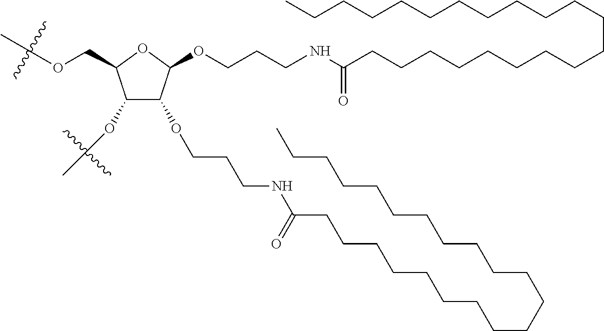 [SMILES: *OC[C@H]1O[C@@H](OCCCNC(=O)CCCCCCCCCCCCCCCCCCCCC)[C@H](OCCCNC(=O)CCCCCCCCCCCCCCCCCCCCC)[C@@H]1O*] | Compound 15′ |
| 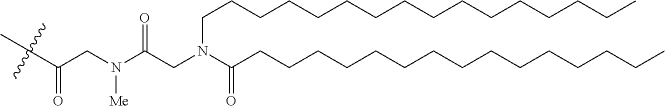 [SMILES: *C(=O)CN(C)C(=O)CN(CCCCCCCCCCCCCCCC)C(=O)CCCCCCCCCCCCCCC] | Compound 16′ |
| (L18) | |
| 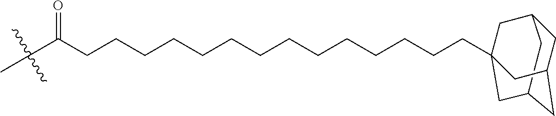 [SMILES: *C(=O)CCCCCCCCCCCCCCC12CC3CC(CC(C3)C1)C2] | Compound 17′ |
| (L17) | |
| 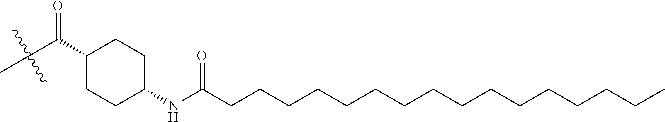 [SMILES: *C(=O)[C@H]1CC[C@@H](NC(=O)CCCCCCCCCCCCCCCC)CC1] | Compound 18′ |
| (L14) | |
| 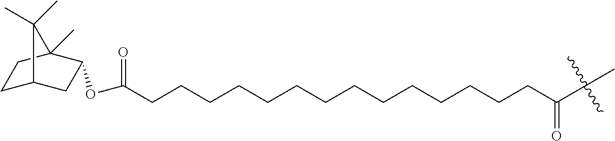 [SMILES: *C(=O)CCCCCCCCCCCCCCC(=O)O[C@@H]1CC2CCC1(C)C2(C)C] | Compound 19′ |
| (L15) | |
| 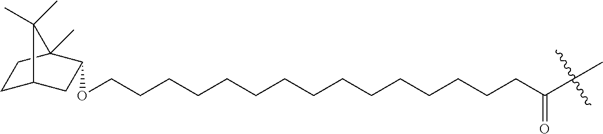 [SMILES: *C(=O)CCCCCCCCCCCCCCCO[C@@H]1CC2CCC1(C)C2(C)C] | Compound 20′ |
| (L16) | |
| 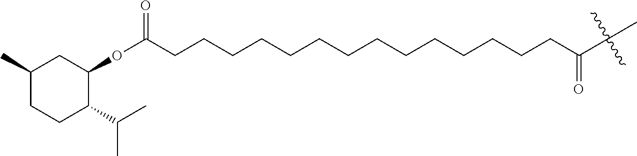 [SMILES: *C(=O)CCCCCCCCCCCCCCC(=O)O[C@@H]1C[C@H](C)CC[C@H]1C(C)C] | Compound 22′ |
| (L19) | |
| 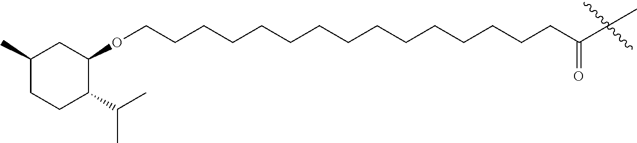 [SMILES: *C(=O)CCCCCCCCCCCCCCCO[C@@H]1C[C@H](C)CC[C@H]1C(C)C] | Compound 23′ |
| (L20) | |
| 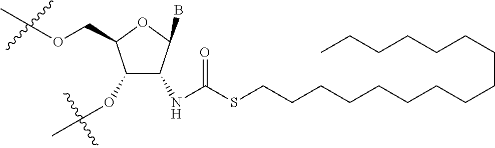 | Compound 24′ |
| 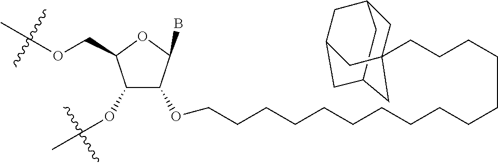 | Compound 25′ |
| (L13 when B is U) | |
| 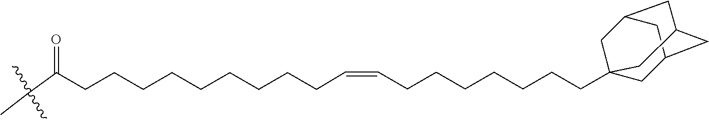 [SMILES: *C(=O)CCCCCCCCC/C=C\CCCCCCCC12CC3CC(CC(C3)C1)C2] | Compound 26′ |
| 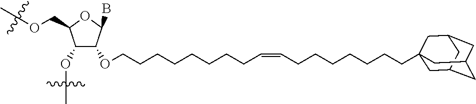 | Compound 27′ |
| 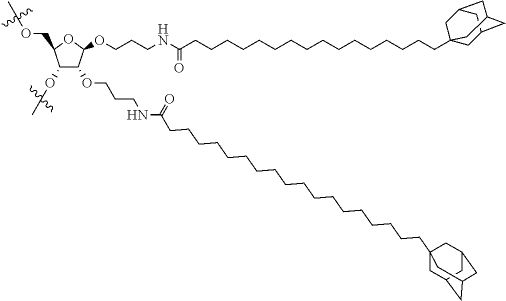 [SMILES: *OC[C@H]1O[C@@H](OCCCNC(=O)CCCCCCCCCCCCCCCCC23CC4CC(CC(C4)C2)C3)[C@H](OCCCNC(=O)CCCCCCCCCCCCCCCCCCC23CC4CC(CC(C4)C2)C3)[C@@H]1O*] | Compound 28′ |
| 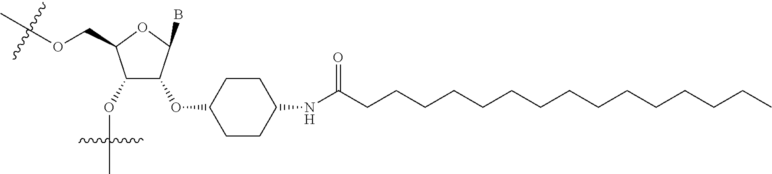 | Compound 29′ |
| 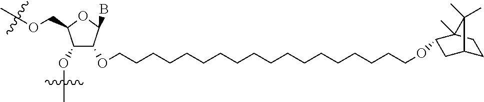 | Compound 30′ |
| 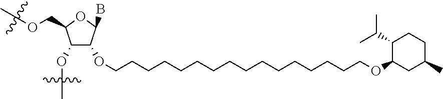 | Compound 31′ |
| 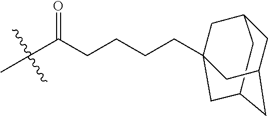 [SMILES: *C(=O)CCCCC12CC3CC(CC(C3)C1)C2] | Compound 32′ |
| 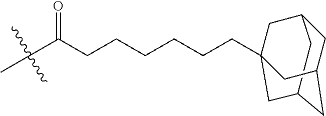 [SMILES: *C(=O)CCCCCCC12CC3CC(CC(C3)C1)C2] | Compound 33′ |
| 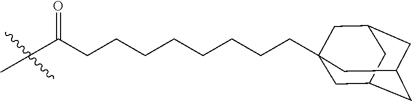 [SMILES: *C(=O)CCCCCCCCC12CC3CC(CC(C3)C1)C2] | Compound 34′ |
| 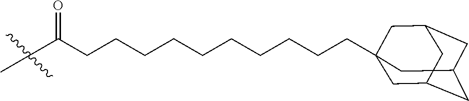 [SMILES: *C(=O)CCCCCCCCCCC12CC3CC(CC(C3)C1)C2] | Compound 35′ |
| 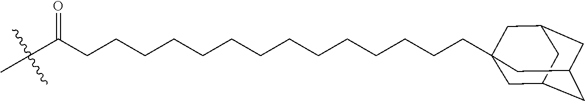 [SMILES: *C(=O)CCCCCCCCCCCCCCC12CC3CC(CC(C3)C1)C2] | Compound 36′ |
| 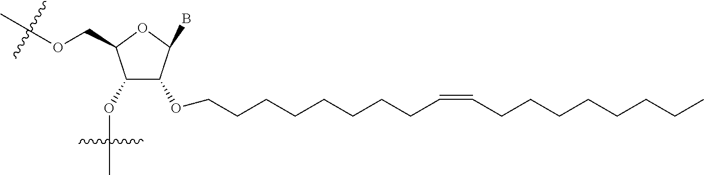 | Compound 37′ |
| (L1 when B is U) | |
| 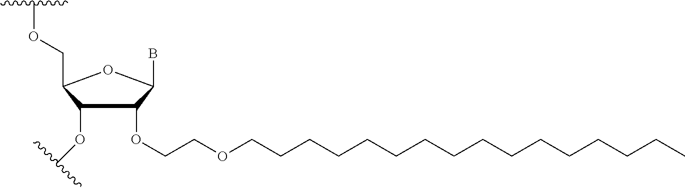 | Compound 38′ |
| (L6 when B is U) | |
| 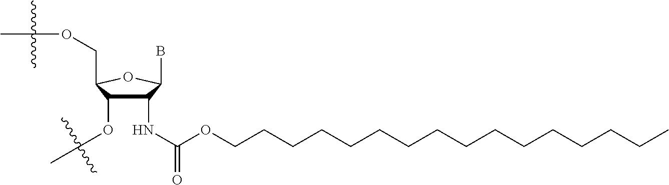 | Compound 39′ |
| (L7 when B is U) | |
| 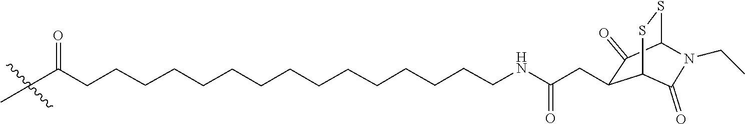 [SMILES: *C(=O)CCCCCCCCCCCCCCCNC(=O)CC1C(=O)C2SSC1C(=O)N2CC] | Compound 40′ |
| 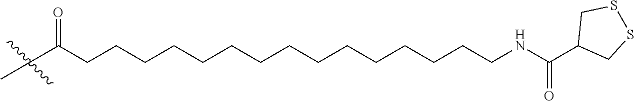 [SMILES: *C(=O)CCCCCCCCCCCCCCCNC(=O)C1CSSC1] | Compound 41′ |
| 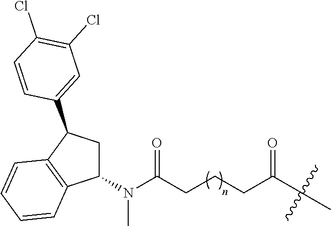 | Compound 42′ |
| (n = 1 or n = 12) | |
| 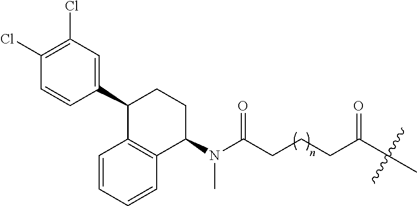 | Compound 43′ |
| (n = 1 or n = 12) | |
| 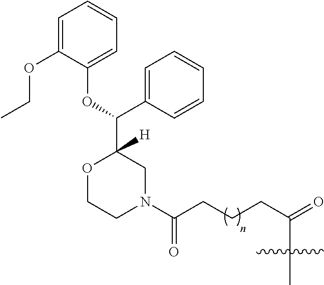 | Compound 44′ |
| (n = 1 or n = 12) | |
| 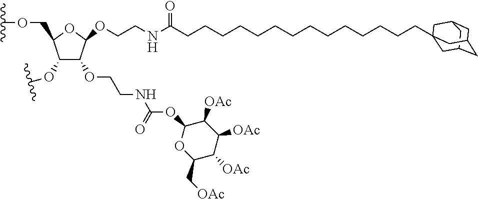 | Compound 45′ |
| 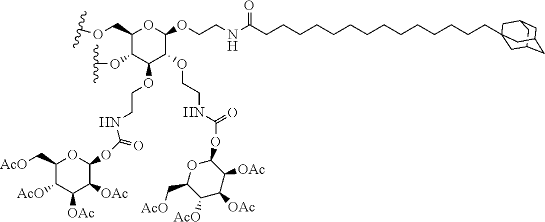 | Compound 46′ |
| 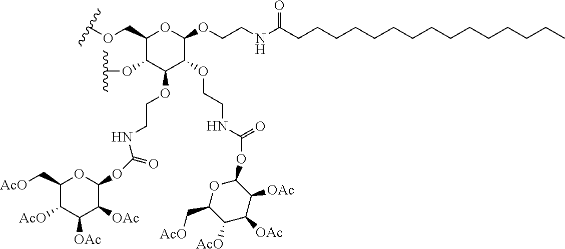 | Compound 47′ |
| 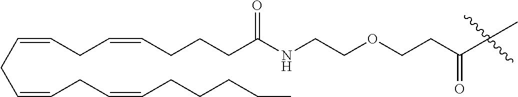 [SMILES: *C(=O)CCOCCNC(=O)CCC/C=C\C/C=C\C/C=C\C/C=C\CCCCC] | Compound 47′ |
| 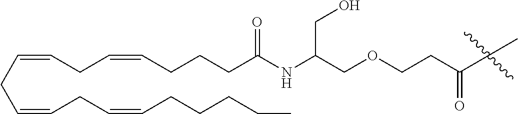 [SMILES: *C(=O)CCOCC(CO)NC(=O)CCC/C=C\C/C=C\C/C=C\C/C=C\CCCCC] | Compound 48′ |
| 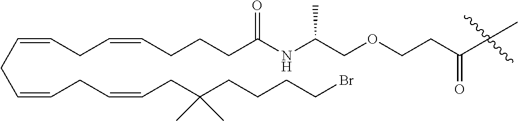 [SMILES: *C(=O)CCOC[C@@H](C)NC(=O)CCC/C=C\C/C=C\C/C=C\C/C=C\CC(C)(C)CCCCBr] | Compound 49′ |
| 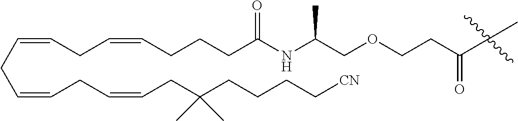 [SMILES: *C(=O)CCOC[C@H](C)NC(=O)CCC/C=C\C/C=C\C/C=C\C/C=C\CC(C)(C)CCCCC#N] | Compound 50′ |
| 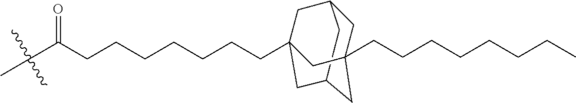 [SMILES: *C(=O)CCCCCCCC12CC3CC(CC(CCCCCCCC)(C3)C1)C2] | Compound 51′ |

For the compounds in Table 2 that have a nucleobase B (e.g., Compound 1′), it is to be understood that the corresponding compounds where the “B” is replaced by a specific nucleobase provided herein are also specifically provided herein. In one embodiment, specifically provided herein are the corresponding compounds where the “B” is replaced by a uracil (U). In one embodiment, specifically provided herein are the corresponding compounds where the “B” is replaced by a cytosine (C). In one embodiment, specifically provided herein are the corresponding compounds where the “B” is replaced by an adenine (A). In one embodiment, specifically provided herein are the corresponding compounds where the “B” is replaced by a guanine (G). In some embodiments, reference to “Table 2” in this application includes these nucleobase-modified compounds.

In one embodiment, the oligonucleotide is an antisense, an antagomir, a microRNA, a siRNA, a pre-microRNA, an antimir, a ribozyme, a RNA activator, a U1 adaptor, an immune stimulator or an aptamer. In one embodiment, the oligonucleotide is a double strand RNA (dsRNA). In one embodiment, the oligonucleotide is a siRNA.

In one embodiment, the oligonucleotide is a siRNA comprising:
an antisense strand which is complementary to a target gene;
a sense strand which is complementary to said antisense strand.

In one embodiment, the lipophilic monomer is present in either the antisense strand or the sense strand. In one embodiment, the lipophilic monomer is present in the sense strand. In one embodiment, the lipophilic monomer is present in the antisense strand.

In one embodiment, the lipophilic monomer is conjugated to a terminal position (e.g., the 3′-end or 5′-end) of the oligonucleotide. In one embodiment, the lipophilic monomer is conjugated to the 3′-end of the oligonucleotide. In one embodiment, the lipophilic monomer is conjugated to the 5′-end of the oligonucleotide. In one embodiment, the lipophilic monomer is conjugated to a terminal position (e.g., the 3′-end or 5′-end) of the sense strand or the antisense strand. In one embodiment, the lipophilic monomer is conjugated to the 3′-end of the sense strand. In one embodiment, the lipophilic monomer is conjugated to the 5′-end of the sense strand. In one embodiment, the lipophilic monomer is conjugated to the 3′-end of the antisense strand. In one embodiment, the lipophilic monomer is conjugated to the 5′-end of the antisense strand.

In one embodiment, the lipophilic monomer is conjugated directly to the 3′-end or 5′-end of the oligonucleotide. In one embodiment, the lipophilic monomer is conjugated to the 3′-end or 5′-end of the oligonucleotide via a linker group.

In one embodiment, a lipophilic monomer of Formula (V) (or any subformula thereof) is conjugated to a terminal position (e.g., the 3′-end or 5′-end) of the oligonucleotide. In one embodiment, the oligonucleotide comprises the following structure:

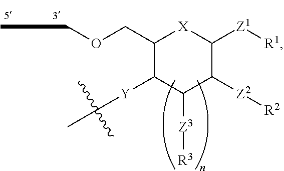

wherein [symbol] comprises one or more nucleotides of the oligonucleotide in the 5′-3′ orientation. In one embodiment, the [symbol] comprises at the 3′-end a linker consisting of 1 to 3 nucleotides (i.e., the O of the lipophilic monomer of Formula (V) is connected directed to the nucleotide linker, which is connected to the remaining of oligonucleotide). In one embodiment, the linker is dTdT. In one embodiment, Y is connected to a hydrogen.

In one embodiment, the oligonucleotide comprises the following structure:

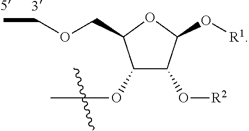

In one embodiment, the oligonucleotide comprises the following structure:

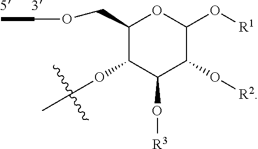

In one embodiment, the oligonucleotide comprises the following structure:

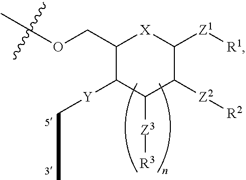

wherein [symbol] comprises one or more nucleotides of the oligonucleotide in the 5′-3′ orientation. In one embodiment, the [symbol] comprises at the 5′-end a linker consisting of 1 to 3 nucleotides (i.e., the Y of the lipophilic monomer of Formula (V) is connected directed to the nucleotide linker, which is connected to the remaining of oligonucleotide). In one embodiment, the linker is dTdT. In one embodiment, O is connected to a hydrogen.

In one embodiment, the oligonucleotide comprises the following structure:

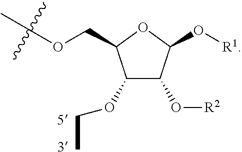

In one embodiment, the oligonucleotide comprises the following structure:

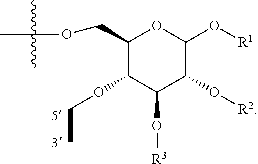

In one embodiment, a lipophilic monomer of Formula (VII) (or any subformula thereof) is conjugated to a terminal position (e.g., the 3′-end or 5′-end) of the oligonucleotide. In one embodiment, the oligonucleotide comprises the following structure:

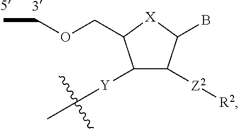

wherein [symbol] comprises one or more nucleotides of the oligonucleotide in the 5′-3′ orientation.

In one embodiment, the oligonucleotide comprises the following structure:

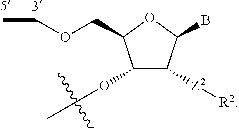

In one embodiment, the oligonucleotide comprises the following structure:

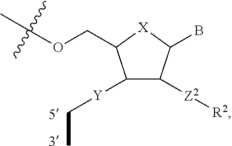

wherein [symbol] comprises one or more nucleotides of the oligonucleotide in the 5′-3′ orientation.

In one embodiment, the oligonucleotide comprises the following structure:

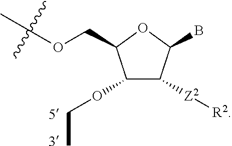

In one embodiment, a lipophilic monomer of Formula (VIII) (or any subformula thereof) is conjugated to a terminal position (e.g., the 3′-end or 5′-end) of the oligonucleotide. In one embodiment, the oligonucleotide comprises the following structure:

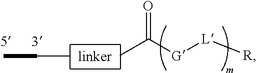

wherein [symbol] comprises one or more nucleotides of the oligonucleotide in the 5′-3′ orientation, and [symbol] linker is a linker.

In one embodiment, the oligonucleotide comprises the following structure:

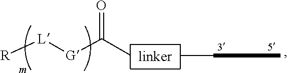

wherein [symbol] comprises one or more nucleotides of the oligonucleotide in the 5′-3′ orientation, and [symbol] linker is a linker.

In one embodiment, the linker is

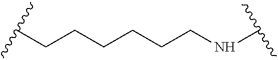 [SMILES: *CCCCCCN*]

(“C6NH”). In one embodiment, the linker is

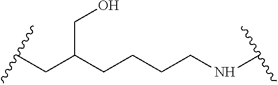 [SMILES: *CC(CO)CCCCN*]

(“C7NH”). NH in the C6NH or C7NH linker is connected to the lipid. In one embodiment, the linker is located at 5′-end of the oligonucleotide. In one embodiment, the linker is located at 3′-end of the oligonucleotide. In one embodiment, the linker is located at an internal position of the oligonucleotide. In one embodiment, a C6NH linker is located at 5′-end of the oligonucleotide. In one embodiment, a C6NH linker is located at 3′-end of the oligonucleotide. In one embodiment, a C6NH linker is located at an internal position of the oligonucleotide. In one embodiment, a C7NH linker is located at 3′-end of the oligonucleotide.

In one embodiment, the lipophilic monomer is located at an internal position of the oligonucleotide. In one embodiment, the lipophilic monomer is located at an internal position of the sense strand or the antisense strand. In one embodiment, the lipophilic monomer is located at an internal position of the sense strand. In one embodiment, the lipophilic monomer is located at an internal position of the antisense strand.

In one embodiment, a lipophilic monomer of Formula (VII) (or any subformula thereof) is located at an internal position of the oligonucleotide. In one embodiment, the oligonucleotide comprises the following structure:

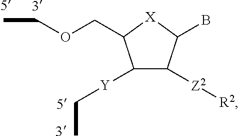

wherein each [symbol] independently comprises one or more nucleotides of the oligonucleotide in the 5′-3′ orientation.

In one embodiment, the oligonucleotide comprises the following structure:

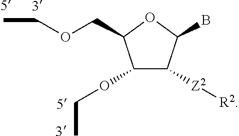

In one embodiment, a lipophilic monomer of Formula (VIII) (or any subformula thereof) is located at an internal position of the oligonucleotide. In one embodiment, the oligonucleotide comprises the following structure:

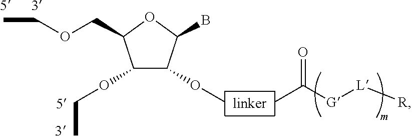

wherein [symbol] each independently comprises one or more nucleotides of the oligonucleotide in the 5′-3′ orientation.

[symbol]

In one embodiment, the sense and antisense strands are each 15 to 30 nucleotides in length. In one embodiment, the sense and antisense strands are each 15 to 25 nucleotides in length. In one embodiment, the sense and antisense strands are each 19 to 25 nucleotides in length. In one embodiment, said sense and antisense strands are each 21 to 23 nucleotides in length.

In one embodiment, the oligonucleotide further comprises a targeting ligand.

In one embodiment, provided herein is a method of delivering an oligonucleotide to a cell, comprising contacting an oligonucleotide provided herein with the cell. In some embodiments, the cells are brain cells. In some embodiments, the cells are in a subject, such as a mouse, non-human primate or human. In one embodiment, the administration results in at an increased amount of the oligonucleotide delivered to the cell, as compared to delivery of an oligonucleotide that is identical but does not contain the lipid conjugate (lipophilic monomer) provided herein. In some embodiments, the increased amount of the oligonucleotide delivered is determined by increase in therapeutic activity of the oligonucleotide.

In one embodiment, provided herein is a method of reducing the expression of a target gene in a cell, comprising contacting said cell with an oligonucleotide provided herein. In one embodiment, provided herein is a method of reducing the expression of a target gene in a subject, comprising administering to the subject an oligonucleotide provided herein.

In one embodiment, without being limited by a particular theory, an oligonucleotide that comprises at least one lipophilic monomer provided herein exhibits improved pharmacodynamics profiles than the corresponding oligonucleotide that does not comprise the lipophilic monomer.

### Chemical Modifications to Nucleotides

In specific embodiments, a dsRNA (e.g., siRNA) agent described herein comprises one or more nucleotide modifications. Nucleotide modifications include, for example, end modifications, e.g., 5′-end modifications (e.g., phosphorylation, conjugation, inverted linkages) or 3′-end modifications (e.g., conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. In some embodiments, a dsRNA agent comprises at least one modification selected from the group consisting of modified internucleoside linkage, modified nucleobase, modified sugar, and any combinations thereof. Without limitations, such a modification can be present anywhere in the dsRNA agent (e.g., in the sense strand, antisense strand, or both strands).

In some embodiments, a dsRNA agent comprises one or more modified sugar modifications, such as one or more substituted sugar moieties. In some embodiments, a dsRNA agent described herein includes one of the following at the 2′-position: H; F; or OCH₃ (OMe).

In some embodiments, a dsRNA agent described herein includes one or more glycol nucleic acids (GNA). Typically, GNA is an acyclic nucleic acid analogue, wherein its repeating glycol units are linked by phosphodiester bonds, differing from RNA's ribose sugar-phosphodiester backbone composition.

In certain embodiments, a dsRNA agent described herein includes one or more terminal modifications, for example, 5′-terminal phosphorylation, conjugation, or inverted linkages. In some embodiments, the terminal modifications include a 5′-phosphate, for example, a 5′-terminal phosphate on the antisense strand of a dsRNA agent.

In some embodiments, a dsRNA agent includes a sense strand and/or an antisense strand with an inverted abasic nucleotide. In one embodiment, the sense strand contains an inverted abasic nucleotide at 3′ end. In another embodiment, the sense strand contains an inverted abasic nucleotide at 5′ end. In some embodiments, the sense strand contains an inverted abasic nucleotide at the 5′ and 3′ end.

In some embodiments, a dsRNA agent comprises a phosphate or phosphate mimic at the 5′-end of the antisense strand. In one embodiment, the phosphate mimic is a 5′-vinyl phosphonate (VP).

In some embodiments, a dsRNA agent comprises one or more modified nucleotides. In specific embodiments, the modified nucleotides are selected from the group consisting of a 2′O-methyl modified nucleotide, a deoxy-nucleotide, a 2′-fluoro modified nucleotide, a 2′-O-methyl-uridine, an inverted abasic nucleotide, a nucleotide comprising S-glycol nucleic acid (GNA), an unlocked nucleotide, a 5′-vinylphosphonate-2′-O-methyl-uridine, and combinations thereof.

In some embodiments, a dsRNA agent comprises one or more modified internucleoside linkages (i.e., a modified RNA backbone). Modified internucleoside linkages include, for example, phosphorothioates (e.g., phosphoromonothioates). Various salts, mixed salts and free acid forms are also included. In some embodiments, a dsRNA agent described herein is in a free acid form. In other embodiments, a dsRNA agent described herein is in a salt form. In one embodiment, a dsRNA agent described herein is in a sodium salt form. In certain embodiments, when a dsRNA agent described herein is in the salt form, cations of the salt (e.g., sodium cations) are present at the agent as counterions for substantially all of the electronegative groups (e.g., phosphodiester and/or phosphorothioate groups) present in the agent. In some embodiments, the counterion is a condensed counterion. In specific embodiments, the counterion is a condensed sodium cation. In some embodiments, the condensed counterion is hydrated. In specific embodiments, the condensed counterion is a hydrated sodium cation. Agents in which substantially all of the phosphodiester and/or phosphorothioate linkages have a counterion include not more than 5, 4, 3, 2, or 1 phosphodiester and/or phosphorothioate linkages without a counterion. In other words, the electronegative potential of the dsRNA agent is neutralized or substantially neutralized by counterion condensation around the dsRNA. In some embodiments, when a dsRNA agent described herein is in the sodium salt form, sodium ions are present around the agent as counterions for substantially all of the phosphodiester and/or phosphorothioate groups present in the agent.

The phosphate group of an internucleoside phosphodiester linkage can be modified by replacing one of the oxygens with a different substituent. One result of this modification can be increased resistance of the oligonucleotide to nucleolytic breakdown. Another result of this modification can be increased stability of hybridized single-stranded RNA (ssRNA) in the dsRNA agent. An example of a modified phosphate group includes phosphorothioates (e.g., phosphoromonothioates),

In some embodiments, a dsRNA agent comprises an RNA mimetic, in which both the sugar and the internucleoside linkage, i.e., the backbone, of the nucleotide units are replaced with alternate groups. In specific embodiments, the base units are maintained for hybridization with an appropriate target sequence.

A dsRNA agent described herein can contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids. Included in a dsRNA agent provided herein are all such possible isomers, as well as their racemic and optically pure forms.

### Moieties Linked to Nucleotide Sequence

In some embodiments, a dsRNA agent described herein is conjugated to one or more non-nucleotide groups. The non-nucleotide group can, e.g., enhance targeting, delivery or attachment of the dsRNA agent. The non-nucleotide group can be covalently linked to the 3′ end, 5′ end, and/or internally to either the sense strand or the antisense strand of the dsRNA agent. The non-nucleotide group can be covalently linked to the 3′ end, 5′ end, both the 3′ end and 5′ end, internally, both the 3′ end and internally, both 5′ end and internally, or at the 3′ end, the 5′ end, and internally of the sense strand and/or the antisense strand of the dsRNA agent. In some embodiments, a dsRNA agent described herein contains a non-nucleotide group linked to the 3′ end, 5′ end, both the 3′ end and 5′ end, internally, both the 3′ end and internally, both 5′ end and internally, or at the 3′ end, the 5′ end and internally of the sense strand. In certain embodiments, a dsRNA agent described herein contains a non-nucleotide group linked to the 5′ end of the sense strand. In certain embodiments, a dsRNA agent described herein contains a non-nucleotide group linked to the 3′ end of the sense strand. In certain embodiments, a dsRNA agent described herein contains a non-nucleotide group linked to the sense strand internally. A non-nucleotide group may be linked directly or indirectly to the dsRNA agent via a linker/linking group.

In some embodiments, a linking group is conjugated to the dsRNA agent. The linking group facilitates covalent linkage of the agent to a targeting ligand or delivery polymer or delivery vehicle. The linking group can be linked to the 3′ end, the 5′ end, and/or internally of the dsRNA agent sense strand. In some embodiments, the linking group is linked to the dsRNA agent sense strand. In some embodiments, the linking group is conjugated to the 5′ end, 3′ end, and/or internally to the sense strand of a dsRNA agent. In some embodiments, a linking group is conjugated to the 5′ end the sense strand of a dsRNA agent. In some embodiments, a linking group is conjugated to the 3′ end of the sense strand of a dsRNA agent. In some embodiments, a linking group is conjugated internally to the sense strand of a dsRNA agent.

Typically, a linker or linking group is a connection between two atoms that links one chemical group (such as a dsRNA agent) or segment of interest to another chemical group (such as a targeting group or delivery polymer) or segment of interest via one or more covalent bonds. A labile linkage contains a labile bond. A linkage may optionally include a spacer that increases the distance between the two joined atoms. A spacer may further add flexibility and/or length to the linkage.

Any of the dsRNA agent nucleotide sequences listed in Table 3, whether modified or unmodified, may contain a 3′ end, 5′ end, and/or internal targeting ligand and/or linking group. Any of the dsRNA agent duplexes listed in Table 1, 2, 3, 4, 5, or 9, whether modified or unmodified, may further comprise a targeting ligand and/or linking group, and the targeting ligand or linking group may be attached to the 3′ end, 5′ end, and/or internally to either the sense strand or the antisense strand of the dsRNA agent duplex.

### Delivery Vehicles

In some embodiments, a delivery vehicle may be used to deliver a dsRNA agent described herein to a cell or tissue.

### Compositions

In one aspect, provided herein are compositions (e.g., pharmaceutical compositions) comprising a dsRNA agent described herein. The composition may further comprises a pharmaceutically acceptable carrier.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### EXAMPLES

Certain embodiments of the invention are illustrated by the following non-limiting examples.

### Experimental Procedure for Synthesis of Oligonucleotides Lipid Conjugates

The synthesis was carried out by phosphoramidite approach on solid phase following standard RNA synthesis protocols. All oligonucleotides were prepared on a MerMade 12 synthesizer on a 10 μmole scale using universal or custom supports. A typical synthetic cycle comprises the following four steps 1. Detritylation; 2. Coupling; 3. Capping; and 4. Oxidation/sulfurization.

Monomers for RNA phosphoramidites with standard protecting groups, 5′-O-dimethoxytrityl-N⁶-benzoyl-2′-O-(tert-butyldimethylsilyl)-adenosine 3′-O—(N,N′-diisopropyl-cyanoethylphosphoramidite), 5′-O-dimethoxytrityl-N⁴-acetyl-2′-O-(tert-butyldimethylsilyl-cytidine-3′-O—(N,N′-diisopropyl-2-cyanoethylphosphoramidite), 5′-O-dimethoxytrityl-N²-isobutryl-2′-O-(tert-butyldimethylsilyl)guanosine 3′-O—(N,N′-diisopropyl-2-cyanoethylphosphoramidite) and 5′-O-dimethoxytrityl-2′-O-(tert-butyldimethylsilyl)uridine 3′-O—(N,N′-diisopropyl-2-cyanoethylphosphoramidite) were used for the synthesis.

The 2′-modified monomers were comprised of 2′-fluoro phosphoramidites and 2′-OMe phosphoramidites, 5′-O-dimethoxytrityl-N⁴-acetyl-2′-fluoro-cytidine 3′-O—(N,N′-diisopropyl-2-cyanoethylphosphoramidite), 5′-O-dimethoxytrityl-2′-fluoro-uridine 3′-O—(N,N′-diisopropyl-2-cyanoethylphosphoramidite), 5′-O-dimethoxytrityl-N⁶-benzoyl-2′-fluoro-adenosine 3′-O—(N,N′-diisopropyl-2-cyanoethylphosphoramidite), 5′-O-dimethoxytrityl-N²-isobutryl-2′-fluoro-guanosine 3′-O—(N,N′-diisopropyl-2-cyanoethylphosphoramidite), 5′-O-dimethoxytrityl-N⁴-acetyl-2′-OMe-cytidine 3′-O—(N,N′-diisopropyl-2-cyanoethylphosphoramidite), 5′-O-dimethoxytrityl-2′-OMe-uridine-3′-O—(N,N′-diisopropyl-2-cyanoethylphosphoramidite), 5′-O-dimethoxytrityl-N⁶-benzoyl-2′-OMe-adenosine 3′-O—(N,N′-diisopropyl-2-cyanoethylphosphoramidite) and 5′-O-dimethoxytrityl-N²-isobutryl-2′-OMe-guanosine 3′-O—(N,N′-diisopropyl-2-cyanoethylphosphoramidite).

All phosphoramidites were used at a concentration of 0.1M in acetonitrile except for guanosine, which was used at 0.1M concentration in 20% DMF/MeCN. Detritylation was performed with 3% dichloroacetic acid in dichloromethane for 3 minutes. Coupling was performed with a 0.45M solution of 5-(ethylthio)tetrazole in MeCN and coupling time was 12 minutes. Oxidation of the internucleotide phosphite to the phosphate was carried out using standard 0.02M iodine in MeCN/pyridine/water (70:20:10). Phosphorothioate was introduced by the oxidation of phosphite to phosphorothioate by using a 0.1M solution of xanthane hydride in pyridine. Capping was performed with THF/acetic anhydride/pyridine (80:10:10) and 1-methylimidazole/acetonitrile (20:80 v/v) solution. A complete synthetic cycle was 30 minutes.

### Lipid Bioconjugation

Lipids were incorporated to the sense strands via on-column and/or post-column synthesis. For on-column synthesis lipid at the terminal ends (3′ and 5′) and at the internal position were synthesized on the solid support. For post-column synthesis the lipids were introduced in solution phase with their corresponding N-hydroxysuccinimide esters and the appended amine linker of the nucleotide.

### On-Column Synthesis

For example, on-column introduction of compound 17 at the 3′ end of the sense strand was performed using 3′-amino modifier such as 2-dimethoxytrityloxymethyl-6-fluorenylmethoxycarbonylamino-hexane-1-succinoyl)-long chain alkylamino-CPG S1-1 (Scheme 1). Following the deprotection of the Fmoc group with 20% piperidine in DMF the CPG was thoroughly washed with DMF, MeCN, diethyl ether, and dried. A solution of 80 gmol of compound 17 in 2 ml of 1,4-dioxane was added to the above CPG followed by 30 μl of DIPEA and shaken for 12 hours. The CPG was washed with DCM, MeCN, and Et₂O. A solution of 1 ml of CAP A and CAP B was added and shaken for 30 minutes. S1-2 was washed with DCM, MeCN, Et₂O, and dried and used for the oligo assembly.

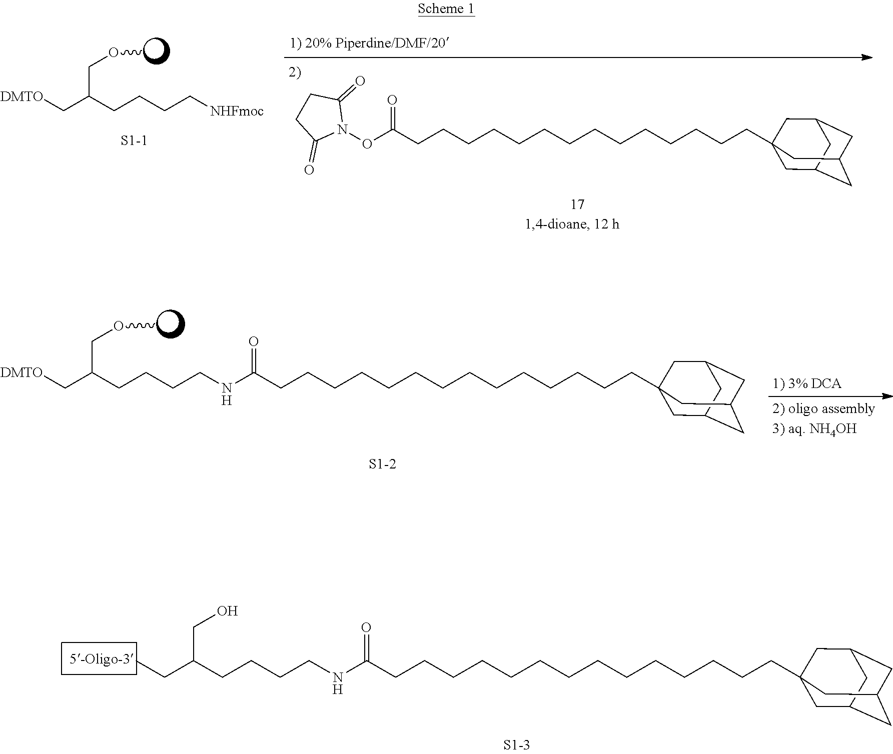

Introduction of compound 17 at the 5′ end of the sense strand was carried out using 5′-amino modifier such as 6-(4-monomethoxytritylamino)hexyl-(2-cyanoethyl)-(N,N′-diisopropyl)-phosphoramidite. After the assembly of the sense strand on the CPG following the standard procedure, in the penultimate step the 5′ end of the sense strand was coupled to 6-(4-monomethoxytritylamino)hexyl-(2-cyanoethyl)-(N,N-diisopropyl)-phosphoramidite followed by deblocking of 4-monomethoxytrityl group with 3% DCA/DCM for 30 minutes. The CPG was subsequently washed with DCM, ACN and coupled with compound 17 for 12 hours (Scheme 2).

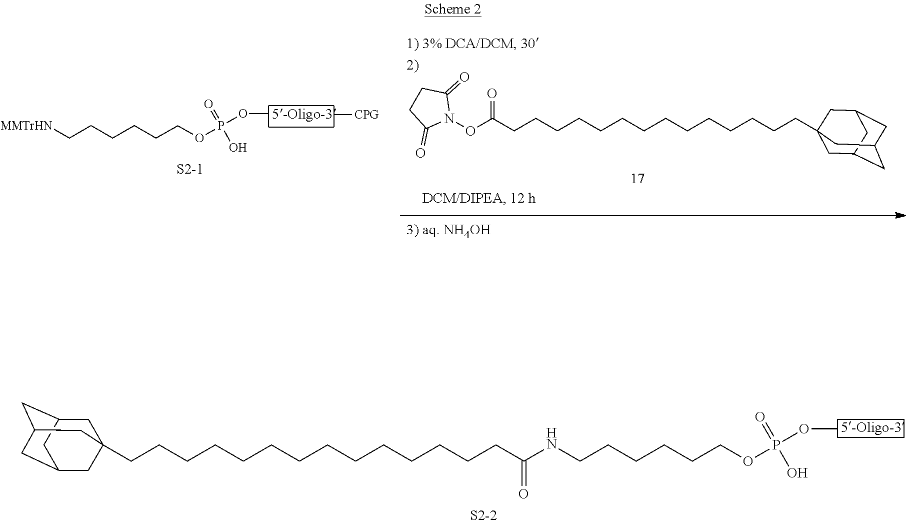

The adamantyl lipid at the internal position was introduced during the sense strand assembly with corresponding phosphoramidite monomer. For example, Scheme 3 illustrates this with uridine based phosphoramidite monomer 25.

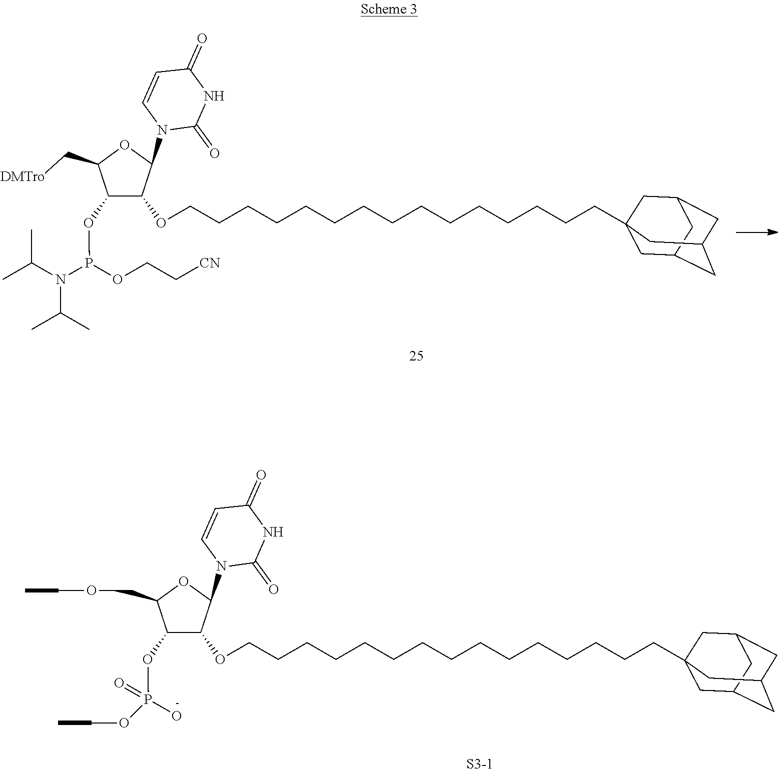

### Cleavage, Purification and Desalting

After completion of synthesis, the oligonucleotide was cleaved from the support with simultaneous deprotection of nucleobase and phosphate groups using ammonia solution for 7 hours at 55° C. The CPG was filtered and washed with ethanol/acetonitrile/water (3:1:1 v/v). After reducing the volume, the oligonucleotide was purified by reversed-phase or ion-exchange chromatography. The buffers for reverse phase were 0.1M sodium acetate in 90/10% water/acetonitrile (Buffer A) and acetonitrile (Buffer B) and the buffers for ion-exchange are 20 mM sodium phosphate (pH 11) in 90/10% water/acetonitrile (buffer A) and 20 mM sodium phosphate (pH 11) in 90/10% water/acetonitrile, 1.8M sodium bromide (buffer B). Fractions containing full-length oligonucleotides were pooled, desalted, and the compounds were analyzed by liquid chromatography-mass spectrometry (LC-MS).

### Post-Column Synthesis

Lipids were bioconjugated at the terminal position (3′,5′ end) and at the internal position of the sense strand in the solution phase after the purification and desalting of the corresponding oligonucleotide.

For example, incorporation at the internal position was carried out with phosphoramidite monomer derivatized with an amine linker at the 2′ position of respective nucleotide. Scheme 4 illustrates this with uridine based phosphoramidite monomer S4-1. After incorporation of S4-1 at the internal position and complete assembly, cleavage, purification and desalting process, the free oligonucleotide with the pendant amine was coupled with corresponding lipid-NHS esters in solution phase.

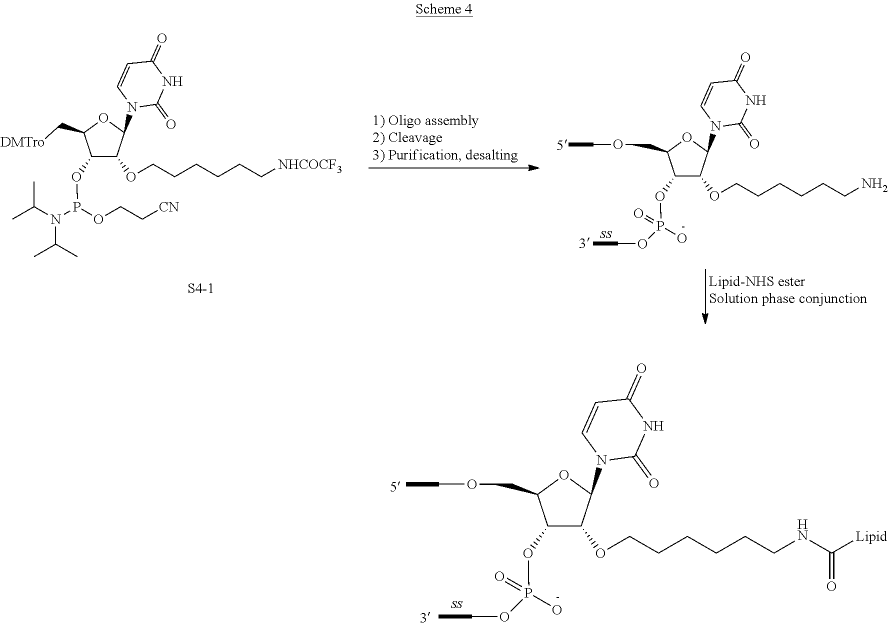

Bioconjugation at the terminal/internal positions was carried out with 0.15 mM solution of deprotected and desalted oligonucleodite in 0.1M solution of NaHCO₃ (pH 8.4) with 1.5 mM solution of the corresponding lipid-NHS ester in DMF at 60° C. To a solution (0.25 mL) of oligonucleotide was added DMF solution of lipid-NHS ester (0.6 mL) and the resulting mixture heated at 63° C. Progress was monitored by RP-HPLC (C-8 column, A: 50 mM TEAA, B: MeCN; gradient 5-100% B at 30° C.). Reaction was >90% completed usually in less than one hour. Reaction mixture was diluted with water and purified by RP-HPLC (C-8 Xbridge Waters column; A: 50 mM NaOAc, B: MeCN; 5-100% B gradient, at 60° C.). Isolated yields of lipid conjugated oligonucleotides were 60-70%. Duplex formation

For duplex formation, equimolar amounts of sense and antisense strand were mixed and kept at room temperature for 30 minutes. The integrity of duplex was confirmed by denaturing and non-denaturing HPLC analysis.

### Example 1

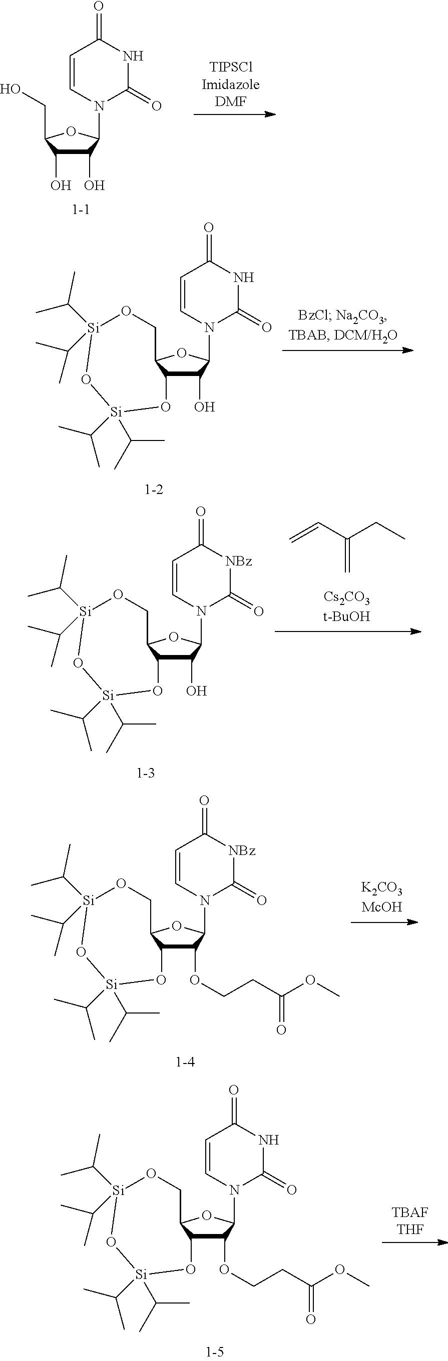

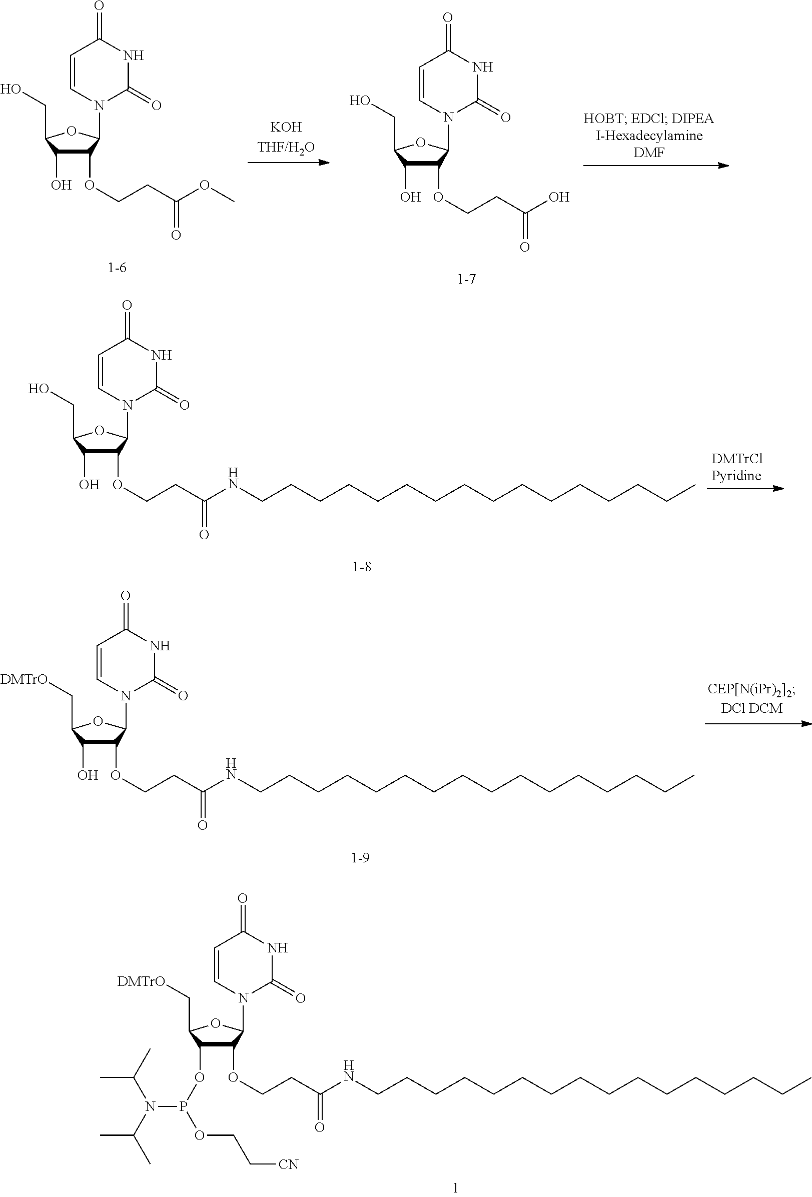

### Preparation of 1-2

To a suspension of 1-1 (72.0 g, 295.0 mmol) in DMF (720 mL) with an inert atmosphere of nitrogen was added TIPDSCl₂ (111.0 g, 354.0 mmol) at 0° C. Then imidazole (50.1 g, 737.5 mmol) was added, and the resulting mixture stirred for 14 h at room temperature. The mixture was diluted with EtOAc (2.5 L) and washed with H₂O, saturated aqueous sodium bicarbonate and brine and dried over Na₂SO₄. The evaporated residue was purified by column chromatography (SiO₂, 0-10% MeOH in DCM) to give 1-2 (115.0 g, 236 mmol, 80% yield) as a white solid. MS: m/z 487 [M+H]⁺.

### Preparation of 1-3

A mixture of 1-2 (45 g, 92.5 mmol), Na₂CO₃ (39 g, 370 mmol), and tetrabutylammoniumbromide (1.2 g, 3.7 mmol) was dissolved in a two-phase solution of DCM/H₂O (1.0/2.0 L), then added BzCl (16 mL, 138.7 mmol) to the mixture with vigorous stirring. Vigorous stirring was continued at 30° C. until 2 had disappeared on TLC. Then the mixture was transferred into a separatory funnel. The organic phase was collected, and the aqueous phase was extracted with DCM. The combined organic extract was dried over Na₂SO₄ and concentrated. The evaporated residue was purified by silica gel column chromatography (PE/EtOAc 3/1). This resulted 1-3 (29.0 g, 53.0% yield) as a white solid. MS: m/z 591 [M+H]⁺. ¹H NMR (DMSO-d₆): δ 8.00 (d, J=7.4 Hz, 2H), 7.86 (d, J=8.0 Hz, 1H), 7.79 (t, J=7.4 Hz, 1H), 7.60 (t, J=7.8 Hz, 2H), 5.82 (d, J=8.0 Hz, 1H), 5.66 (d, J=4.4 Hz, 1H, exchanged with D₂O), 5.57 (s, 1H), 4.21-4.15 (m, 3H), 4.03 (m, 1H), 3.96-3.92 (m, 1H), 1.09-0.99 (m, 28H).

### Preparation of 1-4

To a solution of 1-3 (10.5 g, 17.8 mmol), methyl methacrylate (30.5 g, 356 mmol) in t-BuOH (50 mL) was added Cs₂CO₃ (2.9 g, 8.9 mmol). The reaction mixture was stirred for 4 h at 30° C. and then diluted with EtOAc. The mixture was washed with H₂O twice, dried over Na₂SO₄ and concentrated. The evaporated residue was purified by column chromatography (SiO₂, PE/EtOAc 6/1) to give 1-4 (8.7 g, 72% yield) as a white solid. MS: m/z 677 [M+H]⁺.

### Preparation of 1-5

To a solution of 1-4 (8.7 g, 12.8 mmol) in MeOH (87 mL) was added K₂CO₃ (3.5 g, 25.6 mmol). The reaction mixture was stirred for 2 h at 30° C. It was then diluted with EtOAc/water (1/1), organic layer washed with water, dried over Na₂SO₄, and concentrated. The residue was purified by Flash-Prep-HPLC (C₁₈ column, mobile phase CH₃CN and H₂O (0.5% NH₄HCO₃), gradient 50% to 100% MeCN) to yield 1-5 (6.1 g, 82%) as a white solid. MS: m/z 573 [M+H]⁺.

### Preparation of 1-6

To a solution of 1-5 (6.1 g, 10.6 mmol) in THF (60 mL) was added TBAF (12.7 mL, 12.7 mmol). The reaction mixture was stirred for 1 h at room temperature then concentrated and the residue was purified by column chromatography (SiO₂, 0-5% MeOH in EtOAc). This resulted in 1-6 (3.2 g, 90% yield) as a white solid. MS: m/z 331 [M+H]+; ¹H NMR (DMSO-d₆): δ 11.32 (s, 1H, exchanged with D₂O), 7.91 (d, J=8.0 Hz, 1H), 5.81 (d, J=5.0 Hz, 1H), 5.65 (d, J=8.0 Hz, 1H), 5.13 (s, 1H exchanged with D₂O), 5.03 (d, J=5.0 Hz, 1H, exchanged with D₂O), 4.10 (m, 1H), 3.92 (m, 1H), 3.84-3.70 (m, 3H), 3.65-3.53 (m, 3H), 2.56 (m, 2H).

### Preparation of 1-7

To a solution of 1-6 (3.2 g, 9.6 mmol) in THF/H₂O (30/15 mL) was added KOH (1.6 g, 28.8 mmol). The reaction mixture was stirred for 1 h at 20° C. The solution was neutralized to PH 7 with H⁺ exchange resin, filtered and concentrated to get the 1-7 as a white solid. MS: m/z 317 [M+H]⁺.

### Preparation of 1-8

To a solution of 1-7 (3.0 g, 9.6 mmol) in DMF (30 mL) was added EDCI (1.8 g, 9.6 mmol), HOBT (1.3 g, 9.6 mmol), hexadecan-1-amine (2.3 g, 9.6 mmol) and DIPEA (2.5 g, 19.2 mmol). The reaction mixture was stirred for 24 h at 20° C. and then diluted with EtOAc. The mixture was washed with H₂O and 5% citric acid, dried over Na₂SO₄ and concentrated. The residue was purified by precipitating with EtOAc to give 1-8 (4.7 g, 78% yield over two steps) as a white solid. MS: m/z 540 [M+H]⁺. ¹H NMR (DMSO-d₆): δ 11.32 (s, 1H, exchanged with D₂O), 7.91 (d, J=8.0 Hz, 1H), 7.88 (t, J=5.2 Hz, 1H, exchanged with D₂O), 5.81 (d, J=5.3 Hz, 1H), 5.65-5.62 (dd, J=8.0, 1.8 Hz, 1H), 5.16 (s, 2H, exchanged with D₂O), 4.17 (m, 1H), 3.94-3.54 (m, 6H), 3.00 (m, 2H), 2.34 (m, 2H), 1.37-1.17 (m, 28H), 0.87-0.82 (m, 3H).

### Preparation of 1-9

To a solution of 1-8 (3.2 g, 5.9 mmol) in pyridine (30 mL) was added DMTrCl (2.4 g, 7.1 mmol) under N₂ atmosphere. The reaction mixture was stirred at room temperature for 3 h. Then the reaction mixture was diluted with EtOAc washed with H₂O and dried over Na₂SO₄. The evaporated residue was purified by silica gel column chromatography (PE/EtOAc 2/1 to 1/2) to give 1-9 (3.3 g, 62% yield) as a white solid. MS: m/z 840 [M−H]⁻; ¹H-NMR (DMSO-d₆): δ 11.38 (s, 1H, exchanged with D₂O), 7.87 (t, J=5.2 Hz, 1H, exchanged with D₂O), 7.70 (d, J=8.0 Hz, 1H), 7.39-7.21 (m, 9H), 6.91-6.89 (m, 4H), 5.77 (d, J=3.4 Hz, 1H), 5.29 (d, J=8.0 Hz, 1H), 5.26 (d, J=6.3 Hz, 1H, exchanged with D₂O), 4.24 (m, 1H), 4.03-3.92 (m, 2H), 3.79 (m, 2H), 3.74 (s, 6H), 3.30-3.20 (m, 2H), 3.01 (m, 2H), 2.37 (m, 2H), 1.38-1.17 (m, 28H), 0.87-0.81 (m, 3H).

### Preparation of 1

To a solution of 1-9 (2.9 g, 3.4 mmol) in DCM (30 mL) were added DCI (418 mg, 2.9 mmol) and CN(CH₂)₂OP[N(iPr)₂]₂ (1.2 g, 4.1 mmol) under N₂ atmosphere. The reaction mixture was stirred at 30° C. for 2 h. Then the solution was diluted with DCM and washed with H₂O. Organic layer was washed with brine, dried over Na₂SO₄ and concentrated. The residue was purified by Flash-Prep-HPLC (C₁₈ column; mobile phase H₂O with 0.5% NH₄HCO₃, CH₃CN; gradient 0-100% MeCN) to give compound 1 (2.6 g, 71% yield) as a white solid. LCMS: m/z 1040[M−H]⁻; ¹H-NMR (DMSO-d₆): δ 11.38 (s, 1H), 7.79-7.72 (m, 2H), 7.40-7.21 (m, 9H), 6.91-6.86 (m, 4H), 5.80 (m, 1H), 5.30-5.23 (dd, J=19.6, 8.0 Hz, 1H), 4.44-4.32 (m, 1H), 4.13-4.05 (m, 2H), 3.86-3.46 (m, 12H), 3.36-3.25 (m, 2H), 2.98 (m, 2H), 2.78 (m, 1H), 2.61 (m, 1H), 2.33 (m, 2H), 1.35-1.17 (m, 28H), 1.15-0.95 (m, 12H), 0.86-0.83 (m, 3H); ³¹P-NMR (DMSO-d₆): δ 149.22, 148.69.

### Example 2

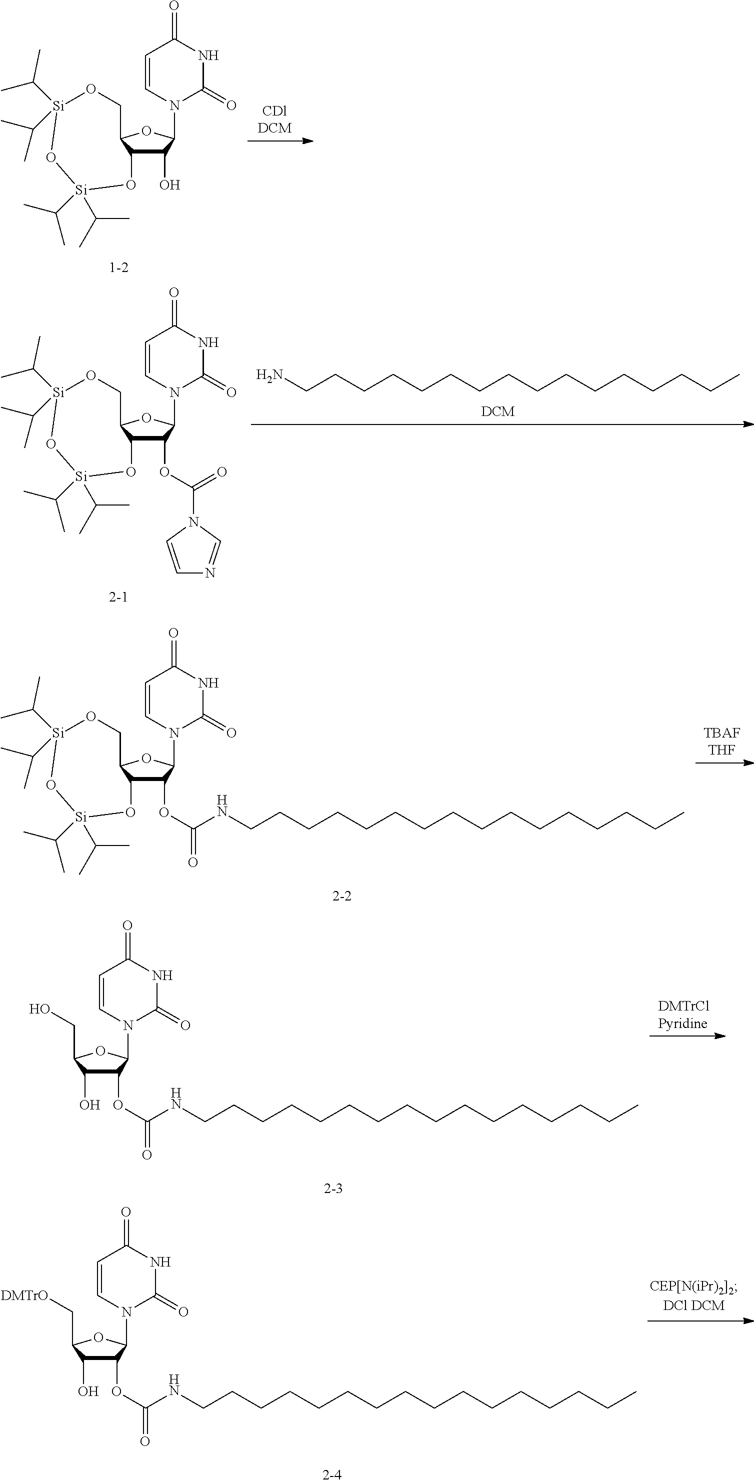

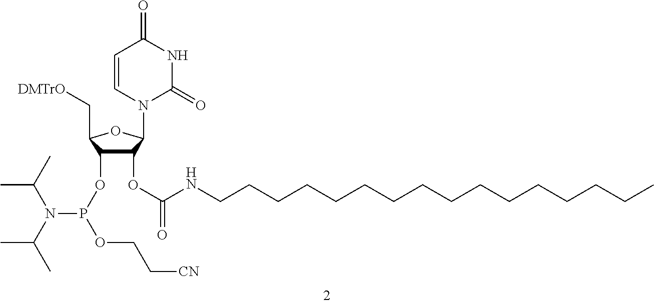

### Preparation of 2-1

To a solution of 1-2 (9.7 g, 19.9 mmol) in anhydrous DCM (100 mL) was added CDI (3.5 g, 19.9 mmol) and the reaction mixture stirred for 1 h at room temperature. The mixture was used for next step without purification. ESI-LCMS: m/z 581 [M+H]⁺.

### Preparation of 2-2

To a solution of 2-1 (11.5 g, 19.9 mmol) in anhydrous DCM (100 mL) was added dropwise hexadecan-1-amine (4.8 g, 19.9 mmol) in anhydrous DCM (100 mL). The reaction mixture was stirred for 16 h at 30° C. then quenched with H₂O and diluted with DCM. The organic phase was washed with H₂O and 5% citric acid and dried over Na₂SO₄. The evaporated residue was purified by silica gel column chromatography (0-20% acetone in EtOAc/DCM 1/1) to give 2-2 (11.1 g, 70% yield) over two steps) as a white solid. MS: m/z 754 [M+H]+; ¹H NMR (DMSO-d₆): δ 11.41 (s, 1H, exchanged with D₂O), 7.69 (d, J=8.0 Hz, 1H), 7.33 (t, J=5.5 Hz, 1H, exchanged with D₂O), 5.66 (d, J=1.6 Hz, 1H), 5.59 (d, J=8.0 Hz, 1H), 5.33-5.31 (m, 1H), 4.51-4.47 (m, 1H), 4.09-4.05 (m, 1H), 3.95-3.82 (m, 2H), 3.05-2.85 (m, 2H), 1.37-1.22 (m, 28H), 1.05-0.96 (m, 24H), 0.87-0.82 (m, 3H).

### Preparation of 2-3

To a solution of 2-2 (4.7 g, 6.2 mmol) in THF (50 mL) was added 1M TBAF in THF (7.4 mL, 7.4 mmol). The reaction mixture was stirred for 1 h at room temperature. Then EtOAc was added, and the mixture was washed with H₂O and dried over Na₂SO₄. The residue was purified by precipitating from EtOAc to give 2-3 (3.1 g, 92% yield) as a white solid. MS: m/z 512 [M+H]+; ¹H NMR (DMSO-d₆): δ 11.33 (s, 1H, exchanged with D₂O), 7.90 (d, J=8.0 Hz, 1H), 7.27 (t, J=5.5 Hz, 1H, exchanged with D₂O), 5.97 (d, J=6.0 Hz, 1H), 5.66 (d, J=8.0 Hz, 1H), 5.46 (d, J=4.9 Hz, 1H, exchanged with D₂O), 5.19 (m, 1H, exchanged with D₂O), 5.01 (t, J=5.7 Hz, 1H), 4.19 (m, 1H), 3.88 (m, 1H), 3.62-3.57 (m, 2H), 2.91 (m, 2H), 1.37-1.17 (m, 28H), 0.87-0.82 (m, 3H).

### Preparation of 2-4

To a solution of 2-3 (3.1 g, 6.0 mmol) in pyridine (30 mL) was added DMTrCl (2.2 g, 6.6 mmol) under N₂ atmosphere. The reaction mixture was stirred at room temperature for 3 h, then diluted with EtOAc, washed with H₂O and dried over Na₂SO₄. The evaporated residue was purified by silica gel column chromatography (PE/EtOAc 2/1 to 1/2) to give 2-4 (3.1 g, 60% yield) as a yellow solid. MS: m/z 812 [M−H]⁻; ¹H-NMR (DMSO-d₆): δ 11.39 (s, 1H, exchanged with D₂O), 7.71 (d, J=8.0 Hz, 1H), 7.39-7.21 (m, 10H), 6.91-6.89 (m, 4H), 5.91 (d, J=4.8 Hz, 1H), 5.52 (d, J=5.6 Hz, 1H, exchanged with D₂O), 5.37 (d, J=8.0 Hz, 1H), 5.14 (t, J=5.1 Hz, 1H), 4.35 (m, 1H), 3.96 (m, 1H), 3.74 (s, 6H), 3.32-3.19 (m, 2H), 2.95 (m, 2H), 1.38-1.15 (m, 28H), 0.86-0.83 (m, 3H).

### Preparation of 2

To a solution of 2-4 (1.9 g, 2.3 mmol) in DCM (20 mL) were added DCI (234 mg, 1.9 mmol) and CN(CH₂)₂OP[N(iPr)₂](843 mg, 2.8 mmol) under N₂ atmosphere. The reaction mixture was stirred at 30° C. for 2 h. Then the solution was diluted with DCM and washed with H₂O. The organic layer was washed with brine, dried over Na₂SO₄, and evaporated. Purification of the residue by silica gel column chromatography (PE/EtOAc 2/1 to 1/2) resulted in compound 2 (2.1 g, 86% yield) as a white solid. MS: m/z 1012[M−H]⁻; ¹H-NMR (DMSO-d₆): δ 11.25 (s, 1H), 7.67 (d, J=8.0 Hz, 1H), 7.43-7.21 (m, 10H), 6.90-6.87 (m, 4H), 5.97 (m, 1H), 5.42-5.32 (m, 2H), 4.60 (m, 1H), 4.20-4.14 (m, 2H), 3.81-3.34 (m, 12H), 2.98 (m, 2H), 2.73 (m, 1H), 2.58 (m, 1H), 1.43-1.19 (m, 28H), 1.15-1.00 (m, 12H), 0.86-0.83 (m, 3H); ³¹P-NMR (DMSO-d₆): δ 149.64, 149.60.

### Example 3

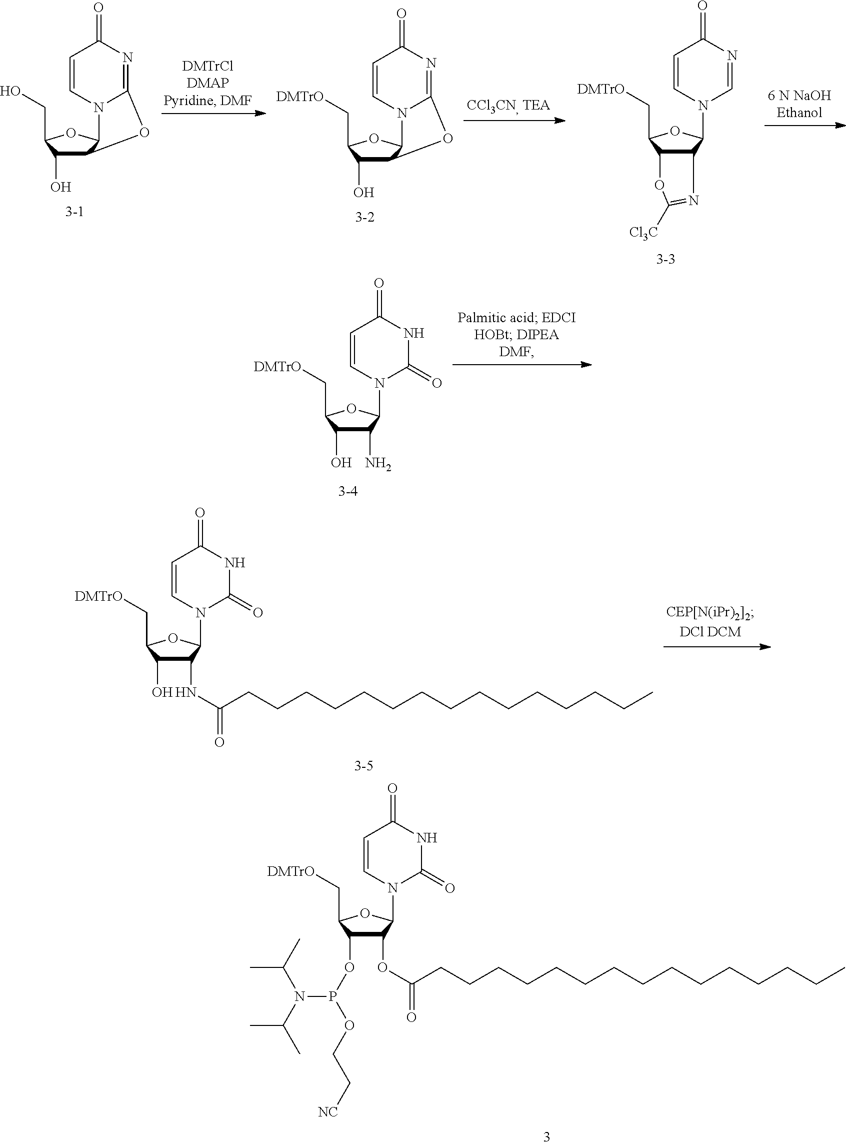

### Preparation of 3-3

The suspension of 3-1 (31.0 g, 137.1 mmol), DMTrCl (51.0 g, 150.8 mmol) and DMAP (0.2 g, catalytic) in a mixture of pyridine (110 mL) and DMF (78 mL) was stirred for 15 h at room temperature and then concentrated. The residue was partitioned between dichloromethane and water. Organic layer was washed with aqueous sodium bicarbonate and brine and dried over MgSO₄. The evaporated residue was co-evaporated with toluene to afford compound 3-2 as a sticky oil. CCl₃CN (140.0 g) and TEA (3 mL) were added, and the solution was heated at 90° C. for 16 h. The black reaction mixture was concentrated, and the residual oil purified by silica gel column chromatography (DCM:MeOH 100/1 to 20/1) to afford the compound 3-3 as a foam (89.1 g, 97% yield). MS: m/z 670 [M−H]⁻.

### Preparation of 3-4

To a solution of 3-3 (89.1 g, 132.8 mmol) in ethanol (360 mL) was added 6 N NaOH solution (180 mL), and the reaction was refluxed for 16 h at 70° C., then cooled, and concentrated. The residue was partitioned between dichloromethane and saturated ammonium chloride. The aqueous phase was washed with dichloromethane, and the combined organic extract was dried over magnesium sulfate, filtered, and evaporated. The residue was purified by silica gel column chromatography (DCM containing 1% TEA/MeOH 30/1 to 10/1) to afford compound 3-4 (42.0 g, 77.1 mmol, 56.2% yield over 3 step) as a white foam. MS: 544 m/z [M−H]⁻. ¹H NMR (DMSO-d₆) δ 7.62 (dd, J=8.3, 4.8 Hz, 1H), 7.43-7.19 (m, 9H), 6.97-6.83 (m, 4H), 5.79-5.61 (m, 2H), 5.39 (dd, J=8.0, 2.7 Hz, 1H), 3.98 (d, J=7.6 Hz, 2H), 3.74 (s, 6H), 3.42-3.11 (m, 4H), 2.60 (q, J=7.2 Hz, 2H).

### Preparation of 3-5

To a solution of 3-4 (4.2 g, 7.7 mmol) in dry DMF (42 mL) were added palmitic acid (2.0 g, 7.7 mmol), EDCI (1.5 g, 7.7 mmol), HOBT (1.1 g, 7.7 mmol), and DIPEA (2.0 g, 15.4 mmol). The reaction mixture was stirred for 15 h at room temperature, then diluted with water and extracted with EtOAc. Organic phase was dried over magnesium sulfate, filtered, and evaporated. The residue was purified by silica gel column chromatography (PE/EtOAc, 20% to 100%) to give 3-5 (4.8 g, 6.1 mmol, 79% yield, 98% purity) as a solid. MS: m/z 782 [M−H]⁻. ¹H NMR (DMSO-d₆): δ 11.31 (d, J=2.1 Hz, 1H), 7.82 (d, J=8.6 Hz, 1H), 7.63 (d, J=8.1 Hz, 1H), 7.50-7.16 (m, 9H), 6.90 (d, J=8.4 Hz, 4H), 5.88 (d, J=8.1 Hz, 1H), 5.69 (d, J=4.7 Hz, 1H), 5.40 (dd, J=8.1, 2.0 Hz, 1H), 4.65 (q, J=7.9 Hz, 1H), 4.01 (d, J=5.8 Hz, 1H), 3.74 (s, 6H), 3.22 (m, 1H), 2.14 (td, J=7.1, 4.0 Hz, 2H), 1.49-1.40 (m, 2H), 1.22 (d, J=7.6 Hz, 26H), 0.85 (t, J=6.6 Hz, 3H).

### Preparation of 3

To a suspension of 3-5 (2.7 g, 3.4 mmol) in dry DCM (30 mL) were added CN(CH₂)₂OP[N(iPr)₂](1.3 g, 4.4 mmol) and DCI (341 mg, 2.9 mmol) under N₂ atmosphere. The mixture was stirred at room temperature for 2.5 h. Then the solution was washed with water, brine and dried over Na₂SO₄. The concentrated residue was purified by silica gel column chromatography (PE/EtOAc 5/1 to 1/3) to give compound 3 (2.4 g, 2.4 mmol, 70% yield) as a white solid. MS: m/z 982 [M−H]⁻. ¹H NMR (DMSO-d₆): δ 11.41 (d, J=4.4 Hz, 1H), 7.99 (t, J=9.5 Hz, 1H), 7.66 (dd, J=8.2, 1.5 Hz, 1H), 7.50-7.13 (m, 9H), 6.89 (dd, J=9.0, 2.4 Hz, 4H), 5.98-5.91 (m, 1H), 5.42 (dd, J=8.3, 2.6 Hz, 1H), 4.88 (td, J=8.5, 6.0 Hz, 1H), 4.52-4.09 (m, 2H), 3.74 (s, 7H), 3.63-3.44 (m, 2H), 3.28 (d, J=3.4 Hz, 2H), 2.75 (t, J=6.0 Hz, 1H), 2.66 (t, J=6.4 Hz, 1H), 2.13 (ddd, J=17.1, 14.2, 7.0 Hz, 2H), 1.22 (d, J=9.3 Hz, 27H), 1.15-0.91 (m, 12H), 0.85 (t, J=6.6 Hz, 3H). ³¹P NMR (DMSO-d₆): δ 148.88, 147.48.

### Example 4

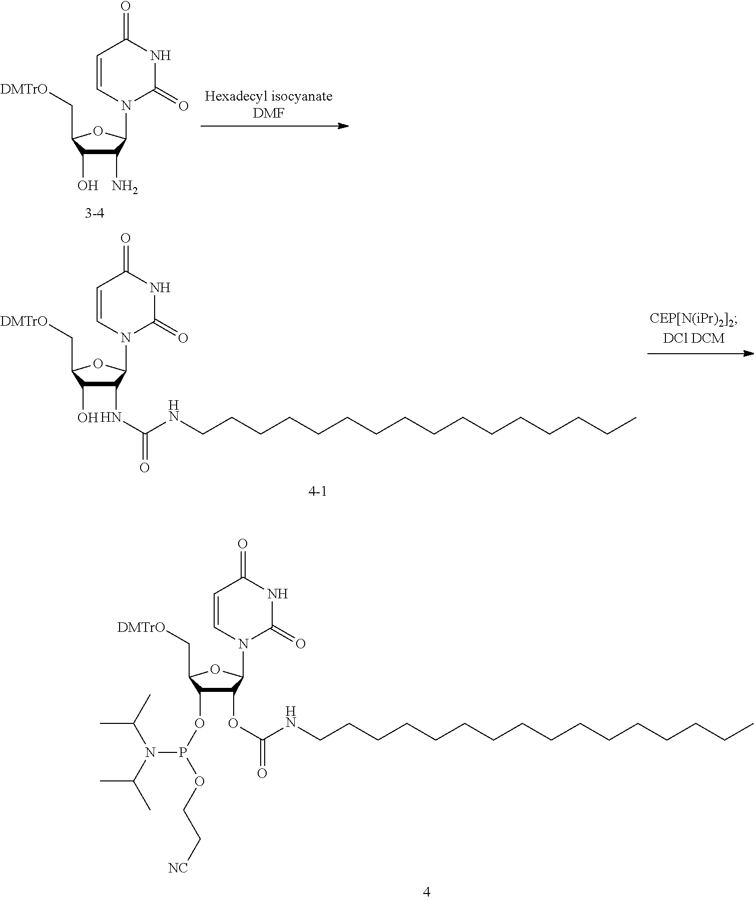

### Preparation of 4-1

A solution of 3-4 (2.1 g, 3.9 mmol) and hexadecyl isocyanate (1.1 g, 3.9 mmol) in DMF (20 mL) was stirred for 2 h at 30° C. The resulting mixture was diluted with EtOAc, washed with H₂O and dry over by Na₂SO₄. The concentrated residue was purified by silica gel column chromatography (DCM/MeOH 100/1 to 10/1) to give 4-1 (2.7 g, 3.2 mmol, 82% yield) as a yellow solid. EST-LCMS: m/z 813 [M+H]⁺. ¹H-NMR (DMSO-d₆): δ 11.30 (s, 1H, exchanged with D₂O), 7.63 (d, J=8 Hz, 1H), 7.41-7.21 (m, 9H), 6.90 (d, J=8 Hz, 4H), 6.27 (t, J=5 Hz, 1H, exchanged with D₂O), 5.98 (d, J=8 Hz, 1H, exchanged with D₂O), 5.86 (d, J=5 Hz, 1H), 5.78 (d, J=8 Hz, 1H, exchanged with D₂O), 5.41-5.39 (m, 1H), 4.45 (dd, J=8 Hz, 1H), 4.09 (m, 1H), 3.98 (s, 1H), 3.74 (s, 6H), 3.26-3.15 (m, 2H), 2.98-2.88 (m, 2H), 1.37-1.15 (m, 29H), 0.85 (m, 3H).

### Preparation of 4

To a solution of 4-1 (2.1 g, 2.6 mmol) in DCM (20 mL) were added DCI (313 mg, 2.2 mmol) and CN(CH₂)₂OP[N(iPr)₂](932 mg, 3.1 mmol) under N₂ atmosphere. The mixture was stirred at 30° C. for 2 h, then diluted with DCM, washed with H₂O and dried over Na₂SO₄. The evaporated residue was purified by silica gel column chromatography (PE/EtOAc 2/1 to 1/2) to give compound 4 (2.3 g, 86% yield) as a white solid. MS: m/z 1013 [M+H]⁺. ¹H-NMR (DMSO-d₆): δ 11.41 (s, 1H), 7.65 (t, J=8 Hz, 1H), 7.40-7.21 (m, 9H), 6.89 (d, J=8 Hz, 4H), 6.26 (dt, J=37.9, 5.5 Hz, 1H), 5.92 (d, J=9 Hz, 1H), 5.87-5.84 (m, 1H), 5.42 (d, J=8 Hz, 1H), 4.80-4.61 (m, 1H), 4.44-4.13 (m, 2H), 3.90-3.47 (m, 10H), 3.31-3.16 (m, 2H), 3.05-2.89 (m, 2H), 2.77-2.68 (m, 2H), 1.34-1.19 (m, 29H), 1.15-0.99 (m, 12H), 0.86-0.83 (m, 3H); ³¹P-NMR (DMSO-d₆): δ 148.94, 147.04.

### Example 5

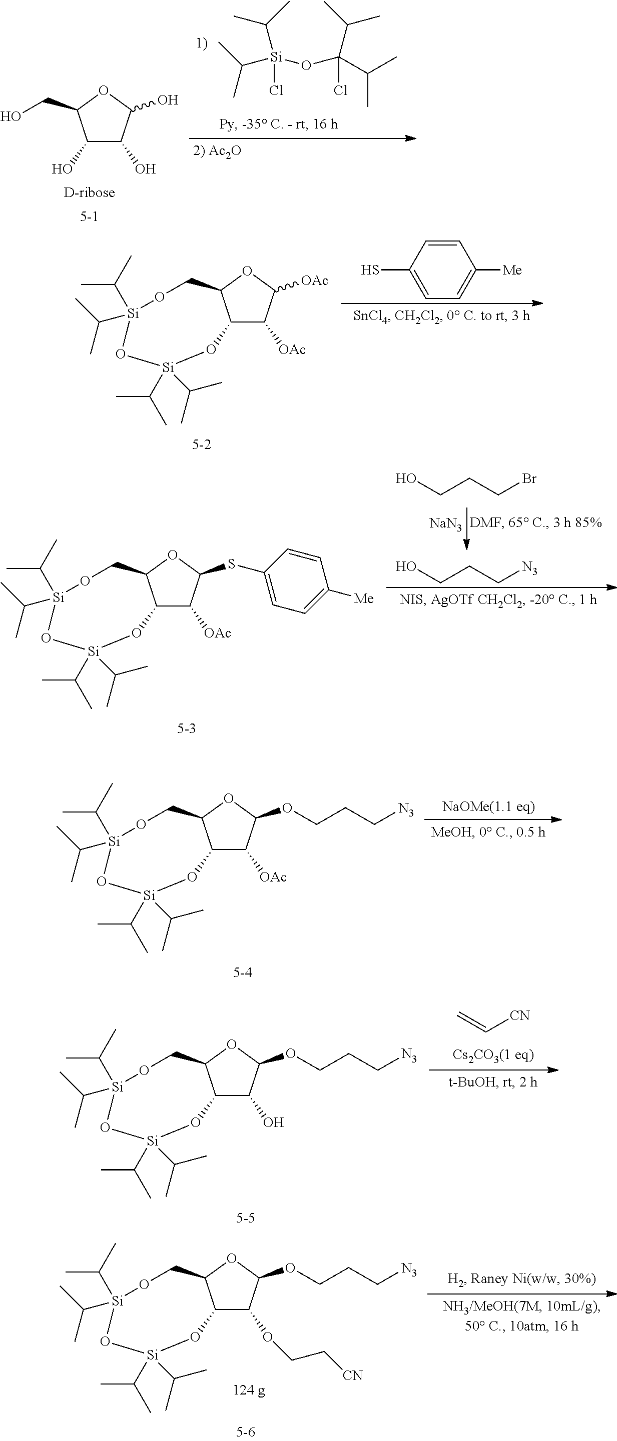

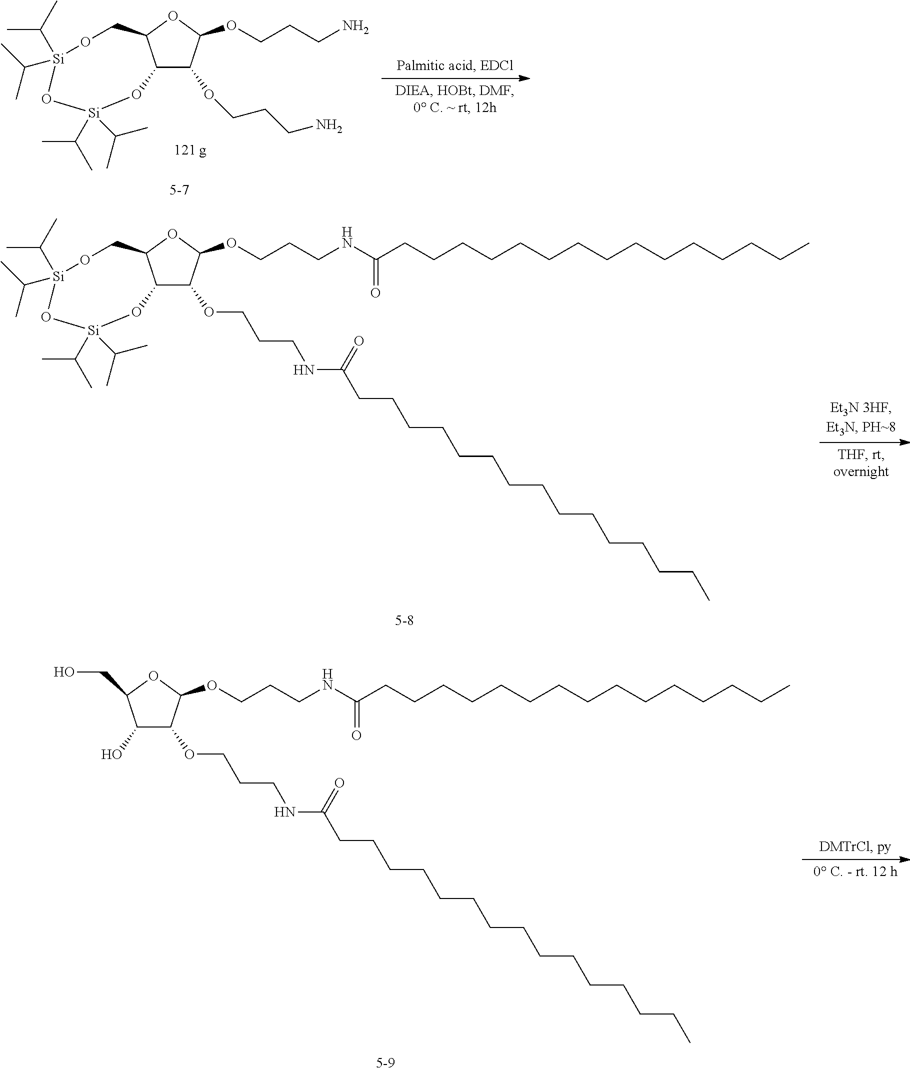 [SMILES: CC(C)[Si]1(C(C)C)OC[C@H]2O[C@@H](OCCCN)[C@H](OCCCN)[C@@H]2O[Si](C(C)C)(C(C)C)O1 ; CCCCCCCCCCCCCCCC(=O)NCCCO[C@@H]1O[C@@H]2CO[Si](C(C)C)(C(C)C)O[Si](C(C)C)(C(C)C)O[C@H]2[C@H]1OCCCNC(=O)CCCCCCCCCCCCCCC ; CCCCCCCCCCCCCCCC(=O)NCCCO[C@@H]1O[C@H](CO)[C@@H](O)[C@H]1OCCCNC(=O)CCCCCCCCCCCCCCC]

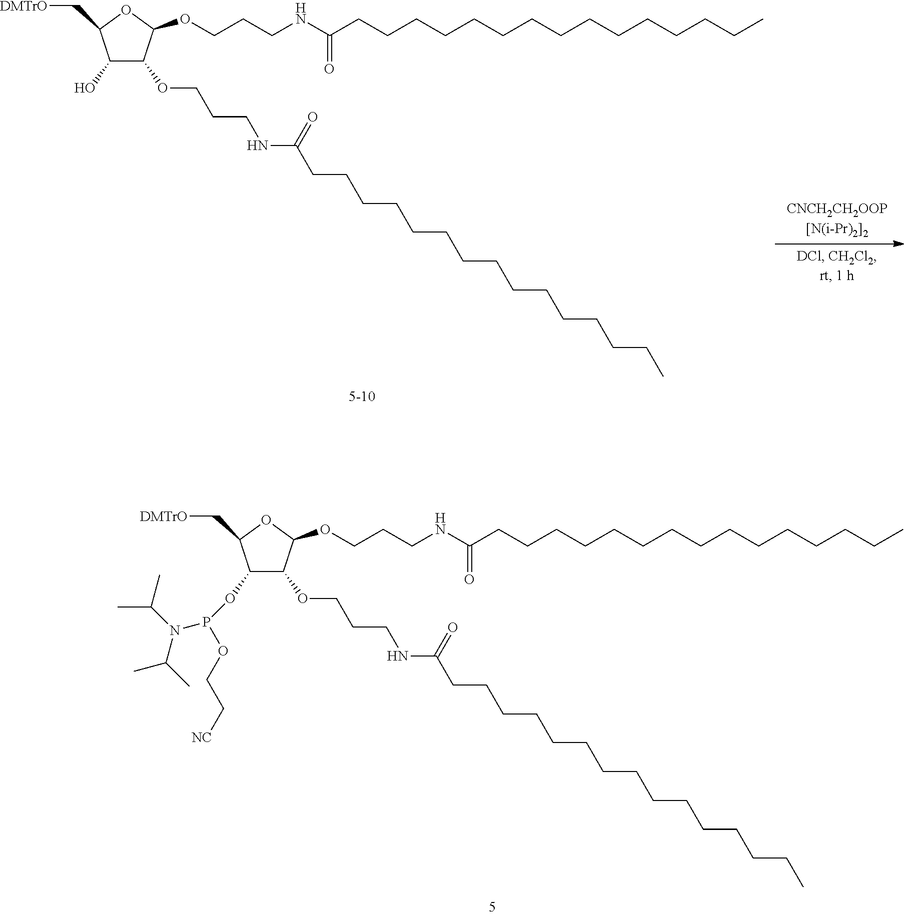

### Preparation of 5-2

To a solution of (3R,4S,5R)-5-(hydroxymethyl) tetrahydrofuran-2,3,4-triol (100 g, 666 mmol) in pyridine (1000 mL) under Ar was added tetra isopropyl 1,3-dichlorodisiloxane at −35° C. The reaction mixture was allowed to warm up and stirred at room temperature for 16 h. Then acetic anhydride (330 mL) was added and the reaction mixture was stirring for another 6 h at room temperature. The reaction mixture was quenched with water (300 mL) at 0° C. and concentrated. The aqueous residue was diluted with EtOAc (1000 mL) and washed with water (2×500 mL), 2 N hydrochloric acid (2×200 mL), saturated aqueous NaHCO₃ (2×400 mL), water (2×500 mL), and saturated brine (2×400 mL). The organic layer was dried (anhydrous Na₂SO₄), filtered and concentrated under reduced pressure. The crude product was purified on a silica gel column with petroleum ether/ethyl acetate (50:1-20:1) to yield 160 g (50%) of 5-2 was as a yellow liquid. MS: m/z 499.30 [M+Na]⁺.

### Preparation of 5-3

To a solution of 5-2 (160 g, 336 mmol), p-thiocresol (50 g, 403 mmol, 1.2 equiv) in anhydrous dichloromethane (1600 mL) under argon atmosphere was added dropwise the solution of tin tetrachloride in dichloromethane (1M, 74 mL) at 0° C. The resulting solution was stirred for 3 h at room temperature. The reaction was quenched with trimethylamine (50 mL) and concentrated. The crude residue was purified on a silica gel column with petroleum ether/ethyl acetate (100:1-80:1) to yield 100 g (55%) of 5-3 as a light yellow oil. MS: m/z 563.20 [M+Na]⁺. ¹H NMR (CDCl₃): δ 7.45-7.39 (m, 2H), 7.13 (d, J=7.9 Hz, 2H), 5.38 (dd, J=5.2, 1.5 Hz, 1H), 5.20 (d, J=1.5 Hz, 1H), 4.06-3.98 (m, 2H), 3.92-3.82 (m, 2H), 2.32 (d, J=5.1 Hz, 3H), 2.09 (s, 3H), 1.12-0.95 (m, 28H).

### Preparation of 5-4

To a solution of 5-3 (80 g, 148 mmol) and 3-azido-1-propanol (37.4 g, 370.2 mmol, 2.5 equiv) in dichloromethane (800 mL) under Ar were added N-iodosuccinimide (36.7 g, 162.9 mmol, 1.1 equiv) and silver trifluoromethanesulfonate (3.8 g, 14.8 mmol, 0.1 equiv) at −20° C. The resulting solution was stirred at −20° C. for 30 min, quenched with trimethylamine (30 mL) and concentrated. The crude product was purified on a silica gel column with petroleum ether/ethyl acetate (50:1-20:1) to give 52 g (68% yield) of 5-4 as a yellow oil. MS: 540.40 m/z [M+Na]⁺. ¹H NMR (CDCl₃): δ 5.22 (d, J=4.8 Hz, 1H), 4.86 (s, 1H), 4.52 (dd, J=7.1, 4.8 Hz, 1H), 4.05-3.92 (m, 2H), 3.87-3.70 (m, 2H), 3.49-3.40 (m, 1H), 3.36 (t, J=6.7 Hz, 2H), 2.11 (s, 3H), 1.87-1.76 (m, 2H), 1.12-0.96 (m, 28H).

### Preparation of 5-5

To a solution of 5-4 (34 g, 65.8 mmol) in methanol (340 mL) under Ar atmosphere at 0° C. was added NaOMe in MeOH (5 M, 19.7 mL). The resulting solution was stirred at 0° C. for 1 h then quenched with glacial acetic acid and concentrated. The crude product was applied onto a silica gel column and eluted with petroleum ether/ethyl acetate (40:1-10:1) to give 24.5 g (78% yield) of 5-5 as a yellow oil. MS: m/z 498.25 [M+Na]⁺.

### Preparation of 5-6

To a solution of 5-5 (25 g, 52.6 mmol) in tert-butanol (250 mL) under Ar were added acrylonitrile (56.4 g, 1052.6 mmol, 20 equiv) and cesium carbonate (17.2 g, 52.6 mmoL, 1.0 equiv) and stirred at room temperature for 2 h. The resulting mixture was filtered and filtrate concentrated. The crude product was purified on a silica gel column with petroleum ether/ethyl acetate (30:1-8:1) to give 20 g (74% yield) of 5-6 as a yellow oil. MS: 546.40 m/z [M+NH₄]⁺. ¹H NMR (CDCl₃): δ 4.86 (s, 1H), 4.50 (dd, J=8.2, 4.0 Hz, 1H), 4.24-4.13 (m, 1H), 4.04-3.88 (m, 3H), 3.84-3.73 (m, 3H), 3.52-3.43 (m, 1H), 3.38 (t, J=6.8 Hz, 2H), 2.73-2.56 (m, 2H), 1.89-1.77 (m, 2H), 1.16-1.01 (m, 28H).

### Preparation of 5-7

To a solution of 5-6 (10 g, 18.9 mmol) in 100 mL of 7 M NH₃ in methanol was added Raney Ni (50%, 5 g). The mixture was hydrogenated at 50° C. under 5 atm of hydrogen pressure for 24 h. The reaction mixture was cooled to room temperature and filtered. The filtrate was concentrated to give 7.6 g (77% yield) of 5-7 as a yellow solid. It was used in next step without further purification. MS m/z [M+H]⁺ (ESI): 507.30.

### Preparation of 5-8

To a solution of 5-7 (5.6 g, 11.1 mmol) and palmitic acid (6.2 g, 24.4 mmoL, 2.2 equiv) in dichloromethane (56 mL) at 0° C. under Ar were added 1-(3-dimethylaminopropyl)-3-ethylcarbodiimide hydrochloride (5.3 g, 27.6 mmol, 2.5 equiv), N,N-diisopropylethylamine (7.2 g, 55.3 mmoL, 5.0 equiv), and 1-hydroxybenzotriazole (3.73 g, 27.65 mmol, 2.5 equiv). The reaction mixture was stirred for 12 h at room temperature. The reaction mixture was diluted with dichloromethane (400 mL) and washed with water. The organic layer was dried, filtered and concentrated. The residue was applied onto a silica gel column and eluted with dichloromethane/methanol (100:1-80:1) to yield 6.0 g (55%) of 5-8 as a white solid. MS: m/z 983.90 [M+H]⁺. ¹H NMR (CDCl₃): δ 6.24 (d, J=24.1 Hz, 2H), 4.83 (s, 1H), 4.45 (dd, J=8.3, 4.3 Hz, 1H), 3.98-3.94 (m, 3H), 3.92-3.85 (m, 1H), 3.78-3.69 (m, 2H), 3.67 (d, J=4.3 Hz, 1H), 3.54-3.41 (m, 3H), 3.28 (dd, J=13.0, 6.1 Hz, 1H), 3.21-3.07 (m, 1H), 2.20-2.09 (m, 4H), 1.85-1.69 (m, 4H), 1.65-1.57 (m, 4H), 1.26 (s, 48H), 1.09-1.02 (m, 28H), 0.90-0.85 (m, 6H).

### Preparation of 5-9

To a solution of triethylamine trihydrofluoride (2.95 g, 18.3 mmol) in anhydrous THF (60 mL) under Ar was added triethylamine (3.69 g, 36.6 mmoL) and stirred at room temperature for 10 min. A solution of 5-8 (6 g, 6.1 mmoL) in anhydrous THF (60 mL) was added and the reaction mixture stirred at room temperature for 1 h. It was then diluted with dichloromethane (300 mL), washed with water and saturated brine. The organic layer was dried (Na₂SO₄), filtered and concentrated. The residue was dissolved with 10 mL dichloromethane and poured slowly into 200 mL of acetonitrile with stirring. The solids were collected by filtration. 3 g (66% yield) of 5-9 was obtained as a white solid. MS: m/z 742.00 [M+H]⁺.

### Preparation of 5-10

To a solution of 5-9 (2.8 g, 3.78 mmoL) in pyridine (60 mL) at 0° C. under Ar was added 4,4′-dimethoxytrityl chloride (1.54 g, 1.53 mmoL, 1.2 equiv) and stirred for 4 h at 25° C. The reaction mixture was quenched with methanol (10 mL), diluted with dichloromethane (200 mL), washed with saturated aqueous sodium bicarbonate and saturated brine. The organic layer was dried (Na₂SO₄), filtered and concentrated. The residue was purified onto a silica gel column with dichloromethane/methanol (100:1-60:1) to get 3.1 g (79% yield) of 5-10 as a light yellow oil. MS: m/z 1041.75 [M−H]⁻. ¹H NMR (CDCl₃): δ 7.55-7.41 (m, 1H), 7.37 (d, J=1.0 Hz, 1H), 7.34 (d, J=1.0 Hz, 1H), 7.32-7.27 (m, 3H), 7.20-7.14 (m, 3H), 6.87-6.76 (m, 4H), 6.14-5.84 (m, 2H), 5.03-4.93 (m, 1H), 4.34-4.17 (m, 1H), 4.16-4.01 (m, 2H), 3.86-3.81 (m, 1H), 3.80 (s, 4H), 3.78 (s, 2H), 3.77-3.74 (m, 1H), 3.72-3.61 (m, 2H), 3.58-3.43 (m, 2H), 3.41-3.26 (m, 3H), 3.23-3.06 (m, 1H), 2.75 (s, 1H), 2.19-2.04 (m, 4H), 1.95-1.70 (m, 4H), 1.69-1.54 (m, 4H), 1.26 (s, 48H), 0.93-0.81 (m, 6H).

### Preparation of 5

To a solution of 5-10 (3.1 g, 3 mmol) in dichloromethane (31 mL) under Ar were added 3-(bis(diisopropylamino)phosphinooxy)propanenitrile (1.1 g, 3.6 mmoL, 1.2 equiv) 4,5-dicyanoimidazole (386 mg, 3.3 mmol, 1.1 equiv) and stirred for 40 min at room temperature. The resulting solution was diluted with dichloromethane (500 mL), washed with saturated aqueous sodium bicarbonate and saturated brine. The organic layer was dried (Na₂SO₄), filtered and concentrated. The crude product was purified by Prep-Flash (C18 column; mobile phase, water and tetrahydrofuran, gradient 30% to 100% THF) to give 2.17 g (60% yield) of compound 5 as a white solid. MS: m/z 1265.95 [M+Na]⁺. ¹H NMR (DMSO-d₆): δ 7.72-7.65 (m, 2H), 7.47-7.41 (m, 2H), 7.33-7.25 (m, 6H), 7.23-7.16 (m, 1H), 6.87 (dd, J=8.6, 4.7 Hz, 4H), 4.98 (d, J=5.8 Hz, 1H), 4.35-4.13 (m, 1H), 4.08-4.01 (m, 1H), 3.77-3.66 (m, 9H), 3.63-3.40 (m, 6H), 3.30-3.17 (m, 1H), 3.16-3.09 (m, 2H), 3.09-3.00 (m, 2H), 2.94 (dd, J=10.3, 5.4 Hz, 1H), 2.75 (t, J=6.0 Hz, 1H), 2.57-2.53 (m, 1H), 2.06-1.98 (m, 4H), 1.69-1.55 (m, 4H), 1.52-1.42 (m, 4H), 1.23 (s, 48H), 1.12-1.04 (m, 9H), 0.91 (d, J=6.8 Hz, 3H), 0.85 (t, J=6.6 Hz, 6H). ³¹P NMR (DMSO-d₆): δ 148.86, 148.57.

### Example 6

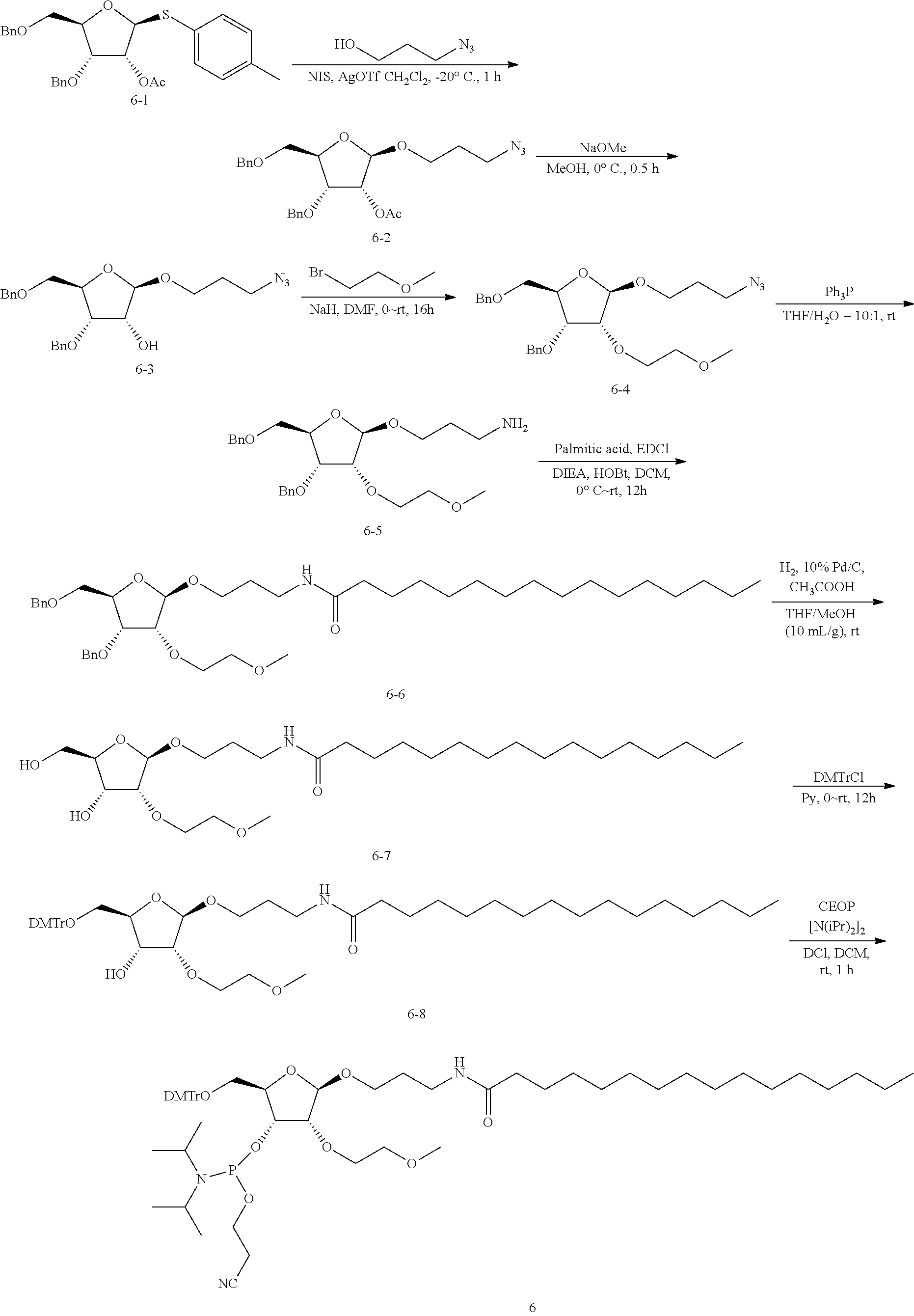

### Preparation of 6-2

To a solution of 6-1 (45 g, 94.1 mmol) and 3-azidopropan-1-ol (23.8 g, 235 mmol, 2.5 equiv) in dichloromethane (450 mL) at −20° C. under Ar were added NIS (23.3 g, 103.6 mmol, 1.1 equiv) and silver trifluoromethanesulfonate (2.4 g, 9.4 mmol, 0.1 equiv). The resulting solution was stirring at −20° C. for 1 h then quenched with 20 mL of trimethylamine and concentrated. The crude product was applied onto a silica gel column and eluted with petroleum ether/ethyl acetate (100:1-10:1) to give 25 g (58% yield) of 6-2 as a yellow oil. MS: m/z 473.30 [M+NH₄]⁺.

### Preparation of 6-3

To a solution of 6-2 (25 g, 54.9 mmol) in MeOH (250 mL) at 0° C. under Ar was added methanolic solution of NaOMe (5 M, 13.2 mL). The resulting mixture was stirring at 0° C. for 0.5 h then quenched with glacial acetic acid and concentrated. The crude product was applied onto a silica gel column and eluted with petroleum ether/ethyl acetate (100:1 to 8:1) to get 20 g (88% yield) of 6-3 as a yellow oil. MS: m/z 431.30 [M+Na]⁺.

### Preparation of 6-4

To a solution of 6-3 (20 g, 48.42 mmol) in DMF (200 mL) at 0° C. was added sodium hydride (60% dispersion in oil, 9.68 g, 242 mmol, 5.0 equiv) and stirred 30 min at 0° C. 1-Bromo-2-methoxyethane (13.3 g, 96.9 mmol, 2.0 equiv) was added and the resulting solution was stirred for 16 h at room temperature. The reaction mixture was quenched with 200 mL of saturated ammonium chloride solution, and then extracted with ethyl acetate (2×200 mL). The combined organic layers were washed with water and brine, dried (anhydrous Na₂SO₄), filtered and concentrated. The crude product was applied onto a silica gel column and eluted with dichloromethane/methanol (100:1-80:1) to give 16 g (70% yield) of 6-4 as a yellow oil. MS: m/z 489.30 [M+Na]⁺. ¹H NMR (DMSO-d₆) δ 7.40-7.22 (m, 10H), 4.94 (d, J=1.1 Hz, 1H), 4.63-4.42 (m, 4H), 4.10-4.01 (m, 1H), 4.00-3.92 (m, 1H), 3.90-3.84 (m, 1H), 3.74-3.51 (m, 4H), 3.49-3.35 (m, 4H), 3.25 (s, 5H), 1.74-1.63 (m, 2H).

### Preparation of 6-5

To a solution of 6-4 (16 g, 34 mmol) in 320 mL of the mixture of tetrahydrofuran and water (10:1) was added triphenylphosphine (17.8 g, 67.9 mmol, 2.0 equiv). The reaction mixture was stirred for 12 h at room temperature and then concentrated. The residue was purified by Flash-Prep-HPLC: C18 column; mobile phase, water (containing 0.05% NH₄HCO₃) and MeCN, gradient 20% to 100% MeCN) to obtain 13 g (86% yield) of 6-5 as a yellow oil. MS: m/z): 446.25 [M+H]⁺.

### Preparation of 6-6

To a solution of 6-5 (13 g, 29.2 mmol) and palmitic acid (11.2 g, 43.8 mmol, 1.5 equiv) in dichloromethane (130 mL) under Ar at 0° C. were added 1-(3-dimethylaminopropyl)-3-ethylcarbodiimide hydrochloride (8.4 g, 43.8 mmol, 1.5 equiv), N,N-diisopropylethylamine (11.3 g, 87.6 mmol, 3 equiv), and 1-hydroxybenzotriazole (5.91 g, 43.8 mmol, 1.5 equiv). The reaction mixture was stirred for 12 h at room temperature then diluted with dichloromethane (200 mL) and washed with water. The organic layer was dried (anhydrous Na₂SO₄), filtered and concentrated under reduced pressure. The residue was purified on a silica gel column with dichloromethane/methanol (100:1-60:1) to get 11 g (55% yield) of 6-6 as a yellow oil. MS m/z 706.45 [M+Na]⁺.

### Preparation of 6-7

To a solution of 6-6 (11 g, 16.10 mmoL) in MeOH/THF (1:1, 110 mL) were added Pd/C (10%, 1.1 g) and acetic acid (5 mL) and stirred under H₂ atmosphere for 8 h. The mixture was filtered and filtrate concentrated. The crude product was purified on a silica gel column with petroleum ether/ethyl acetate (20:1-4:1) to get 7.5 g (92% yield) of 6-7 as a white solid. MS: m/z): 526.40 [M+Na]⁺.

### Preparation of 6-8

To a solution of 6-7 (7.5 g, 14.9 mmol) in pyridine (75 mL) at 0° C. under Ar was added 4,4′-dimethoxytrityl chloride (6.04 g, 17.9 mmol, 1.2 equiv). The resulting solution was stirred for 12 hours at room temperature. The reaction mixture was quenched MeOH (10 mL), diluted with dichloromethane (10 mL), washed with saturated aqueous sodium bicarbonate and saturated brine. The organic layer was dried (anhydrous Na₂SO₄), filtered and concentrated. The residue was applied onto a silica gel column and eluted with dichloromethane/methanol (100:1-40:1) to give 8 g (66% yield) of 6-8 as a light yellow oil. MS: m/z 804.45 [M−H]⁻. ¹H NMR (DMSO-d₆) δ 7.74-7.63 (m, 1H), 7.47-7.40 (m, 2H), 7.34-7.15 (m, 6H), 6.92-6.83 (m, 4H), 4.90 (d, J=1.1 Hz, 1H), 4.07-3.87 (m, 3H), 3.79-3.58 (m, 9H), 3.53-3.35 (m, 3H), 3.26 (s, 3H), 3.17-2.88 (m, 4H), 1.99 (d, J=3.1 Hz, 3H), 1.64-1.36 (m, 4H), 1.23 (s, 24H), 0.95-0.80 (m, 3H).

### Preparation of 6

To a solution of 6-8 (8 g, 9.9 mmol) in dichloromethane (80 mL) under Ar were added 3-((bis(diisopropylamino) phosphaneyl)oxy)propanenitrile (4.2 g, 13.9 mmol, 1.4 equiv) and 4, 5-dicyanoimidazole (1.4 g, 11.9 mmol, 1.2 equiv). The reaction mixture was stirred for 1 h at room temperature then diluted with dichloromethane (80 mL), washed with saturated aqueous sodium bicarbonate and saturated brine. The organic phase was dried (anhydrous Na₂SO₄), filtered and concentrated. The residue was purified by Flash-Prep-HPLC (C18 column; mobile phase, water containing 0.05% NH₄HCO₃ and THF; gradient 20% to 100% THF) to give 5.07 g (49% yield) of compound 6 as a colorless oil. MS: m/z 1006.85 [M+H]⁺. ¹H NMR (DMSO-d₆) δ 7.66 (t, J=5.6 Hz, 1H), 7.51-7.40 (m, 2H), 7.35-7.13 (m, 7H), 6.95-6.81 (m, 4H), 5.01-4.90 (m, 1H), 4.38-4.11 (m, 1H), 4.03 (d, J=6.4 Hz, 1H), 3.81-3.63 (m, 11H), 3.57-3.38 (m, 6H), 3.25 (d, J=3.0 Hz, 3H), 3.20-3.13 (m, 1H), 3.01 (d, J=3.2 Hz, 2H), 2.97-2.87 (m, 1H), 2.79-2.70 (m, 1H), 2.60-2.52 (m, 1H), 2.04-1.94 (m, 2H), 1.58 (d, J=6.1 Hz, 2H), 1.46 (d, J=7.5 Hz, 2H), 1.22 (d, J=2.2 Hz, 24H), 1.13-1.01 (m, 9H), 0.90 (d, J=6.7 Hz, 3H), 0.87-0.81 (m, 3H). ³¹P NMR (DMSO-d₆) δ 148.92, 148.41.

### Example 7

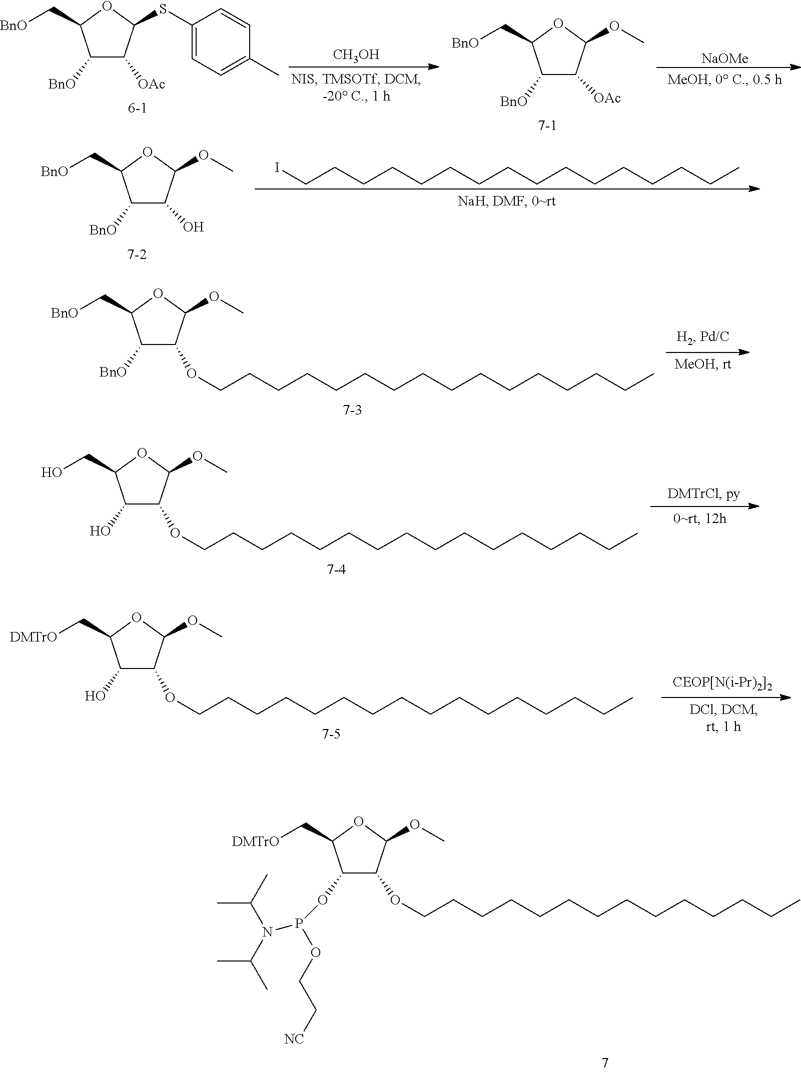

### Preparation of 7-1

To a solution of 6-1 (6 g, 12.5 mmol) in dichloromethane (180 mL) at −20° C. under N₂, were added MeOH (4 g, 125.3 mmol, 10 equiv), NIS (3.1 g, 13.8 mmol, 1.1 equiv) and trimethylsilyl trifluoromethanesulfonate (557 mg, 2.5 mmol, 0.2 equiv). The resulting solution was stirred for 1 h at −20° C. then quenched with aqueous sodium thiosulfate (300 mL). The mixture was extracted with dichloromethane (3×600 mL) and the combined organic extract dried (anhydrous Na₂SO₄), filtered and concentrated. The residue was purified on a silica gel column with ethyl acetate/petroleum ether (1:50 to 1:1) to obtain 4 g (72% yield) of 7-1 as a yellow oil.

### Preparation of 7-2

To a solution of 7-1 (6 g, 15.5 mmol) in MeOH (100 mL) under Ar at 0° C. was added NaOMe (1 g, 18.6 mmol, 1.2 equiv) and stirred at 0° C. for 0.5 h. The resulting solution was diluted with dichloromethane (300 mL) and washed with water. Organic layer was dried (anhydrous Na₂SO₄), filtered and concentrated. The residue was applied onto a silica gel column and eluted with ethyl acetate/petroleum ether (1:10-5:1) to yield 4.5 g (84%) of 7-2 as a yellow oil. MS: m/z 386.17 [M+H]⁺.

### Preparation of 7-3

To a solution of 7-2 (4.5 g, 13.1 mmol) in DMF (90 mL) was added sodium hydride (60% in mineral oil, 2.6 g) at 0° C., and the resulting mixture stirred for 15 min. 1-Iodohexadecane (9.21 g, 26.1 mmol, 2.0 equiv) was added and the mixture stirred at room temperature for 2 h. The reaction mixture was quenched with water (200 mL) and extracted with dichloromethane (3×500 mL). The combined organic fractions were dried (anhydrous Na₂SO₄), filtered and concentrated. The residue was purified on a silica gel column with ethyl acetate/petroleum ether (1:50-1:1) to obtain 5.5 g (74% yield) of 7-3 as a yellow oil. MS: m/z 568.41 [M+Na]⁺. ¹H NMR (DMSO-d₆) δ 7.47-7.20 (m, 10H), 4.83 (d, J=1.1 Hz, 1H), 4.60-4.39 (m, 4H), 4.10-4.01 (m, 1H), 3.96-3.90 (m, 1H), 3.80-3.73 (m, 1H), 3.60-3.36 (m, 4H), 3.22 (s, 3H), 1.60-1.37 (m, 2H), 1.23 (d, J=2.3 Hz, 27H), 0.95-0.77 (m, 3H).

### Preparation of 7-4

To a solution of 7-3 (5.5 g, 9.7 mmol) in MeOH (550 mL) was added 10% Pd/C (2.25 g) and the resulting mixture stirred under H₂ for 16 h. The solid was filtered off and the filtrate was concentrated to get 3 g (80% yield) 7-4 as a white solid. It was used in next step without further purification. MS: m/z 388.32 [M+H]⁺.

### Preparation of 7-5

To a solution of 7-4 (3.2 g, 8.2 mmol) in pyridine (64 mL) under N₂ was added 4,4′-(chloro(phenyl)methylene)bis(methoxybenzene) (2.93 g, 8.6 mmol, 1.1 equiv) at 0° C. The resulting solution was stirred at room temperature and for 2 h then diluted with dichloromethane (200 mL) and washed with water. The organic layer was dried (anhydrous Na₂SO₄), filtered and concentrated. The residue was purified on silica gel column with ethyl acetate/petroleum ether (1:50 to 1:1) to yield 4.1 g (72%) of 7-5 as a yellow oil. MS: m/z 690.45 [M+H]⁺. ¹H NMR (DMSO-d₆) δ 7.47-7.38 (m, 2H), 7.33-7.20 (m, 7H), 6.92-6.82 (m, 4H), 4.80 (d, J=1.0 Hz, 1H), 4.07-3.86 (m, 2H), 3.74 (s, 6H), 3.63-3.43 (m, 3H), 3.27 (s, 3H), 3.10 (dd, J=10.0, 2.6 Hz, 1H), 2.96 (dd, J=9.9, 5.5 Hz, 1H), 1.50 (d, J=6.9 Hz, 2H), 1.25 (d, J=8.1 Hz, 26H), 0.87-0.84 (m, 3H).

### Preparation of 7

To a solution of 7-5 (3 g, 4.3 mmol) in dichloromethane (30 mL) was added 3-((bis(diisopropylamino)phosphaneyl)oxy)propanenitrile (1.7 g, 5.6 mmol, 1.3 equiv) and 4,5-dicyanoimidazole (564 mg, 4.8 mmol, 1.1 equiv) and the resulting solution was stirred at room temperature for 1 h. It was diluted then with dichloromethane and washed with aqueous sodium bicarbonate. Organic layer was dried (anhydrous Na₂SO₄), filtered and concentrated. The residue was purified on a silica gel column with ethyl acetate/petroleum ether (1:50 to 1:10) to yield 1.9 g (48%) of compound 7 as a light yellow oil. MS: m/z 891.60 [M+H]⁺. ¹H NMR (DMSO-d₆) δ 7.49-7.37 (m, 2H), 7.37-7.14 (m, 7H), 6.88 (dd, J=8.9, 2.9 Hz, 4H), 4.87 (d, J=2.3 Hz, 1H), 4.36-4.09 (m, 1H), 4.04 (d, J=6.3 Hz, 1H), 3.74 (d, J=1.4 Hz, 9H), 3.62-3.44 (m, 5H), 3.25-3.11 (m, 1H), 2.96 (dd, J=9.9, 5.3 Hz, 1H), 2.80-2.66 (m, 1H), 2.56 (d, J=5.9 Hz, 1H), 1.50 (s, 2H), 1.24 (s, 27H), 1.13-1.03 (m, 8H), 0.95-0.82 (m, 7H). ³¹P NMR (DMSO-d₆) δ 148.87, 148.5.

### Example 8

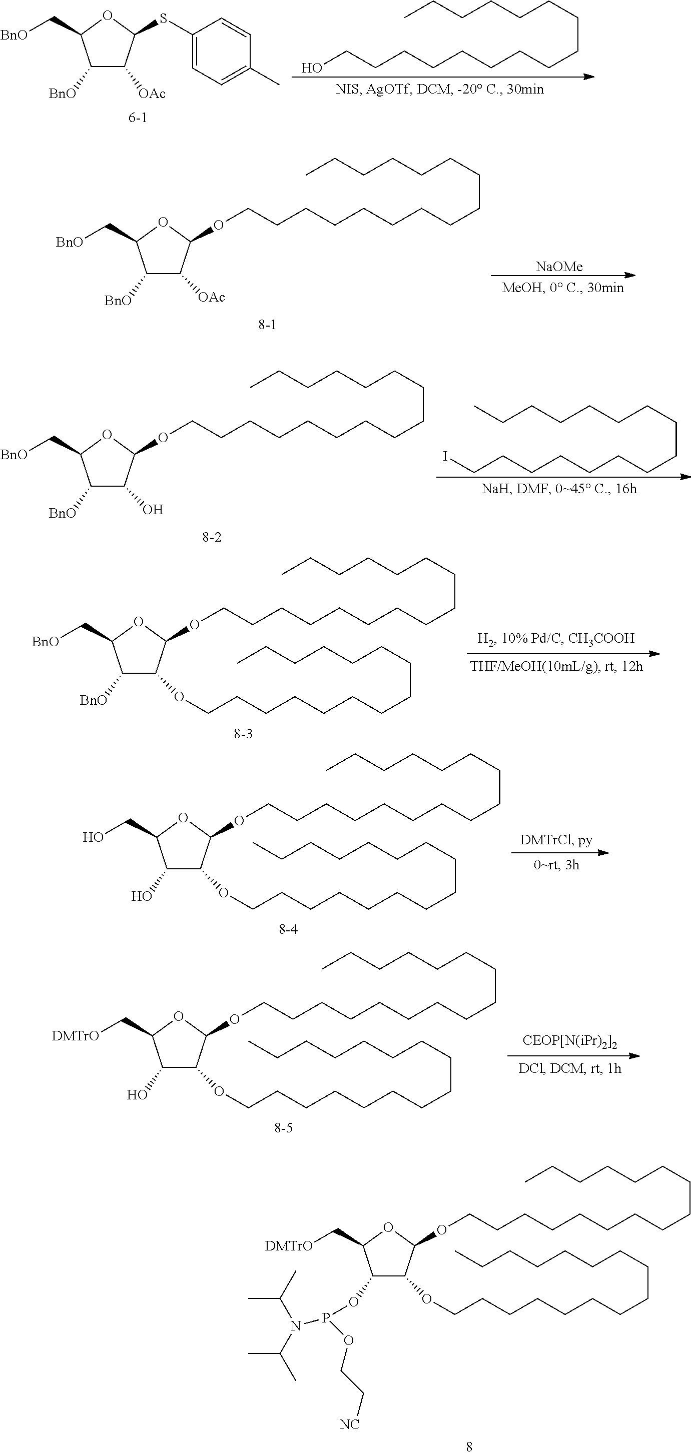

### Preparation of 8-1

To a solution of 6-1 (72 g, 150.6 mmol) and 1-hexadecanol (44 g, 180.58 mmol, 1.2 equiv) in dichloromethane (720 mL) with an inert atmosphere of argon was added N-iodosuccinimide (37 g, 151.1 mmol, 1.1 equiv) and silver trifluoromethanesulfonate (4 g, 15.06 mmol, 0.1 equiv) at −20° C. The resulting solution was stirring at −20° C. for 30 min. The reaction mixture was quenched by 15 mL of trimethylamine and concentrated under reduced pressure. The crude product was applied onto a silica gel column and eluted with petroleum ether/ethyl acetate (50:1-20:1) to yield 70 g (79%) of 8-1 as a light yellow oil. MS: m/z 619.45 [M+Na]⁺. ¹H NMR (CDCl₃) δ 7.46-7.18 (m, 10H), 5.25 (d, J=4.4 Hz, 1H), 5.00 (s, 1H), 4.61-4.58 (m, 2H), 4.46 (d, J=11.4 Hz, 1H), 4.29-4.22 (m, 1H), 4.17 (dd, J=7.5, 4.5 Hz, 1H), 3.76-3.61 (m, 2H), 3.54 (dd, J=10.5, 6.0 Hz, 1H), 3.38 (dt, J=9.4, 6.6 Hz, 1H), 2.15 (s, 3H), 1.51 (q, J=6.7 Hz, 2H), 1.29 (d, J=3.8 Hz, 26H), 0.95-0.88 (m, 4H).

### Preparation of 8-2

To a solution of 8-1 (70 g, 117.4 mmol, 1.0 equiv) was dissolved in 700 mL of methanol with an inert atmosphere of argon and the mixture was added 35 mL of the solution of 5 M sodium methanolate in methanol at 0° C. The resulting solution was stirring at 0° C. for 30 min. The resulting mixture was quenched by 40 mL of 3 M hydrochloric acid and concentrated under reduced pressure. The residue was applied purified on silica gel column with petroleum ether/ethyl acetate (80:1-50:1) to yield 51 g (78%) of 8-2 as a light yellow oil. MS: m/z 577.45 [M+Na]⁺.

### Preparation of 8-3

To a solution of 8-2 (51 g, 92.1 mmol) in 510 mL of DMF was added sodium hydride (11 g, 460 mmol, 5.0 equiv) at 0° C. The resulting solution was stirring for 30 min at 0° C. Then 1-iodohexadecane (47 g, 138 mmol, 1.5 equiv) was added at 0° C. and stirred for 16 h at room temperature. The reaction mixture was quenched by 200 mL of saturated ammonium chloride solution, diluted with 3000 mL of ethyl acetate, washed with 2×1800 mL water, 1×1800 mL of the saturated aqueous solution of sodium thiosulfate, saturated aqueous sodium bicarbonate, and saturated aqueous sodium chloride. The crude product was purified on a silica gel column with petroleum ether/ethyl acetate (100:1-80:1) to yield 49 g (76%) of 8-3 as a yellow solid. MS: m/z 796.85 [M+NH₄]⁺.

### Preparation of 8-4

To a solution of 8-3 (49 g, 61.8 mmol) in 392 mL of tetrahydrofuran and 98 mL of methanol under atmosphere of hydrogen was added 10% Pd/C (49 g) and acetic acid (50 mL) at room temperature and stirred for 12 h. The mixture was filtered and the filtrate concentrated under reduced pressure. 32 g (86% yield) of 8-4 was obtained as a white solid. It was used in next step without further purification. MS: m/z 630.65 [M+NH₄]⁺.

### Preparation of 8-5

To a solution of 8-4 (30 g, 48.9 mmol) in 300 mL of pyridine under an inert atmosphere of argon was added 4,4′-dimethoxytrityl chloride (20 g, 58.8 mmol, 1.2 equiv) and stirred for 3 hours at room temperature. The reaction mixture was quenched by 150 mL of methanol, diluted with 2500 mL of dichloromethane, washed with 2×2000 mL of saturated aqueous sodium bicarbonate and 2×2000 mL of saturated aqueous sodium chloride. The organic layer was dried over anhydrous sodium sulfate, filtered and concentrated under reduced pressure. The crude product was applied onto a silica gel column and eluted with petroleum ether/ethyl acetate with 0.05% TEA (60:1-30:1). 39.1 g (88% yield) of 8-5 was obtained as a light yellow oil. MS: m/z 899.60 [M−H]⁻. ¹H NMR (CDCl₃) δ 7.59-7.47 (m, 2H), 7.41 (d, J=8.7 Hz, 3H), 7.34-7.26 (m, 3H), 6.85 (dd, J=8.8, 6.4 Hz, 4H), 5.02 (dd, J=7.2, 1.4 Hz, 1H), 4.24-4.14 (m, 1H), 4.14-4.06 (m, 1H), 3.82 (d, J=4.7 Hz, 6H), 3.78-3.63 (m, 3H), 3.60-3.51 (m, 1H), 3.46-3.35 (m, 1H), 3.30 (dd, J=9.9, 3.8 Hz, 1H), 3.17 (dd, J=9.9, 5.6 Hz, 1H), 2.60 (d, J=8.3 Hz, 1H), 1.70-1.60 (m, 2H), 1.52 (d, J=13.7 Hz, 2H), 1.28 (d, J=8.8 Hz, 52H), 0.91 (t, J=6.7 Hz, 6H).

### Preparation of 8

To a solution of 8-5 (35 g, 38.9 mmol) in 350 mL of dichloromethane under an inert atmosphere of argon was added 3-(bis(diisopropylamino)phosphinooxy)propanenitrile (14 g, 46.7 mmol, 1.2 equiv) 4,5-dicyanoimidazole (5 g, 42.8 mmol, 1.1 equiv) at room temperature. The resulting solution was stirred for 1 h then diluted with 500 mL of dichloromethane, washed with 3×2000 mL of saturated aqueous sodium bicarbonate and 3×2000 mL of saturated aqueous sodium chloride. The organic layer was dried over anhydrous sodium sulfate, filtered and concentrated under reduced pressure. The crude product was applied onto a silica gel column and eluted with hexane/ethyl acetate with 0.05% TEA (80:1-30:1) to obtain 21 g (51% yield) of compound 8 as a colorless oil. MS: m/z 1101.80 [M+H]⁺. ¹H NMR (CDCl₃) δ 7.52 (dd, J=7.9, 4.0 Hz, 2H), 7.40 (dd, J=8.5, 6.0 Hz, 4H), 7.31 (s, 1H), 7.28-7.18 (m, 2H), 6.83 (dd, J=8.5, 6.3 Hz, 4H), 5.03 (d, J=5.3 Hz, 1H), 4.44-4.18 (m, 2H), 3.81 (d, J=3.6 Hz, 9H), 3.62-3.57 (m, 5H), 3.45-3.27 (m, 2H), 3.10 (dd, J=9.9, 5.0 Hz, 1H), 2.63 (q, J=6.7, 6.1 Hz, 1H), 2.36 (t, J=6.6 Hz, 1H), 1.62-1.60 (m, 1H), 1.50 (d, J=6.4 Hz, 3H), 1.26 (d, J=14.8 Hz, 52H), 1.19-1.13 (m, 8H), 0.99 (d, J=6.7 Hz, 3H), 0.91 (t, J=6.6 Hz, 7H). ³¹P NMR (CDCl₃) δ 149.84, 149.38.

### Example 9

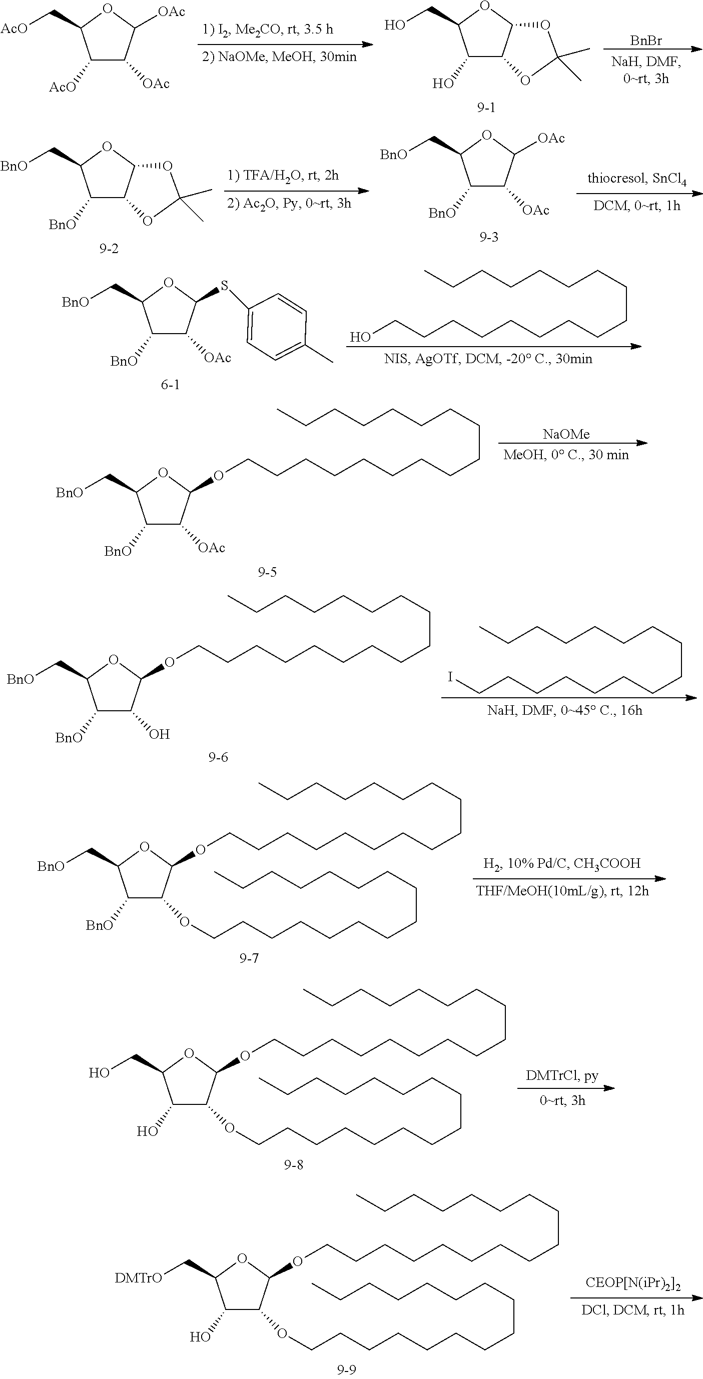

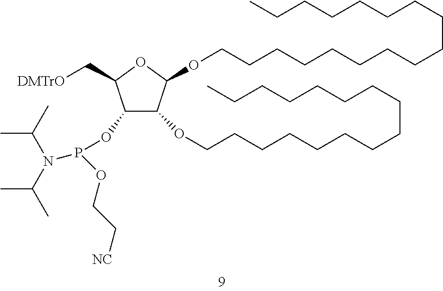

### Preparation of 9-1

To a solution of (3R,4R,5R)-5-(acetoxymethyl) tetrahydrofuran-2,3,4-triyl triacetate (100 g, 314.2 mmoL) in 1000 mL of acetone with an inert atmosphere of argon was added iodine at 0° C. The resulting solution was stirred for 3.5 h at room temperature. The reaction mixture was diluted with 2000 mL of ethyl acetate, washed with 2×500 mL of saturated aqueous sodium thiosulfate and 2×500 mL of saturated aqueous sodium bicarbonate respectively. The organic layer was dried over anhydrous sodium sulfate, filtered and concentrated under reduced pressure. The residue was dissolved in 700 mL of methanol and cooled to 0° C. Then 54 mL of 5 M sodium methanolate in methanol was added and stirred for 30 min at 0° C. with an inert atmosphere of argon. The reaction mixture was quenched by 90 mL of 3 N hydrochloric acid and concentrated under reduced pressure. The crude product was applied onto a silica gel column and eluted with dichloromethane/methanol (120:1-40:1) to yield 37 g (62%) of 9-1 as a white solid. MS: m/z 189.05 [M−H]⁻.

### Preparation of 9-2

To a solution of 9-1 (22 g, 115.8 mmoL) in anhydrous DMF (220 mL) under an inert atmosphere of argon was added sodium hydride (8.34 g, 347.4 mmol, 3.0 equiv) slowly at 0° C. followed by dropwise addition of benzyl bromide (49.5 g, 289.5 mmoL, 2.5 equiv) at 0° C. The resulting solution was stirred for 3 h at room temperature. The reaction mixture was quenched with 20 mL of saturated ammonium chloride solution, diluted with 500 mL of ethyl acetate and washed with 2×150 mL water and 2×150 mL of saturated aqueous sodium chloride. The crude product was applied onto a silica gel column and eluted with petroleum ether/ethyl acetate (80:1-20:1) to give 35.2 g (82% yield) of 9-2 as a yellow oil. MS: m/z 393.25 [M+Na]⁺. ¹H NMR (DMSO-d₆): δ 7.30-7.05 (m, 10H), 5.62 (d, J=3.7 Hz, 1H), 4.67-4.47 (m, 2H), 4.38 (dd, J=12.1, 6.1 Hz, 3H), 3.94-3.85 (m, 1H), 3.70-3.49 (m, 2H), 3.38 (dd, J=11.2, 5.1 Hz, 1H), 1.34 (s, 3H), 1.18 (s, 3H).

### Preparation of 9-3

A solution of 9-2 (30 g, 81.1 mmoL) in 150 mL of trifluoroacetic acid and 150 mL of water was stirred for 2 h at room temperature. The reaction mixture was concentrated under reduced pressure. The residue was dissolved in 300 mL of pyridine and cooled to 0° C. Then 60 mL of acetic anhydride was added and stirred for 3 h at room temperature. The reaction mixture was quenched with 60 mL of water and concentrated under reduced pressure. The residue was diluted with 1000 mL of ethyl acetate and washed by 2×500 mL of water, 1×200 mL of 2 N hydrochloric acid, 1×400 mL of saturated aqueous sodium bicarbonate, 2×500 mL of water and 2×400 mL of saturated aqueous sodium chloride. The organic layer was dried over anhydrous sodium sulfate, filtered and concentrated under reduced pressure. The crude product was applied onto a silica gel column and eluted with petroleum ether /ethyl acetate (20:1-8:1) to yield 21.5 g (64%) 9-3 as a light yellow oil. MS: m/z 437.20 [M+Na]⁺.

### Preparation of 6-1

To a solution of 9-3 (21.5 g, 51.9 mmoL) and (7.7 g, 62.3 mmoL, 1.2 equiv) in dichloromethane (215 mL) under an inert atmosphere of argon was added 10 mL of 1 M stannic chloride pentahydrate in dichloromethane at 0° C. and stirred for 1 h at room temperature. The reaction mixture was quenched with 50 mL of trimethylamine and concentrated under reduced pressure. The crude product was applied onto a silica gel column and eluted with petroleum ether/ethyl acetate (60:1-20:1) to yield 16 g (64%) of 6-1 as a light yellow oil. MS: m/z 496.20 [M+Na]⁺.

### Preparation of 9-5

To a solution of 6-1 (5 g, 10.5 mmol) and 1-heptadecanol (3.2 g, 12.6 mmol, 1.2 equiv) in dichloromethane (50 mL) under an inert atmosphere of argon was added N-iodosuccinimide (2.6 g, 11.5 mmol, 1.1 equiv) and silver trifluoromethanesulfonate (270 mg, 1.1 mmol, 0.1 equiv) at −20° C. The resulting solution was stirred at −20° C. for 30 min. The reaction mixture was quenched with 1 mL of trimethylamine and concentrated under reduced pressure. The crude product purified on a silica gel column with petroleum ether/ethyl acetate (50:1-20:1) to obtain 4.3 g (67% yield) of 9-5 as a light yellow oil. MS: m/z 633.45 [M+Na]⁺.

### Preparation of 9-6

To a solution of 9-5 (4.30 g, 7.1 mmol) in methanol (43 mL) under Ar at 0° C. was added 1.4 mL of the solution of 5 M sodium methanolate in methanol. The resulting solution was stirred at 0° C. for 30 min then the reaction quenched with hydrochloric acid (3 M, 2 mL) and concentrated. The crude product was applied onto a silica gel column and eluted with petroleum ether/EtOAc (80/1 to 50/1) to yield 3.2 g (80%) of 9-6 as a light yellow oil. MS: m/z 591.45 [M+Na]⁺. ¹H NMR (DMSO-d₆): δ 7.36-7.24 (m, 10H), 5.03 (s, 1H), 4.78 (s, 1H), 4.63 (d, J=11.9 Hz, 1H), 4.56-4.46 (m, 2H), 4.43 (d, J=11.9 Hz, 1H), 4.11-4.04 (m, 1H), 3.98 (d, J=4.4 Hz, 1H), 3.83 (dd, J=7.3, 4.4 Hz, 1H), 3.58-3.49 (m, 2H), 3.42 (dd, J=10.6, 6.4 Hz, 1H), 3.31-3.22 (m, 1H), 1.45-1.34 (m, 2H), 1.22 (d, J=12.3 Hz, 28H), 0.88-0.82 (m, 3H).

### Preparation of 9-7

To a solution of 9-6 (4 g, 7.04 mmol) in 40 mL of DMF was added sodium hydride (850 mg, 35.2 mmol, 5.0 equiv) at 0° C. The resulting solution was stirred for 30 min at 0° C. Then 1-iodohexadecane (3.7 g, 10.56 mmol, 1.5 equiv) was added at 0° C. and stirred for 16 h at room temperature. The reaction mixture was quenched with 10 mL of saturated ammonium chloride solution, diluted with 300 mL of ethyl acetate, washed with 2×150 mL water, 1×150 mL of the solution of saturated aqueous sodium thiosulfate and saturated aqueous sodium bicarbonate, and 1×150 mL of saturated aqueous sodium chloride. The crude product was applied onto a silica gel column and eluted with petroleum ether/ethyl acetate (100:1-80:1) to yield 4.1 g (74%) of 9-7 as a yellow solid. MS: m/z 810.85 [M+NH₄]⁺. ¹H NMR (CDCb₃): δ 7.39-7.29 (m, 10H), 5.02 (d, J=1.3 Hz, 1H), 4.68-4.53 (m, 4H), 4.36-4.28 (m, 1H), 4.04 (dd, J=6.9, 4.7 Hz, 1H), 3.80-3.50 (m, 6H), 3.44-3.34 (m, 1H), 1.70-1.58 (m, 2H), 1.53 (t, J=6.8 Hz, 2H), 1.29 (s, 55H), 0.92 (t, J=6.8 Hz, 6H).

### Preparation of 9-8

To a solution of 9-7 (4.1 g, 5.2 mmol) in 32 mL of tetrahydrofuran and 8 mL of methanol under atmosphere of hydrogen was added 10% Pd/C (410 mg) and acetic acid (4 mL) at room temperature. The reaction mixture was stirred for 12 h then filtered and the filtrate concentrated under reduced pressure to yield 2.3 g (73%) of 9-8 as a white solid. It was used in next step without further purification. MS: m/z 630.65 [M+NH₄]⁺.

### Preparation of 9-9

To a solution of 9-8 (2.1 g, 3.4 mmol) in 21 mL of pyridine under an inert atmosphere of argon was added 4,4′-dimethoxytrityl chloride (1.58 g, 4.1 mmol, 1.2 equiv) at room temperature. The resulting solution was stirred for 3 h at 25° C. The reaction mixture was quenched with 10 mL of methanol, diluted with 200 mL of dichloromethane and washed with 2×150 mL of saturated aqueous sodium bicarbonate and 2×150 mL of saturated aqueous sodium chloride. The organic layer was dried over anhydrous sodium sulfate, filtered, and concentrated under reduced pressure. The crude product was applied onto a silica gel column and eluted with petroleum ether/ethyl acetate with 0.05% TEA (60:1-30:1) to obtain 2.3 g (73% yield) of 9-9 as a light yellow oil. MS: m/z 913.60 [M−H]⁻. ¹H NMR (CD₂Cl₂): δ 7.56-7.49 (m, 2H), 7.43-7.37 (m, 4H), 7.37-7.21 (m, 3H), 6.91-6.82 (m, 4H), 5.01 (d, J=1.4 Hz, 1H), 4.19-4.10 (m, 1H), 4.07-3.96 (m, 1H), 3.82 (s, 6H), 3.78-3.63 (m, 3H), 3.63-3.51 (m, 1H), 3.46-3.36 (m, 1H), 3.26 (dd, J=9.9, 3.7 Hz, 1H), 3.13 (dd, J=9.9, 5.7 Hz, 1H), 2.55 (d, J=8.4 Hz, 1H), 1.70-1.61 (m, 2H), 1.56-1.49 (m, 2H), 1.30 (d, J=10.9 Hz, 55H), 0.95-0.90 (m, 6H).

### Preparation of 9

To a solution of 9-9 (2.3 g, 2.5 mmol) in dichloromethane (23 mL) under argon were added 3-(bis(diisopropylamino)phosphinooxy)propanenitrile (910 mg, 3.0 mmol, 1.2 equiv) and 4,5-dicyanoimidazole (330 mg, 2.8 mmol, 1.1 equiv) at room temperature and stirred for 1 h at room temperature. The resulting solution was diluted with 500 mL of dichloromethane, washed with 3×300 mL of saturated aqueous sodium bicarbonate and 3×300 mL of saturated aqueous sodium chloride. The organic layer was dried over anhydrous sodium sulfate, filtered and concentrated under reduced pressure. The crude product was purified on a silica gel column with hexane/ethyl acetate with 0.05% TEA (80:1-30:1) to obtain 2.04 g (73% yield) of compound 9 as a light yellow oil. MS: m/z 1115.85 [M+H]⁺. ¹H NMR (CD₂Cl₂): δ 7.44-7.36 (m, 2H), 7.33-7.24 (m, 4H), 7.23-7.16 (m, 2H), 7.15-7.08 (m, 1H), 6.81-6.54 (m, 4H), 4.90-4.85 (m, 1H), 4.29-4.05 (m, 2H), 3.89-3.71 (m, 1H), 3.69 (d, J=3.1 Hz, 6H), 3.67-3.61 (m, 2H), 3.59-3.38 (m, 5H), 3.35-3.13 (m, 2H), 3.00-2.88 (m, 1H), 2.59-2.45 (m, 1H), 2.35-2.22 (m, 1H), 1.54-1.46 (m, 2H), 1.45-1.35 (m, 2H), 1.26-1.14 (m, 54H), 1.08-1.01 (m, 9H), 0.89 (d, J=6.8 Hz, 3H), 0.84-0.76 (m, 6H). ³¹P NMR (CD₂Cl₂): δ 149.67, 149.39.

### Example 10

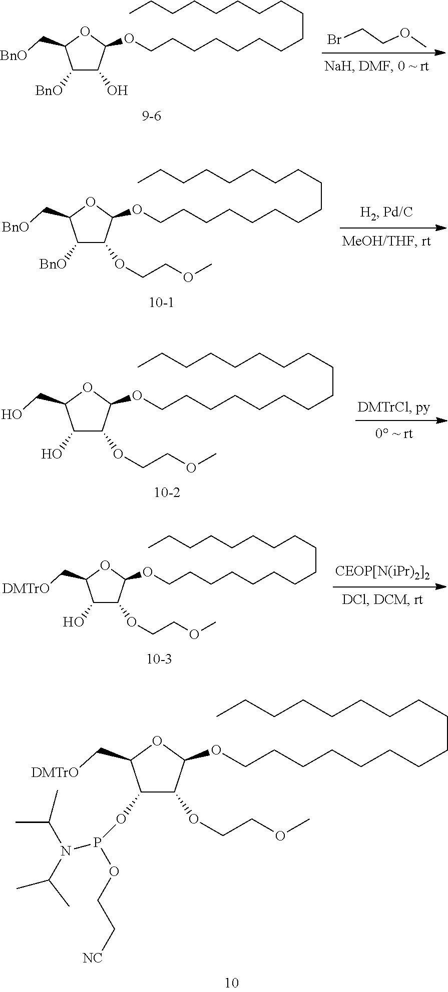

### Preparation of 10-1

To a solution of 9-6 (5 g, 8.8 mmol) in 50 mL of N,N-dimethylformamide was added sodium hydride (60% dispersion in oil, 1.7 g, 43.9 mmoL, 5.0 equiv) at 0° C. and stirred 30 min at 0° C. Then 1-bromo-2-methoxyethane (2.4 g, 17.4 mmoL, 2.0 equiv) was added and the resulting solution was stirred for 16 h at room temperature. The reaction mixture was quenched with 100 mL of saturated ammonium chloride solution and then extracted with 2×200 mL of ethyl acetate. The combined organic layers were washed with 2×100 mL of water and 100 mL of saturated brine, and dried over anhydrous sodium sulfate. The crude evaporated residue was purified on a silica gel column with dichloromethane/methanol (100/1 to 80/1) to give 5 g (90% yield) of 10-1 as a colorless oil. MS: m/z 649.45 [M+Na]⁺. ¹H NMR (DMSO-d₆): δ 7.39-7.23 (m, 10H), 4.92 (d, J=1.1 Hz, 1H), 4.63-4.43 (m, 4H), 4.04 (td, J=6.4, 3.5 Hz, 1H), 3.93 (dd, J=7.0, 4.6 Hz, 1H), 3.85 (dd, J=4.6, 1.1 Hz, 1H), 3.72-3.61 (m, 2H), 3.58-3.49 (m, 2H), 3.48-3.40 (m, 3H), 3.30 (dt, J=9.5, 6.4 Hz, 1H), 3.25 (s, 3H), 1.41 (t, J=6.5 Hz, 2H), 1.21 (d, J=10.0 Hz, 28H), 0.89-0.80 (m, 3H).

### Preparation of 10-2

To a solution of 10-1 (5 g, 7.9 mmoL) in methanol (50 mL) and tetrahydrofuran (50 mL) under the hydrogen atmosphere were added 10% Pd/C (500 mg) and acetic acid (5 mL, 87.4 mmol). The resulting mixture was stirred for 8 h at room temperature then filtered and filtrate concentrated. The crude product was purified on a silica gel column with petroleum ether/ethyl acetate (20/1 to 4/1) to give 10-2 (2.7 g, 76% yield) as a white solid. MS: m/z 469.30 [M+Na]⁺. ¹H NMR (DMSO-d₆): δ 4.81 (d, J=1.9 Hz, 1H), 3.92 (dd, J=6.1, 4.7 Hz, 1H), 3.76-3.67 (m, 2H), 3.66-3.56 (m, 3H), 3.50-3.47 (m, 1H), 3.47-3.45 (m, 2H), 3.44 (dd, J=6.7, 1.4 Hz, 1H), 3.36-3.27 (m, 3H), 3.26 (s, 3H), 1.45 (d, J=6.6 Hz, 2H), 1.24 (s, 28H), 0.91-0.78 (m, 3H).

### Preparation of 10-3

To a solution of 10-2 (2.7 g, 6.0 mmol) in pyridine (27 mL) with an inert atmosphere of argon was added 4,4′-dimethoxytrityl chloride (2.4 g, 7.2 mmoL, 1.2 equiv) at 0° C. The resulting solution was stirred for 6 h at 25° C. The reaction mixture was quenched with 10 mL of methanol, diluted with 100 mL of dichloromethane, washed with 2×100 mL of saturated sodium bicarbonate solution and 2×100 mL of saturated brine. The organic layer was dried (Na₂SO₄), filtered and concentrated. The crude product was purified on a silica gel column with petroleum ether/ethyl acetate (40/1 to 10/1). 3.5 g (77% yield) of 10-3 was obtained as a colorless oil. MS: m/z 747.45 [M−H]⁻. ¹H NMR (DMSO-d₆): δ 7.49-7.39 (m, 2H), 7.37-7.14 (m, 7H), 6.94-6.80 (m, 4H), 4.89 (d, J=1.1 Hz, 1H), 4.07-3.88 (m, 2H), 3.73 (s, 7H), 3.69 (d, J=4.2 Hz, 1H), 3.68-3.65 (m, 1H), 3.64-3.56 (m, 2H), 3.46 (ddd, J=5.6, 4.2, 1.5 Hz, 2H), 3.33 (dt, J=9.5, 6.5 Hz, 1H), 3.26 (s, 3H), 3.10 (dd, J=10.0, 2.6 Hz, 1H), 2.97 (dd, J=9.8, 5.6 Hz, 11H), 1.48-1.34 (m, 2H), 1.19 (d, J=20.9 Hz, 28H), 0.90-0.78 (m, 3H).

### Preparation of 10

To a solution of 10-3 (2.5 g, 3.3 mmol) in 24 mL of dichloromethane was added 3-((bis (diisopropylamino)phosphaneyl)oxy) propanenitrile (1.13 g, 3.96 mmoL, 1.2 equiv), 4,5-dicyano imidazole (427 mg, 3.63 mmoL, 1.1 equiv) at room temperature. The solution was stirred for 45 min at 25° C. The resulting mixture was diluted with 100 mL of dichloromethane, washed with 2×100 mL of saturated sodium bicarbonate solution and 2×100 mL of saturated brine. The organic layer was dried (Na₂SO₄), filtered and concentrated. The crude product was purified on a silica gel column with hexane/ethyl acetate (20/1 to 6/1). The fractions containing product were concentrated to get 2.0 g (63% yield) of compound 10 as a light yellow oil. MS: m/z 949.60 [M+H]⁺. ¹H NMR (DMSO-d₆): δ 7.44 (tdd, J=5.0, 4.3, 3.4, 2.0 Hz, 2H), 7.35-7.24 (m, 6H), 7.24-7.17 (m, 1H), 6.92-6.80 (m, 4H), 5.03-4.92 (m, 1H), 4.35-4.00 (m, 2H), 3.81-3.60 (m, 11H), 3.60-3.34 (m, 6H), 3.26 (d, J=3.9 Hz, 3H), 3.15 (dd, J=10.0, 2.4 Hz, 1H), 2.96 (dt, J=10.5, 5.4 Hz, 1H), 2.79-2.71 (m, 1H), 2.56 (td, J=5.8, 2.1 Hz, 1H), 1.52-1.34 (m, 2H), 1.27-1.01 (m, 37H), 0.92-0.82 (m, 6H). ³¹P NMR (DMSO-d₆): δ 148.67, 148.33.

### Example 11

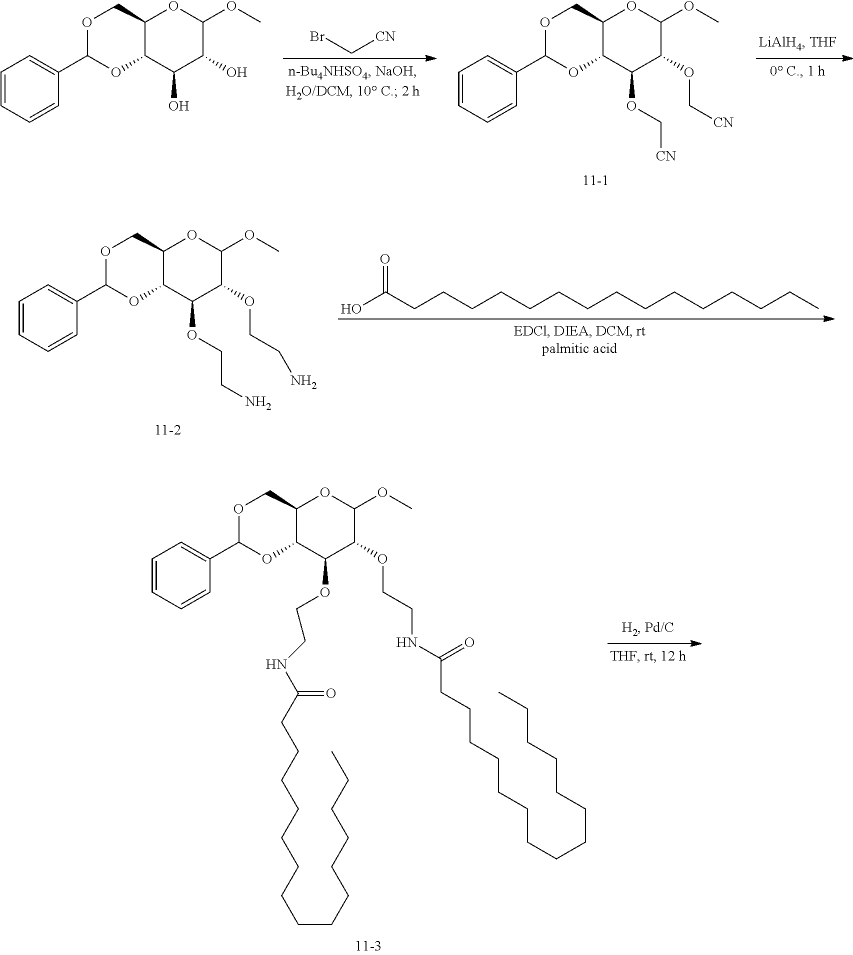

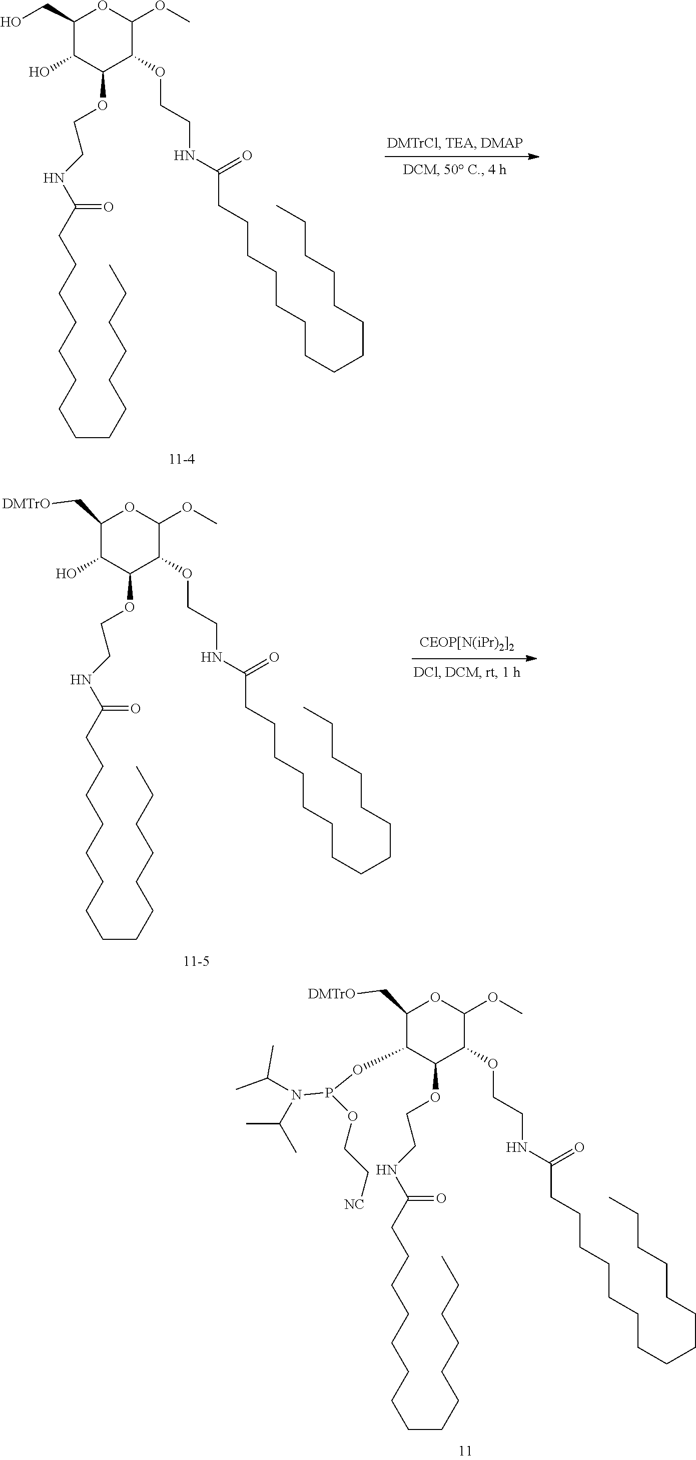

### Preparation of 11-1

To a solution of (4aR,7R,8R,8aS)-6-methoxy-2-phenylhexahydropyrano[3,2-d][1,3]dioxine-7,8-diol (48 g, 170 mmol) in dichloromethane (480 mL) at 0° C. were added tetrabutylammonium hydrogen sulfate (11.5 g, 34.0 mmol, 0.2 equiv), 1M NaOH (850 mL), and bromoacetonitrile (61.3 g, 510.7 mmol, 3.0 equiv). The resulting mixture was stirred for 2 h at room temperature then diluted with dichloromethane (400 mL), washed with water and saturated brine. The organic layer was dried over Na₂SO₄, filtered and concentrated. The residue was purified on a silica gel column with petroleum ether/ethyl acetate (80/1 to 20/1) to obtain 30.1 g (50% yield) of 11-1 as a white solid. MS: m/z 361.20 [M+H]⁺. ¹H NMR (CDCl₃): δ 7.53-7.47 (m, 2H), 7.43-7.38 (m, 3H), 5.58 (s, 1H), 4.95 (d, J=3.8 Hz, 1H), 4.61-4.45 (m, 4H), 4.33 (dd, J=9.7, 4.1 Hz, 1H), 4.04 (t, J=9.1 Hz, 1H), 3.92-3.73 (m, 2H), 3.72-3.61 (m, 2H), 3.49 (s, 3H).

### Preparation of 11-2

To a solution of 11-1 (20 g, 55.6 mmol) in THF (200 mL) at 0° C. under Ar was added LiAlH₄ (4.2 g, 111.1 mmol, 2.0 equiv). The resulting mixture was stirred for 1 h at 0° C. and then quenched with EtOAc. The solids were filtered off. The filtrate was diluted with dichloromethane, washed with water, and saturated brine. The organic layer was dried over Na₂SO₄, filtered, and concentrated. The residue was purified on a silica gel column with dichloromethane/methanol (50/1 to 10/1) to obtain 16.2 g (79% yield) of 11-2 as a white solid. MS: m/z 369.20 [M+H]⁺.

### Preparation of 11-3

To a solution of 11-2 (10 g, 27.1 mmol) in dichloromethane (200 mL) at 0° C. under Ar were added 1-(3-dimethylaminopropyl)-3-ethylcarbodiimide hydrochloride (11.4 g, 59.6 mmol, 2.2 equiv), 1H-benzo[d][1,2,3]triazol-1-ol (18.3 g, 135.5 mmol, 5.0 equiv), N,N-diisopropylethylamine (7.7 g, 59.6 mmol, 2.2 equiv), and palmitic acid (20.8 g, 81.3 mmol, 3.0 equiv). The mixture was stirred for 16 h at room temperature then diluted with dichloromethane, washed with water, and saturated brine. The organic layer was dried over Na₂SO₄, filtered, and concentrated. The residue was purified on a silica gel column with dichloromethane/methanol (80/1 to 20/1) to give 8.2 g (36% yield) of 11-3 as a white solid. MS: m/z 845.75 [M+H]⁺.

### Preparation of 11-4

To a solution of 11-3 (8.2 g, 9.70 mmol) in tetrahydrofuran (164 mL) at room temperature under H₂ was added 10% Pd/C (800 mg). The mixture was stirred for 12 h at 30° C., then filtered and concentrated. The crude product was diluted with dichloromethane (200 mL), filtered, and concentrated. 11-4 was obtained (4.5 g, 61% yield) as a colorless oil. MS: m/z 757.70 [M+H]⁺.

### Preparation of 11-5

To a solution of 11-4 (4.5 g, 5.94 mmol) in CH₂Cl₂ (180 mL) at room temperature under Ar was added 4,4′-dimethoxytrityl chloride (2.2 g, 6.53 mmol, 1.10 equiv), Et₃N (1.7 mL, 12 mmol) and DMAP (146 mg, 1.2 mmol). The resulting solution was stirred at 50° C. for 4 h and quenched with methanol. The mixture was then diluted with dichloromethane, washed with saturated aqueous NaHCO₃ and saturated brine. The organic layer was dried over anhydrous Na₂SO₄, filtered, and concentrated. The residue was purified on a silica gel column with petroleum ether/ethyl acetate (60/1 to 30/1) to obtain 3.2 g (50%) of 11-5 as light-yellow oil. MS: m/z 1081.85 [M+Na]⁺. ¹H NMR (DMSO-d₆) δ 7.79 (t, J=5.7 Hz, 1H), 7.68 (t, J=5.6 Hz, 1H), 7.41 (d, J=7.7 Hz, 2H), 7.35-7.21 (m, 7H), 6.88 (d, J=8.6 Hz, 4H), 5.18 (d, J=6.0 Hz, 1H), 4.85 (d, J=3.3 Hz, 1H), 3.74 (s, 6H), 3.70-3.48 (m, 5H), 3.40 (s, 3H), 3.31-2.97 (m, 9H), 2.09-1.98 (m, 4H), 1.46 (s, 4H), 1.23 (s, 48H), 0.90-0.79 (m, 6H).

### Preparation of 11

To a solution of 11-5 (3 g, 2.83 mmol) in dichloromethane (30 mL) at room temperature under Ar were added 2-cyanoethyl 3-((bis(diisopropylamino)phosphaneyl)oxy) propanenitrile (1.2 g, 3.96 mmol, 1.4 equiv) and 4, 5-dicyanoimidazole (401 mg, 3.39 mmol, 1.2 equiv). The reaction mixture was stirred for 1 h then diluted with dichloromethane, washed with saturated aqueous NaHCO₃ and saturated brine. The organic layer was dried over anhydrous Na₂SO₄, filtered, and concentrated. The residue was purified on a Flash-Prep-HPLC with the following conditions: Column, C18; mobile phase, water (containing 0.05% NH₄HCO₃) and tetrahydrofuran (20% Tetrahydrofuran up to 100% in 10 min and 100% Tetrahydrofuran hold 5 min). The aqueous layer was separated and extracted with dichloromethane. The combined organic layers were combined and dried over anhydrous Na₂SO₄, filtered, and concentrated to obtain 1.9413 g (53%) of compound 11 as a white solid. MS: m/z 1259.90 [M+H]⁺. ¹H NMR (400 MHz, DMSO-d₆) δ 7.89-7.77 (m, 1H), 7.71-7.60 (m, 1H), 7.41 (d, J=7.7 Hz, 2H), 7.34-7.17 (m, 7H), 6.93-6.81 (m, 4H), 4.92 (t, J=3.1 Hz, 1H), 3.81-3.34 (m, 19H), 3.32-3.11 (m, 8H), 3.01 (t, J=9.3 Hz, 1H), 2.68 (d, J=11.9 Hz, 1H), 2.16-1.99 (m, 4H), 1.56-1.40 (m, 4H), 1.23 (s, 48H), 1.09-0.91 (m, 10H), 0.89-0.76 (m, 8H). ³¹P NMR (DMSO-d₆) δ 149.17, 148.15.

### Example 12

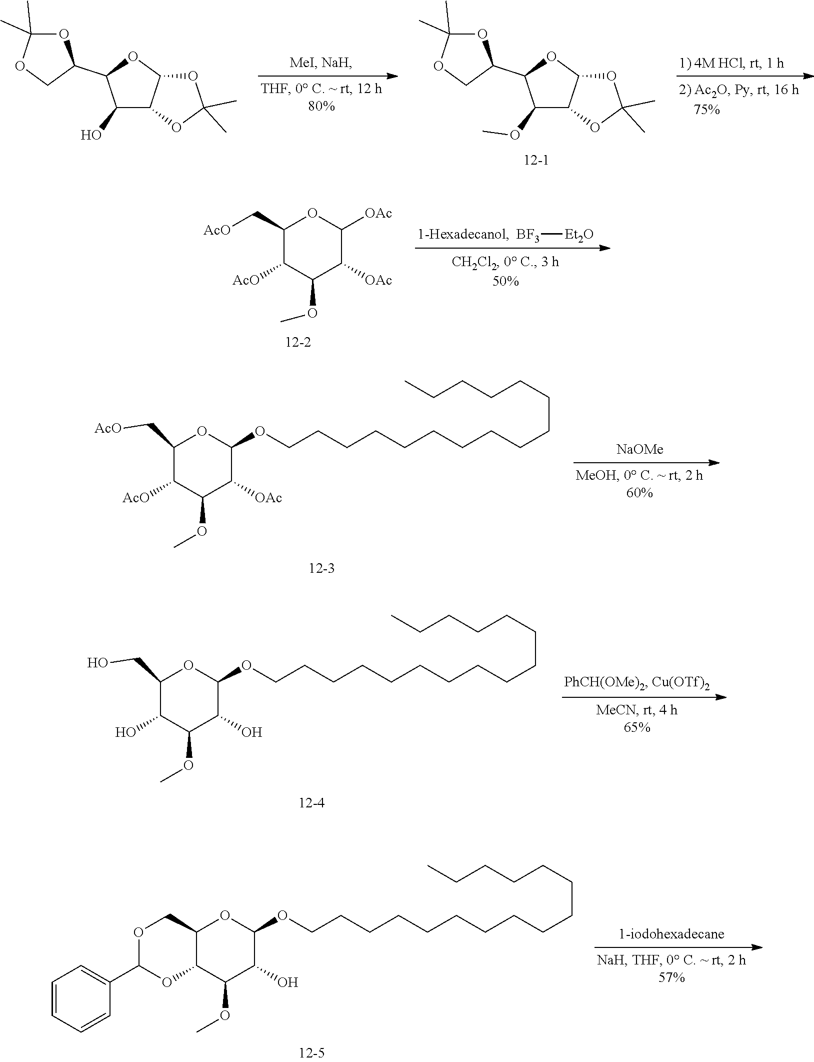

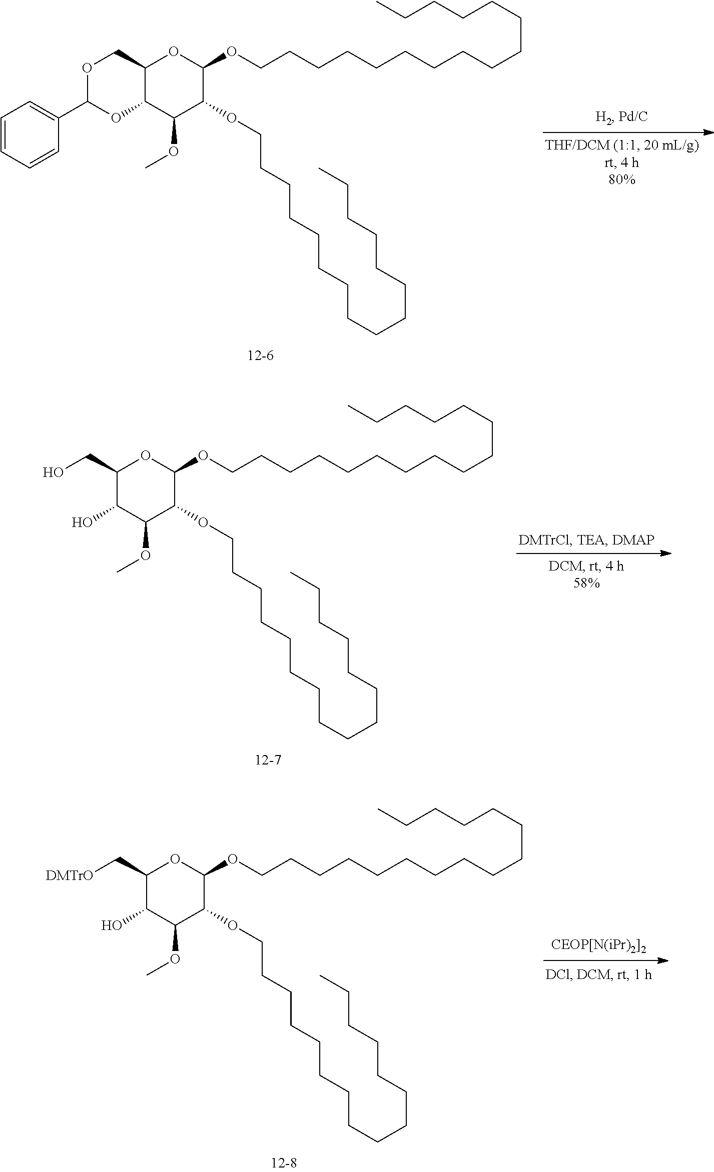

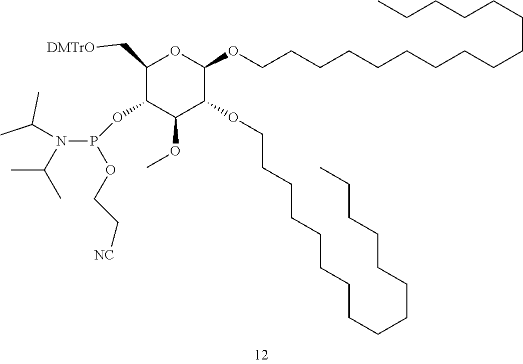

### Preparation of 12-1

To a solution of diacetone-D-glucose (80 g, 307.35 mmol) in tetrahydrofuran (800 mL) at 0° C. under N₂ was added sodium hydride (60% dispersion in mineral oil) (18.4 g, 461.53 mmol, 1.5 equiv). The resulting solution was stirred for 20 min, iodomethane (65.4 g, 461.53 mmol, 1.5 equiv) was added at 0° C., and stirred overnight at room temperature. The reaction was quenched at 0° C. with saturated NH₄Cl solution and extracted with ethyl acetate. The combined organic layers were washed with saturated brine, dried over anhydrous Na₂SO₄, filtered, and concentrated. The residue was purified on a silica gel column with petroleum ether/ethyl acetate (30/1 to 20/1) to obtain 67.2 g (80% yield) of 12-1 as a yellow oil. MS: m/z 275.14 [M+H]⁺.

### Preparation of 12-2

A solution of 12-1 (70 g, 255.474 mmol) in 4M HCl (700 mL) was stirred at room temperature for 1 h, then concentrated. The residue was dissolved in pyridine (550 mL) at 0° C. under N₂ and acetic anhydride (270 mL) was added and stirred overnight at room temperature. The reaction mixture was concentrated and purified on silica gel column with petroleum ether/ethyl acetate (30/1 to 5/1) to obtain 69 g (75% yield) of 12-2 as a yellow solid. MS: m/z 363.15 [M+H]⁺.

### Preparation of 12-3

To a solution of 12-2 (50 g, 138.12 mmol) in dichloromethane (750 mL) at 0° C. under N₂ was added 1-hexadecanol (50.1 g, 207.18 mmol, 1.5 equiv). The resulting solution was stirred for 20 min and boron trifluoride etherate (58.8 g, 414.36 mmol, 3 equiv) was added dropwise and stirred at room temperature for 3 h. The reaction was quenched with saturated NH₄Cl and extracted with dichloromethane. The combined organic layers were washed with saturated brine, dried over anhydrous Na₂SO₄, filtered, and concentrated. The residue was purified on a silica gel column with petroleum ether/ethyl acetate (30/1 to 5/1) to obtain 37.5 g (50% yield) of 12-3 as a yellow solid. MS: m/z 562.50 [M+NH₄]⁺. ¹H NMR (CDCl₃) δ 5.13-4.89 (m, 2H), 4.39 (d, J=7.9 Hz, 1H), 4.26-4.05 (m, 3H), 3.85 (m, 1H), 3.65-3.31 (m, 6H), 2.11-2.06 (m, 8H), 1.25 (d, J=2.0 Hz, 28H), 0.87 (m, 3H).

### Preparation of 12-4

To a solution of 12-3 (35 g, 64.33 mmol) in methanol (350 mL) at 0° C. under N₂ was added sodium methoxide (12.8 g, 321.69 mmol, 5 equiv) and stirred for 2 h at room temperature. The pH of reaction mixture was adjusted to 7 with acetic acid then concentrated. The residue was purified on a silica gel column with dichloromethane/methanol (100/1 to 20/1) to obtain 16 g (60% yield) of 12-4 as a white solid. MS m/z [M+NH₄]+(ESI): 436.40.

### Preparation of 12-5

To a solution of 12-4 (18 g, 43.06 mmol, 1.0 equiv) in chloroform (180 mL) at 0° C. under N₂ were added (dimethoxymethyl) benzene (7.85 g, 51.67 mmol, 1.2 equiv) and copper (II) trifluoromethanesulfonate (779 mg, 2.15 mmol, 0.05 equiv) and stirred for 4 h at room temperature. The reaction mixture was concentrated and purified on a silica gel column with petroleum ether/ethyl acetate (30/1 to 5/1) to obtain 14.1 g (65% yield) of 12-5 as a white solid. MS: m/z 507.50 [M+NH₄]⁺.

### Preparation of 12-6

To a solution of 12-5 (13 g, 25.69 mmol) in tetrahydrofuran (200 mL) at 0° C. under N₂ was added sodium hydride (60% dispersion in mineral oil) (1.5 g, 38.53 mmol, 1.5 equiv). The resulting solution was stirred at 0° C. for 30 min, 1-iodohexadecane (18 g, 51.38 mmol, 2.0 equiv) was added and the reaction was stirred for 2 h at room temperature. The reaction was quenched with saturated NH₄Cl solution and extracted with ethyl acetate. The combined organic layers were washed with saturated brine, dried over anhydrous Na₂SO₄, filtered, and concentrated. The residue was purified on a silica gel column with petroleum ether/ethyl acetate (30/1 to 5/1) to obtain 10.7 g (57% yield) of 12-6 as a white solid. MS: m/z 731.60 [M+H]⁺.

### Preparation of 12-7

To a solution of 12-6 (11 g, 15.06 mmol, 1.0 equiv) in tetrahydrofuran (100 mL) and dichloromethane (100 mL) was added 10% Palladium on activated carbon (11 g) at room temperature. After flushing the reaction mixture with five cycles of H₂, the resulting solution was stirred at room temperature for 4 h, then filtered and concentrated. The residue was purified on a silica gel column with ethyl acetate/petroleum ether (1/5 to 1/3) to obtain 7.7 g (80% yield) of 12-7 as a white solid. MS: m/z 660.60 [M+NH₄]⁺.

### Preparation of 12-8

To a solution of 12-7 (7.7 g, 11.99 mmol, 1 equiv) in dichloromethane (100 mL) at 0° C. under N₂ were added triethylamine (1.8 g, 17.99 mmol, 1.5 equiv), 4-dimethylaminopyridine (146.3 mg, 1.19 mmol, 0.1 equiv), and 1-[chloro(4-methoxyphenyl)benzyl]-4-methoxybenzene (4.8 g, 14.39 mmol, 1.2 equiv). The resulting solution was stirred at room temperature for 4 h, then quenched with methanol and diluted with dichloromethane. The organic layers were washed with saturated brine, dried over anhydrous Na₂SO₄, filtered, and concentrated. The residue was purified on a silica gel column with petroleum ether/ethyl acetate (30/1 to 5/1 with 0.5% triethylamine) to obtain 12-8 in 94% purity. To further enhance the purity, it was recrystallized by dissolving with dichloromethane (1 mL/g) and added to acetonitrile (20 mL/g) dropwise, then filtered to obtain 6 g (58% yield) of 12-8 in 97% purity as a white solid. MS: m/z 943.55 [M−H]⁻. ¹H NMR (CDCl₃) δ 7.50-7.42 (m, 1H), 7.37-7.27 (m, 6H), 7.23-7.17 (m, 2H), 6.89-6.78 (m, 4H), 4.33-4.30 (m, 1H), 3.90-3.85 (m, 2H), 3.81-3.75 (d, J=6.5 Hz, 6H), 3.66 (d, J=3.1 Hz, 3H), 3.64-3.58 (m, 1H), 3.52 (m, 2H), 3.42-3.30 (m, 2H), 3.20-3.07 (m, 2H), 1.68-1.53 (m, 4H), 1.28 (s, 50H), 0.90 (t, J=6.7 Hz, 6H).

### Preparation of 12

To a solution of 12-8 (6 g, 6.15 mmol) in dichloromethane (60 mL) at room temperature under N₂ were added bis(diisopropylamino) (2-cyanoethoxy)phosphine (2.2 g, 7.38 mmol, 1.2 equiv) and 4,5-dicyanoimidazole (800 mg, 6.77 mmol, 1.1 equiv) and stirred for 1 h. The reaction mixture was diluted with dichloromethane and washed with saturated aqueous NaHCO₃ and saturated brine. The organic layer was dried over anhydrous Na₂OS₄, filtered, and concentrated. The residue was purified on a Flash-Prep-HPLC with the following conditions: C-18 column, mobile phase, water (containing 0.04% NH₄HCO₃) and THF, gradient 30-100% THF. The fractions were diluted with dichloromethane and the organic layer was dried over anhydrous Na₂SO₄, filtered, and concentrated. The residue was purified on a silica gel column with hexane/ethyl acetate (30/1 to 5/1) to obtain 2.8291 g (39% yield) of compound 12 as a colorless oil. MS: m/z 1145.90 [M+H]⁺. ¹H-NMR (CDCl₃) δ 7.46-7.37 (m, 2H), 7.36-7.32 (m, 4H), 7.26-7.19 (m, 3H), 6.82-6.77 (m, 4H), 4.34-4.32 (m, 1H), 4.10-3.95 (m, 1H), 3.90-3.80 (m, 1H), 3.77 (s, 6H), 3.62-3.12 (m, 15H). 2.54-2.23 (m, 2H), 1.80-1.70 (mm, 4), 1.69-1.25 (m, 52H), 1.07-10.2 (m, 10H), 0.90-0.81 (m, 8H). ³¹P-NMR (CDCl₃) δ 150.60, 149.83.

### Example 13

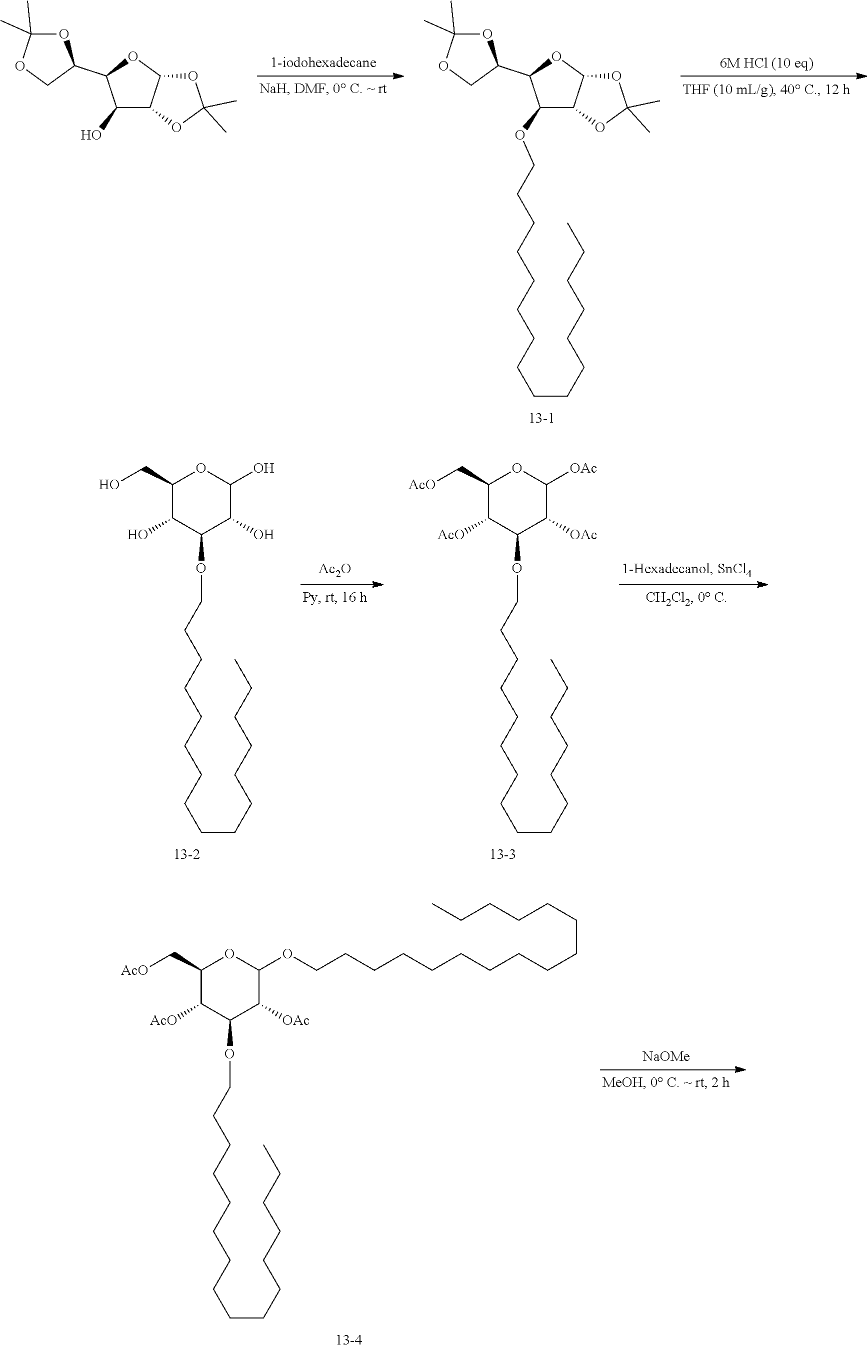

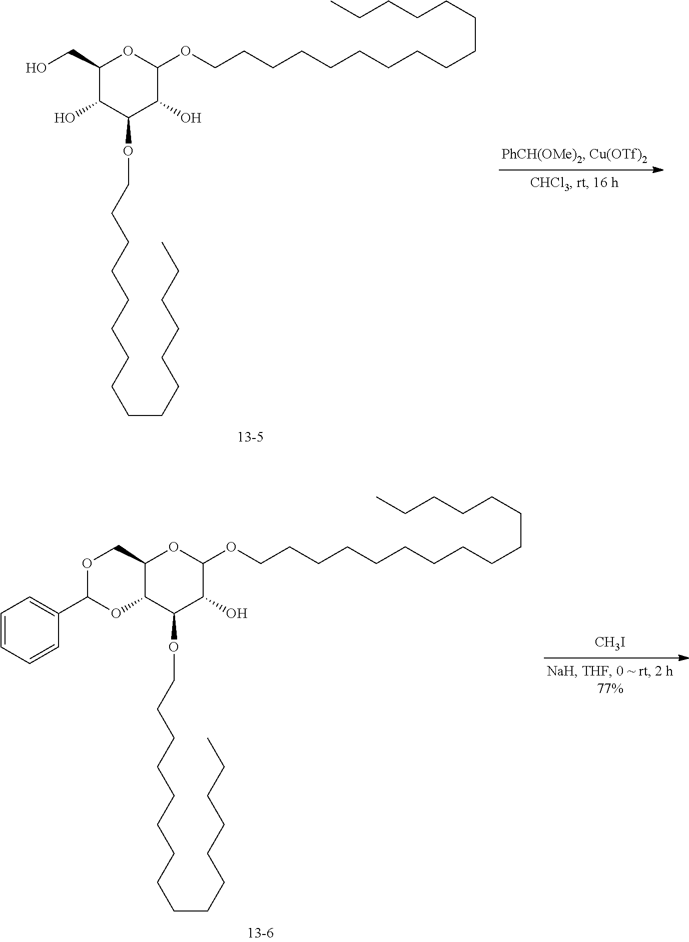

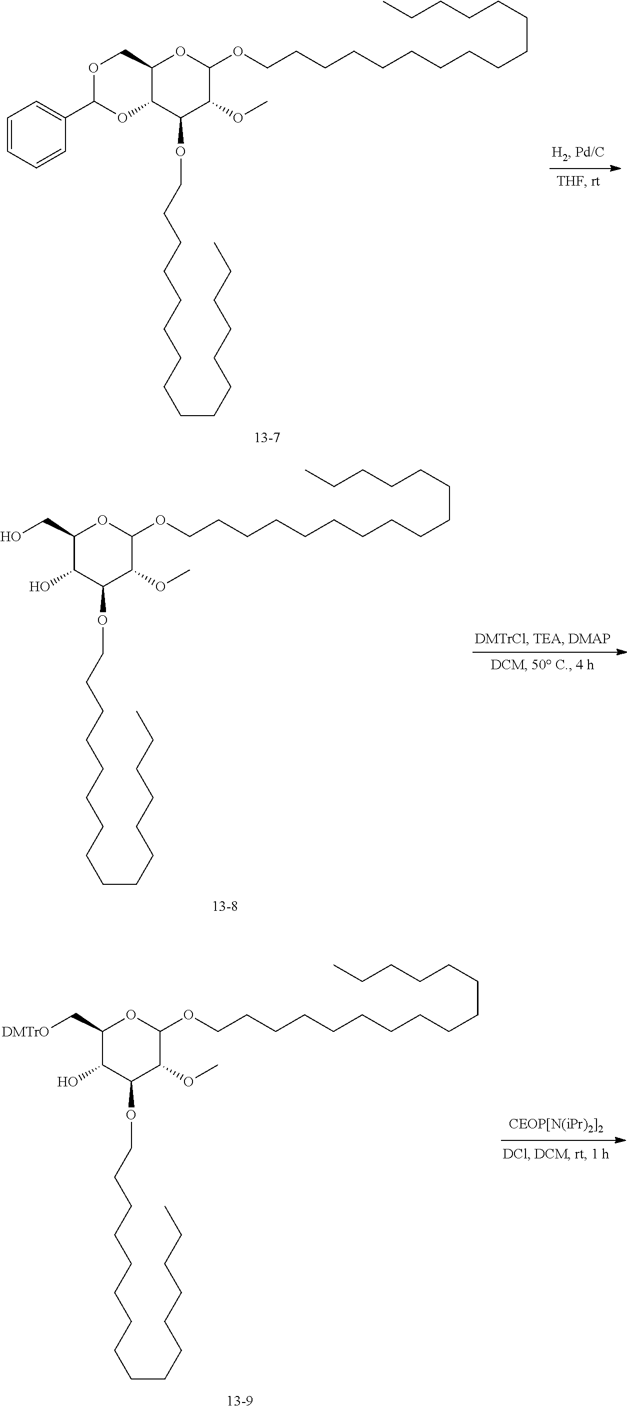

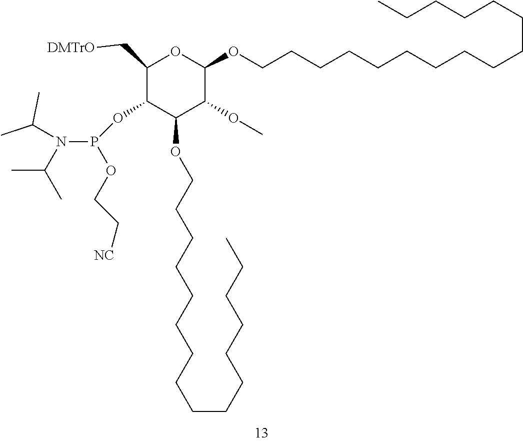

### Preparation of 13-1

To a solution of (3aR,5S,6S,6aR)-5-((R)-2,2-dimethyl-1,3-dioxolan-4-yl)-2,2-dimethyltetrahydrofuro[2,3-d][1,3]dioxol-6-ol (80 g, 307.6 mmol) in DMF (800 mL) at 0° C. under N₂ was added sodium hydride (60%, 24.6 g, 615.4 mmol, 2.0 equiv) and stirred for 15 min. 1-Iodohexadecane (162 g, 461.4 mmol, 1.5 equiv) was added to the reaction mixture at room temperature and stirred for 2 h. The reaction mixture was quenched with saturated NH₄Cl and extracted with dichloromethane. The combined organic layers were washed with water and saturated brine, then the organic layer was dried over anhydrous Na₂SO₄, filtered, and concentrated. The residue was purified on a silica gel column with ethyl acetate/petroleum ether (1/70 to 1/20) to obtain 110 g (75% yield) of 13-1 as a white solid. MS: m/z 485.30 [M+H]⁺.

### Preparation of 13-2

To a solution of 13-1 (110 g, 227.2 mmol) in tetrahydrofuran (1100 mL) at 0° C. was added 6 M HCl (378 mL, 2.27 mol, 10 equiv). The resulting solution was stirred at 40° C. for 12 h then quenched with pyridine (201 mL, 2.5 mol, 11 equiv). The resulting solution was concentrated and co-evaporated with dry pyridine to obtain 110 g (crude) of 13-2 as a yellow oil. It was used in next step without further purification. MS: m/z 422.35 [M+NH₄]⁺.

### Preparation of 13-3

To a solution of 13-2 (110 g, crude) in pyridine (1100 mL) at 0° C. under N₂ was added acetic anhydride (550 mL) and stirred for 16 h. The reaction mixture was then quenched with water and concentrated. It was then diluted with ethyl acetate and washed with water and saturated brine. The organic layer was dried over anhydrous Na₂SO₄, filtered, and concentrated. The residue was purified on a silica gel column with petroleum ether/ethyl acetate (20/1 to 8/1) to obtain 100 g (70% yield over two steps) of 13-3 as a yellow oil. MS: m/z 590.45 [M+NH₄]⁺.

### Preparation of 13-4

To a solution of 13-3 (100 g, 104.8 mmol) and 1-hexadecanol (55.3 g, 157.2 mmoL, 1.5 equiv) in dichloromethane (215 mL) at 0° C. under Ar was added 1 M tin(IV) chloride in dichloromethane (314 mL) and stirred for 1 h at room temperature. The reaction mixture was quenched with trimethylamine and concentrated. The residue was purified on a silica gel column with petroleum ether/ethyl acetate (60/1 to 20/1) to obtain 51 g (64% yield) of 13-4 as a light-yellow oil. MS: m/z 772.65 [M+NH₄]⁺. ¹H NMR (CDCl₃) δ 5.08-4.98 (m, 2H), 4.78 (dd, J=10.0, 3.7 Hz, 1H), 4.22 (dd, J=12.3, 4.9 Hz, 1H), 4.08 (dd, J=12.2, 2.3 Hz, 1H), 3.93-3.91 (m, J=10.3, 5.0, 2.3 Hz, 1H), 3.79 (t, J=9.6 Hz, 1H), 3.72-3.62 (m, 2H), 3.56-3.52 (m, 1H), 3.44 (dt, J=10.0, 6.6 Hz, 1H), 2.11 (d, J=7.5 Hz, 9H), 1.61 (t, J=6.9 Hz, 2H), 1.48 (d, J=6.5 Hz, 2H), 1.27 (d, J=5.2 Hz, 52H), 0.90 (t, J=6.7 Hz, 6H).

### Preparation of 13-5

To a solution of 13-4 (50 g, 66.26 mmol) in methanol (500 mL) at 0° C. under Ar was added 5 M sodium methanolate in methanol (20 mL) at 0° C. and stirred at room temperature for 2 h. The resulting mixture was quenched with 3 M hydrochloric acid (20 mL) and concentrated. The residue was purified on a silica gel column with petroleum ether/ethyl acetate (50/1 to 1/1) to obtain 25 g (80% yield) of 13-5 as a light-yellow oil. MS: m/z 646.60 [M+NH₄]⁺.

### Preparation of 13-6

To a solution of 13-5 (25 g, 39.80 mmol) in chloroform (250 mL) at 0° C. under N₂ was added benzaldehyde dimethyl acetal (9 g, 59.71 mmol, 1.5 equiv) and coper (II) trifluoromethanesulfonate (2.8 g, 7.96 mmol, 0.2 equiv) and stirred for 16 h at room temperature. The reaction mixture was quenched with trimethylamine and concentrated. The residue was purified on a silica gel column with petroleum ether/ethyl acetate (20/1 to 10/1) to obtain 17 g (65%) of 13-6 as a yellow oil. MS: m/z 717.65 [M+H]⁺. ¹H NMR (CDCl₃) δ 7.52-7.50 (m, 2H), 7.43-7.35 (m, 3H), 5.57 (s, 1H), 4.91 (d, J=2.7 Hz, 1H), 4.29 (dd, J=10.0, 4.6 Hz, 1H), 3.93-3.81 (m, 2H), 3.79-3.70 (m, 3H), 3.68-3.62 (m, 2H), 3.59-3.46 (m, 2H), 2.36 (s, 1H), 2.32-2.09 (m, 1H), 1.64 (dt, J=15.4, 7.4 Hz, 4H), 1.27 (d, J=8.9 Hz, 52H), 0.93-0.88 (m, 6H).

### Preparation of 13-7

To a solution of 13-6 (17 g, 23.74 mmol) in DMF (170 mL) at 0° C. under N₂ was added sodium hydride (60%, 1.9 g, 47.48 mmol, 2.0 equiv) and stirred for 15 min. Iodomethane (5 g, 35.61 mmol, 1.5 equiv) was added to the reaction mixture and stirred at room temperature for 2 h. The reaction mixture was quenched with saturated NH₄Cl and extracted with dichloromethane. The combined organic layers were washed with water and saturated brine. The organic layer was dried over anhydrous Na₂SO₄, filtered, and concentrated. The residue was purified on a silica gel column with petroleum ether/ethyl acetate (20/1 to 8/1) to obtain 12 g (77% yield) of 13-7 as a yellow oil. MS: m/z 731.55 [M+H]⁺.

### Preparation of 13-8

To a solution of 13-7 (12 g, 23.28 mmol) in tetrahydrofuran (120 mL) at room temperature under was added 10% Pd/C (12 g) and flushed with five cycles of H₂. The resulting solution was stirred at room temperature for 16 h, then filtered and concentrated to obtain 9 g (80% yield) of 13-8 as a white solid. MS: m/z 660.65 [M+NH₄]⁺.

### Preparation of 13-9

To a solution of 13-8 (9 g, 13.99 mmoL) in CH₂Cl₂ (500 mL) at 0° C. under Ar were added 4,4′-dimethoxytrityl chloride (5.6 g, 16.8 mmoL, 1.2 equiv), Et₃N (3.9 mL, 28 mmol), and DMAP (340 mg, 2.8 mmol) and stirred at 50° C. for 4 hours at room temperature. The reaction mixture was quenched with methanol, diluted with dichloromethane, washed with saturated NaHCO₃ and saturated brine. The organic layer was dried over anhydrous Na₂SO₄, filtered, and concentrated. The residue was purified on a silica gel column with petroleum ether/ethyl acetate (40/1 to 2/1) to obtain 6 g (75% yield) of 13-9 as a colorless oil. MS: m/z 943.65 [M−H]⁻. ¹H NMR (CDCl₃) δ 7.49-7.43 (m, 2H), 7.40-7.32 (m, 4H), 7.32-7.27 (m, 2H), 7.24-7.17 (m, 1H), 6.88-6.77 (m, 4H), 4.97 (d, J=3.6 Hz, 1H), 3.92-3.62 (m, 10H), 3.57-3.46 (m, 6H), 3.38-3.20 (m, 3H), 2.47 (d, J=1.9 Hz, 1H), 1.68-1.60 (m, 4H), 1.26 (d, J=2.6 Hz, 52H), 0.90 (d, J=6.4 Hz, 6H).

### Preparation of 13

To a solution of 13-9 (6 g, 6.35 mmol) in dichloromethane (60 mL) at room temperature under Ar was added 3-(bis(diisopropylamino)phosphinooxy)propanenitrile (2.3 g, 7.62 mmol, 1.2 equiv) and 4,5-dicyanoimidazole (824 mg, 6.98 mmol, 1.1 equiv) and stirred for 1 h. The resulting solution was diluted with dichloromethane, washed with saturated aqueous NaHCO₃ and saturated brine. The organic layer was dried over anhydrous Na₂SO₄, filtered, and concentrated. The residue was purified on a Prep-Flash with the following conditions: C-18 column, mobile phase, water and tetrahydrofuran, gradient 30% to 100% THF. The fractions containing product were diluted with equal volume of dichloromethane and the aqueous layer was separated and extracted with dichloromethane. The combined organic layers were dried over anhydrous Na₂SO₄, filtered, and concentrated. The residue was purified on a silica gel column with hexane/ethyl acetate (10/1 to 2/1) to obtain 2.0 g (30% yield) of compound 13 as a colorless oil. MS: m/z 1145.90 [M+H]⁺. ¹H NMR (CDCl₃) δ 7.46 (d, J=7.6 Hz, 2H), 7.37-7.33 (m, 4H), 7.29 (s, 1H), 7.25-7.15 (m, 2H), 6.80 (dd, J=8.9, 3.8 Hz, 4H), 5.01 (d, J=3.6 Hz, 1H), 3.92 (t, J=9.1 Hz, 2H), 3.78 (s, 6H), 3.76-3.53 (m, 6H), 3.50 (s, 4H), 3.39-3.18 (m, 4H), 3.08 (q, J=10.1, 9.0 Hz, 1H), 2.58-2.20 (m, 2H), 1.76 (q, J=7.5 Hz, 2H), 1.42 (d, J=6.7 Hz, 4H), 1.33-1.17 (m, 50H), 1.03 (dd, J=8.9, 6.6 Hz, 10H), 0.87 (q, J=5.9 Hz, 8H). ³¹P NMR (DMSO-d₆): δ 150.69, 149.57.

### Example 14

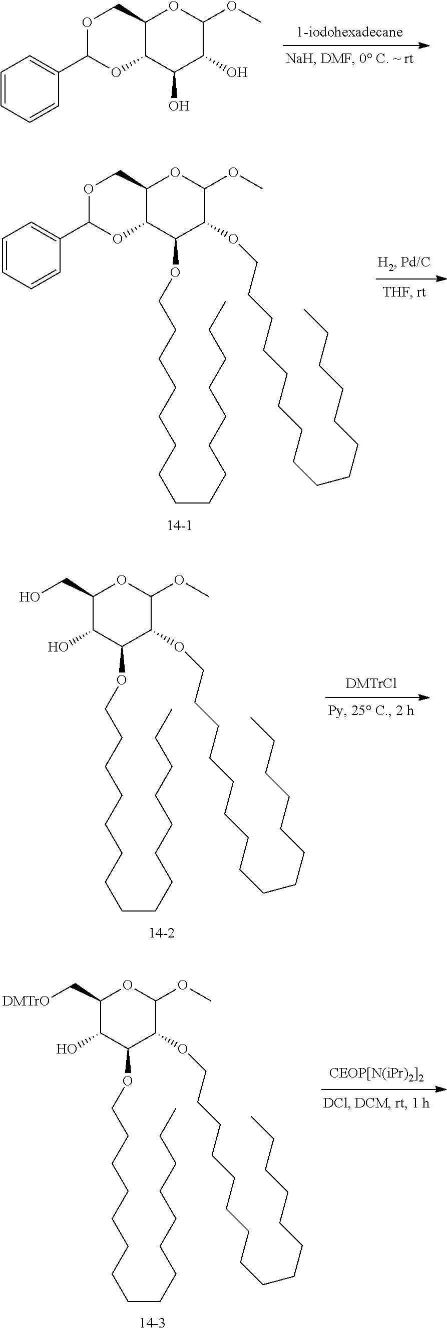

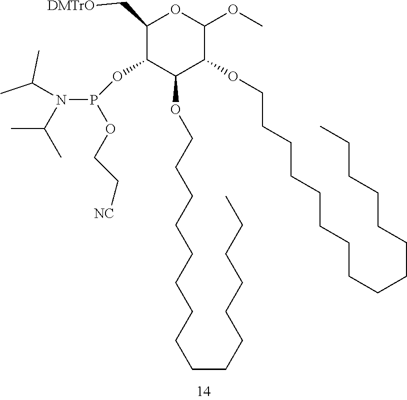

### Preparation of 14-1

To a solution of (4aR,7R,8R,8aS)-6-methoxy-2-phenylhexahydropyrano[3,2-d][1,3]dioxine-7,8-diol (5 g, 17.73 mmol) in DMF (50 mL) at 0° C. under N₂ was added sodium hydride (60%, 3 g, 70.92 mmol, 4.0 equiv) and stirred for 15 min. 1-Iodohexadecane (12 g, 35.46 mmol, 2.0 equiv) was added to the reaction mixture and stirred at room temperature for 2 h. The reaction mixture was quenched with saturated NH₄Cl and extracted with dichloromethane. The combined organic layers were washed with water and saturated brine, then dried over anhydrous Na₂SO₄, filtered, and concentrated. The residue was purified on a silica gel column with ethyl acetate/petroleum ether (1/50 to 1/1) to obtain 5 g (50% yield) of 14-1 as a white solid. MS: m/z 731.60 [M+H]⁺.

### Preparation of 14-2

To a solution of 14-1 (5 g, 6.84 mmol, 1.0 equiv) in tetrahydrofuran (50 mL) at room temperature was added 10% Palladium on activated carbon (wₜ/wₜ=100%, 5 g) and flushed five cycles with H₂. The resulting solution was stirred at room temperature for 16 h, then filtered and concentrated to obtain 3.5 g (80% yield) of 14-2 as a white solid. MS: m/z 643.6 [M+H]⁺.

### Preparation of 14-3

To a solution of 14-2 (3.5 g, 5.45 mmol, 1 equiv) in pyridine (40 mL) at 0° C. under N₂ was added 4,4′-(chloro(phenyl)methylene)bis(methoxybenzene) (2.3 g, 7.08 mmol, 1.3 equiv) and stirred for 2 h at room temperature. The reaction mixture was diluted with dichloromethane and washed with saturated aqueous NaHCO₃ and saturated brine. The organic layer was dried over anhydrous Na₂SO₄, filtered, and concentrated. The residue was purified on a silica gel column with ethyl acetate/petroleum ether (1/50 to 2/1) to obtain 4 g (78% yield) of 14-3 as a white solid. MS: m/z 943.70 [M−H]⁻. ¹H NMR (CDCl₃) δ 7.39-7.32 (m, 2H), 7.29-7.22 (m, 3H), 7.22-7.11 (m, 3H), 7.10-7.04 (m, 1H), 6.73 (dd, J=9.0, 2.6 Hz, 4H), 4.70 (t, J=3.7 Hz, 1H), 3.86-3.71 (m, 1H), 3.69 (d, J=4.1 Hz, 6H), 3.63-3.37 (m, 6H), 3.33 (d, J=3.3 Hz, 3H), 3.30-3.17 (m, 3H), 2.36 (dd, J=6.8, 1.8 Hz, 1H), 1.49 (q, J=7.5, 6.4 Hz, 4H), 1.15 (d, J=2.9 Hz, 52H), 0.86-0.71 (m, 6H).

### Preparation of 14

To a solution of 14-3 (4 g, 4.23 mmol, 1.0 equiv) in dichloromethane (40 mL) at 0° C. under Ar was added 3-(bis(diisopropylamino)phosphinooxy)propanenitrile (1.5 g, 5.07 mmol, 1.2 equiv) and 4,5-dicyanoimidazole (549 mg, 4.65 mmol, 1.1 equiv) and stirred for 1 h at room temperature. The reaction mixture was diluted with dichloromethane, washed with saturated aqueous NaHCO₃ and saturated brine. The organic layer was dried over anhydrous Na₂SO₄, filtered, and concentrated. The residue was purified on a Prep-Flash with the following conditions: column, C18 silica gel; mobile phase, water and tetrahydrofuran (30% tetrahydrofuran up to 100% in 15 min and hold 100% for 5 min); Detector, UV 254 nm. The fraction was diluted with equal volume of dichloromethane. The organic layer was separated, and the aqueous layer was extracted with dichloromethane. The combined organic layers were dried over anhydrous Na₂SO₄, filtered, and concentrated. The residue was purified on a silica gel column with hexane/ethyl acetate (10/1 to 2/1) to obtain 2.2169 g (50% yield) of compound 14 as a colorless oil. MS: m/z 1145.85 [M+H]⁺. ¹H NMR (CDCl₃) δ 7.52-7.46 (m, 2H), 7.40-7.36 (m, 4H), 7.32-7.29 (m, 1H), 7.27 (d, J=4.8 Hz, 1H), 7.26-7.19 (m, 1H), 6.88-6.80 (m, 4H), 4.89 (d, J=3.6 Hz, 1H), 3.91-3.87 (m, 1H), 3.92-3.87 (d, J=1.5 Hz, 7H), 3.73-3.50 (m, 10H), 3.43-3.09 (m, 5H), 2.58-2.20 (m, 2H), 1.63 (d, J=6.7 Hz, 2H), 1.52 (d, J=6.5 Hz, 2H), 1.28-1.26 (m, 52H), 1.10-1.01 (m, 10H), 0.95-0.83 (m, 8H). ³¹P NMR (DMSO-d₆): δ 150.61, 149.21.

### Example 15

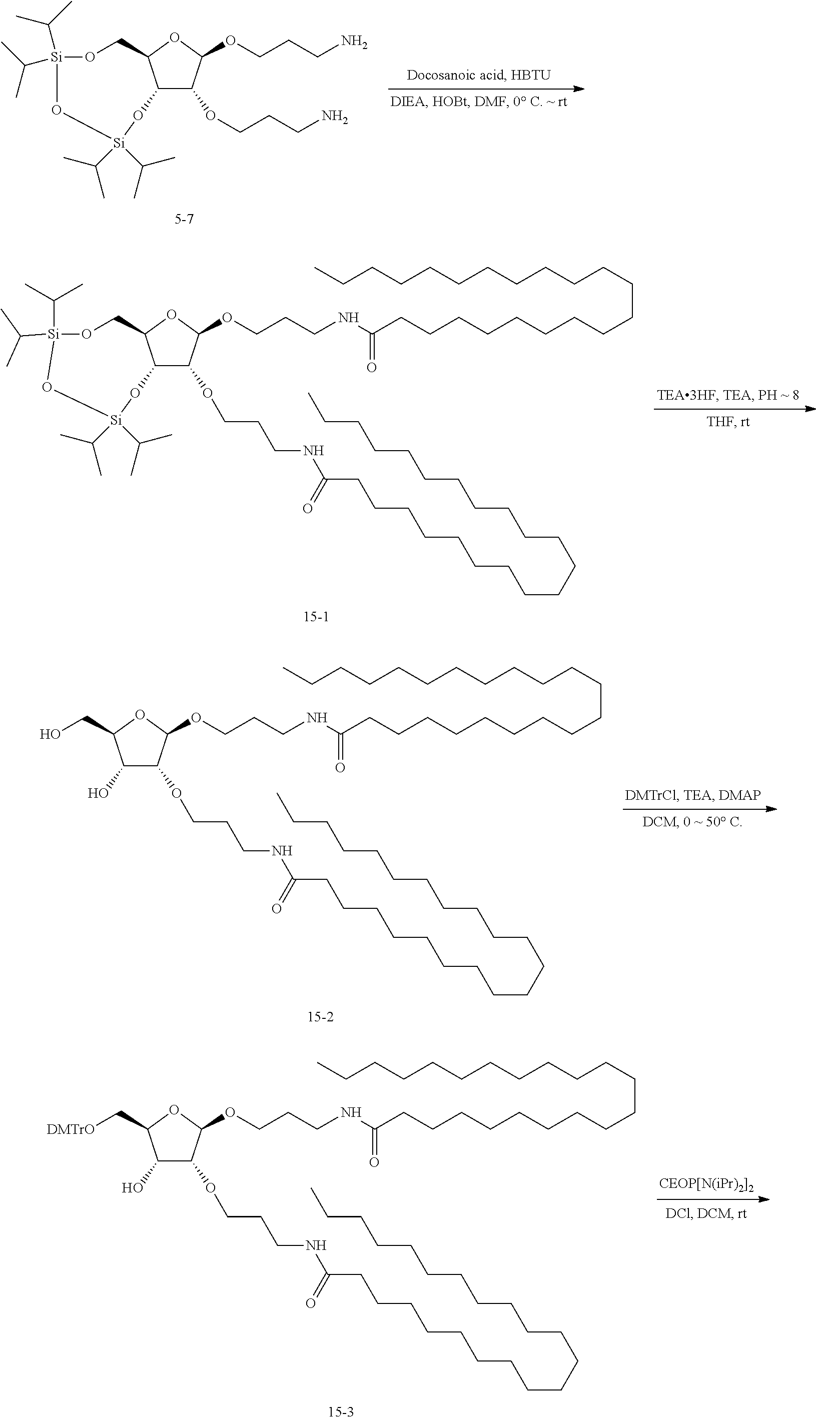

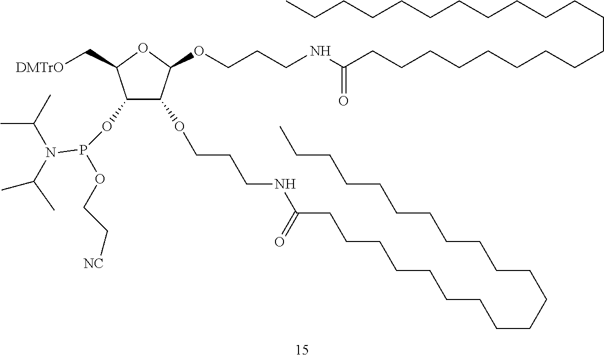

### Preparation of 15-1

To a solution of 5-7 (2.5 g, 5.1 mmol), docosanoic acid (4.3 g, 12.7 mmoL, 2.5 equiv), and N,N-diisopropylethylamine (3.9 g, 30.4 mmoL, 6 equiv) in N,N-dimethyl formamide (25 mL) at 0° C. was added O-benzotriazole-N,N,N′,N′-tetramethyl-uronium-hexafluorophosphate (5.8 g, 15.2 mmoL, 3 equiv) and 1-hydroxybenzotrizole (1.7 g, 12.7 mmoL, 2.5 equiv). The solution was stirred for 12 h at room temperature, then diluted with dichloromethane. The organic layer was washed with water and saturated brine, then dried over anhydrous Na₂SO₄, filtered, and concentrated. The residue was purified on a silica gel column with dichloromethane/methanol (100/1 to 40/1) to obtain 3 g (52% yield) of 15-1 as a white solid. MS: m/z 1192.05 [M+Na+H₂O]⁺.

### Preparation of 15-2

To a solution of triethylamine trihydrofluoride (3.6 g, 22.6 mmoL, 10 equiv) in tetrahydrofuran (52 mL) at room temperature was added triethylamine (4.6 g, 45.1 mmoL, 20 equiv) and 15-1 (2.6 g, 2.3 mmoL, 1 equiv). The solution was stirred for 12 h at room temperature, then precipitated with methanol/water (10:1) and filtered. The solid was washed with acetonitrile to obtain 1.6 g (78% yield) 15-2 as a white solid. MS: m/z 909.82 [M+H]⁺.

### Preparation of 15-3

To a solution of 15-2 (1 g, 1.1 mmoL) in dichloromethane (100 mL) at 0° C. under N₂ was added 4,4′-(chloro (phenyl)methylene)bis(methoxybenzene) (447 mg, 1.3 mmoL, 1.2 equiv), triethylamine (222 mg, 2.2 mmoL, 2 equiv), and 4-dimethylaminopyridine (13 mg, 0.1 mmoL, 0.1 equiv) and stirred for 1 h at 50° C. The reaction mixture was precipitated by acetonitrile and filtrated. The residue was purified on a silica gel column with dichloromethane/methanol (100/1 to 20/1) to obtain 692 mg (52% yield) 15-3 as a white solid. MS: m/z 1209.85 [M−H]⁻.

### Preparation of 15

To a solution of 15-3 (2.8 g, 2.3 mmoL) of dichloromethane (56 mL) was added 3-((bis(diisopropylamino)phosphaneyl)oxy) propanenitrile (0.9 g, 3.0 mmol, 1.3 equiv) and 4,5-4,5-dicyanoimidazole (300 mg, 2.5 mmoL, 1.1 equiv) and stirred at room temperature for 45 minutes. The reaction mixture was diluted with dichloromethane and washed with saturated aqueous NaHCO₃ and saturated brine. The organic layer was dried over anhydrous Na₂SO₄, filtered, and concentrated. The residue was purified by C18 Flash chromatography (mobile phase, THF in water (0.05% ammonium hydrogen carbonate), 20% to 100% gradient in 25 min). The fraction was diluted with equal volume of dichloromethane. The organic phase layer was separated, and the aqueous layer was extracted with of dichloromethane. The combined organic layer was dried over anhydrous Na₂SO₄, filtered, and concentrated. The residue was purified on a silica gel column with n-hexane/ethyl acetate (100/1 to 1/1) to obtain 2.09 g (61% yield of compound 15 as a white solid. MS: m/z 1412.05 [M+H]⁺. ¹H-NMR (CDCl₃): δ 7.49-7.47 (m, 2H), 7.38-7.34 (m, 4H), 7.26-7.21 (m, 4H), 6.81 (t, J=6 Hz, 4H), 5.66-5.49 (m, 1H), 5.00 (s, 1H), 4.41-4.19 (m, 1H), 4.09 (s, 1H), 3.78 (s, 9H), 3.71-3.66 (m, 1H), 3.69-3.34 (m, 8H), 3.19-3.03 (m, 2H), 2.62 (t, J=6 Hz, 1H), 2.35 (t, J=6 Hz, 1H), 2.17-1.90 (m, 4H), 1.82-1.68 (m, 4H), 1.63-1.45 (m, 4H), 1.25 (s, 72H), 1.14 (t, J=7.5 Hz, 9H), 0.98 (d, J=6 Hz, 3H), 0.88 (t, J=7.5 Hz, 6H); ³¹P-NMR (CD₂Cl₂): δ 149.81, 149.51.

### Example 16

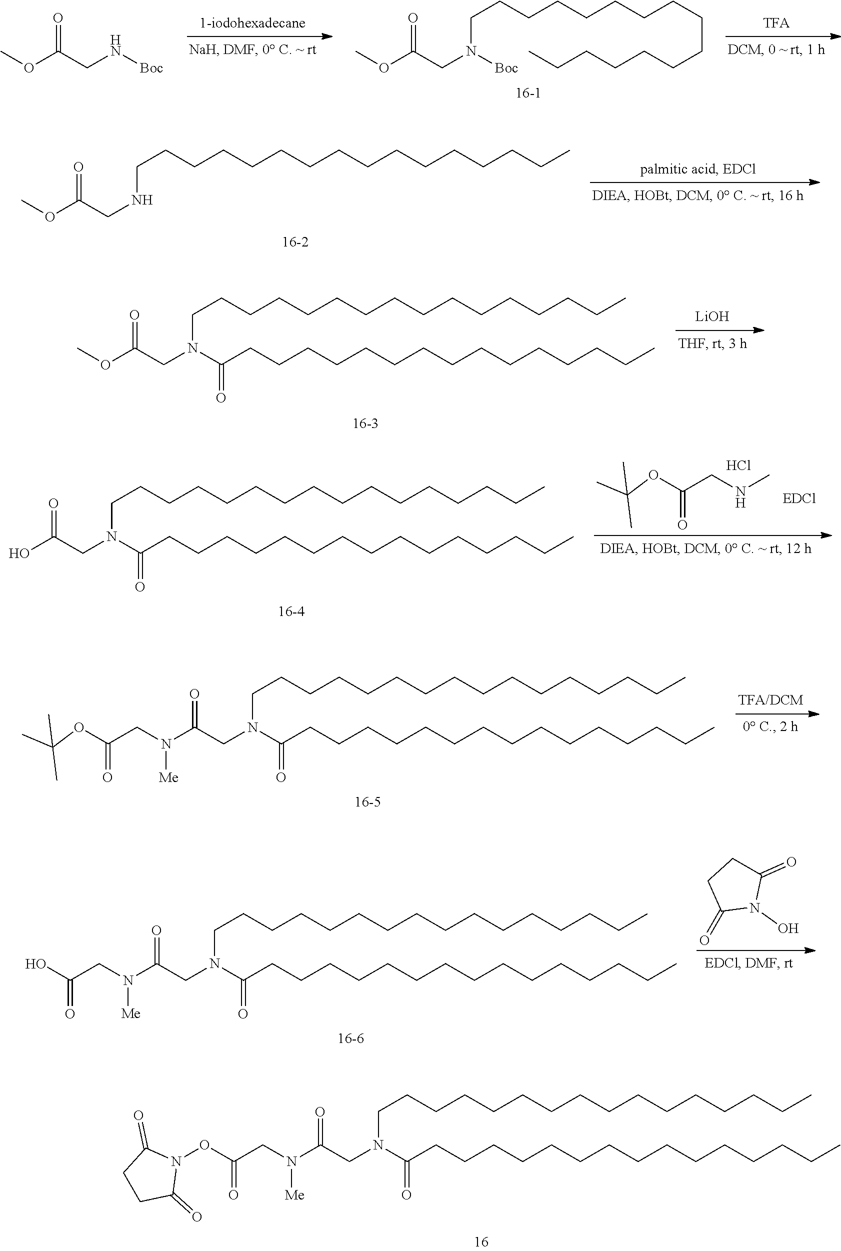

### Preparation of 16-1

To a solution of methyl (tert-butoxycarbonyl)glycinate (22 g, 116.40 mmol) in 220 mL of N,N-dimethylformamide was added sodium hydride (5 g, 127.5 mmol, 1.1 equiv) at 0° C. The resulting solution was stirring for 30 min at 0° C. Then 1-iodohexadecane (49 g, 139.58 mmol, 1.2 equiv) was added at 0° C. and stirred for 16 h at room temperature. The reaction mixture was quenched with 50 mL of saturated ammonium chloride solution, diluted with 1500 mL of ethyl acetate, washed with 2×300 mL water, 1×300 mL of the solution of saturated aqueous sodium thiosulfate and saturated aqueous sodium bicarbonate, and 1×300 mL of saturated brine. The crude product was purified on a silica gel column with petroleum ether/ethyl acetate (70/1 to 20/1) to obtain 22 g (52% yield) of 16-1 as a yellow solid. MS: m/z 436.35 [M+Na]⁺.

### Preparation of 16-2

To a solution of 16-1 (22 g, 53.14 mmol) in 110 mL of dichloromethane was added 110 mL of trifluoroacetic acid at 0° C. The resulting solution was stirring for 1 hour at 0° C. The solvent was removed under reduced pressure, and the crude product was purified on a silica gel column with hexane/ethyl acetate (70/1 to 1/2) to get 13 g (77% yield) of 16-2 as a white solid. MS: m/z 314.30 [M+H]⁺.

### Preparation of 16-3

To a solution of 16-2 (13 g, 42.17 mmol) and N,N-diisopropylethylamine (16 g, 122.40 mmol, 3.0 equiv) in 130 mL of dichloromethane with an inert atmosphere of argon was added palmitic acid (13 g, 48.86 mmol, 1.2 equiv) at 0° C. The mixture was stirred for 10 min then added N-(3-dimethylaminopropyl)-N′-ethylcarbodiimide hydrochloride (12 g, 61.15 mmol, 1.5 equiv) and 1-hydroxybenzotriazole (9 g, 60.79 mmol, 1.5 equiv) at 0° C. The resulting solution was stirring for 16 h at room temperature, then was diluted with 1000 mL of dichloromethane and washed with 2×300 mL of water and 2×300 mL of saturated brine. The organic layer was dried over anhydrous sodium sulfate and concentrated under reduced pressure. The crude product was purified on a silica gel column with hexane/ethyl acetate (70/1 to 2/1) to obtain 13 g (58% yield) of 16-3 as a white solid. MS: m/z 552.60 [M+H]⁺. ¹H NMR (CDCl₃) δ 4.05 (d, J=8.5 Hz, 2H), 3.76 (d, J=18.8 Hz, 3H), 3.40-3.29 (m, 2H), 2.40-2.34 (m, 2H), 1.71-1.53 (m, 4H), 1.35-1.25 (m, 50H), 0.89 (d, J=7.0 Hz, 6H).

### Preparation of 16-4

To a solution of 16-3 (13 g, 23.91 mmol) in 130 mL of tetrahydrofuran with an inert atmosphere of argon was added 5M lithium hydroxide (24 mL, 119.55 mmol, 5.0 equiv) at 0° C. The resulting solution was stirring for 3 h at room temperature. The mixture was diluted with 1000 mL of dichloromethane and adjust pH to 6 with 2M hydrochloric acid then washed with 2×300 mL of water and 2×300 mL of saturated brine. The organic layer was dried over anhydrous sodium sulfate and concentrated under reduced pressure. The crude product was applied onto a silica gel column and purified with hexane/ethyl acetate (70/1 to 1/2) to afford 11 g (83% yield) of 16-4 as a white solid. MS: m/z 538.55 [M+H]⁺.

### Preparation of 16-5

To a solution of 16-4 (11 g, 19.89 mmol) and N,N-diisopropylethylamine (10 g, 79.6 mmol, 4.0 equiv) in 100 mL of dichloromethane with an inert atmosphere of argon was added tert-butyl methylglycinate (4 g, 23.6 mmol, 1.2 equiv) at 0° C. The mixture was stirred for 10 min then added N-(3-dimethylaminopropyl)-N′-ethylcarbodiimide hydrochloride (6 g, 29.7 mmol, 1.5 equiv) and 1-hydroxybenzotriazole (4 g, 29.5 mmol, 1.5 equiv) at 0° C. The resulting solution was stirring for 12 h at room temperature, then was diluted with 1000 mL of dichloromethane and washed with 2×300 mL of water and 2×300 mL of saturated brine. The organic layer was dried over anhydrous sodium sulfate and concentrated under reduced pressure. The crude product was applied onto a silica gel column and purified with hexane/ethyl acetate (70/1 to 2/1) to obtain 8 g (57% yield) of 16-5 as a white solid. MS: m/z 665.65 [M+H]⁺.

### Preparation of 16-6

To a solution of 16-5 (8 g, 7.04 mmol) in 50 mL of dichloromethane was added 25 mL of trifluoroacetic acid at 0° C. The resulting solution was stirring for 2 hours at 0° C. The solvent was removed under reduced pressure, and the crude product purified on a silica gel column with hexane/ethyl acetate (70/1 to 1/2) to obtain 5 g (70% yield) of 16-6 as a white solid. MS: m/z 609.55 [M+H]⁺.

### Preparation of 16

To a solution of 16-6 (4 g, 6.25 mmol, 1.0 equiv) in 38 mL of DMF with an inert atmosphere of argon, were added 1-hydroxypyrrolidine-2,5-dione (2 g, 18.72 mmol, 3.0 equiv) and 1-ethyl-3-(3-dimethylaminopropyl)carbodiimide hydrochloride (2 g, 9.36 mmol, 1.5 equiv) at 0° C. The reaction mixture was stirred for 16 h at 25° C. then cooled to 0° C. The resulting solution was filtered and the filter cake washed with cold DMF and cold acetonitrile. The solid was dissolve in dichloromethane and then concentrated to obtain 1.9 g (43% yield) of compound 16 as a white solid. MS: m/z 706.70 [M+H]⁺. ¹H NMR (CDCl₃) δ 4.56 (d, J=35.8 Hz, 2H), 4.15 (d, J=42.3 Hz, 2H), 3.36 (t, J=7.6 Hz, 2H), 3.14 (s, 2H), 3.04 (s, 1H), 2.85 (d, J=9.7 Hz, 4H), 2.36 (t, J=7.6 Hz, 2H), 1.64 (d, J=6.8 Hz, 6H), 1.26 (s, 48H), 0.88 (t, J=6.7 Hz, 6H).

### Example 17

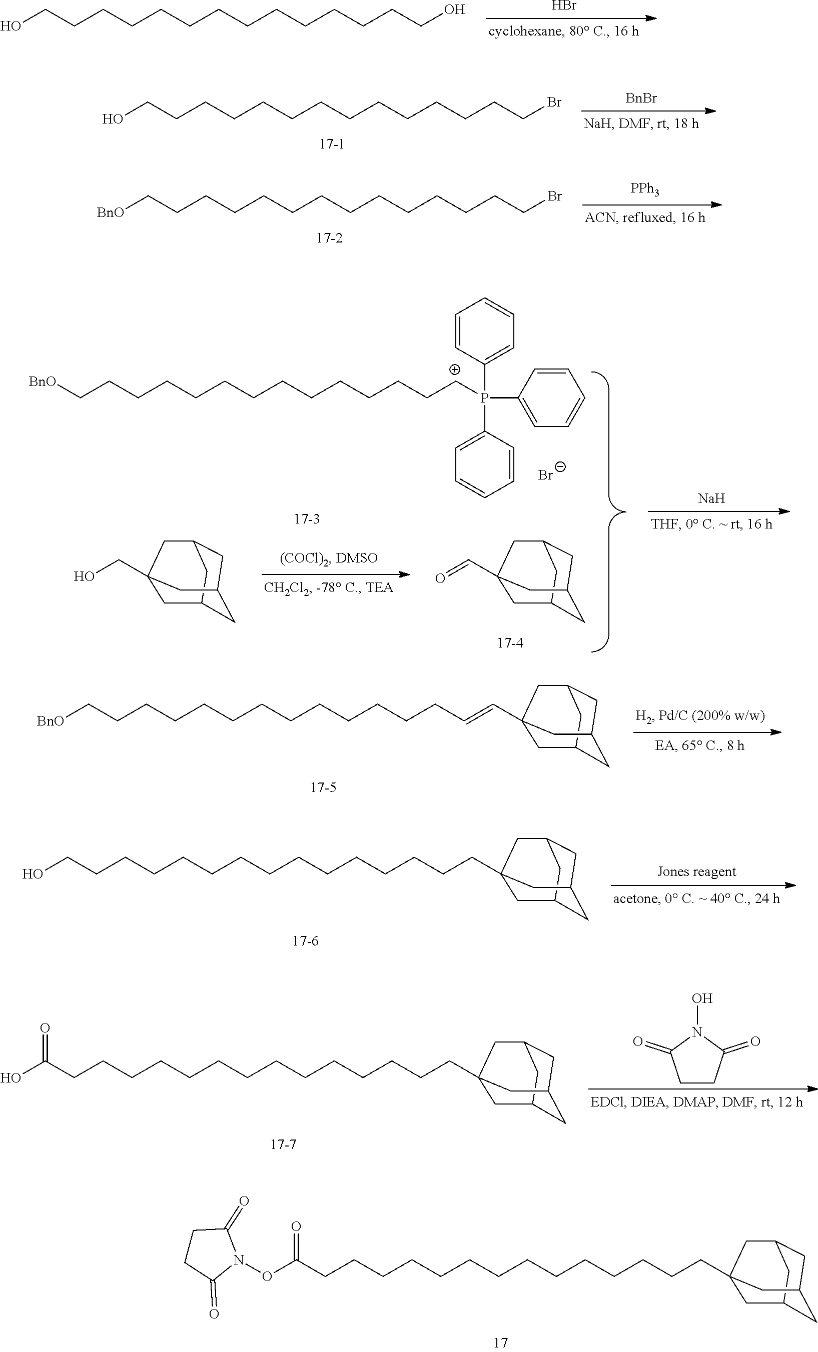

### Preparation of 17-1

To a solution of tetradecane-1,14-diol (54 g, 234.388 mmol) in 540 mL of cyclohexane was added hydrogen bromide (20.9 g, 257.827 mmol, 1.1 equiv) at room temperature, then solution was stirred for 16 h at 80° C. The reaction was quenched by the addition of saturated sodium bicarbonate solution (500 mL), the resulting solution was extracted by 3×800 mL of dichloromethane. The combined organic layers were washed with 2×500 mL of water and 500 mL of brine. The organic layer was dried over anhydrous sodium sulfate, filtered and concentrated under reduced pressure. The crude product was applied onto a silica gel column and elute with petroleum ether/ethyl acetate (100/1 to 4/1) to obtain 42 g (61% yield) of 17-1 as a white solid.

### Preparation of 17-2

To a solution of 17-1 (22 g, 75.0 mmol) in 440 mL of dimethyl formamide was added sodium hydride (3.6 g, 150.02 mmol, 2.0 equiv) at 0° C., the solution was stirred for 30 min at 0° C., then added (bromomethyl)benzene (19.2 g, 112.5 mmol, 1.5 equiv) at 0° C. Then solution was stirred for 18 h at room temperature. The reaction was quenched by the addition of saturated ammonium chloride solution (500 mL), the resulting solution was extracted by 3×500 mL of dichloromethane. The combined organic layers were washed with 2×300 mL of water and 300 mL of saturated brine. The organic layer was dried over anhydrous sodium sulfate, filtered and concentrated under reduced pressure. The crude product was purified on a silica gel column with petroleum ether/ethyl acetate (100/1 to 4/1) to obtain 21.0 g (73% yield) of 17-2 as a yellow oil.

### Preparation of 17-3

To a solution of 17-2 (50 g, 130.4 mmol) in 400 mL of acetonitrile was added triphenylphosphane (68.4 g, 260.8 mmol, 2.0 equiv) at room temperature and the resulting mixture was stirred reflux temperature for 16 h. The product was precipitated with n-hexane and filtrated. Yield of 17-3 as a white solid was 64.0 g (76%). MS: m/z 565 [M+H]⁺.

### Preparation of 17-4

To a solution of oxalyl chloride (572 mg, 4.51 mmol, 1.5 equiv) in dry tetrahydrofuran (5 mL) under inert atmosphere was added a solution of dimethylsulfoxide (704 mg, 9.02 mmol, 3.0 equiv) in tetrahydrofuran (3 mL) dropwise with stirring at −80° C. After 30 min, the ((3r,5r,7r)-adamantan-1-yl) methanol (500.0 mg, 3.007 mmol, 1.0 equiv) dissolved in tetrahydrofuran (3 mL) was added dropwise with stirring at −80° C. After 2 h triethylamine (1.9 mL, 13.5 mmol, 4.5 equiv) was added and stirring at −80° C. continued for 30 min. The mixture was allowed to warm to room temperature and diluted with dichloromethane (20 mL). The organic layer was successively washed with 2×20 mL of saturated ammonium chloride solution and 20 mL of brine. The organic layer was dried over anhydrous sodium sulfate, filtered and concentrated. The crude product was purified on a silica gel column with petroleum ether/ethyl acetate (100/1 to 50/1) to obtain 450 mg (88% yield) of 17-4 as a white solid.

### Preparation of 17-5

To a solution of 17-3 (50.0 g, 77.43 mmol) in 500 mL of tetrahydrofuran was added sodium hydride (7.4 g, 309.7 mmol, 4.0 equiv) at 0° C. and the resulting mixture was stirred for 30 min at 0° C. Then 17-4 (10.2 g, 61.9 mmol, 0.8 equiv) was added at 0° C. ant the mixture was stirred for 16 h at room temperature. The reaction was quenched by the addition of saturated ammonium chloride solution (1000 mL) and extracted with 3×1500 mL of dichloromethane. The combined organic layers were washed with 2×1000 mL of water and 1000 mL of brine. The organic layer was dried over anhydrous sodium sulfate, filtered and concentrated. The crude product was applied onto a silica gel column and eluted with petroleum ether/ethyl acetate (100/1 to 3/1) to obtain 31.0 g (88% yield) of 17-5 as a yellow oil. ¹H-NMR (CDCb₃): δ 7.30-7.12 (m, 5H), 5.11-4.89 (m, 2H), 4.41 (s, 2H), 3.37 (t, J=6.6 Hz, 2H), 2.09 (q, J=6.8 Hz, 2H), 1.85 (dd, J=6.1, 3.1 Hz, 3H), 1.71-1.56 (m, 11H), 1.50 (dt, J=13.5, 6.8 Hz, 2H), 1.19 (d, J=12.7 Hz, 21H).

### Preparation of 17-6

To a solution of 17-5 (12.5 g, 27.7 mmol) in 250 mL of ethyl acetate was added palladium on carbon (25 g) and the resulting mixture stirred under hydrogen at 20 atm pressure and at 65° C. for 8 h. The mixture was then filtered, the filtrate concentrated, and the residue purified on a silica gel column with petroleum ether/ethyl acetate (100/1 to 3/1). 17-6 (5.25 g, 52% yield) was obtained as a white solid. ¹H-NMR (300 MHz, DMSO-d₆) δ 4.21-4.13 (m, 1H), 3.24 (td, J=6.4, 5.0 Hz, 2H), 1.79 (s, 3H), 1.51 (dt, J=15.1, 10.8 Hz, 7H), 1.30 (d, J=2.9 Hz, 9H), 1.12 (s, 24H), 0.89 (d, J=7.3 Hz, 2H).

### Preparation of 17-7

To a solution of 17-6 (3.5 g, 9.7 mmol) in 70 mL of acetone was added Jones reagent (11.2 ml) at 0° C. and the resulting mixture stirred for 24 h at 40° C. The mixture was then filtrated and the filtrate was precipitated with acetonitrile to obtain 2.7 g (74% yield) of 17-7 as a white solid. ¹H-NMR (DMSO-d₆): δ 11.95 (s, 1H), 2.18 (t, J=7.3 Hz, 2H), 1.91 (s, 3H), 1.62 (q, J=12.3 Hz, 6H), 1.52-1.33 (m, 8H), 1.24 (s, 18H), 1.18 (d, J=9.2 Hz, 4H), 1.01 (d, J=7.2 Hz, 2H).

### Preparation of 17

To a solution of 17-7 (3 g, 8 mmol) in DMF (60 mL) at 0° C. were added 1-hydroxypyrrolidine-2, 5-dione (1.8 g, 15.9 mmol, 2.0 equiv), 4-dimethylaminopyridine (194 mg, 1.6 mmol, 0.2 equiv), N, N-diisopropylethylamine (3.1 g, 23.9 mmol, 3.0 equiv), and 3-(((ethylimino)methylene)amino)-N, N-dimethylpropan-1-amine hydrochloride (2.3 g, 11.94 mmol, 1.5 equiv). The resulting mixture was stirred for 12 h at room temperature and then extracted with 3×500 mL of dichloromethane. The combined organic layers were washed with 2×500 mL of water and 500 mL of saturated brine. The organic layer was dried (Na₂SO₄), filtered and concentrated. The crude residue was applied onto a silica gel column and eluted with n-hexane/ethyl acetate (100/1 to 3/1) to obtain 2.1 g (53.3% yield) of compound 17 as a white solid. MS: m/z 491 [M+NH₄]⁺. ¹H-NMR: (CDCl₃): δ 2.83 (s, 4H), 2.62-2.57 (t, J=7.5 Hz, 2H), 1.92 (s, 3H), 1.79-1.59 (m, 8H), 1.45-1.35 (m, 8H), 1.33-1.24 (m, 16H), 1.23-1.18 (m, 4H), 1.04-0.96 (m, 2H).

### Example 18

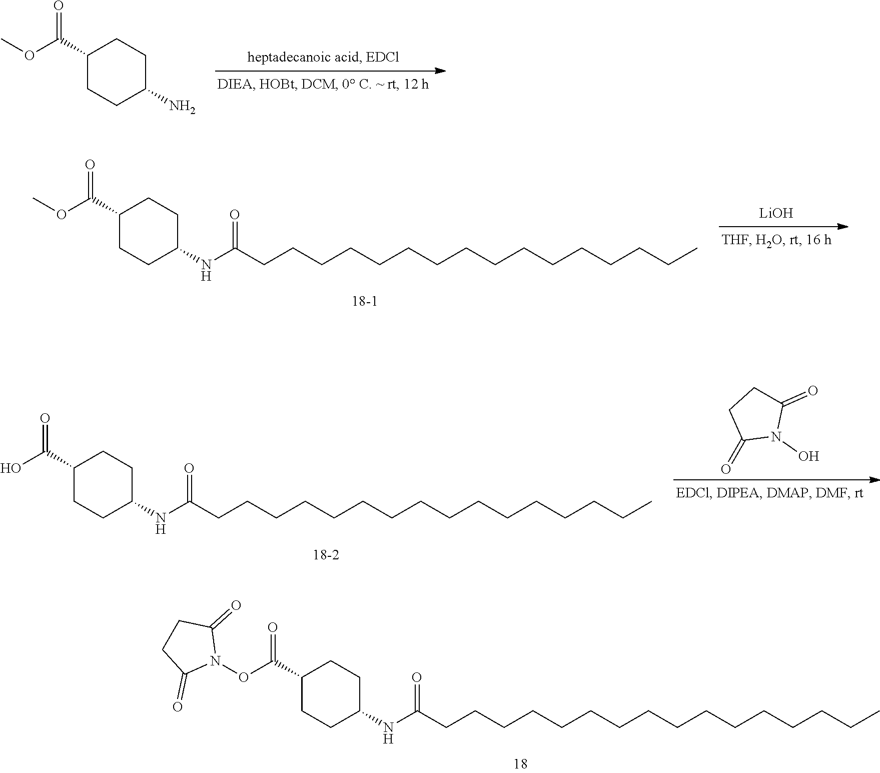 [SMILES: COC(=O)[C@H]1CC[C@@H](N)CC1 ; CCCCCCCCCCCCCCCCC(=O)N[C@H]1CC[C@@H](C(=O)OC)CC1 ; "LiOH" ; CCCCCCCCCCCCCCCCC(=O)N[C@H]1CC[C@@H](C(=O)O)CC1 ; O=C1CCC(=O)N1O ; CCCCCCCCCCCCCCCCC(=O)N[C@H]1CC[C@@H](C(=O)ON2C(=O)CCC2=O)CC1]

### Preparation of 18-1

To a solution of methyl trans-4-aminocyclohexanecarboxylate hydrochloride (5 g, 25.9 mmol, 1.2 equiv) and N,N-diisopropylethylamine (6.68 g, 51.8 mmol, 2.4 equiv) in 90 mL of dichloromethane with an inert atmosphere of argon was added heptadecanoic acid (5.8 g, 21.6 mmol, 1.0 equiv) at 0° C. and the mixture was stirred for 10 min. Then N-(3-dimethylaminopropyl)-N′-ethylcarbodiimide hydrochloride (4.97 g, 25.9 mmol, 1.2 equiv) and 1-hydroxybenzotriazole (3.5 g, 25.9 mmol, 1.2 equiv) were added at 0° C. The resulting solution was stirred for 12 h at room temperature and then diluted with 200 mL of dichloromethane. The organic layer was washed with 2×100 mL of water and 2×100 mL of saturated brine. The organic layer was dried over anhydrous sodium sulfate and concentrated. The residue was purified on a silica gel column with hexane/ethyl acetate (70/1 to 2/1) to obtain 7.1 g (79% yield) of 18-1 as a white solid. MS: m/z 410.50 [M+H]⁺. ¹H NMR (CDCl₃) δ 5.35 (s, 1H), 3.92-3.78 (m, 1H), 3.59 (s, 3H), 2.41 (s, 1H), 2.11-2.03 (m, 2H), 1.86-1.69 (m, 2H), 1.69-1.39 (m, 8H), 1.15 (s, 26H), 0.81-0.74 (m, 3H).

### Preparation of 18-2

To a solution of 18-1 (7.1 g, 17.4 mmol) in 71 mL of tetrahydrofuran with an inert atmosphere of argon was added lithium hydroxide (5M, 43.4 mL, 87 mmol, 5.0 equiv) at 0° C. The resulting solution was stirred for 16 h at room temperature then neutralized to pH 6 with hydrochloric acid (2 M). The mixture was diluted with 200 mL of dichloromethane, then washed with 2×100 mL of water and 2×100 mL of saturated brine. The organic layer was dried over anhydrous sodium sulfate and concentrated. The crude residue was purified on a silica gel column with hexane/ethyl acetate (70/1 to 1/2) to obtain 5 g (72% yield) of 18-2 as a white solid. MS: m/z 396.40 [M+H]⁺. ¹HNMR (CDCl₃): δ 5.29 (s, 1H), 3.85 (s, 1H), 2.48 (s, 1H), 2.05 (t, J=7.6 Hz, 2H), 1.82 (d, J=7.3 Hz, 2H), 1.66 (d, J=10.2 Hz, 4H), 1.43 (s, 4H), 1.15 (s, 26H), 0.81-0.74 (m, 3H).

### Preparation of 18

To a solution of 18-2 (5 g, 12.6 mmol) and N-hydroxysuccinimide (2.9 g, 25.2 mmol, 2.0 equiv) and 4-dimethylaminopyridine (310 mg, 2.5 mmol, 0.2 equiv) in DMF (50 mL) with an inert atmosphere of nitrogen was added N-(3-dimethylaminopropyl)-N′-ethylcarbodiimide hydrochloride (3.63 g, 18.9 mmol, 1.5 equiv) at 0° C. The resulting solution was stirred for 16 h at room temperature then diluted with 100 mL of dichloromethane. The organic layer was washed with 2×100 mL of water and 2×100 mL of saturated brine. The organic layer was dried over anhydrous sodium sulfate, filtered and concentrated. The residue was purified on a silica gel column with hexane/ethyl acetate (70/1 to 1/1). The fractions containing product were concentrated and the residue crystallized from dichloromethane/diethyl ether (30/1) to afford 2.41 g (38% yield) of compound 18 as a white solid. MS: m/z 493.20 [M+H]⁺. ¹H NMR (CDCl₃:) 6 5.44 (d, J=8.1 Hz, 1H), 4.00-3.90 (m, 1H), 2.98-2.91 (m, 1H), 2.87 (d, J=2.4 Hz, 4H), 2.20-2.07 (m, 4H), 1.91-1.78 (m, 4H), 1.68-1.52 (m, 4H), 1.27 (s, 26H), 0.90 (t, J=6.8 Hz, 3H).

### Example 19

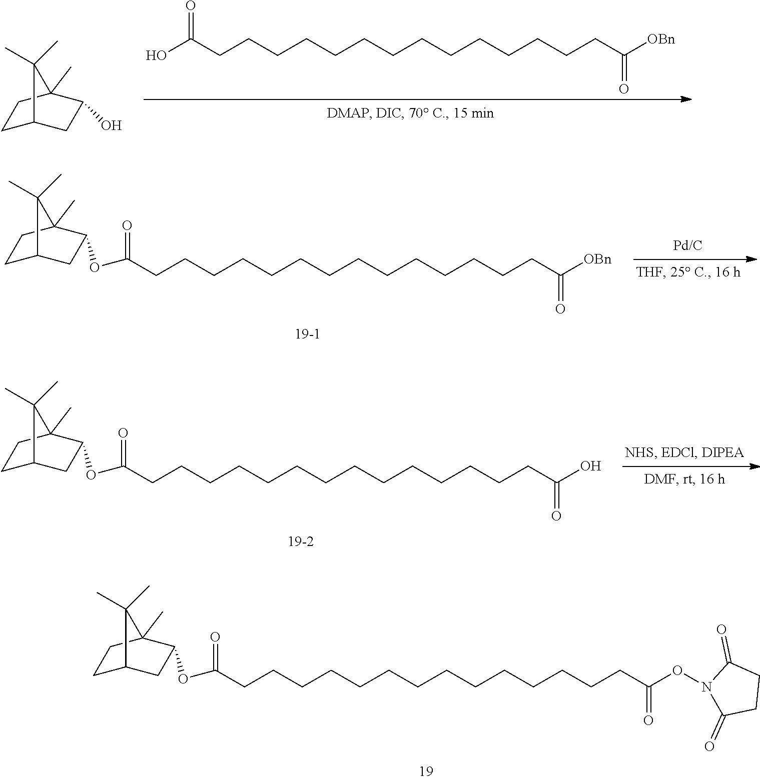

### Preparation of 19-1

To a solution of (−)-borneol (2 g, 12.7 mmol) in dichloromethane (30 mL) under N₂ were added 16-(benzyloxy)-16-oxohexadecanoic acid (7.3 g, 19.44 mmol, 1.5 equiv), 4-dimethylaminopyridine (0.48 g, 3.89 mmol, 0.3 equiv), and dicyclohexylcarbodiimide (13.3 g, 64.83 mmol, 5.0 equiv) and stirred overnight. The reaction mixture was then diluted with water and extracted with dichloromethane. The combined organic layers were dried over anhydrous Na₂SO₄, filtered, and concentrated. The residue was purified on a silica gel column with petroleum ether/ethyl acetate (100/0 to 50/1) to obtain 6.5 g (97% yield) of 19-1 as a yellow solid. MS: m/z 535.20 [M+Na]⁺.

### Preparation of 19-2

To a solution of 19-1 (6.7 g, 13.06 mmol) in tetrahydrofuran (67 mL) was added 10% palladium on activated carbon (1.4 g) and flushed with five cycles of H₂. The resulting mixture was stirred for 16 h then filtered, the solid washed with tetrahydrofuran, and combined filtrate concentrated. The residue was purified on a silica gel column with petroleum ether/ethyl acetate (50/1 to 10/1) to obtain 4 g (75% yield) of 19-2 as a white solid. MS: m/z 445.40 [M+Na]⁺.

### Preparation of 19

To a solution of 19-2 (5.5 g, 13.0 mmol) in N, N-dimethylformamide (55 mL) was added 1-ethyl-3-(3-dimethylaminopropyl)carbodiimide (2.42 g, 15.6 mmol, 1.2 equiv), N-hydroxysuccinimide (1.80 g, 16.6 mmol, 1.2 equiv), and N,N-diisopropylethylamine (5.04 g, 39.1 mmol, 3 equiv) and stirred for 16 h. The mixture was diluted with ethyl acetate. The organic layer was washed with water and brine and dried over anhydrous Na₂SO₄. Evaporated residue was purified on a silica gel column with hexane/ethyl acetate (100/0 to 15/1) to obtain 2.1 g (40% yield) of compound 19 as an off-white solid. MS: m/z 542.20 [M+Na]⁺. ¹H-NMR (DMSO-d₆): δ 4.81 (t, J=5.2 Hz, 1H), 2.81 (s, 4H), 2.65 (t, J=7.2 Hz, 2H), 2.72-2.31 (m, 3H), 1.88-1.84 (m, 1H), 1.72-1.51 (m, 6H), 1.34-1.14 (m, 23H), 0.92-0.78 (m, 9H).

### Example 20

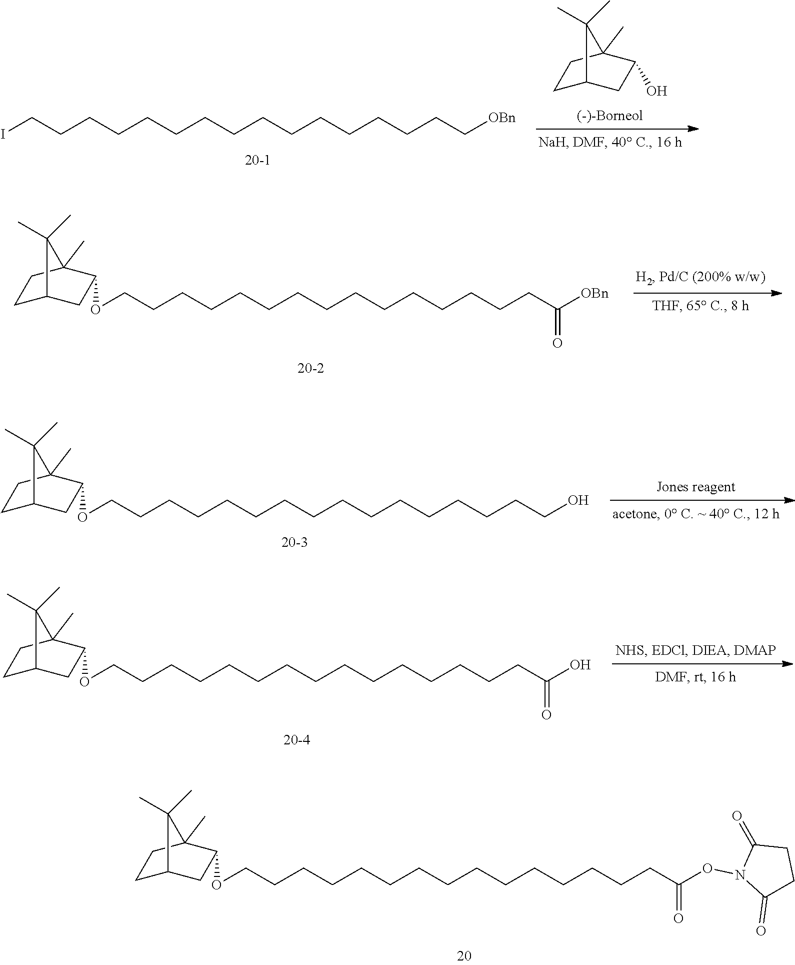

### Preparation of 20-2

To a solution of (1S,2R,4S)-1,7,7-trimethylbicyclo [2.2.1]heptan-2-ol (16.8 g, 109.06 mmol, 2.0 equiv) in N, N-dimethylformamide (250 mL) at 0° C. was added sodium hydride (2.6 g, 109.1 mmol, 2.0 equiv) and stirred for 30 min. 20-1 (25 g, 54.5 mmol, 1.0 equiv) was then added at 0° C. and the solution was stirred for 16 h at 40° C. The reaction was quenched with saturated NH₄Cl and extracted with dichloromethane. The combined organic layers were washed with water and brine. The organic layer was dried over anhydrous Na₂SO₄, filtered, and concentrated. The residue was purified on a silica gel column with petroleum ether/ethyl acetate (100/1 to 10/1) to obtain 4.5 g (17% yield) of 20-2 as a yellow oil.

### Preparation of 20-3

To a solution of 20-2 (2.3 g, 4.74 mmol) in THF (46 mL) was added palladium on carbon (4.6 g) and the mixture was stirred under H₂ (20 atm pressure) at 65° C. for 8 h. The resulting mixture was filtered, and the filtrate concentrated. The residue was purified on a silica gel column with petroleum ether/ethyl acetate (100/1 to 6/1) to obtain 1.8 g (96% yield) of 20-3 as a white solid. ¹HNMR (CDCl₃): δ 3.63 (t, J=6.6 Hz, 2H), 3.53 (ddd, J=9.5, 3.4, 1.9 Hz, 1H), 3.46-3.27 (m, 2H), 2.16-1.91 (m, 2H), 1.63-1.47 (m, 7H), 1.27 (d, J=8.2 Hz, 25H), 0.99 (dd, J=13.0, 3.4 Hz, 1H), 0.85 (d, J=7.5 Hz, 9H).

### Preparation of 20-4

To a solution of 20-3 (3.46 g, 8.8 mmol) in acetone (121 mL) at 0° C. was added Jones reagent (10.5 mL) and stirred at 40° C. for 12 h. The reaction mixture was then filtered, concentrated, and purified on a silica gel column with petroleum ether/ethyl acetate (100/1 to 6/1) to obtain 2.87 g (80% yield) of 20-4 as a white solid. ¹HNMR (DMSO-d₆): δ 12.29-11.36 (m, 1H), 3.56-3.13 (m, 3H), 2.15 (t, J=7.4 Hz, 2H), 2.10-1.90 (m, 2H), 1.68-1.54 (m, 2H), 1.47 (h, J=6.6 Hz, 4H), 1.24 (d, J=11.0 Hz, 22H), 1.11 (ddt, J=17.3, 11.8, 4.9 Hz, 2H), 0.97-0.88 (m, 1H), 0.79 (d, J=2.5 Hz, 9H).

### Preparation of 20

To a solution of 20-4 (3.5 g, 8.6 mmol) in N, N-dimethylformamide (70 mL) at 0° C. were added 1-hydroxypyrrolidine-2,5-dione (1.9 g, 17.1 mmol, 2.0 equiv), 4-dimethylaminopyridine (0.21 g, 1.7 mmol, 0.2 equiv), N, N-diisopropylethylamine (3.3 g, 25.7 mmol, 3.0 equiv), and 3-(((ethylamine)methylene) amino)-N, N-dimethylpropan-1-amine hydrochloride (2.4 g, 12.8 mmol, 1.5 equiv) and stirred for 16 hours at room temperature. The resulting mixture was extracted with dichloromethane. The organic layer was washed with water and brine, dried (anhydrous Na₂SO₄), filtered, and concentrated. The residue was purified on a silica gel column with n-hexane/ethyl acetate (100/1 to 6/1) to obtain 2.0 g (44% yield) of compound 20 as an off-white solid. MS: m/z 528.4 [M+Na]⁺. ¹HNMR (CDCb₃): δ 3.58-3.48 (m, 1H), 3.46-3.26 (m, 2H), 2.84 (d, J=2.3 Hz, 4H), 2.66-2.54 (m, 2H), 2.16-1.91 (m, 2H), 1.79-1.50 (m, 7H), 1.39 (d, J=8.8 Hz, 2H), 1.27 (d, J=7.5 Hz, 20H), 1.20-1.12 (m, 1H), 1.02-0.95 (m, 1H), 0.86 (s, 3H), 0.83 (d, J=1.1 Hz, 6H).

### Example 21

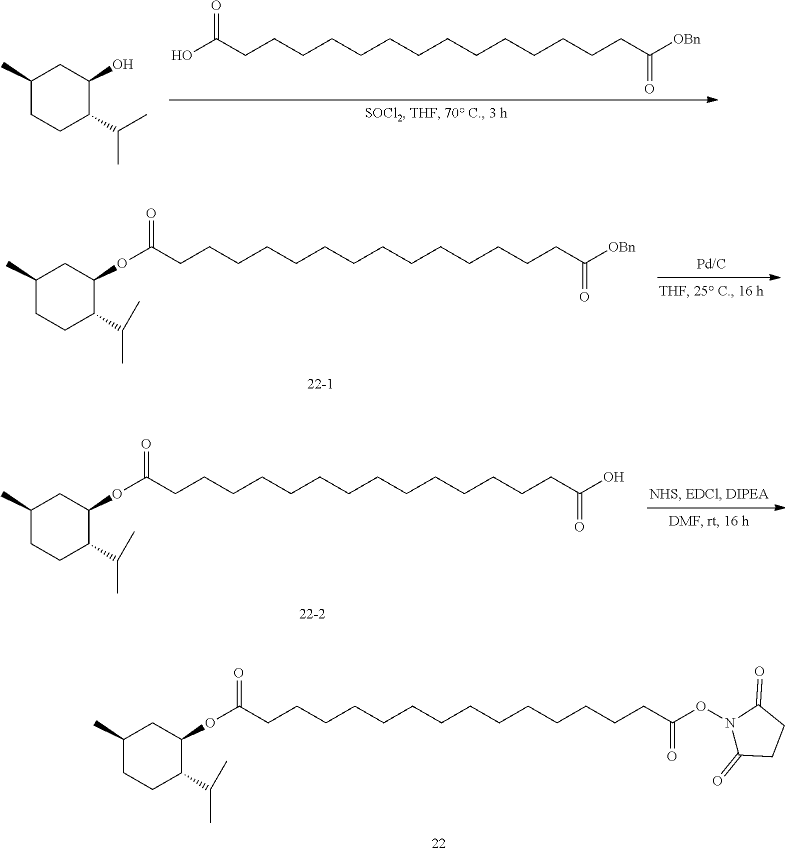 [SMILES: CC(C)[C@@H]1CC[C@@H](C)C[C@H]1O ; O=C(O)CCCCCCCCCCCCCCC(=O)OCc1ccccc1 ; CC(C)[C@@H]1CC[C@@H](C)C[C@H]1OC(=O)CCCCCCCCCCCCCCC(=O)OCc1ccccc1 ; CC(C)[C@@H]1CC[C@@H](C)C[C@H]1OC(=O)CCCCCCCCCCCCCCC(=O)O ; CC(C)[C@@H]1CC[C@@H](C)C[C@H]1OC(=O)CCCCCCCCCCCCCCC(=O)ON1C(=O)CCC1=O]

### Preparation of 22-1

A solution of 16-(benzyloxy)-16-oxohexadecanoic acid (4.8 g, 12.8 mmol) in sulfuryl dichloride (40 mL) was stirred for one hour at 70° C. It was then cooled to room temperature and concentrated. The crude was added into a solution of (1R,2S,5R)-2-isopropyl-5-methylcyclohexan-1-ol (2 g, 12.79 mmol, 1.0 equiv) in tetrahydrofuran (10 mL) at room temperature and stirred for 2 hours. The pH was adjusted to 7 with 1 M NaOH solution and extracted with ethyl acetate. The organic layer was washed with brine, dried over anhydrous Na₂SO₄, filtered, and concentrated. The residue was purified on a silica gel column (petroleum ether/ethyl acetate, 100/0 to 80/1) to obtain 5 g (78% yield) of 22-1 as a colorless oil. MS: m/z 537.50 [M+Na]⁺.

### Preparation of 22-2

To a solution of 22-1 (5 g, 7.41 mmol) in tetrahydrofuran (50 mL) was added 10% palladium on activated carbon (1 g) under N₂. The flask was flushed with five cycles of H₂ and stirred for 16 hours under H₂. The reaction mixture was then filtered, concentrated, and the residue was purified on a silica gel column with petroleum ether/ethyl acetate (100/0 to 40/1) to obtain 3.5 g (74% yield) of 22-2 as a white solid. MS: m/z 447.40 [M+Na]⁺.

### Preparation of 22

To a solution of 22-2 (5.5 g, 11.656 mmol) in N, N-dimethylformamide (55 mL) was added 1-hydroxypyrrolidine-2,5-dione (2 g, 17.48 mmol, 1.5 equiv), 1-ethyl-3-(3-dimethylaminopropyl)-carbodiimide (3.3 g, 17.48 mmol, 1.5 equiv), and N,N-diisopropylethylamine (4.5 g, 34.96 mmol, 3 equiv) and stirred for 16 hours. The reaction mixture was then diluted with ethyl acetate and the organic layer was washed with water and brine. The resulting solution was then dried over anhydrous Na₂SO₄, filtered, and concentrated. The residue was purified on a silica gel column with hexane/ethyl acetate (100/0 to 15/1) to obtain 2.7 g of compound 22 as a white solid. MS: m/z 544.40 [M+Na]⁺. ¹H NMR (DMSO-d₆) δ 4.61-4.54 (m, 1H), 2.81 (s, 4H), 2.65 (t, J=7.2 Hz, 2H), 2.25 (t, J=7.2 Hz, 2H), 1.88-1.80 (m, 2H), 1.66-1.49 (m, 6H) 1.44-1.24 (m, 22H), 1.11-0.83 (m, 9H), 0.71 (d, J=6.9 Hz, 3H).

### Example 22

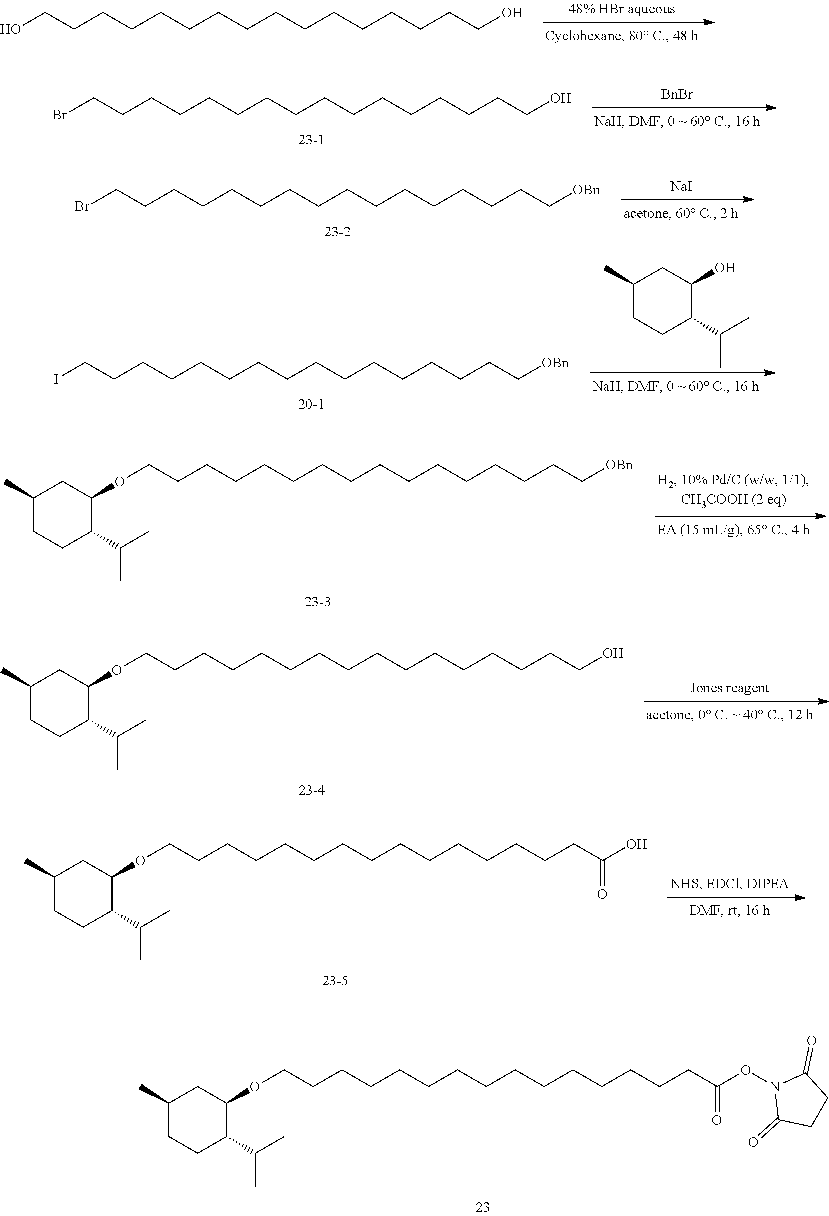

### Preparation of 23-1

To a solution of 1,16-hexadecanediol (60 g, 232.56 mmol) in cyclohexane (600 mL) was added hydrogen bromide (43.2 mL, 255.8 mmol, 1.1 equiv). The mixture was warmed to reflux and stirred for 48 h. The resulting mixture was cooled to room temperature, filtered, and concentrated. The residue was purified on a silica gel column with petroleum ether/ethyl acetate (60/1 to 30/1) to obtain 35 g (47% yield) of 23-1 as a white solid.

### Preparation of 23-2

To a solution of 23-1 (35 g, 109.4 mmol) in anhydrous N,N-dimethylformamide (700 mL) at 0° C. under Ar was added sodium hydride (60% dispersion in oil, 8.75 g, 218.8 mmol, 2.0 equiv) and stirred for 30 minutes. Benzyl bromide (28.06 g, 164.1 mmol, 1.5 equiv) was added to the reaction mixture at 0° C. and stirred for 16 h at room temperature. The reaction mixture was quenched with saturated NH₄Cl, diluted with ethyl acetate, and washed with water and brine. The organic layer was dried over anhydrous Na₂SO₄, filtered, and concentrated. The residue was purified on a silica gel column with petroleum ether/ethyl acetate (500/1 to 200/1) to obtain 30 g (67% yield) of 23-2 as a white solid. ¹H NMR (DMSO-d₆): δ 7.42-7.23 (m, 5H), 4.48 (d, J=39.7 Hz, 2H), 3.58-3.36 (m, 4H), 1.78 (p, J=6.8 Hz, 2H), 1.52 (p, J=6.7 Hz, 2H), 1.23 (s, 24H).

### Preparation of 20-1

To a mixture of 23-2 (30 g, 93.75 mmol, 1.0 equiv) in acetone (600 ml) was added sodium iodide (28.125 g, 187.5 mmol, 2.0 equiv) and refluxed for 2 h with vigorous stirring. The reaction mixture was cooled, filtered, and concentrated. The residue was purified on a silica gel column with petroleum ether/ethyl acetate (100/1 to 60/1) to obtain 30 g (89.5% yield) of 20-1 as a white solid. ¹H NMR (CDCl₃): δ 7.35-7.21 (m, 5H), 4.50 (d, J=2.1 Hz, 2H), 3.46 (t, J=6.6 Hz, 2H), 3.18 (t, J=7.0 Hz, 2H), 1.82 (p, J=7.1 Hz, 2H), 1.61 (p, J=6.8 Hz, 2H), 1.26 (s, 24H).

### Preparation of 23-3

To a solution of L(−)-menthol (20.44 g, 131.0 mmoL, 2.0 equiv) in anhydrous N,N-dimethylformamide (409 mL) at 0° C. under Ar was added sodium hydride (60% dispersion in oil, 5.24 g, 131.0 mmol, 2.0 equiv) and stirred for 30 min. 20-1 (30 g, 65.50 mmoL, 1.0 equiv) was then added to the reaction mixture at 0° C. and stirred for 16 h at room temperature. The reaction mixture was quenched with saturated NH₄Cl, diluted with ethyl acetate, and washed with water and brine. The organic layer was dried over anhydrous Na₂SO₄, filtered, and concentrated. The residue was purified on a silica gel column with petroleum ether/ethyl acetate (200/1-100/1) to obtain 9.8 g (30% yield) of 23-3 as a colorless oil.

¹H NMR (CDCl₃): δ 7.39-7.22 (m, 5H), 4.50 (s, 2H), 3.60 (dt, J=9.1, 6.4 Hz, 1H), 3.46 (t, J=6.6 Hz, 2H), 3.38 (t, J=6.7 Hz, 1H), 3.25 (dt, J=9.2, 6.9 Hz, 1H), 2.98 (td, J=10.5, 4.1 Hz, 1H), 2.23 (dqd, J=14.1, 7.2, 2.7 Hz, 1H), 2.14-2.00 (m, 1H), 1.69-1.51 (m, 6H), 1.35-1.23 (m, 27H), 0.94-0.75 (m, 10H).

### Preparation of 23-4

To a solution of 23-3 (7.2 g, 14.81 mmoL, 1.0 equiv) in ethyl acetate (144 mL) under H₂ was added palladium on activated carbon (10%, 14.4 g) and acetic acid (0.89 g, 14.8 mmol, 1.0 equiv) and stirred for 8 h at 65° C. The solution was filtered, concentrated and the residue was purified on a silica gel column with petroleum ether/ethyl acetate (100/1 to 50/1) to obtain 5.2 g (88.6% yield) of 23-4 as a white solid. ¹H NMR (CDCl₃) δ 3.62 (dt, J=15.5, 6.5 Hz, 3H), 3.25 (dt, J=9.2, 6.9 Hz, 1H), 2.99 (td, J=10.6, 4.1 Hz, 1H), 2.21 (pt, J=9.6, 4.8 Hz, 1H), 2.14-2.04 (m, 1H), 1.70-1.47 (m, 6H), 1.38-1.14 (m, 27H), 1.04-0.74 (m, 12H).

### Preparation of 23-5

To a solution of 23-4 (5.2 g, 13.13 mmoL, 1.0 equiv) in acetone (182 mL) at 0° C. under Ar was added Jones reagent (2.67 M, 15.74 mL, 42.02 mmoL, 3.2 equiv) and stirred for 12 h at 40° C. The reaction mixture was filtered and concentrated, and the residue was diluted with dichloromethane, then washed with water and brine. The organic layer was dried over anhydrous Na₂SO₄, filtered, and concentrated. The residue was purified on a silica gel column with petroleum ether/ethyl acetate (40/1 to 8/1) to obtain 3.5 g (65% yield) of 23-5 as a water solid. MS: m/z 409.30 [M−H]⁻. ¹H NMR (CDCl₃) δ 3.62-2.82 (m, 3H), 2.27 (t, J=7.5 Hz, 2H), 2.21-1.96 (m, 2H), 1.65-1.39 (m, 6H), 1.21 (d, J=13.8 Hz, 25H), 0.94-0.66 (m, 11H).

### Preparation of 23

To a solution of 23-5 (3.5 g, 8.54 mmol, 1.0 equiv) and N-hydroxysuccinimide (1.96 g, 17.07 mmol, 2.0 equiv) in N,N-dimethylformamide (70 mL) at 0° C. under Ar was added 4-dimethylaminopyridine (0.21 g, 1.71 mmol, 0.2 equiv) and N,N-diisopropylethylamine (3.3 g, 25.61 mmol, 3.0 equiv) and stirred for 30 minutes. N-(3-dimethylaminopropyl)-N′-ethylcarbodiimide hydrochloride (2.46 g, 12.81 mmoL, 1.5 equiv) was then added and stirred for 12 h at room temperature. The reaction mixture was diluted with ethyl acetate and washed with water and brine. The organic layer was dried over anhydrous Na₂SO₄, filtered, and concentrated. The residue was purified on a silica gel column with hexane/ethyl acetate (50/1 to 6/1) to obtain 2.0 g (46.9% yield) of compound 23 as a white solid. MS: m/z 508.45 [M+H]⁺. ¹H NMR (CDCl₃) δ 3.60 (dt, J=9.1, 6.3 Hz, 1H), 3.24 (dt, J=9.1, 6.8 Hz, 1H), 2.98 (td, J=10.5, 4.1 Hz, 1H), 2.83 (d, J=2.5 Hz, 4H), 2.60 (t, J=7.5 Hz, 2H), 2.22 (pd, J=6.9, 2.7 Hz, 1H), 2.14-2.02 (m, 1H), 1.74 (p, J=7.4 Hz, 2H), 1.67-1.47 (m, 4H), 1.26 (d, J=5.7 Hz, 24H), 1.04-0.83 (m, 8H), 0.82-0.73 (m, 4H).

### Example 23

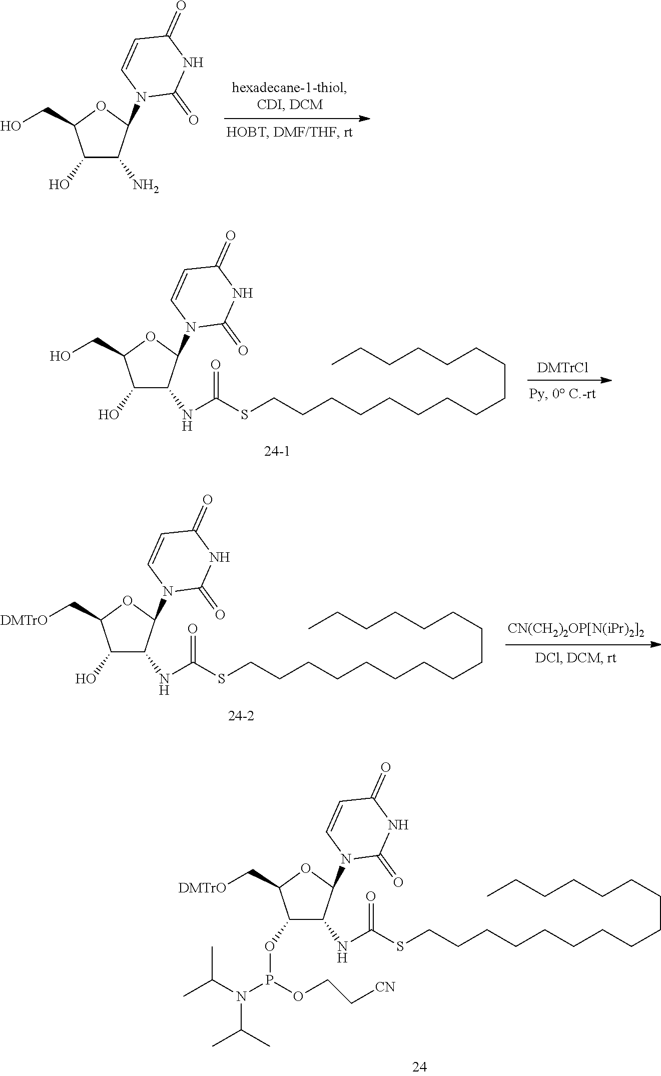

### Preparation of 24-1

To a solution of 1,1′-carbonyldiimidazole (4.8 g, 19.6 mmol, 1.8 equiv) in 50 mL of dichloromethane with an inert atmosphere of argon was added hexadecane-1-thiol (6.38 g, 24.69 mmol, 1.5 equiv) at room temperature. The resulting solution was stirred for 1 h at room temperature. The reaction mixture was concentrated under reduced pressure. The crude product was dissolved with dichloromethane (10 mL), then added dropwise to acetonitrile (100 mL). The solids were collected by filtration. The cake was dissolved in 20 mL of N, N-dimethylformamide and 20 mL of tetrahydrofuran. Then 2′-amino-D-uridine (4 g, 16.5 mmol, 1.0 equiv) and 1-hydroxybenzotriazole (3.33 g, 24.7 mmol, 1.5 equiv) were added at room temperature. The resulting solution was stirred for 3 h at room temperature. The reaction mixture was concentrated under reduced pressure. The crude product was purified by Prep-Flash with the following conditions: C18-column, mobile phase, water and tetrahydrofuran (30% to 100% THF gradient). The fractions containing product were concentrated to get 6.9 g (80% yield) of 24-1 as a white solid. MS: m/z 528.15 [M+H]⁺. ¹H NMR (DMSO-d₆) δ 11.30 (d, J=2.3 Hz, 1H), 8.05 (d, J=8.4 Hz, 1H), 7.87 (d, J=8.1 Hz, 1H), 5.90 (d, J=8.2 Hz, 1H), 5.75-5.59 (m, 2H), 5.16 (t, J=5.0 Hz, 1H), 4.55-4.40 (m, 1H), 4.08 (t, J=5.7 Hz, 1H), 3.90 (q, J=2.8 Hz, 1H), 3.66-3.49 (m, 2H), 2.84-2.63 (m, 2H), 1.45 (d, J=7.0 Hz, 2H), 1.23 (d, J=2.8 Hz, 26H), 0.92-0.79 (m, 3H).

### Preparation of 24-2

To a solution of 24-1 (4 g, 7.59 mmol) in 40 mL of pyridine with an inert atmosphere of argon was added 4,4′-dimethoxytrityl chloride (3.1 g, 9.1 mmol, 1.2 equiv) at 0° C. The resulting solution was stirred for 3 h at 25° C. The reaction was diluted with 300 mL of dichloromethane and quenched with saturated aqueous sodium bicarbonate. The mixture was washed with 2×150 mL of saturated aqueous sodium bicarbonate and 2×150 mL of saturated brine. The organic layer was dried over Na₂SO₄, filtered and concentrated under reduced pressure. The crude product was purified by Prep-Flash with the following conditions: C18-column, mobile phase, water and THF, gradient 30% to 100% THF. The fractions containing product were combined and dilute with equal volume of dichloromethane. The organic layer was separated. The aqueous layer was extracted with 3×200 mL of dichloromethane. The combined organic extract was dried (anhydrous Na₂SO₄), filtered, and concentrated to get 3.9 g (62% yield) of 24-2 as a white solid. MS: m/z 852.45 [M+Na]⁺. ¹H NMR (DMSO-d₆): δ 11.36 (s, 1H), 8.18 (d, J=8.3 Hz, 1H), 7.65 (d, J=8.1 Hz, 1H), 7.45-7.17 (m, 9H), 6.96-6.84 (m, 4H), 5.89 (d, J=7.6 Hz, 1H), 5.68 (d, J=4.9 Hz, 1H), 5.41 (d, J=8.1 Hz, 1H), 4.64 (d, J=6.4 Hz, 1H), 4.19 (d, J=7.1 Hz, 1H), 4.00 (d, J=3.5 Hz, 1H), 3.75 (s, 6H), 3.31-3.15 (m, 2H), 2.84-2.70 (m, 2H), 1.58-1.40 (m, 2H), 1.24 (s, 26H), 0.91-0.75 (m, 3H).

### Preparation of 24

To a solution of 24-2 (3.1 g, 3.74 mmol) in dichloromethane (31 mL) with an inert atmosphere of argon was added 3-(bis(diisopropylamino)phosphinooxy)propanenitrile (1.35 g, 4.48 mmol, 1.2 equiv), 4,5-dicyanoimidazole (485 mg, 4.1 mmol, 1.1 equiv) and the reaction mixture stirred for 2 h at room temperature. The resulting solution was diluted with 500 mL of dichloromethane, washed with 300 mL of saturated aqueous sodium bicarbonate and 300 mL of saturated brine. The organic layer was dried (anhydrous Na₂SO₄), filtered, and concentrated. The crude product was purified by Prep-Flash with the following conditions: C18-column, mobile phase, water and THF, gradient 30% to 100% THF. The fraction containing product were combined and dilute with equal volume of dichloromethane. The organic layer was separated. The aqueous phase was extracted with 3×200 mL of dichloromethane. Combined organic extract was dried (anhydrous Na₂SO₄), filtered, and concentrated. Crude residue was purified on a silica gel column with hexane/ethyl acetate (5/1 to 2/1) to afford 2.624 g (69% yield) of compound 24 as a white solid. MS m/z 1030.55 [M+H]⁺. ¹H NMR (DMSO-d₆) δ 11.43 (s, 1H), 8.44 (dd, J=33.8, 8.3 Hz, 1H), 7.69 (dd, J=8.2, 5.0 Hz, 1H), 7.40 (dd, J=7.7, 2.5 Hz, 2H), 7.35-7.21 (m, 7H), 6.89 (dd, J=9.0, 3.0 Hz, 4H), 5.94 (t, J=7.0 Hz, 1H), 5.45 (d, J=7.9 Hz, 1H), 4.90-4.70 (m, 1H), 4.47-4.28 (m, 1H), 4.26-4.11 (m, 1H), 3.93-3.77 (m, 1H), 3.74 (s, 6H), 3.72-3.61 (m, 1H), 3.60-3.43 (m, 2H), 3.30-3.18 (m, 2H), 2.92-2.79 (m, 1H), 2.78-2.68 (m, 2H), 2.63 (t, J=6.0 Hz, 1H), 1.58-1.46 (m, 2H), 1.23 (s, 26H), 1.15-1.05 (m, 9H), 0.96 (d, J=6.7 Hz, 3H), 0.88-0.82 (m, 3H). ³¹P NMR (DMSO-d₆) δ 149.13, 147.57.

### Example 24

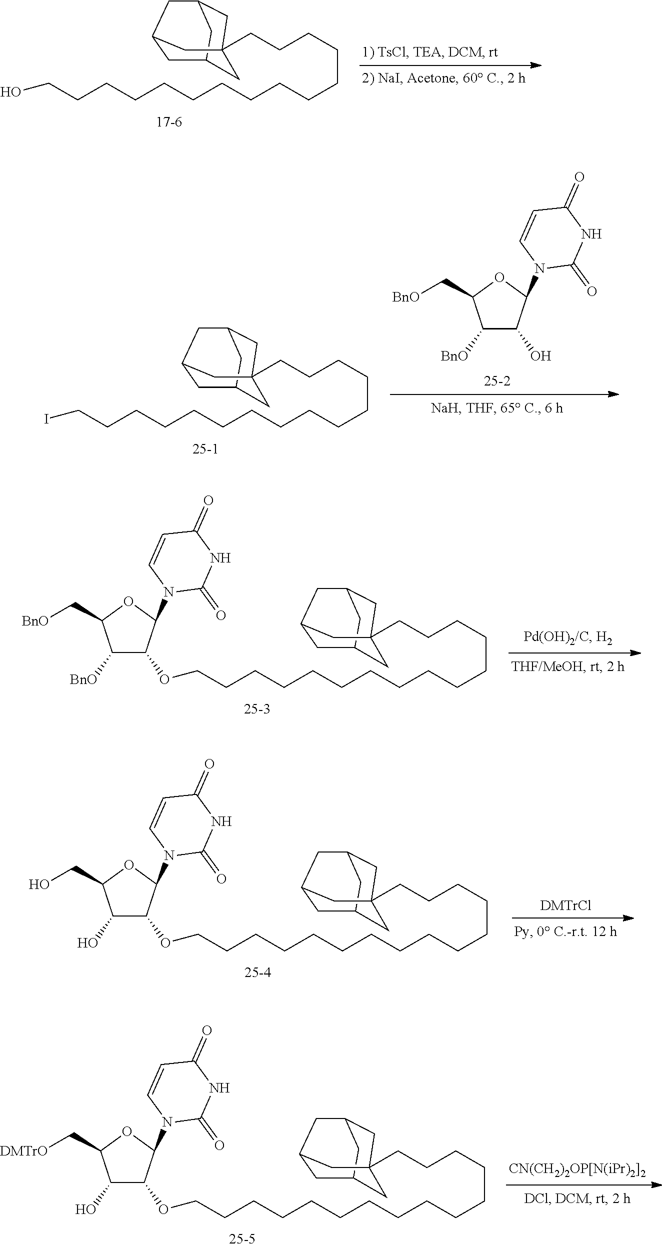

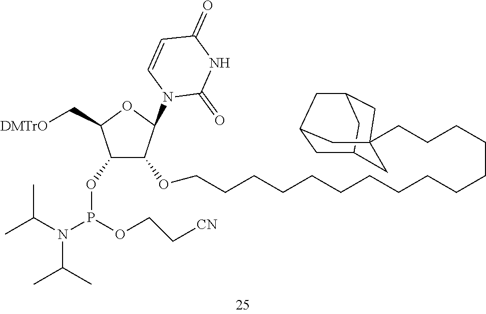

### Preparation of 25-1

To a solution of 17-6 (14 g, 38.60 mmol), triethylamine (11.7 g, 115.8 mmol, 3.0 equiv) and dimethylaminopyridine (2.4 g, 19.30 mmol, 0.5 equiv) in 140 mL of dichloromethane was added 4-methylbenzenesulfonyl chloride (8.1 g, 42.5 mmol, 1.1 equiv) at ° C. The resulting solution was stirred for 2 h at room temperature. The reaction was quenched by the addition of 500 mL of saturated sodium bicarbonate solution, the resulting mixture was extracted with 3×800 mL of dichloromethane. The combined organic layers were washed with 2×500 mL of water and 500 mL of brine, dried over anhydrous Na₂SO₄, filtered and concentrated. A mixture of the crude product and sodium iodide (28.9 g, 193.0 mmol, 5.0 equiv) in 150 mL of acetone was refluxed for 2 h with vigorous stirring, then cooled to room temperature and extracted with 3×800 mL of dichloromethane. The combined organic layers were washed with 2×500 mL of water and 500 mL of saturated brine, dried over anhydrous Na₂SO₄, filtered and concentrated. The crude product was purified on a silica gel column with petroleum ether/ethyl acetate (100/1 to 10/1) to obtain 14.2 g (78% yield) of 25-1 as a colorless oil. ¹H-NMR (CDCl₃): δ 3.09 (t, J=7.0 Hz, 2H), 1.83 (t, J=3.2 Hz, 3H), 1.71 (q, J=7.1 Hz, 2H), 1.63-1.40 (m, 6H), 1.38-1.05 (m, 30H), 0.93 (d, J=7.2 Hz, 2H).

### Preparation of 25-3

To a solution of 25-2 (3.1 g, 7.4 mmol) in 70 mL of tetrahydrofuran at 0° C. was added sodium hydride (0.71 g, 29.6 mmol, 4.0 equiv) and the resulting mixture was stirred for 30 min at 0° C. Then 25-1 (13.9 g, 29.6 mmol, 4.0 equiv) was added at 0° C. and the reaction mixture was stirred for 6 h at 65° C. The reaction was quenched by the addition of saturated ammonium chloride solution (500 mL) and extracted with dichloromethane (3×800 mL). The combined organic layers were washed with water and brine, dried over anhydrous Na₂SO₄, filtered and concentrated. The crude product was purified on a silica gel column with petroleum ether/ethyl acetate (100/1 to 1/1) to obtain 2.40 g (42% yield) of 25-3 as a white solid. MS m/z 791.50 [M+Na]⁺.

### Preparation of 25-4

To a solution of 25-3 (3.7 g, 4.811 mmol, 1.0 equiv) in methanol (37 mL) and tetrahydrofuran (37 mL) was added palladium hydroxide (3.7 g) and stirred under H₂ atmosphere for 2 hours. The mixture was filtered, and the filtrate concentrated. The crude product was purified on a silica gel column with petroleum ether/ethyl acetate (100/1 to 3/1) to afford 2.1 g (74% yield) of 25-4 as a white solid. ¹H-NMR (DMSO-d₆): δ 11.32 (s, 1H), 7.94 (d, J=8.1 Hz, 1H), 5.84 (d, J=5.1 Hz, 1H), 5.63 (d, J=8.1 Hz, 1H), 5.12 (t, J=5.0 Hz, 1H), 5.02 (d, J=5.8 Hz, 1H), 4.12-4.02 (m, 1H), 3.90-3.80 (m, 2H), 3.69-3.51 (m, 3H), 3.44 (dddd, J=11.9, 6.9, 5.0, 1.7 Hz, 1H), 3.17 (d, J=5.2 Hz, 1H), 1.90 (p, J=2.9, 2.3 Hz, 3H), 1.70-1.53 (m, 6H), 1.53-1.38 (m, 8H), 1.22 (d, J=1.8 Hz, 23H), 1.00 (dt, J=12.8, 4.2 Hz, 2H).

### Preparation of 25-5

To a solution of 25-4 (1.0 g, 1.698 mmol) in pyridine (10 mL) was added dimethoxytrityl chloride (748 mg, 2.21 mmol, 1.3 equiv) at 0° C. and then stirred for 12 h at room temperature. The resulting solution was extracted with 3×100 mL of dichloromethane. The combined extract was washed with 2×100 mL of water and 100 mL of saturated brine, dried over anhydrous Na₂SO₄, filtered and concentrated. The crude product was purified on a silica gel column with petroleum ether/ethyl acetate (100/1 to 1/2) to obtain 1.3 g of (85% yield) of 25-5 as a white solid. ¹H-NMR (DMSO-d₆): δ 11.38 (d, J=1.9 Hz, 1H), 7.73 (d, J=8.1 Hz, 1H), 7.47-7.10 (m, 9H), 6.95-6.78 (m, 4H), 5.80 (d, J=3.7 Hz, 1H), 5.28 (dd, J=8.1, 1.8 Hz, 1H), 5.11 (d, J=6.5 Hz, 1H), 4.22-3.84 (m, 4H), 3.74 (s, 6H), 3.57 (qt, J=9.2, 6.3 Hz, 2H), 3.26 (td, J=12.3, 10.7, 3.5 Hz, 2H), 1.88 (q, J=3.1 Hz, 3H), 1.70-1.45 (m, 8H), 1.41 (d, J=2.8 Hz, 6H), 1.20 (d, J=12.8 Hz, 23H), 1.00 (d, J=7.2 Hz, 2H).

### Preparation of 25

To a solution of 25-5 (3.3 g, 3.7 mmol) in dichloromethane (33 mL) were added 2-cyanoethyl N, N, N′, N′-tetraisopropylphosphorodiamidite (1.9 g, 6.3 mmol, 1.7 equiv), and 4,5-dicyanoimidazole (0.5 g, 4.1 mmol, 1.1 equiv). The resulting solution was stirred for 2 h at room temperature and then extracted with 3×500 mL of dichloromethane. The combined organic extract was washed with 2×500 mL of saturated brine, dried over anhydrous Na₂SO₄, filtered and concentrated. The crude product was purified on a silica gel column with n-hexane/ethyl acetate (100/1 to 1/1) to obtain 2.38 g (57% yield) of compound 25 as a white solid. MS: m/z 1091.55 [M+H]⁺. ¹H-NMR (DMSO-d₆): δ 11.39 (s, 1H), 7.86-7.73 (m, 1H), 7.45-7.17 (m, 9H), 6.95-6.81 (m, 4H), 5.80 (t, J=3.1 Hz, 1H), 5.24 (t, J=8.9 Hz, 1), 4.47-4.26 (m, 1H), 4.18-3.97 (m, 2H), 3.83-3.43 (m, 12H), 3.23-3.15 (m, 2H), 2.82-2.56 (m, 2H), 1.88 (s, 3H), 1.71-1.35 (m, 14H), 1.29-1.04 (m, 33H), 0.98 (d, J=6.8 Hz, 5H). ³¹PNMR (DMSO-d₆): δ 149.13, 148.53.

### Example 25

The following compounds were prepared in analogous fashion as the compounds provided in preceding examples, using corresponding starting material.

| Compound | Characterization |
|---|---|
| 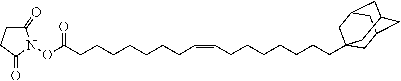 [SMILES: O=C(CCCCCCC/C=C\CCCCCCCC12CC3CC(CC(C3)C1)C2)ON1C(=O)CCC1=O] | ¹³C-NMR (CDCl₃): δ 22.39, 24.58, 25.60, 27.17, 27.24, 28.78, 28.82, 28.92, 29.01, 29.35, 29.64, 29.65, 29.80, 30.67, 30.95, 32.24, 37.35, 42.57, 44.83, 129.70, 130.06, 168.68, 169.17. |
| Compound 26 | |
| 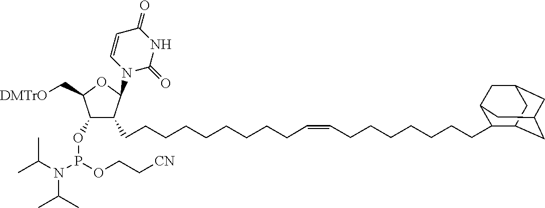 | MS: m/z 1119 [M + H]⁺; ³¹P-NMR (CDCl₃): δ 149.16, 148.63. |
| Compound 27 | |
| 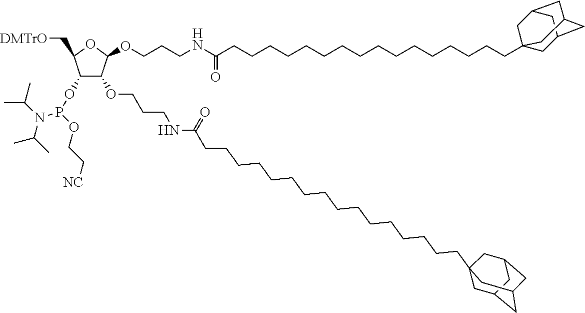 | ³¹P-NMR (CDCl₃): δ 149.80, 149.61. |
| Compound 28 | |
| 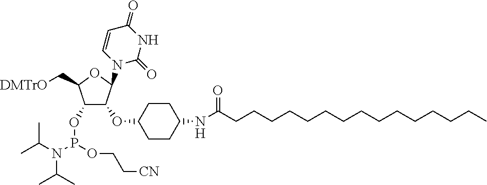 | MS: m/z 1082 [M + H]⁺; ³¹P-NMR (DMSO- d₆): δ 149.17, 148.08. |
| Compound 29 | |
| 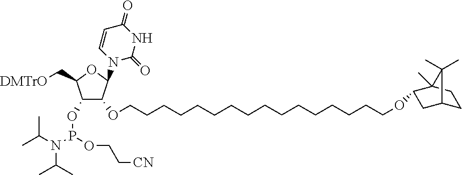 | MS: m/z 1124 [M + H]⁺; ³¹P-NMR (DMSO- d₆): δ 149.12, 148.45. |
| Compound 30 | |
| 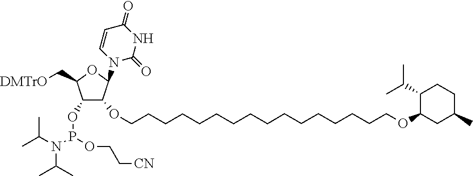 | MS: m/z 1126 [M + H]⁺; ³¹P-NMR (DMSO- d₆): δ 149.15, 148.65. |
| Compound 31 | |
| 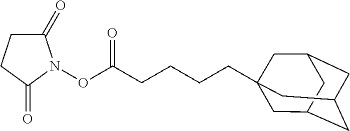 [SMILES: O=C(CCCCC12CC3CC(CC(C3)C1)C2)ON1C(=O)CCC1=O] | ¹³C-NMR (CDCl₃): δ 21.80, 25.58, 25.60, 28.74, 31.00, 32.20, 37.24, 42.41, 44.11, 77.04, 77.25, 168.72, 169.21. |
| Compound 32 | |
| 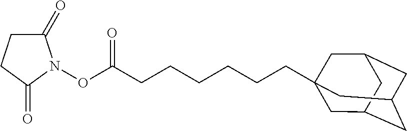 [SMILES: O=C(CCCCCCC12CC3CC(CC(C3)C1)C2)ON1C(=O)CCC1=O] | MS: m/z 361 [M + H]⁺; ¹³C-NMR (CDCl₃): δ 22.14, 24.59, 25.60, 28.79, 28.88, 30.08, 30.96, 32.21, 37.31, 42.53, 44.64, 76.82, 77.03, 77.24, 168.71, 169.19. |
| Compound 33 | |
| 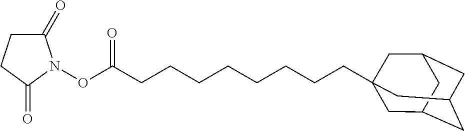 [SMILES: O=C(CCCCCCCCC12CC3CC(CC(C3)C1)C2)ON1C(=O)CCC1=O] | MS: m/z 316 [M (acid) + Na]⁺; ¹³C-NMR (CDCl₃): δ 22.32, 24.58, 25.59, 28.81, 29.10, 29.40, 30.53, 30.95, 32.22, 37.33, 42.55, 44.76, 76.83, 77.04, 77.26, 168.71, 169.21. |
| Compound 34 | |
| 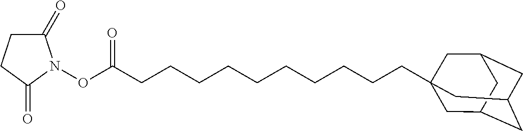 [SMILES: O=C(CCCCCCCCCCC12CC3CC(CC(C3)C1)C2)ON1C(=O)CCC1=O] | MS: m/z 430 [M + Na]⁺; ¹³C-NMR (CDCl₃): δ 22.38, 24.58, 25.60, 28.80, 28.82, 29.09, 29.38, 29.57, 29.68, 30.66, 30.95, 32.23, 37.35, 42.56, 44.81, 76.83, 77.04, 77.26, 168.70, 169.19. |
| Compound 35 | |
| 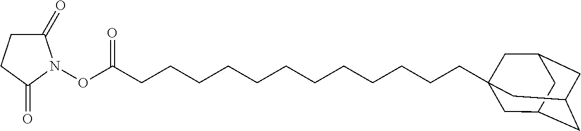 [SMILES: O=C(CCCCCCCCCCCCC12CC3CC(CC(C3)C1)C2)ON1C(=O)CCC1=O] | MS: m/z 468 [M + Na]⁺; ¹³C-NMR (CDCl₃): δ 22.39, 24.58, 25.59, 28.80, 28.82, 29.10, 29.36, 29.56, 29.65, 29.68, 29.75, 30.69, 30.95, 32.23, 37.35, 42.57, 44.83, 76.84, 77.05, 77.26, 168.70, 169.20. |
| Compound 36 | |
| 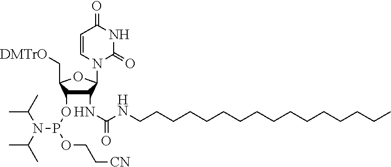 | MS: m/z 1012 [M − H]⁻, ³¹P-NMR(DMSO-d₆): δ 149.69, 149.0. |
| Compound 39 | |
| 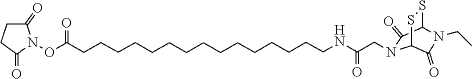 [SMILES: CCN1C(=O)C2SSC1C(=O)N2CC(=O)NCCCCCCCCCCCCCCCC(=O)ON1C(=O)CCC1=O] | MS: m/z 611 [M − H]⁻. |
| Compound 40 | |
| 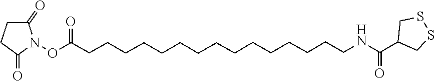 [SMILES: O=C(CCCCCCCCCCCCCCCNC(=O)C1CSSC1)ON1C(=O)CCC1=O] | MS: m/z 501 [M + H]⁺. |
| Compound 41 | |
| 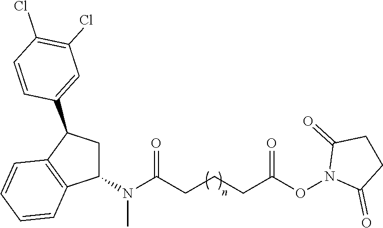 | n = 1; MS: m/z 503 [M + H]⁺. n = 12; MS: m/z 658 [M + H]⁺. |
| Compound 42 | |
| 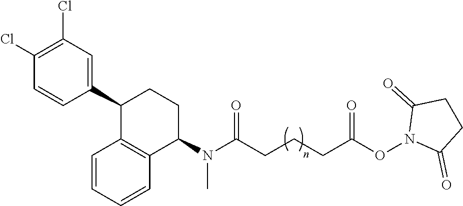 | n = 1; MS: m/z 517 [M + H]⁺. n = 12; MS: m/z 671 [M + H]⁺. |
| Compound 43 | |
| 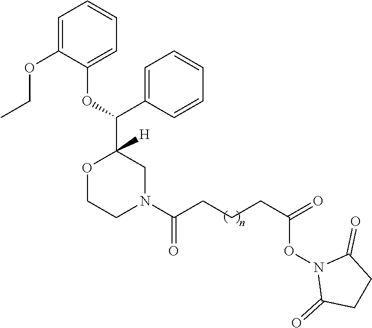 | n = 1; MS: m/z 525 [M + H]⁺. |
| Compound 44 | n = 12; MS: m/z 679 [M + H]⁺. |

### Example 26. Knockdown Efficiency of MAPT siRNA in Human iPSC-Neurons

MAPT siRNA agents conjugated with certain lipophilic monomers provided herein were synthesized and the sequences are listed in Table 3.

TABLE3
The modified sense and antisense strand sequences of MAPT siRNAs conjugated
with different lipids (5′ to 3′)

| | | SEQ ID | | SEQ ID |
|---|---|---|---|---|
| NAME | Senses trand | NO | Antisense strand | NO |
| M28_V | (L2)mApsmApsfGmUmGmUfGfGf | 179 | (VPmU)psfGpsmCmCmUfAmAmU | 180 |
| ar29 | CmUmCfAmUmUmAmGfGpsmCp | | mGmAmGmCmCfAmCfAmCmUm | |
| | smA | | UpsmGpsmG | |
| M28_V | mApsmApsfGmUmGmUfGfGfCm | 181 | (VPmU)psfGpsmCmCmUfAmAmU | 182 |
| ar30 | UmCfAmUmUmAmGfGpsmCpsm | | mGmAmGmCmCfAmCfAmCmUm | |
| | AdTdT(L2) | | UpsmGpsmG | |
| M28V | (L2)mApsmApsfGmUmGmUfGfGf | 187 | (VPmU)psfGpsmCmCmUfA(GNA- | 188 |
| ar33 | CmUmCfAmUmUmAmGfGpsmCp | | A)mUmGmAmGmCmCfAmCfAm | |
| | smA | | CmUmUpsmGpsmG | |
| M28_V | mApsmApsfGmUmGmUfGfGfCm | 189 | (VPmU)psfGpsmCmCmUfA(GNA- | 190 |
| ar34 | UmCfAmUmUmAmGfGpsmCpsm | | A)mUmGmAmGmCmCfAmCfAm | |
| | AdTdT(L2) | | CmUmUpsmGpsmG | |
| M28_V | mApsmApsfGmUmGmUfGfGfCm | 191 | (VPmU)psfGpsmCmCmUfAmAmU | 192 |
| ar35 | UmCfAmUmUmAmGfGpsmCpsm | | mGmAmGmCmCfAmCfAmCmUm | |
| | AdTdT(Chol4) | | UpsmGpsmG | |
| M28_V | (Chol4)mApsmApsfGmUmGmUfG | 193 | (VPmU)psfGpsmCmCmUfAmAmU | 194 |
| ar36 | fGfCmUmCfAmUmUmAmGfGpsm | | mGmAmGmCmCfAmCfAmCmUm | |
| | CpsmA | | UpsmGpsmG | |
| M28_V | mApsmApsfGmUmG(L1)fGfGfCm | 195 | (VPmU)psfGpsmCmCmUfAmAmU | 196 |
| ar37 | UmCfAmUmUmAmGfGpsmCpsm | | mGmAmGmCmCfAmCfAmCmUm | |
| | A | | UpsmGpsmG | |
| M28_V | mApsmApsfGmUmG(L3)fGfGfCm | 197 | (VPmU)psfGpsmCmCmUfAmAmU | 198 |
| ar38 | UmCfAmUmUmAmGfGpsmCpsm | | mGmAmGmCmCfAmCfAmCmUm | |
| | A | | UpsmGpsmG | |
| M28_V | mApsmApsfGmUmG(L4)fGfGfCm | 199 | (VPmU)psfGpsmCmCmUfAmAmU | 200 |
| ar39 | UmCfAmUmUmAmGfGpsmCpsm | | mGmAmGmCmCfAmCfAmCmUm | |
| | A | | UpsmGpsmG | |
| M28_V | mApsmApsfGmUmG(L7)fGfGfCm | 201 | (VPmU)psfGpsmCmCmUfAmAmU | 202 |
| ar4( | UmCfAmUmUmAmGfGpsmCpsm | | mGmAmGmCmCfAmCfAmCmUm | |
| | A | | UpsmGpsmG | |
| M28_V | mApsmApsfGmUmG(L5)fGfGfCm | 203 | (VPmU)psfGpsmCmCmUfAmAmU | 204 |
| ar41 | UmCfAmUmUmAmGfGpsmCpsm | | mGmAmGmCmCfAmCfAmCmUm | |
| | A | | UpsmGpsmG | |
| M28_V | mApsmApsfGmUmG(L6)fGfGfCm | 205 | (VPmU)psfGpsmCmCmUfAmAmU | 206 |
| ar42 | UmCfAmUmUmAmGfGpsmCpsm | | mGmAmGmCmCfAmCfAmCmUm | |
| | A | | UpsmGpsmG | |
| M28_V | mApsmApsfGmUmGmUfGfGfCm | 207 | (VPmU)psfGpsmCmCmUfAmAmU | 208 |
| ar43 | UmCfAmUmUmAmGfGpsmCpsm | | mGmAmGmCmCfAmCfAmCmUm | |
| | AdTdT(L10) | | UpsmGpsmG | |
| M28_V | mApsmApsfGmUmGmUfGfGfCm | 209 | (VPmU)psfGpsmCmCmUfAmAmU | 210 |
| ar44 | UmCfAmUmUmAmGfGpsmCpsm | | mGmAmGmCmCfAmCfAmCmUm | |
| | A(L9) | | UpsmGpsmG | |
| M28_V | (L9)mApsmApsfGmUmGmUfGfGf | 211 | (VPmU)psfGpsmCmCmUfAmAmU | 212 |
| ar45 | CmUmCfAmUmUmAmGfGpsmCp | | mGmAmGmCmCfAmCfAmCmUm | |
| | smA | | UpsmGpsmG | |
| M28_V | mApsmApsfGmUmGmUfGfGfCm | 213 | (VPmU)psfGpsmCmCmUfAmAmU | 214 |
| ar46 | UmCfAmUmUmAmGfGpsmCpsm | | mGmAmGmCmCfAmCfAmCmUm | |
| | A(L8) | | UpsmGpsmG | |
| M28_V | (L9)mApsmApsfGmUmGmUfGfGf | 215 | (VPmU)psfGpsmCmCmUfAmAmU | 216 |
| ar47 | CmUmCfAmUmUmAmGfGpsmCp | | mGmAmGmCmCfAmCfAmCmUm | |
| | smA(L9) | | UpsmGpsmG | |
| M28_V | (L8)mApsmApsfGmUmGmUfGfGf | 217 | (VPmU)psfGpsmCmCmUfAmAmU | 218 |
| ar48 | CmUmCfAmUmUmAmGfGpsmCp | | mGmAmGmCmCfAmCfAmCmUm | |
| | smA(L8) | | UpsmGpsmG | |
| M28_V | mApsmApsfGmUmGmUfGfGfCm | 219 | (VPmU)psfGpsmCmCmUfAmAmU | 220 |
| ar49 | UmCfAmUmUmAmGfGpsmCpsm | | mGmAmGmCmCfAmCfAmCmUm | |
| | A(C7NH-L17) | | UpsmGpsmG | |
| M25_V | (InvAb)psmApsmUmAmGmUmCm | 221 | (VPmU)psfUpsmCpsmAmAfCmUf | 222 |
| ar46 | UmAfCfAfAmAmCmCmAmGmU | | GfGmUmUmUmGfUmAfGmAmC | |
| | mUmGmApsmAps(InvAb)(C7NH- | | mUpsmApsmU | |
| | L20) | | | |
| M25_V | (InvAb)psmApsmUmAmGmUmCm | 223 | (VPmU)psfUpsmCpsmAmAfCmUf | 224 |
| ar47 | UmAfCfAfAmAmCmCmAmGmU | | GfGmUmUmUmGfUmAfGmAmC | |
| | mUmGmApsmAps(InvAb)(C7NH- | | mUpsmApsmU | |
| | L18) | | | |
| M25_V | (InvAb)psmApsmUmAmGmUmCm | 225 | (VPmU)psfUpsmCpsmAmAfCmUf | 226 |
| ar48 | UmAfCfAfAmAmCmCmAmGmU | | GfGmUmUmUmGfUmAfGmAmC | |
| | mUmGmApsmAps(InvAb)(C7NH- | | mUpsmApsmU | |
| | L16) | | | |
| M25_V | (InvAb)psmApsmUmAmGmUmCm | 227 | (VPmU)psfUpsmCpsmAmAfCmUf | 228 |
| ar49 | UmAfCfAfAmAmCmCmAmGmU | | GfGmUmUmUmGfUmAfGmAmC | |
| | mUmGmApsmAps(InvAb)(C7NH- | | mUpsmApsmU | |
| | L17) | | | |
| M25_V | (InvAb)psmApsmUmAmGmUmCm | 229 | (VPmU)psfUpsmCpsmAmAfCmUf | 230 |
| ar50 | UmAfCfAfAmAmCmCmAmGmU | | GfGmUmUmUmGfUmAfGmAmC | |
| | mUmGmApsmAps(InvAb)(L12) | | mUpsmApsmU | |
| M25_V | (InvAb)psmApsmUmAmGmUmCm | 231 | (VPmU)psfUpsmCpsmAmAfCmUf | 232 |
| ar51 | UmAfCfAfAmAmCmCmAmGmU | | GfGmUmUmUmGfUmAfGmAmC | |
| | mUmGmApsmAps(InvAb)(L11) | | mUpsmApsmU | |
| M25_V | (InvAb)psmApsmUmAmGmUmCm | 233 | (VPmU)psfUpsmCpsmAmAfCmUf | 234 |
| ar52 | UmAfCfAfAmAmCmCmAmGmU | | GfGmUmUmUmGfUmAfGmAmC | |
| | mUmGmApsmAps(InvAb)(L24) | | mUpsmApsmU | |
| M25_V | (InvAb)psmApsmUmAmGmUmC( | 235 | (VPmU)psfUpsmCpsmAmAfCmUf | 236 |
| ar53 | C6NH- | | GfGmUmUmUmGfUmAfGmAmC | |
| | L15U)mAfCfAfAmAmCmCmAmG | | mUpsmApsmU | |
| | mUmUmGmApsmAps(InvAb) | | | |
| M25V | (InvAb)psmApsmUmAmGmUmC( | 237 | (VPmU)psfUpsmCpsmAmAfCmUf | 238 |
| ar54 | C6NH- | | GfGmUmUmUmGfUmAfGmAmC | |
| | L18U)mAfCfAfAmAmCmCmAmG | | mUpsmApsmU | |
| | mUmUmGmApsmAps(InvAb) | | | |
| M25_V | (InvAb)psmApsmUmAmGmUmC( | 239 | (VPmU)psfUpsmCpsmAmAfCmUf | 240 |
| ar55 | C6NH- | | GfGmUmUmUmGfUmAfGmAmC | |
| | L19U)mAfCfAfAmAmCmCmAmG | | mUpsmApsmU | |
| | mUmUmGmApsmAps(InvAb) | | | |
| M25_V | (InvAb)psmApsmUmAmGmUmC( | 241 | (VPmU)psfUpsmCpsmAmAfCmUf | 242 |
| ar56 | C6NH- | | GfGmUmUmUmGfUmAfGmAmC | |
| | L17U)mAfCfAfAmAmCmCmAmG | | mUpsmApsmU | |
| | mUmUmGmApsmAps(InvAb) | | | |
| M25_V | (InvAb)psmApsmUmAmGmUmC( | 243 | (VPmU)psfUpsmCpsmAmAfCmUf | 244 |
| ar57 | C6NH- | | GfGmUmUmUmGfUmAfGmAmC | |
| | L4U)mAfCfAfAmAmCmCmAmG | | mUpsmApsmU | |
| | mUmUmGmApsmAps(InvAb) | | | |
| M25_V | (InvAb)psmApsmUmAmGmUmC( | 247 | (VPmU)psfUpsmCpsmAmAfCmUf | 248 |
| ar59 | C6NH- | | GfGmUmUmUmGfUmAfGmAmC | |
| | L16U)mAfCfAfAmAmCmCmAmG | | mUpsmApsmU | |
| | mUmUmGmApsmAps(InvAb) | | | |
| M25_V | (InvAb)psmApsmUmAmGmUmC( | 249 | (VPmU)psfUpsmCpsmAmAfCmUf | 250 |
| ar60 | C6NH- | | GfGmUmUmUmGfUmAfGmAmC | |
| | L20U)mAfCfAfAmAmCmCmAmG | | mUpsmApsmU | |
| | mUmUmGmApsmAps(InvAb) | | | |
| M25_V | (InvAb)psmApsmUmAmGmUmCm | 253 | (VPmU)psfUpsmCpsmAmAfCmUf | 254 |
| ar62 | UmAfCfAfAmAmCmCmAmGmU | | GfGmUmUmUmGfUmAfGmAmC | |
| | mUmGmApsmAps(InvAb)(C7NH- | | mUpsmApsmU | |
| | L14) | | | |
| M25_V | (InvAb)psmApsmUmAmGmUmCm | 255 | (VPmU)psfUpsmCpsmAmAfCmUf | 256 |
| ar63 | UmAfCfAfAmAmCmCmAmGmU | | GfGmUmUmUmGfUmAfGmAmC | |
| | mUmGmApsmAps(InvAb)(C7NH- | | mUpsmApsmU | |
| | L15) | | | |
| M25_V | (InvAb)psmApsmUmAmGmUmCm | 257 | (VPmU)psfUpsmCpsmAmAfCmUf | 258 |
| ar64 | UmAfCfAfAmAmCmCmAmGmU | | GfGmUmUmUmGfUmAfGmAmC | |
| | mUmGmApsmAps(InvAb)(C7NH- | | mUpsmApsmU | |
| | L19) | | | |
| M25_V | (InvAb)psmApsmUmAmGmUmC( | 261 | (VPmU)psfUpsmCmAmAfCmUfGf | 262 |
| ar66 | L13)mAfCfAfAmAmCmCmAmGm | | GmUmUmUmGfUmAfGmAmCmU | |
| | UmUmGmApsmAps(InvAb) | | psmApsmU | |
| M25_V | (InvAb)psmApsmUmAmGmUmC( | 263 | (VPmU)psfUpsmCpsmAmAfCmUf | 264 |
| ar68 | L4)mAfCfAfAmAmCmCmAmGm | | GfGmUmUmUmGfUmAfGmAmC | |
| | UmUmGmApsmAps(InvAb) | | mUpsmApsmU | |
| M25_V | (InvAb)psmAps(L5)mAmGmUmC | 265 | (VPmU)psfUpsmCpsmAmAfCmUf | 266 |
| ar73 | mUmAfCfAfAmAmCmCmAmGm | | GfGmUmUmUmGfUmAfGmAmC | |
| | UmUmGmApsmAps(InvAb) | | mUpsmApsmU | |
| M25_V | (InvAb)psmApsmUmAmG(L5)mC | 267 | (VPmU)psfUpsmCpsmAmAfCmUf | 268 |
| ar74 | mUmAfCfAfAmAmCmCmAmGm | | GfGmUmUmUmGfUmAfGmAmC | |
| | UmUmGmApsmAps(InvAb) | | mUpsmApsmU | |
| M25_V | (InvAb)psmApsmUmAmGmUmC( | 269 | (VPmU)psfUpsmCpsmAmAfCmUf | 270 |
| ar75 | L5)mAfCfAfAmAmCmCmAmGm | | GfGmUmUmUmGfUmAfGmAmC | |
| | UmUmGmApsmAps(InvAb) | | mUpsmApsmU | |
| M25_V | (InvAb)psmApsmUmAmGmUmCm | 271 | (VPmU)psfUpsmCpsmAmAfCmUf | 272 |
| ar76 | UmAfCfAfAmAmCmCmAmG(L5) | | GfGmUmUmUmGfUmAfGmAmC | |
| | mUmGmApsmAps(InvAb) | | mUpsmApsmU | |
| M25_V | (InvAb)psmApsmUmAmGmUmCm | 273 | (VPmU)psfUpsmCpsmAmAfCmUf | 274 |
| ar77 | UmAfCfAfAmAmCmCmAmGmU( | | GfGmUmUmUmGfUmAfGmAmC | |
| | L5)mGmApsmAps(InvAb) | | mUpsmApsmU | |
| M25_V | (InvAb)psmAps(L4)mAmGmUmC | 275 | (VPmU)psfUpsmCpsmAmAfCmUf | 276 |
| ar78 | mUmAfCfAfAmAmCmCmAmGm | | GfGmUmUmUmGfUmAfGmAmC | |
| | UmUmGmApsmAps(InvAb) | | mUpsmApsmU | |
| M25_V | (InvAb)psmApsmUmAmG(L4)mC | 277 | (VPmU)psfUpsmCpsmAmAfCmUf | 278 |
| ar79 | mUmAfCfAfAmAmCmCmAmGm | | GfGmUmUmUmGfUmAfGmAmC | |
| | UmUmGmApsmAps(InvAb) | | mUpsmApsmU | |
| M25_V | (InvAb)psmApsmUmAmGmUmC( | 279 | (VPmU)psfUpsmCpsmAmAfCmUf | 280 |
| ar68 | L4)mAfCfAfAmAmCmCmAmGm | | GfGmUmUmUmGfUmAfGmAmC | |
| | UmUmGmApsmAps(InvAb) | | mUpsmApsmU | |
| M25_V | (InvAb)psmApsmUmAmGmUmCm | 281 | (VPmU)psfUpsmCpsmAmAfCmUf | 282 |
| ar80 | UmAfCfAfAmAmCmCmAmG(L4) | | GfGmUmUmUmGfUmAfGmAmC | |
| | mUmGmApsmAps(InvAb) | | mUpsmApsmU | |
| M25_V | (InvAb)psmApsmUmAmGmUmCm | 283 | (VPmU)psfUpsmCpsmAmAfCmUf | 284 |
| ar81 | UmAfCfAfAmAmCmCmAmGmU( | | GfGmUmUmUmGfUmAfGmAmC | |
| | L4)mGmApsmAps(InvAb) | | mUpsmApsmU | |
| M25_V | (InvAb)psmAps(L13)mAmGmUmC | 285 | (VPmU)psfUpsmCpsmAmAfCmUf | 286 |
| ar82 | mUmAfCfAfAmAmCmCmAmGm | | GfGmUmUmUmGfUmAfGmAmC | |
| | UmUmGmApsmAps(InvAb) | | mUpsmApsmU | |
| M25_V | (InvAb)psmApsmUmAmG(L13)mC | 287 | (VPmU)psfUpsmCpsmAmAfCmUf | 288 |
| ar83 | mUmAfCfAfAmAmCmCmAmGm | | GfGmUmUmUmGfUmAfGmAmC | |
| | UmUmGmApsmAps(InvAb) | | mUpsmApsmU | |
| M25_V | (InvAb)psmApsmUmAmGmUmC( | 289 | (VPmU)psfUpsmCpsmAmAfCmUf | 290 |
| ar84 | L13)mAfCfAfAmAmCmCmAmGm | | GfGmUmUmUmGfUmAfGmAmC | |
| | UmUmGmApsmAps(InvAb) | | mUpsmApsmU | |
| M25_V | (InvAb)psmApsmUmAmGmUmCm | 291 | (VPmU)psfUpsmCpsmAmAfCmUf | 292 |
| ar85 | UmAfCfAfAmAmCmCmAmG(L13 | | GfGmUmUmUmGfUmAfGmAmC | |
| | )mUmGmApsmAps(InvAb) | | mUpsmApsmU | |
| M25_V | (InvAb)psmApsmUmAmGmUmCm | 293 | (VPmU)psfUpsmCpsmAmAfCmUf | 294 |
| ar86 | UmAfCfAfAmAmCmCmAmGmU( | | GfGmUmUmUmGfUmAfGmAmC | |
| | L13)mGmApsmAps(InvAb) | | mUpsmApsmU | |
| M28_V | mApsmApsfGmUmGmUfGfGfCm | 317 | (VPmU)psfGpsmCmCmUfAmAmU | 318 |
| ar1 | UmCfAmUmUmAmGfGmCmA(Ch | | mGmAmGmCmCfAmCfAmCmUm | |
| | o14) | | UpsmGpsmG | |
| M28_V | mApsmApsfGmUmG(L4)fGfGfCm | 319 | (VPmU)psfGpsmCmCmUfAmAmU | 320 |
| ar39 | UmCfAmUmUmAmGfGpsmCpsm | | mGmAmGmCmCfAmCfAmCmUm | |
| | A | | UpsmGpsmG | |
| M28_V | mApsmApsfGmUmG(L7)fGfGfCm | 321 | (VPmU)psfGpsmCmCmUfAmAmU | 322 |
| ar40 | UmCfAmUmUmAmGfGpsmCpsm | | mGmAmGmCmCfAmCfAmCmUm | |
| | A | | UpsmGpsmG | |
| M28_V | mApsmApsfGmUmG(L5)fGfGfCm | 323 | (VPmU)psfGpsmCmCmUfAmAmU | 324 |
| ar41 | UmCfAmUmUmAmGfGpsmCpsm | | mGmAmGmCmCfAmCfAmCmUm | |
| | A | | UpsmGpsmG | |
| M28_V | mApsmApsfGmUmG(L6)fGfGfCm | 325 | (VPmU)psfGpsmCmCmUfAmAmU | 326 |
| ar42 | UmCfAmUmUmAmGfGpsmCpsm | | mGmAmGmCmCfAmCfAmCmUm | |
| | A | | UpsmGpsmG | |
| M28_V | mApsmApsfGmUmG(L13)fGfGfC | 327 | (VPmU)psfGpsmCmCmUfAmAmU | 328 |
| ar50 | mUmCfAmUmUmAmGfGpsmCps | | mGmAmGmCmCfAmCfAmCmUm | |
| | mA | | UpsmGpsmG | |
| M96_V | (InvAb)psmGpsmAmAmGmA(L4)f | 329 | (VPmU)psfUpsmAmCmGfUmCfCf | 330 |
| ar1 | CmAfCfGfCmUmGmGmGmAmC | | CmAmGmCmGfUmGfAmUmCmU | |
| | mGmUpsmApsmA | | mUmCpsmCpsmA | |
| M96_V | (InvAb)psmGpsmAmAmGmAmU( | 331 | (VPmU)psfUpsmAmCmGmUdCm | 332 |
| ar5 | L22)mAfCfGfCmUmGmGmGmAm | | CmCmAmGmCmGfUmGfAmUmC | |
| | CmGmUmApsmAps(InvAb) | | mUpsmUpsmC | |
| M165 | (InvAb)psmCpsmAmAmGmUmC( | 333 | (VPmU)psfUpsmGmGmAmGdCm | 334 |
| Var5 | L22)mAfAfGfAmUmCmGmGmCm | | CmGmAmUmCmUfUmGfGmAmC | |
| | UmCmCmApsmAps(InvAb) | | mUpsmUpsmG | |
| M177 | (InvAb)psmUpsmGmCmAmA(L21) | 335 | (VPmU)psfUpsmGmGmUfUmUfGf | 336 |
| Var1 | fUmAfGfUfCmUmAmCmAmAmA | | UmAmGmAmCfUmAfUmUmUmG | |
| | mCmCpsmApsmA | | mCmApsmCpsmA | |
| M177 | (InvAb)psmUpsmGmCmAmAmA( | 337 | (VPmU)psfUpsmGpsfGmUfUmUf | 338 |
| Var2 | L4)mAfGfUfCmUmAmCmAmAm | | GmUfAmGfAmCfUmAfUmUfUm | |
| | AmCmCmApsmAps(InvAb) | | GmCpsmA | |
| M177 | (InvAb)psmUpsmGmCmAmAmA( | 339 | (VPmU)psfUpsmGmGmUfUmUfGf | 340 |
| Var3 | L4)mAfGfUfCmUmAmCmAmAm | | UmAmGmAmCfUmAfUmUmUmG | |
| | AmCmCmApsmAps(InvAb) | | psmCpsmA | |
| M177 | (InvAb)psmUpsmGmCmAmAmA( | 341 | (VPmU)psfUpsmGmGmUmUdTm | 342 |
| Var5 | L4)mAfGfUfCmUmAmCmAmAm | | GmUmAmGmAmCfUmAfUmUmU | |
| | AmCmCmApsmAps(InvAb) | | mGpsmCpsmA | |
| M177 | (InvAb)psmUpsmGmCmAmAmA( | 343 | (VPmU)psfUpsmGmGmUfUmUfGf | 344 |
| Var6 | L4)mAfGfUfCmUmAmCmAmAm | | UmAmGmAmCfUdAfUmUmUmG | |
| | AmCmCmApsmAps(InvAb) | | psmCpsmA | |
| M25_V | (InvAb)psmApsmUmAmG(L4)mC | 345 | (VPmU)psfUpsmCmAmAfCmUfGf | 346 |
| ar93 | mUmAfCfAfAmAmCmCmAmGm | | GmUmUmUmGfUmAfGmAmCmU | |
| | UmUmGmApsmAps(InvAb) | | psmApsmU | |
| M25_V | (InvAb)psmApsmUmAmGmU(L22) | 347 | (VPmU)psfUpsmCmAmAfCmUfGf | 348 |
| ar94 | mUmAfCfAfAmAmCmCmAmGm | | GmUmUmUmGfUmAfGmAmCmU | |
| | UmUmGmApsmAps(InvAb) | | psmApsmU | |
| M25_V | (InvAb)psmApsmUmAmGmUmC( | 349 | (VPmU)psfUpsmCmAmAfCmUfGf | 350 |
| ar95 | L4)mAfCfAfAmAmCmCmAmGm | | GmUmUmUmGfUmAfGmAmCmU | |
| | UmUmGmApsmAps(InvAb) | | psmApsmU | |
| M25_V | (InvAb)psmApsmUmAmGmUmCm | 351 | (VPmU)psfUpsmCmAmAfCmUfGf | 352 |
| ar98 | UmAfCfAfAmA(L22)mCmAmGm | | GmUmUmUmGfUmAfGmAmCmU | |
| | UmUmGmApsmAps(InvAb) | | psmApsmU | |
| M25_V | (InvAb)psmApsmUmAmGmUmCm | 353 | (VPmU)psfUpsmCmAmAfCmUfGf | 354 |
| ar99 | UmAfCfAfAmAmC(L22)mAmGm | | GmUmUmUmGfUmAfGmAmCmU | |
| | UmUmGmApsmAps(InvAb) | | psmApsmU | |
| M25V | (InvAb)psmApsmUmAmGmUmCm | 355 | (VPmU)psfUpsmCmAmAfCmUfGf | 356 |
| ar101 | UmAfCfAfAmAmCmCmA(L23)m | | GmUmUmUmGfUmAfGmAmCmU | |
| | UmUmGmApsmAps(InvAb) | | psmApsmU | |

Modification: mN = 2′OMe; fN = 2′F; ps = phosphorothioate; VP = vinyl phosphonate; invAb = inverse abasic Chol4 = Cholesterol-triethylene glycol

The in vitro knockdown efficiencies of lipid-siRNA conjugates were assessed in human iPSC-derived cortical neurons following the procedure described as below.

Differentiation of Human iPSCs into Cortical Neurons

The human iPSC line (Sigma #iPSC0028) was derived with OSKM retroviral reprogramming of epithelial cells from a 24-years old Caucasian female donor. iPSCs were first differentiated into cortical neural stem cells (NSCs) following a dual SMAD inhibitor protocol (Shi et al., 2012, 2012, Nat. Proc. 7(10):1836-46) with some modifications. Briefly, iPSCs were plated at 500,000 cells/cm² on wells coated with Matrigel (Corning 354230) in mTeSR medium (StemCell Technologies 5850) supplemented with 10 μM ROCK inhibitor (Sigma-Aldrich Y0503) and cultured at 37° C. and with 5% O₂. The media were replaced with mTeSR the next day (day −1). From day 0 till day 12, cell media were changed every day with cortical Neural Induction Medium comprised of 10 μM SB43142 (Tocris 1614) and 1 μM Dorsomorphin (Tocris 3093) supplemented in Neural Maintenance Medium (1:1 DMEM:F12 Glutamax (ThermoFisher Scientific 10565108), Neurobasal (ThermoFisher Scientific 21103049), 2.5 μg/mL Insulin (Sigma-Aldrich I9278-5ML), 50 uM 2-mercaptoethanol (ThermoFisher Scientific, 31350010), 0.5% Non-Essential Amino Acids (ThermoFisher Scientific 11140035), 0.5% GlutaMAX supplement (ThermoFisher Scientific, 10565018), 0.5 mM Sodium Pyruvate (ThermoFisher Scientific 11360070), 1% Penicillin-Streptomycin (Sigma P4333), 0.5% N2 supplement (ThermoFisher Scientific 17502048), 1% B27 supplement (ThermoFisher Scientific 17504044). At day 12, the neuroepithelial sheet was gently detached into large aggregates of 300 to 500 cells using a needle and lifter and the clumps were collected in a 15 mL falcon by centrifugation at 160 g for 2 min. Cell pellets were gently resuspended in Neural Induction Medium for a 1/2 or 1/3 passage into wells coated with 10 μg/mL laminin (Sigma-Aldrich L2020) in a total volume of 2 mL of Neural Induction Medium per well of the 6-well plate. Media were changed at day 13 and day 15 into Neural Maintenance Medium supplemented with 20 ng/mL of FGF2 (Stemcell Technologies 2634). At day 17, the neural rosettes were detached with dispase (ThermoFisher Scientific 17105041) for a 1/3 passage and plated into laminin-coated wells. Perform 1 or 2 extra dispase steps for another week. At around days 25-30, neural stem cells were dissociated with Accutase (ThermoFisher Scientific A1110501) into single-cell suspension and cryopreserved in freshly prepared Neural Freezing Medium containing Neural Maintenance Medium supplemented with 10% (V/V) DMSO and 20 ng/mL FGF2. The frozen vials of neural stem cells were store in liquid nitrogen till use.

To generate iPSC-neurons, frozen vials of neural stem cells (NSCs) were thawed and plated on laminin-coated wells at 70,000 cells/cm² in Neural Maintenance Medium supplemented with 10 μM ROCK inhibitor and 20 ng/mL FGF2. In the following two days, media were replaced daily with neural maintenance medium. At around day 4 after thawing, cells were dissociated with Accutase and placed at 28.000 cells per well in 96-well plates pre-coated with poly-L-ornithine and laminin in Neural Maintenance Medium supplemented with 10 μM ROCK inhibitor. The next day after re-plating, culture media were replaced with Neural Differentiation Medium comprised of Neural Maintenance Medium supplemented with 20 ng/mL BDNF (R&D Systems 212-BD-050/CF), 20 ng/mL GDNF (R&D Systems 212-GD-050/CF), 500 μM DB-cAMP (Sigma D0627) and 20 mM Ascorbic Acid (Sigma A4403). Cultures were differentiated in Neural Differentiation Medium with 50% medium change twice per week. Two-to-three weeks after differentiation from neural stem cells (NSCs), neurons were treated with siRNA for 7 days or 14 days for RNA analysis by Reverse transcription and Real time PCR and protein analysis by MSD immunoassay.

### MSD Immunoassay

Human iPSC-neurons differentiated on 96-well plate were lysed in 100 μL ice-cold RIPA buffer (Sigma) supplemented with cOmplete™ Protease inhibitor cocktail (Roche) and PhosSTOP™ (Roche) with slow orbital shaking for 30 min at 4° C. The plates were centrifuged at 1,000×g for 5 min, and the cell lysates were collected and diluted for different (20, 50 or 100) folds for protein measurement using MSD immunoassays following a standardized procedure. Briefly, MSD plates were coated with 30 μL per well of coating antibody diluted in PBS at 1 μg/mL overnight at 4° C. The next day, plates were inverted on absorbent tissue to dry and afterwards incubated with 150 μL per well blocking buffer 0.1% Casein for 2 hours at RT with orbital shaking at 300 rpm. After blocking, the plates were incubated with cell lysate diluted in RIPA buffer supplemented with cOmplete™ Protease inhibitor cocktail (Roche) overnight with slow orbital shaking at 4° C. Plates were washes 5× times using Titertek Aquamax 4000 and inverted on absorbent tissue to dry. Afterwards, 25 μL/per secondary or detection antibody diluted in 0.1% casein were added to MSD plates and incubated for 2 hours at room temperature. After antibody incubation, plates were washed 5× times and developed with 150 μL/well 2×MSD reading buffer (Meso Scale Discovery) diluted in milliQ H2O. Plates were read immediately using MSD instrument. For measurement of Tau protein, hTau43 antibody (Janssen) was used for 96-well MSD plate coating and SULFO-TAG™ labeled hTau60 antibody (Janssen) was used for detection. For measurement of Histone H3 protein, recombinant rabbit monoclonal antibody 17H2L9 (ThermoFisher) was used for plate coating, mouse mAb 14221BF (Cell Signaling Technology) was used as primary detection and SULFP-TAG labeled anti-mouse antibody (Meso Scale Discovery) was used as secondary detection antibody.

Tables 4-6 summarize the efficiency of MAPT siRNAs in conjugation with different lipids in knocking down MAPT mRNA in human iPSC-neurons after incubation for 7 days. Three concentrations of siRNA conjugates at 1 μM, 200 nM and 40 nM were tested. Data are presented as remaining MAPT mRNA relative to control. Mean±S.D., n=3.

TABLE 4
MAPT mRNA knockdown efficiency of MAPT siRNA
M28 lipid conjugates in human iPSC neurons.

| | Remaining MAPT mRNA/control | | | | | |
|---|---|---|---|---|---|---|
| | 1000 nM | | 200 nM | | 40 nM | |
| NAME | mean | SD | mean | SD | mean | SD |
| M28-Chol | 0.024 | 0.003 | 0.038 | 0.005 | 0.121 | 0.018 |
| M28_Var1-Chol | 0.025 | 0.002 | 0.032 | 0.004 | 0.067 | 0.011 |
| M28_Var2-Chol | 0.026 | 0.002 | 0.053 | 0.006 | 0.146 | 0.009 |
| M28_Var29 | 0.042 | 0.004 | 0.127 | 0.016 | 0.568 | 0.015 |
| M28_Var30 | 0.030 | 0.001 | 0.065 | 0.009 | 0.300 | 0.061 |
| M28_Var33 | 0.109 | 0.008 | 0.429 | 0.015 | / | / |
| M28_Var34 | 0.037 | 0.001 | 0.164 | 0.046 | 0.568 | 0.024 |
| M28_Var35 | 0.029 | 0.003 | 0.042 | 0.007 | 0.124 | 0.019 |
| M28_Var36 | 0.034 | 0.004 | 0.080 | 0.006 | 0.255 | 0.003 |
| M28_Var37 | 0.322 | 0.025 | 0.645 | 0.051 | 0.884 | 0.049 |
| M28_Var38 | 0.347 | 0.041 | 0.648 | 0.016 | 0.913 | 0.085 |
| M28_Var39 | 0.305 | 0.053 | 0.562 | 0.059 | 0.916 | 0.135 |
| M28_Var40 | 0.353 | 0.022 | 0.651 | 0.040 | 0.787 | 0.025 |
| M28_Var41 | 0.416 | 0.053 | 0.751 | 0.062 | 0.887 | 0.076 |
| M28_Var42 | 0.428 | 0.057 | 0.683 | 0.111 | 0.850 | 0.052 |
| M28_Var43 | 0.038 | 0.004 | 0.168 | 0.053 | 0.681 | 0.144 |
| M28_Var44 | 0.188 | 0.011 | 0.605 | 0.047 | 0.884 | 0.124 |
| M28_Var45 | 0.403 | 0.020 | 0.814 | 0.166 | 0.922 | 0.075 |
| M28_Var46 | 0.286 | 0.018 | 0.723 | 0.029 | 0.882 | 0.057 |
| M28_Var47 | 0.390 | 0.022 | 0.831 | 0.087 | 1.002 | 0.056 |
| M28_Var48 | 0.450 | 0.026 | 0.791 | 0.050 | 0.927 | 0.087 |
| M28_Var49 | 0.027 | 0.003 | 0.072 | 0.016 | 0.340 | 0.018 |

TABLE 5
MAPT mRNA knockdown efficiency of MAPT siRNA
M25 lipid conjugates in human iPSC neurons.

| | Remaining MAPT mRNA/control | | | | | |
|---|---|---|---|---|---|---|
| | 1000 nM | | 200 nM | | 40 nM | |
| NAME | mean | SD | mean | SD | mean | SD |
| M25_Var46 | 0.187 | 0.064 | 0.649 | 0.040 | 0.968 | 0.193 |
| M25_Var47 | 0.034 | 0.007 | 0.039 | 0.006 | 0.220 | 0.067 |
| M25_Var48 | 0.182 | 0.015 | 0.682 | 0.080 | 0.708 | 0.047 |
| M25_Var49 | 0.679 | 0.053 | 0.954 | 0.095 | 0.864 | 0.120 |
| M25_Var50 | 0.041 | 0.009 | 0.218 | 0.032 | 0.662 | 0.062 |
| M25_Var51 | 0.038 | 0.004 | 0.240 | 0.056 | 0.728 | 0.075 |
| M25_Var52 | 0.068 | 0.018 | 0.390 | 0.054 | 0.889 | 0.213 |
| M25_Var53 | 0.641 | 0.041 | 0.841 | 0.066 | 1.034 | 0.052 |
| M25_Var54 | 0.149 | 0.036 | 0.529 | 0.063 | 0.882 | 0.028 |
| M25_Var55 | 0.718 | 0.060 | 0.979 | 0.084 | 0.948 | 0.052 |
| M25_Var56 | 0.503 | 0.028 | 0.878 | 0.006 | 1.077 | 0.034 |
| M25_Var57 | 0.548 | 0.056 | 0.868 | 0.060 | 0.929 | 0.043 |
| M25_Var59 | 0.626 | 0.055 | 0.884 | 0.118 | 0.984 | 0.077 |
| M25_Var60 | 0.621 | 0.031 | 0.897 | 0.109 | 1.051 | 0.180 |
| M25_Var62 | 0.300 | 0.034 | 0.706 | 0.058 | 0.910 | 0.103 |
| M25_Var63 | 0.331 | 0.040 | 0.701 | 0.031 | 1.001 | 0.065 |
| M25_Var64 | 0.202 | 0.030 | 0.718 | 0.086 | 0.882 | 0.106 |
| M25_Var66 | 0.381 | 0.032 | 0.755 | 0.058 | 0.925 | 0.066 |
| M25_Var68 | 0.590 | 0.103 | 0.778 | 0.087 | 0.935 | 0.126 |

TABLE 6
MAPT mRNA knockdown efficiency of MAPT siRNA M25 lipid
conjugates (lipid walk) in human iPSC neurons.

| | Remaining MAPT mRNA/control | | | | | |
|---|---|---|---|---|---|---|
| | 1000 nM | | 200 nM | | 40 nM | |
| NAME | mean | SD | mean | SD | mean | SD |
| M25_Var73 | 0.566 | 0.051 | 0.801 | 0.061 | 0.928 | 0.046 |
| M25_Var74 | 0.706 | 0.017 | 0.774 | 0.085 | 1.003 | 0.061 |
| M25_Var75 | 0.532 | 0.080 | 0.757 | 0.025 | 1.021 | 0.076 |
| M25_Var76 | 0.536 | 0.056 | 0.819 | 0.152 | 1.037 | 0.160 |
| M25_Var77 | 0.572 | 0.073 | 0.759 | 0.090 | 0.841 | 0.160 |
| M25_Var78 | 0.608 | 0.014 | 0.919 | 0.159 | 0.944 | 0.082 |
| M25_Var79 | 0.556 | 0.055 | 0.763 | 0.096 | 0.826 | 0.063 |
| M25_Var68 | 0.553 | 0.044 | 0.870 | 0.070 | 0.986 | 0.050 |
| M25_Var80 | 0.502 | 0.116 | 0.717 | 0.059 | 0.986 | 0.068 |
| M25_Var81 | 0.551 | 0.059 | 0.749 | 0.145 | 0.822 | 0.091 |
| M25_Var82 | 0.572 | 0.060 | 0.841 | 0.060 | 0.911 | 0.150 |
| M25_Var83 | 0.662 | 0.030 | 0.962 | 0.064 | 1.090 | 0.153 |
| M25_Var84 | 0.507 | 0.046 | 0.721 | 0.127 | 0.830 | 0.081 |
| M25_Var85 | 0.432 | 0.026 | 0.831 | 0.087 | 0.891 | 0.173 |
| M25_Var86 | 0.577 | 0.059 | 0.777 | 0.147 | 1.053 | 0.071 |

### Example 27. Stability Study

The in vitro stability of siRNA conjugates was assessed in various matrices including mouse brain homogenates, human liver lysosomes and rat liver tritosomes using the methods described as below. The results of the analyses are provided in Table 7 and Table 8.

Table 7 and Table 8 summarize the LC-MS measurement of antisense strand and sense strand stability of siRNA conjugates in mouse brain homogenates (Table 7 and Table 10), and human liver lysosomes (Table 8 and Table 11) and rat liver tritosomes (Table 12) at 37° C. after 24 hours incubation with shaking at 450 rpm. A reference compound MSC-2-V was added in the assays for benchmarking the stringency of the assay conditions.

TABLE 7
LC-MS measurement of MAPT siRNA stability
in mouse brain homogenates

| | % Stability | | | | | |
|---|---|---|---|---|---|---|
| | Antisense strand | | | Sense strand | | |
| NAME | Mean | SD | N | Mean | SD | N |
| M25_Var15-Chol | 88.8 | 7.7 | 9 | 64.2 | 12.7 | 9 |
| M25_Var49 | 57.3 | 2.6 | 3 | 55.5 | 3.2 | 3 |
| M25_Var84 | 78.6 | 5.1 | 3 | 98.7 | 4.7 | 3 |
| M25_Var68 | 78.0 | 4.4 | 3 | 86.5 | 1.0 | 3 |

TABLE 8
LC-MS measurement of MAPT siRNA stability
in human liver tritosome

% Stability

| | Antisense strand | | | Sense strand | | |
|---|---|---|---|---|---|---|
| NAME | Mean | SD | N | Mean | SD | N |
| M25_Var66 | 97.4 | 13.1 | 3 | 90.8 | 7.4 | 3 |

TABLE 9
In vivo knockdown efficiency of lipid-M28Var conjugates in hTAU KI mice
at 7 days after ICV injection (15 nmol siRNA conjugate). Data are pretend
as remaining MAPT mRNA (%) levels; n = 6 mice per treatment group.

| | Remaining MAPT mRNA (%) | | | | | | | |
|---|---|---|---|---|---|---|---|---|
| | Cortex | | Hippocampus | | Cerebellum | | Spinal cord | |
| Name | mean | SD | mean | SD | mean | SD | mean | SD |
| Vehicle | 100.0 | 4.6 | 100.0 | 10.6 | 100.0 | 6.0 | 100.0 | 10.6 |
| M28_Var1-Chol | 58.6 | 4.8 | 35.5 | 4.8 | 53.3 | 6.3 | 30.8 | 1.8 |
| M28_Var39 | 41.0 | 10.8 | 28.8 | 5.9 | 38.2 | 6.7 | 21.4 | 2.7 |
| M28_Var41 | 36.7 | 7.0 | 25.4 | 3.6 | 36.5 | 7.2 | 19.9 | 0.8 |
| M28_Var40 | 33.2 | 8.1 | 23.5 | 6.1 | 37.1 | 7.4 | 21.3 | 2.1 |
| M28_Var42 | 40.2 | 5.3 | 26.3 | 2.3 | 35.6 | 1.3 | 22.8 | 4.4 |
| M28_Var50 | 34.9 | 9.3 | 26.8 | 6.4 | 38.7 | 14.2 | 21.0 | 1.3 |

| | Remaining MAPT mRNA (%) | | | | | |
|---|---|---|---|---|---|---|
| | Striatum | | Midbrain | | Brainstem | |
| NAME | mean | SD | mean | SD | mean | SD |
| Vehicle | 100.0 | 5.7 | 100.0 | 7.6 | 100.0 | 6.7 |
| M28_Var1-Chol | 60.6 | 9.4 | 42.0 | 3.2 | 41.6 | 5.2 |
| M28_Var39 | 39.6 | 4.1 | 31.2 | 3.9 | 29.7 | 2.1 |
| M28_Var41 | 36.4 | 3.1 | 27.4 | 2.2 | 26.1 | 2.4 |
| M28_Var40 | 36.0 | 7.8 | 26.3 | 3.2 | 29.5 | 2.7 |
| M28_Var42 | 40.4 | 5.6 | 28.4 | 3.1 | 26.2 | 2.2 |
| M28_Var50 | 38.2 | 9.9 | 29.9 | 5.9 | 27.8 | 5.4 |

TABLE 10
LC-MS measurement of MAPT siRNA stability
in mouse brain homogenate.

% Stability

| | Antisense strand | | | Sense strand | | |
|---|---|---|---|---|---|---|
| NAME | Mean | SD | N | Mean | SD | N |
| M96_Var1 | 53.9 | 6.2 | 6 | 73.2 | 10.2 | 6 |
| M96_Var5 | 89.2 | 17.7 | 6 | 90.0 | 20.5 | 6 |
| M165_Var5 | 87.6 | 13.1 | 6 | 91.6 | 6.9 | 6 |
| M177_Var1 | 74.2 | 4.3 | 6 | 76.3 | 13.7 | 6 |
| M177_Var2 | 89.5 | 7.4 | 6 | 75.4 | 12.6 | 6 |
| M177_Var3 | 88.7 | 4.5 | 6 | 85.7 | 10.9 | 6 |
| M177_Var5 | 90.4 | 6.6 | 6 | 82.7 | 5.4 | 6 |
| MSC-2-V | 74.1 | 5.0 | 6 | 46.8 | 8.3 | 6 |

TABLE 11
LC-MS measurement of MAPT siRNA stability
in human liver tritosome.

% Stability

| | Antisense strand | | | Sense strand | | |
|---|---|---|---|---|---|---|
| NAME | Mean | SD | N | Mean | SD | N |
| M96_Var1 | 100.7 | 7.9 | 3 | 99.1 | 8.0 | 3 |
| M96_Var5 | 95.8 | 5.7 | 3 | 90.1 | 3.0 | 3 |
| M165_Var5 | 94.4 | 7.5 | 3 | 84.3 | 7.0 | 3 |
| M177_Var1 | 103.3 | 16.2 | 3 | 95.8 | 25.9 | 3 |
| M177_Var2 | 101.7 | 5.9 | 3 | 100.1 | 9.9 | 3 |
| M177_Var3 | 104.7 | 4.6 | 3 | 94.8 | 4.2 | 3 |
| M177_Var5 | 87.7 | 7.2 | 3 | 82.7 | 8.7 | 3 |
| MSC-2-V | 94.6 | 4.4 | 3 | 0.7 | 0.2 | 3 |

TABLE 12
LC-MS measurement of MAPT siRNA
stability in rat liver tritosome.

% Stability

| | Antisense strand | | | Sense strand | | |
|---|---|---|---|---|---|---|
| Name | Mean | SD | N | Mean | SD | N |
| M96_Var1 | 116.3 | 7.8 | 3 | 116.5 | 8.9 | 3 |
| M96_Var5 | 105.9 | 10.0 | 3 | 100.2 | 5.4 | 3 |
| M165_Var5 | 109.0 | 9.5 | 3 | 104.2 | 12.8 | 3 |
| M177_Var1 | 111.3 | 7.6 | 3 | 103.8 | 8.4 | 3 |
| M177_Var2 | 110.3 | 3.1 | 3 | 111.0 | 8.0 | 3 |
| M177_Var3 | 99.4 | 2.0 | 3 | 112.1 | 25.0 | 3 |
| M177_Var5 | 118.3 | 7.8 | 3 | 116.0 | 11.3 | 3 |
| MSC-2-V | 119.3 | 5.0 | 3 | 1.2 | 0.4 | 3 |

### Example 28: In Vivo Knockdown Efficiency Assessment of siRNA Conjugates in Mouse Models

The in vivo knockdown efficiencies of lipid-siRNA conjugates were assessed in mouse models with intracerebroventricular (ICV) injection following the procedure described as below.

MAPT mRNA in seven brain regions (cortex, hippocampus, brainstem, cerebellum, striatum, midbrain, and cervical spinal cord) from hTAU KI mice at 7 days after a single ICV injection of M28_Var1, M28_Var39, M28_Var40, M28_Var41, M28_Var42, M28_Var50 at 15 nmol is assessed. The results in Table 9. Data are presented as remaining MAPT mRNA (%). Mean±S.D., n=6 mice per treatment group.

### Mice and Intracerebroventricular Injection

Male and Female PS19 mice (C57BL6; Prnp-MAPT*P301S) or hTau KI (C57BL6; hMAPT: Knock-In) mice at age of 2-to-3-month are randomly assigned to different treatment groups. Mice are anesthetised with isoflurane (induction: 4-5%; maintenance: 1.8-2.5%). They are then stereotaxically injected using a motorized drill and microinjection robot (Neurostar, Germany, Sterodrive Sofeware v 2019) into the bilateral ventricles at coordinates: AP: −0.62 mm, ML: +/−1.05 mm; and DV: 2.2 mm. Each injection is performed in 5 μL volume over 5 min and following injection the needle is withdrawn in three steps (1.1 mm in 60 sec and kept there for 5 min; 2. another 0.5 mm in 30 sec, wait 5 min; 3. withdrawal out of the brain at very slow speed) to avoid compound reflux along the needle tract. After every step the amount of backflow is checked. At selected post-injection days, animals are sacrificed and different brain regions including cortex, hippocampus, brainstem, cerebellum, striatum, midbrain, and cervical spinal cord are dissected and snap frozen and stored at −80° C. till further analysis.

RNA Extraction from Tissues

Tissues are collected in Lysing Matrix D (MP Biomedicals 6913-500) 2 mL Tubes containing 1.4 mm ceramic spheres and stored in −80° C. freezer till analysis. Place the tissues on ice under laminar flow and immediately add 750 μL Trizol (ThermoFisher 15596026/15596018) per tube. Disrupt and homogenize the tissue using the FastPrep-24™ 5G Grinder with 3 cycles of 30 sec at speed 5 m/sec. Between cycles, cool down the samples on ice for 2 minutes. After homogenization, shortly spin the tube and add 20% volume of Chloroform (150 μL Chloroform when 750 μL Trizol was used). Vortex for 15 sec and centrifuge at 14.000×g for 15 min at 4° C. Transfer the upper aqueous phase to a deep 96 well plate and add 1 volume of 70% ethanol and mix well. The next steps are performed using RNeasy 96 kit (Qiagen) following manufacture's protocol. Place a RNeasy 96 plate on top of the Square-Well Block holder, apply the samples (Trizol/Chloroform extraction) into the wells of the RNeasy 96 plate and seal the plate with AirPore cover to prevent contamination. Centrifuge at 5600×g for 3 min at RT and discard the solution. Serial wash steps including 1 time of 800 μL RW1 buffer, 2 times of 800 μL RPE buffer that are applied to the RNeasy 96 plate, and wash buffers are removed by centrifugation at 5600×g for 3 min for each step. After the last wash, the RNeasy 96 plate is centrifuged at 5600×g for 3 min to remove the residual lipid. RNA is eluted using 60 μL RNase-free water by centrifugation at 5600×g for 3 min at RT. RNA concentration is measured by Nanodrop 8000 (ThermoFisher). The eluted RNA is stored at −80° C. until analysis.

### RT-qPCR

RNA is used as template for reverse transcription using reversed transcribed using High-Capacity cDNA Reverse Transcription Kits (Applied Biosystems) following manufacture's protocol. Briefly, a final reaction of 20 μL mixture is incubated at 25° C. for 10 min, followed by reverse transcription at 37° C. for 2 hours and enzyme inactivation at 85° C. for 5 min. For qPCR reaction, reverse transcribed cDNAs are diluted 10 times and mixed with 2× PowerUp™ SYBR™ Green Mater Mix (ThermoFisher A25743) and 500 nM qPCR primers to a final volume of 10 μL reaction. The qPCR runs are performed with a QuantStudio™ 12K instrument (Applied Biosystems™) using standard thermal cycling protocol. Multiple primers are used to detect MAPT mRNA, and reference primers targeting mouse housekeeping genes AP3D1 and PAK11P1 are included for normalization of gene expression.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

1. A compound of Formula (I): 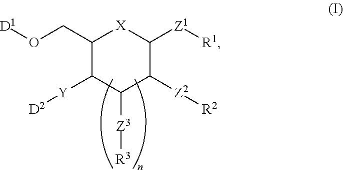
wherein:
X is O or S;
Y is O or NR^{a};
D¹ is H, a hydroxyl protecting group, or a reactive phosphorus group;
D² is H, a hydroxyl protecting group, or a reactive phosphorus group;
or D¹ and D² together form a protecting group for both the oxygen and Y they are attached to;
Z¹, Z², and Z³ are each independently —O—, —NR^{a}—, *—OC(═O)—, *—NR^{a}C(═O)—, —OC(═O)O—, *—NR^{a}C(═O)O—, *—OC(═O)NR^{b}—, *—NR^{a}C(═O)NR^{b}—, *—OC(═S)—, *—NR^{a}C(═S)—, —OC(═S)O—, *—NR^{a}C(═S)O—, *—OC(═S)NR^{b}—, *—NR^{a}C(═S)NR^{b}—, *—OC(═O)S—, *—NR^{a}C(═O)S—, *—OS(O)ₓ—, or *—NR^{a}S(O)ₓ—; wherein * refers to the direction toward the ring containing X;
R¹, R², and R³ are each independently H, C₁-C₆ alkyl optionally substituted with C₁-C₆ alkoxy, or -(G-L)ₘ-R, provided that at least one of R¹, R², and R³ is -(G-L)ₘ-R;
each instance of G is independently C₁-C₈alkylene;
each instance of L is independently —O—, —NR^{a}—, —C(═O)—, *—OC(═O)—, *—C(═O)O—, *—NR^{a}C(═O)—, *—C(═O)NR^{b}—, —OC(═O)O—, *—NR^{a}C(═O)O—, *—OC(═O)NR^{b}—, *—NR^{a}C(═O)NR^{b}—, *—OC(═S)—, *—C(═S)O—, *—NR^{a}C(═S)—, *—C(═S)NR^{b}—, —OC(═S)O—, *—NR^{a}C(═S)O—, *—OC(═S)NR^{b}—, *—NR^{a}C(═S)NR^{b}—, *—OC(═O)S—, *—SC(═O)O—, *—NR^{a}C(═O)S—, *—SC(═O)NR^{b}—, *—OS(O)ₓ—, *—S(O)ₓO—, *—NR^{a}S(O)ₓ—, or *—S(O)ₓNR^{b}—; wherein * refers to the direction toward the ring containing X;
each instance of R^{a} is independently H or C₁-C₆ alkyl;
each instance of R^{b} is independently H or C₁-C₆ alkyl; and
each instance of R is independently C₁₂-C₃₂ alkyl or C₁₂-C₃₂ alkenyl;
x is 1 or 2;
n is 0 or 1;
m is 0, 1, or 2;
or a stereoisomer thereof, or a pharmaceutically acceptable salt thereof.

2. The compound of claim 1, wherein X is O or S.

3. (canceled)

4. (canceled)

5. (canceled)

6. (canceled)

7. (canceled)

8. The compound of claim 1, which is a compound of Formula (I-A): 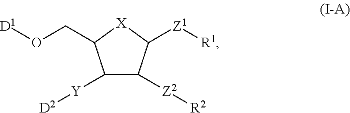
or a stereoisomer thereof, or a pharmaceutically acceptable salt thereof.

9. (canceled)

10. (canceled)

11. (canceled)

12. (canceled)

13. (canceled)

14. (canceled)

15. (canceled)

16. (canceled)

17. The compound of claim 1, which is a compound of Formula (I-B): 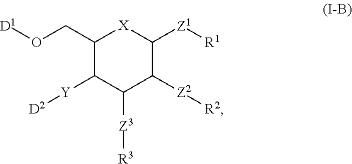
or a stereoisomer thereof, or a pharmaceutically acceptable salt thereof.

18. (canceled)

19. (canceled)

20. (canceled)

21. (canceled)

22. (canceled)

23. (canceled)

24. (canceled)

25. (canceled)

26. A compound of Formula (III): 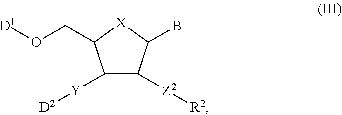
wherein:
B is a modified or unmodified nucleobase;
X is O or S;
Y is O or NR^{a};
D¹ is H, a hydroxyl protecting group, or a reactive phosphorus group;
D² is H, a hydroxyl protecting group, or a reactive phosphorus group;
or D¹ and D² together form a protecting group for both the oxygen and Y they are attached to;
Z² is —O—, —NR^{a}—, *—OC(═O)—, *—NR^{a}C(═O)—, —OC(═O)O—, *—NR^{a}C(═O)O—, *—OC(═O)NR^{b}—, *—NR^{a}C(═O)NR^{b}—, *—OC(═S)—, *—NR^{a}C(═S)—, —OC(═S)O—, *—NR^{a}C(═S)O—, *—OC(═S)NR^{b}—, *—NR^{a}C(═S)NR^{b}—, *—OC(═O)S—, *—NR^{a}C(═O)S—, *—OS(O)ₓ—, or *—NR^{a}S(O)ₓ—; wherein * refers to the direction toward the ring containing X;
R² is -(G-L)ₘ-R;
each instance of G is independently C₁-C₈alkylene or C₃-C₁₀ cycloalkylene;
each instance of L is independently —O—, —NR^{a}—, —C(═O)—, *—OC(═O)—, *—C(═O)O—, *—NR^{a}C(═O)—, *—C(═O)NR^{b}—, —OC(═O)O—, *—NR^{a}C(═O)O—, *—OC(═O)NR^{b}—, *—NR^{a}C(═O)NR^{b}—, *—OC(═S)—, *—C(═S)O—, *—NR^{a}C(═S)—, *—C(═S)NR^{b}—, —OC(═S)O—, *—NR^{a}C(═S)O—, *—OC(═S)NR^{b}—, *—NR^{a}C(═S)NR^{b}—, *—OC(═O)S—, *—SC(═O)O—, *—NR^{a}C(═O)S—, *—SC(═O)NR^{b}—, *—OS(O)ₓ—, *—S(O)ₓO—, *—NR^{a}S(O)ₓ—, or *—S(O)ₓNR^{b}—; wherein * refers to the direction toward the ring containing X;
each instance of R^{a} is independently H or C₁-C₆ alkyl;
each instance of R^{b} is independently H or C₁-C₆ alkyl; and
R is C₁₂-C₃₂ alkyl or C₁₂-C₃₂ alkenyl;
x is 1 or 2; and
m is 0, 1, or 2;
provided that when X is O, Y is O, and Z² is —O—, then
(i) R is substituted with -L″-(CH₂)₀₋₃—R″, wherein:
L″ is absent, —O—, —NR^{a}—, —C(═O)—, *—OC(═O)—, *—C(═O)O—, *—NR^{a}C(═O)—, *—C(═O)NR^{b}—, —OC(═O)O—, *—NR^{a}C(═O)O—, *—OC(═O)NR^{b}—, *—NR^{a}C(═O)NR^{b}—, *—OC(═S)—, *—C(═S)O—, *—NR^{a}C(═S)—, *—C(═S)NR^{b}—, —OC(═S)O—, *—NR^{a}C(═S)O—, *—OC(═S)NR^{b}—, *—NR^{a}C(═S)NR^{b}—, *—OC(═O)S—, *—SC(═O)O—, *—NR^{a}C(═O)S—, *—SC(═O)NR^{b}—, *—OS(O)ₓ—, *—S(O)ₓO—, *—NR^{a}S(O)ₓ—, or *—S(O)ₓNR^{b}—; wherein * refers to the direction toward R; and
R″ is a monocyclic, fused, bridged or spiro ring moiety, wherein the ring moiety is optionally substituted with one or more C₁-C₆ alkyl, oxo, or phenyl, wherein the alkyl or phenyl is optionally substituted with one or more halogen, phenyl, or phenoxy, wherein the phenyl or phenoxy is optionally substituted with one or more halogen or C₁-C₆ alkoxy;
(ii) R is C₁₂-C₃₂ alkenyl;
(iii) m is 1 or 2; and L is —O— or *—C(═O)NR^{b}—; or
(iv) m is 1 or 2, and at least one G is C₃-C₁₀ cycloalkylene;
or a stereoisomer thereof, or a pharmaceutically acceptable salt thereof.

27. (canceled)

28. (canceled)

29. (canceled)

30. (canceled)

31. (canceled)

32. (canceled)

33. The compound of claim 26, wherein Z² is —OC(═O)NH—, —NHC(═O)—, —NHC(═O)NH—, or —NHC(═O)S—.

34. (canceled)

35. (canceled)

36. (canceled)

37. The compound of claim 26, when X is O, Y is O, and Z² is —O—, then R² is —(C₂-C₆ alkylene)-C(═O)NH—R or —(C₂-C₆ alkylene)-O—R.

38. (canceled)

39. (canceled)

40. (canceled)

41. (canceled)

42. (canceled)

43. (canceled)

44. A compound of Formula (IV): 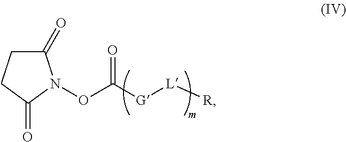
wherein:
each instance of G′ is independently C₁-C₈alkylene or C₃-C₈ cycloalkylene;
each instance of L′ is independently *—NR^{c}C(═O)—; wherein * refers to the direction toward the pyrrolidine-2,5-dione ring;
each instance of R is independently H, C₁-C₃₂ alkyl, or C₂-C₃₂ alkenyl;
R is C₄-C₃₂ alkyl or C₄-C₃₂ alkenyl; and
m is 0, 1, or 2;
or a stereoisomer thereof, or a pharmaceutically acceptable salt thereof.

45. (canceled)

46. (canceled)

47. The compound of claim 44, wherein -(G′-L′)ₘ-R is

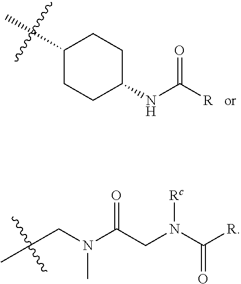

48. (canceled)

49. (canceled)

50. (canceled)

51. (canceled)

52. The compound of claim 1, wherein R is substituted with -L″-(CH₂)₀₋₃—R″, wherein:
L″ is absent, —O—, —NR^{a}—, —C(═O)—, *—OC(═O)—, *—C(═O)O—, *—NR^{a}C(═O)—, *—C(═O)NR^{b}—, —OC(═O)O—, *—NR^{a}C(═O)O—, *—OC(═O)NR^{b}—, *—NR^{a}C(═O)NR^{b}—, *—OC(═S)—, *—C(═S)O—, *—NR^{a}C(═S)—, *—C(═S)NR^{b}—, —OC(═S)O—, *—NR^{a}C(═S)O—, *—OC(═S)NR^{b}—, *—NR^{a}C(═S)NR^{b}—, *—OC(═O)S—, *—SC(═O)O—, *—NR^{a}C(═O)S—, *—SC(═O)NR^{b}—, *—OS(O)ₓ—, *—S(O)ₓO—, *—NR^{a}S(O)ₓ—, or *—S(O)ₓNR^{b}—; wherein * refers to the direction toward R;
each instance of R^{a} is independently H or C₁-C₆ alkyl;
each instance of R^{b} is independently H or C₁-C₆ alkyl;
x is 1 or 2; and
R″ is a monocyclic, fused, bridged or spiro ring moiety, wherein the ring moiety is optionally substituted with one or more C₁-C₆ alkyl, oxo, or phenyl, wherein the alkyl or phenyl is optionally substituted with one or more halogen, phenyl, or phenoxy, wherein the phenyl or phenoxy is optionally substituted with one or more halogen or C₁-C₆ alkoxy.

53. (canceled)

54. (canceled)

55. (canceled)

56. (canceled)

57. (canceled)

58. An oligonucleotide comprising at least one lipophilic monomer of the following formula: 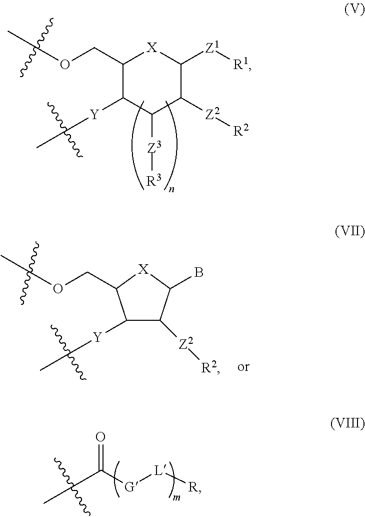
wherein for formula (V) and (VII),
X is O or S;
Y is O or NR^{a};
D¹ is H, a hydroxyl protecting group, or a reactive phosphorus group;
D² is H, a hydroxyl protecting group, or a reactive phosphorus group;
or D¹ and D² together form a protecting group for both the oxygen and Y they are attached to;
Z¹, Z², and Z³ are each independently —O—, —NR^{a}—, *—OC(═O)—, *—NR^{a}C(═O)—, —OC(═O)O—, *—NR^{a}C(═O)O—, *—OC(═O)NR^{b}—, *—NR^{a}C(═O)NR^{b}—, *—OC(═S)—, *—NR^{a}C(═S)—, —OC(═S)O—, *—NR^{a}C(═S)O—, *—OC(═S)NR^{b}—, *—NR^{a}C(═S)NR^{b}—, *—OC(═O)S—, *—NR^{a}C(═O)S—, *—OS(O)ₓ—, or *—NR^{a}S(O)ₓ—; wherein * refers to the direction toward the ring containing X;
R¹, R², and R³ are each independently H, C₁-C₆ alkyl optionally substituted with C₁-C₆ alkoxy, or -(G-L)ₘ-R, provided that at least one of R¹, R², and R³ is -(G-L)ₘ-R;
each instance of G is independently C₁-C₈ alkylene;
each instance of L is independently —O—, —NR^{a}—, —C(═O)—, *—OC(═O)—, *—C(═O)O—, *—NR^{a}C(═O)—, *—C(═O)NR^{b}—, —OC(═O)O—, *—NR^{a}C(═O)O—, *—OC(═O)NR^{b}—, *—NR^{a}C(═O)NR^{b}—, *—OC(═S)—, *—C(═S)O—, *—NR^{a}C(═S)—, *—C(═S)NR^{b}—, —OC(═S)O—, *—NR^{a}C(═S)O—, *—OC(═S)NR^{b}—, *—NR^{a}C(═S)NR^{b}—, *—OC(═O)S—, *—SC(═O)O—, *—NR^{a}C(═O)S—, *—SC(═O)NR^{b}—, *—OS(O)ₓ—, *—S(O)ₓO—, *—NR^{a}S(O)ₓ—, or *—S(O)ₓNR^{b}—; wherein * refers to the direction toward the ring containing X;
each instance of R^{a} is independently H or C₁-C₆ alkyl;
each instance of R^{b} is independently H or C₁-C₆ alkyl; and
each instance of R is independently C₁₂-C₃₂ alkyl or C₁₂-C₃₂ alkenyl;
x is 1 or 2;
n is 0 or 1;
m is 0, 1, or 2; and
for formula (VIII),
each instance of G′ is independently C₁-C₈ alkylene or C₃-C₈ cycloalkylene;
each instance of L′ is independently *—NR^{c}C(═O)—; wherein * refers to the direction toward the pyrrolidine-2,5-dione ring;
each instance of R^{c} is independently H, C₁-C₃₂ alkyl, or C₂-C₃₂ alkenyl;
R is C₄-C₃₂ alkyl or C₄-C₃₂ alkenyl; and
m is 0, 1, or 2.

59. The oligonucleotide of claim 58, wherein the oligonucleotide is an antisense, an antagomir, a microRNA, a siRNA, a pre-microRNA, an antimir, a ribozyme, a RNA activator, a U1 adaptor, an immune stimulator or an aptamer.

60. (canceled)

61. (canceled)

62. (canceled)

63. (canceled)

64. (canceled)

65. (canceled)

66. (canceled)

67. (canceled)

68. The oligonucleotide of claim 58, further comprising a targeting ligand.

69. (canceled)

70. A method of delivering an oligonucleotide to a cell, comprising administering an oligonucleotide of claim 58 to cell, wherein the oligonucleotide comprising at least one lipophilic monomer of formula (V).

71. (canceled)

72. (canceled)

73. A method of reducing the expression of a target gene in a cell, comprising contacting said cell with an oligonucleotide of claim 58, wherein the oligonucleotide comprising at least one lipophilic monomer of formula (V).

74. (canceled)

75. A method of delivering an oligonucleotide to a cell, comprising administering an oligonucleotide of claim 58 to cell, wherein the oligonucleotide comprising at least one lipophilic monomer of formula (VII).

76. A method of delivering an oligonucleotide to a cell, comprising administering an oligonucleotide of claim 58 to cell, wherein the oligonucleotide comprising at least one lipophilic monomer of formula (VIII).

77. A method of reducing the expression of a target gene in a cell, comprising contacting said cell with an oligonucleotide of claim 58, wherein the oligonucleotide comprising at least one lipophilic monomer of formula (VII).

78. A method of reducing the expression of a target gene in a cell, comprising contacting said cell with an oligonucleotide of claim 58, wherein the oligonucleotide comprising at least one lipophilic monomer of formula (VIII).
